SEQUENCE LISTING (108 sequences)

SEQ ID NO: 1 - protein, 202 aa, artificial sequence
  Mouse Lymphotoxin-alpha amino acid sequence with predicted signal sequence underlined
MTLLGRLHLLRVLGTPPVFLLGLLLALPLGAQGLSGVRFSAARTAHPLPQKHLTHGILKPAAHLVGYPSKQNSLLWRASTDRAFLRHGFSLSNNSLLIPTSGLYFVYSQVVFSGESCSPRAIPTPIYLAHEVQLFSSQYPFHVPLLSAQKSVYPGLQGPWVRSMYQGAVFLLSKGDQLSTHTDGISHLHFSPSSVFFGAFAL

SEQ ID NO: 2 - protein, 169 aa, artificial sequence
  Mouse Lymphotoxin-alpha amino acid sequence without predicted signal sequence underlined
LSGVRFSAARTAHPLPQKHLTHGILKPAAHLVGYPSKQNSLLWRASTDRAFLRHGFSLSNNSLLIPTSGLYFVYSQVVFSGESCSPRAIPTPIYLAHEVQLFSSQYPFHVPLLSAQKSVYPGLQGPWVRSMYQGAVFLLSKGDQLSTHTDGISHLHFSPSSVFFGAFAL

SEQ ID NO: 3 - protein, 144 aa, artificial sequence
  Mouse Lymphotoxin-alpha (aa 59-202)
KPAAHLVGYPSKQNSLLWRASTDRAFLRHGFSLSNNSLLIPTSGLYFVYSQVVFSGESCSPRAIPTPIYLAHEVQLFSSQYPFHVPLLSAQKSVYPGLQGPWVRSMYQGAVFLLSKGDQLSTHTDGISHLHFSPSSVFFGAFAL

SEQ ID NO: 4 - protein, 306 aa, artificial sequence
  Mouse Lymphotoxin-beta amino acid sequence
MGTRGLQGLGGRPQGRGCLLLAVAGATSLVTLLLAVPITVLAVLALVPQDQGRRVEKIIGSGAQAQKRLDDSKPSCILPSPSSLSETPDPRLHPQRSNASRNLASTSQGPVAQSSREASAWMTILSPAADSTPDPGVQQLPKGEPETDLNPELPAAHLIGAWMSGQGLSWEASQEEAFLRSGAQFSPTHGLALPQDGVYYLYCHVGYRGRTPPAGRSRARSLTLRSALYRAGGAYGRGSPELLLEGAETVTPVVDPIGYGSLWYTSVGFGGLAQLRSGERVYVNISHPDMVDYRRGKTFFGAVMVG

SEQ ID NO: 5 - protein, 258 aa, artificial sequence
  Mouse Lymphotoxin-beta extracellular domain (aa 49-306) amino acid sequence
QDQGRRVEKIIGSGAQAQKRLDDSKPSCILPSPSSLSETPDPRLHPQRSNASRNLASTSQGPVAQSSREASAWMTILSPAADSTPDPGVQQLPKGEPETDLNPELPAAHLIGAWMSGQGLSWEASQEEAFLRSGAQFSPTHGLALPQDGVYYLYCHVGYRGRTPPAGRSRARSLTLRSALYRAGGAYGRGSPELLLEGAETVTPVVDPIGYGSLWYTSVGFGGLAQLRSGERVYVNISHPDMVDYRRGKTFFGAVMVG

SEQ ID NO: 6 - protein, 158 aa, artificial sequence
  Mouse Lymphotoxin-beta (aa 149-306)
LNPELPAAHLIGAWMSGQGLSWEASQEEAFLRSGAQFSPTHGLALPQDGVYYLYCHVGYRGRTPPAGRSRARSLTLRSALYRAGGAYGRGSPELLLEGAETVTPVVDPIGYGSLWYTSVGFGGLAQLRSGERVYVNISHPDMVDYRRGKTFFGAVMVG

SEQ ID NO: 7 - protein, 460 aa, artificial sequence
  Mouse Lymphotoxin ALPHA-beta-beta single-chain trimer amino acid sequence without a signal sequence
KPAAHLVGYPSKQNSLLWRASTDRAFLRHGFSLSNNSLLIPTSGLYFVYSQVVFSGESCSPRAIPTPIYLAHEVQLFSSQYPFHVPLLSAQKSVYPGLQGPWVRSMYQGAVFLLSKGDQLSTHTDGISHLHFSPSSVFFGAFALLNPELPAAHLIGAWMSGQGLSWEASQEEAFLRSGAQFSPTHGLALPQDGVYYLYCHVGYRGRTPPAGRSRARSLTLRSALYRAGGAYGRGSPELLLEGAETVTPVVDPIGYGSLWYTSVGFGGLAQLRSGERVYVNISHPDMVDYRRGKTFFGAVMVGLNPELPAAHLIGAWMSGQGLSWEASQEEAFLRSGAQFSPTHGLALPQDGVYYLYCHVGYRGRTPPAGRSRARSLTLRSALYRAGGAYGRGSPELLLEGAETVTPVVDPIGYGSLWYTSVGFGGLAQLRSGERVYVNISHPDMVDYRRGKTFFGAVMVG

SEQ ID NO: 8 - protein, 460 aa, artificial sequence
  Mouse Lymphotoxin beta-alpha-beta single-chain trimer amino acid sequence without a signal sequence
LNPELPAAHLIGAWMSGQGLSWEASQEEAFLRSGAQFSPTHGLALPQDGVYYLYCHVGYRGRTPPAGRSRARSLTLRSALYRAGGAYGRGSPELLLEGAETVTPVVDPIGYGSLWYTSVGFGGLAQLRSGERVYVNISHPDMVDYRRGKTFFGAVMVGKPAAHLVGYPSKQNSLLWRASTDRAFLRHGFSLSNNSLLIPTSGLYFVYSQVVFSGESCSPRAIPTPIYLAHEVQLFSSQYPFHVPLLSAQKSVYPGLQGPWVRSMYQGAVFLLSKGDQLSTHTDGISHLHFSPSSVFFGAFALLNPELPAAHLIGAWMSGQGLSWEASQEEAFLRSGAQFSPTHGLALPQDGVYYLYCHVGYRGRTPPAGRSRARSLTLRSALYRAGGAYGRGSPELLLEGAETVTPVVDPIGYGSLWYTSVGFGGLAQLRSGERVYVNISHPDMVDYRRGKTFFGAVMVG

SEQ ID NO: 9 - protein, 460 aa, artificial sequence
  Mouse Lymphotoxin beta-beta-alpha single-chain trimer amino acid sequence without a signal sequence
LNPELPAAHLIGAWMSGQGLSWEASQEEAFLRSGAQFSPTHGLALPQDGVYYLYCHVGYRGRTPPAGRSRARSLTLRSALYRAGGAYGRGSPELLLEGAETVTPVVDPIGYGSLWYTSVGFGGLAQLRSGERVYVNISHPDMVDYRRGKTFFGAVMVGLNPELPAAHLIGAWMSGQGLSWEASQEEAFLRSGAQFSPTHGLALPQDGVYYLYCHVGYRGRTPPAGRSRARSLTLRSALYRAGGAYGRGSPELLLEGAETVTPVVDPIGYGSLWYTSVGFGGLAQLRSGERVYVNISHPDMVDYRRGKTFFGAVMVGKPAAHLVGYPSKQNSLLWRASTDRAFLRHGFSLSNNSLLIPTSGLYFVYSQVVFSGESCSPRAIPTPIYLAHEVQLFSSQYPFHVPLLSAQKSVYPGLQGPWVRSMYQGAVFLLSKGDQLSTHTDGISHLHFSPSSVFFGAFAL

SEQ ID NO: 10 - protein, 205 aa, artificial sequence
  Human Lymphotoxin-alpha amino acid sequence with predicted signal sequence underlined
MTPPERLFLPRVCGTTLHLLLLGLLLVLLPGAQGLPGVGLTPSAAQTARQHPKMHLAHSTLKPAAHLIGDPSKQNSLLWRANTDRAFLQDGFSLSNNSLLVPTSGIYFVYSQVVFSGKAYSPKATSSPLYLAHEVQLFSSQYPFHVPLLSSQKMVYPGLQEPWLHSMYHGAAFQLTQGDQLSTHTDGIPHLVLSPSTVFFGAFAL

SEQ ID NO: 11 - protein, 171 aa, artificial sequence
  Human Lymphotoxin-alpha amino acid sequence without predicted signal sequence
LPGVGLTPSAAQTARQHPKMHLAHSTLKPAAHLIGDPSKQNSLLWRANTDRAFLQDGFSLSNNSLLVPTSGIYFVYSQVVFSGKAYSPKATSSPLYLAHEVQLFSSQYPFHVPLLSSQKMVYPGLQEPWLHSMYHGAAFQLTQGDQLSTHTDGIPHLVLSPSTVFFGAFAL

SEQ ID NO: 12 - protein, 144 aa, artificial sequence
  Human Lymphotoxin-alpha (aa 62-205)
KPAAHLIGDPSKQNSLLWRANTDRAFLQDGFSLSNNSLLVPTSGIYFVYSQVVFSGKAYSPKATSSPLYLAHEVQLFSSQYPFHVPLLSSQKMVYPGLQEPWLHSMYHGAAFQLTQGDQLSTHTDGIPHLVLSPSTVFFGAFAL

SEQ ID NO: 13 - protein, 244 aa, artificial sequence
  Human Lymphotoxin-beta amino acid sequence
MGALGLEGRGGRLQGRGSLLLAVAGATSLVTLLLAVPITVLAVLALVPQDQGGLVTETADPGAQAQQGLGFQKLPEEEPETDLSPGLPAAHLIGAPLKGQGLGWETTKEQAFLTSGTQFSDAEGLALPQDGLYYLYCLVGYRGRAPPGGGDPQGRSVTLRSSLYRAGGAYGPGTPELLLEGAETVTPVLDPARRQGYGPLWYTSVGFGGLVQLRRGERVYVNISHPDMVDFARGKTFFGAVMVG

SEQ ID NO: 14 - protein, 196 aa, artificial sequence
  Human Lymphotoxin-beta extracellular domain (aa 49-244) amino acid sequence
QDQGGLVTETADPGAQAQQGLGFQKLPEEEPETDLSPGLPAAHLIGAPLKGQGLGWETTKEQAFLTSGTQFSDAEGLALPQDGLYYLYCLVGYRGRAPPGGGDPQGRSVTLRSSLYRAGGAYGPGTPELLLEGAETVTPVLDPARRQGYGPLWYTSVGFGGLVQLRRGERVYVNISHPDMVDFARGKTFFGAVMVG

SEQ ID NO: 15 - protein, 162 aa, artificial sequence
  Human Lymphotoxin-beta (aa 83-244)
LSPGLPAAHLIGAPLKGQGLGWETTKEQAFLTSGTQFSDAEGLALPQDGLYYLYCLVGYRGRAPPGGGDPQGRSVTLRSSLYRAGGAYGPGTPELLLEGAETVTPVLDPARRQGYGPLWYTSVGFGGLVQLRRGERVYVNISHPDMVDFARGKTFFGAVMVG

SEQ ID NO: 16 - protein, 468 aa, artificial sequence
  Human Lymphotoxin alpha-beta-beta single-chain trimer amino acid sequence without a signal sequence
KPAAHLIGDPSKQNSLLWRANTDRAFLQDGFSLSNNSLLVPTSGIYFVYSQVVFSGKAYSPKATSSPLYLAHEVQLFSSQYPFHVPLLSSQKMVYPGLQEPWLHSMYHGAAFQLTQGDQLSTHTDGIPHLVLSPSTVFFGAFALLSPGLPAAHLIGAPLKGQGLGWETTKEQAFLTSGTQFSDAEGLALPQDGLYYLYCLVGYRGRAPPGGGDPQGRSVTLRSSLYRAGGAYGPGTPELLLEGAETVTPVLDPARRQGYGPLWYTSVGFGGLVQLRRGERVYVNISHPDMVDFARGKTFFGAVMVGLSPGLPAAHLIGAPLKGQGLGWETTKEQAFLTSGTQFSDAEGLALPQDGLYYLYCLVGYRGRAPPGGGDPQGRSVTLRSSLYRAGGAYGPGTPELLLEGAETVTPVLDPARRQGYGPLWYTSVGFGGLVQLRRGERVYVNISHPDMVDFARGKTFFGAVMVG

SEQ ID NO: 17 - protein, 468 aa, artificial sequence
  Human Lymphotoxin beta-alpha-beta single-chain trimer amino acid sequence without a signal sequence
LSPGLPAAHLIGAPLKGQGLGWETTKEQAFLTSGTQFSDAEGLALPQDGLYYLYCLVGYRGRAPPGGGDPQGRSVTLRSSLYRAGGAYGPGTPELLLEGAETVTPVLDPARRQGYGPLWYTSVGFGGLVQLRRGERVYVNISHPDMVDFARGKTFFGAVMVGKPAAHLIGDPSKQNSLLWRANTDRAFLQDGFSLSNNSLLVPTSGIYFVYSQVVFSGKAYSPKATSSPLYLAHEVQLFSSQYPFHVPLLSSQKMVYPGLQEPWLHSMYHGAAFQLTQGDQLSTHTDGIPHLVLSPSTVFFGAFALLSPGLPAAHLIGAPLKGQGLGWETTKEQAFLTSGTQFSDAEGLALPQDGLYYLYCLVGYRGRAPPGGGDPQGRSVTLRSSLYRAGGAYGPGTPELLLEGAETVTPVLDPARRQGYGPLWYTSVGFGGLVQLRRGERVYVNISHPDMVDFARGKTFFGAVMVG

SEQ ID NO: 18 - protein, 468 aa, artificial sequence
  Human Lymphotoxin beta-beta-alpha single-chain trimer amino acid sequence without a signal sequence
LSPGLPAAHLIGAPLKGQGLGWETTKEQAFLTSGTQFSDAEGLALPQDGLYYLYCLVGYRGRAPPGGGDPQGRSVTLRSSLYRAGGAYGPGTPELLLEGAETVTPVLDPARRQGYGPLWYTSVGFGGLVQLRRGERVYVNISHPDMVDFARGKTFFGAVMVGLSPGLPAAHLIGAPLKGQGLGWETTKEQAFLTSGTQFSDAEGLALPQDGLYYLYCLVGYRGRAPPGGGDPQGRSVTLRSSLYRAGGAYGPGTPELLLEGAETVTPVLDPARRQGYGPLWYTSVGFGGLVQLRRGERVYVNISHPDMVDFARGKTFFGAVMVGKPAAHLIGDPSKQNSLLWRANTDRAFLQDGFSLSNNSLLVPTSGIYFVYSQVVFSGKAYSPKATSSPLYLAHEVQLFSSQYPFHVPLLSSQKMVYPGLQEPWLHSMYHGAAFQLTQGDQLSTHTDGIPHLVLSPSTVFFGAFAL

SEQ ID NO: 19 - protein, 330 aa, artificial sequence [sequence omitted]

SEQ ID NO: 20 - protein, 330 aa, artificial sequence
  Human IgG1 Heavy chain constant region deglycosylated
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 326 aa, artificial sequence
  Human IgG2 Heavy chain constant region
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 22 - protein, 326 aa, artificial sequence
  Human IgG2 Heavy chain constant region deglycosylated
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFASTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 23 - protein, 377 aa, artificial sequence
  Human IgG3 Heavy chain constant region
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 24 - protein, 377 aa, artificial sequence
  Human IgG3 Heavy chain constant region deglycosylated
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYASTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 25 - protein, 327 aa, artificial sequence
  Human IgG4 Heavy chain constant region
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 26 - protein, 327 aa, artificial sequence
  Human IgG4 Heavy chain constant region deglycosylated
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFASTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 27 - protein, 227 aa, artificial sequence
  Human IgG1 Fc region
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 227 aa, artificial sequence
  Human IgG1 Fc region deglycosylated
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 227 aa, artificial sequence
  Human IgG1 Fc region (13A Version)
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDKLTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 30 - protein, 227 aa, artificial sequence
  Human IgG1 Fc region (13B Version)
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVEGFYPSDIAVEWESNGQPENNYKTTPPVLDSDGSFFLYSELTVDKSRWQQGNVFSCSVMHEALHNHYTQKSLSLSPGK

SEQ ID NO: 31 - protein, 224 aa, artificial sequence
  Human IgG2 Fc region
CVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 32 - protein, 224 aa, artificial sequence
  Human IgG2 Fc region deglycosylated
CVECPPCPAPPVAGPSVFLFPPKPKDTLMISRTPEVTCVVVDVSHEDPEVQFNWYVDGVEVHNAKTKPREEQFASTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 33 - protein, 224 aa, artificial sequence
  Human IgG2 Fc region (13A Version)
CVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREKMTKNQVSLTCLVKGFYPSDIAVEWESNGQPENNYKTTPPMLKSDGSFFLYSKLTVDKSRWQQGNVFSCSVMHEALHNHYTQKSLSLSPGK

SEQ ID NO: 34 - protein, 224 aa, artificial sequence
  Human IgG2 Fc region (13B Version)
CVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVEGFYPSDIAVEWESNGQPENNYKTTPPMLDSDGSFFLYSELTVDKSRWQQGNVFSCSVMHEALHNHYTQKSLSLSPGK

SEQ ID NO: 35 - protein, 8 aa, artificial sequence
  FLAG Tag
DYKDDDDK

SEQ ID NO: 36 - protein, 915 aa, artificial sequence
  349B1 Anti-B7-H4 deglycosylated mIgG1-mLT alpha beta beta with signal sequence underlined
MKHLWFFLLLVAAPRWVLSQVQLQQSGSELVRPGASVKLSSKALGYTFTDYEMHWVKQTPVHGLEWIGTIHPGTGGTAYNQKFKGKATLTADKSSSTAYMELSSLTSEDSAVYYFTNLDNWGQGTTLTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFASTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGKKPAAHLVGYPSKQNSLLWRASTDRAFLRHGFSLSNNSLLIPTSGLYFVYSQVVFSGESCSPRAIPTPIYLAHEVQLFSSQYPFHVPLLSAQKSVYPGLQGPWVRSMYQGAVFLLSKGDQLSTHTDGISHLHFSPSSVFFGAFALLNPELPAAHLIGAWMSGQGLSWEASQEEAFLRSGAQFSPTHGLALPQDGVYYLYCHVGYRGRTPPAGRSRARSLTLRSALYRAGGAYGRGSPELLLEGAETVTPVVDPIGYGSLWYTSVGFGGLAQLRSGERVYVNISHPDMVDYRRGKTFFGAVMVGLNPELPAAHLIGAWMSGQGLSWEASQEEAFLRSGAQFSPTHGLALPQDGVYYLYCHVGYRGRTPPAGRSRARSLTLRSALYRAGGAYGRGSPELLLEGAETVTPVVDPIGYGSLWYTSVGFGGLAQLRSGERVYVNISHPDMVDYRRGKTFFGAVMVG

SEQ ID NO: 37 - protein, 896 aa, artificial sequence
  349B1 Anti-B7-H4 deglycosylated mIgG1-mLT alpha beta beta without signal sequence
QVQLQQSGSELVRPGASVKLSSKALGYTFTDYEMHWVKQTPVHGLEWIGTIHPGTGGTAYNQKFKGKATLTADKSSSTAYMELSSLTSEDSAVYYFTNLDNWGQGTTLTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFASTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGKKPAAHLVGYPSKQNSLLWRASTDRAFLRHGFSLSNNSLLIPTSGLYFVYSQVVFSGESCSPRAIPTPIYLAHEVQLFSSQYPFHVPLLSAQKSVYPGLQGPWVRSMYQGAVFLLSKGDQLSTHTDGISHLHFSPSSVFFGAFALLNPELPAAHLIGAWMSGQGLSWEASQEEAFLRSGAQFSPTHGLALPQDGVYYLYCHVGYRGRTPPAGRSRARSLTLRSALYRAGGAYGRGSPELLLEGAETVTPVVDPIGYGSLWYTSVGFGGLAQLRSGERVYVNISHPDMVDYRRGKTFFGAVMVGLNPELPAAHLIGAWMSGQGLSWEASQEEAFLRSGAQFSPTHGLALPQDGVYYLYCHVGYRGRTPPAGRSRARSLTLRSALYRAGGAYGRGSPELLLEGAETVTPVVDPIGYGSLWYTSVGFGGLAQLRSGERVYVNISHPDMVDYRRGKTFFGAVMVG

SEQ ID NO: 38 - protein, 227 aa, artificial sequence
  Human IgG1 Fc region deglycosylated (13A Version)
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDKLTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 39 - protein, 227 aa, artificial sequence
  Human IgG1 Fc region deglycosylated (13B Version)
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVEGFYPSDIAVEWESNGQPENNYKTTPPVLDSDGSFFLYSELTVDKSRWQQGNVFSCSVMHEALHNHYTQKSLSLSPGK

SEQ ID NO: 40 - protein, 224 aa, artificial sequence
  Human IgG2 Fc region deglycosylated (13A Version)
CVECPPCPAPPVAGPSVFLFPPKPKDTLMISRTPEVTCVVVDVSHEDPEVQFNWYVDGVEVHNAKTKPREEQFASTFRVVSVLTVVHQDWLNGKEYKCKVSNKGLPAPIEKTISKTKGQPREPQVYTLPPSREKMTKNQVSLTCLVKGFYPSDIAVEWESNGQPENNYKTTPPMLKSDGSFFLYSKLTVDKSRWQQGNVFSCSVMHEALHNHYTQKSLSLSPGK

SEQ ID NO: 41 - protein, 224 aa, artificial sequence; identical to SEQ ID NO: 34
  Human IgG2 Fc region deglycosylated (13B Version)

SEQ ID NO: 42 - protein, 6 aa, artificial sequence
  278M1 heavy chain CDR1
TSYYMH

SEQ ID NO: 43 - protein, 17 aa, artificial sequence
  278M1 heavy chain CDR2
YVDPFNGGTSYNQKFKG

SEQ ID NO: 44 - protein, 7 aa, artificial sequence
  278M1 heavy chain CDR3
FIAGFAN

SEQ ID NO: 45 - protein, 6 aa, artificial sequence
  278M1 heavy chain CDR3
IAGFAN

SEQ ID NO: 46 - protein, 11 aa, artificial sequence
  278M1 light chain CDR1
KASQDIKSYLS

SEQ ID NO: 47 - protein, 7 aa, artificial sequence
  278M1 light chain CDR2
YATSLAD

SEQ ID NO: 48 - protein, 9 aa, artificial sequence
  278M1 light chain CDR3
LQHGESPYT

SEQ ID NO: 49 - protein, 8 aa, artificial sequence
  278M1 light chain CDR3
LQHGESPY

SEQ ID NO: 50 - protein, 115 aa, artificial sequence
  278M1 heavy chain variable sequence (without signal sequence)
QVQLQQSGAELMKPGASVKISCKASDYSFTSYYMHWVKQSHGKSLEWVGYVDPFNGGTSYNQKFKGKATLTVDKSSSTAYMHLSSLTSEDSGVYYCAFIAGFANWGQGTLVTVSA

SEQ ID NO: 51 - protein, 107 aa, artificial sequence
  278M1 light chain variable sequence (without signal sequence)
DIVMTQSPSSMYASLGERVTITCKASQDIKSYLSWYQQKPWKSPKTLIYYATSLADGVPSRFSGSGSGQDFSLTISSLESDDTATYYCLQHGESPYTFGGGTKLEIK

SEQ ID NO: 52 - protein, 439 aa, artificial sequence
  278M1 heavy chain sequence (without signal sequence)
QVQLQQSGAELMKPGASVKISCKASDYSFTSYYMHWVKQSHGKSLEWVGYVDPFNGGTSYNQKFKGKATLTVDKSSSTAYMHLSSLTSEDSGVYYCAFIAGFANWGQGTLVTVSAAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 53 - protein, 214 aa, artificial sequence
  278M1 light chain sequence (without signal sequence)
DIVMTQSPSSMYASLGERVTITCKASQDIKSYLSWYQQKPWKSPKTLIYYATSLADGVPSRFSGSGSGQDFSLTISSLESDDTATYYCLQHGESPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 54 - protein, 458 aa, artificial sequence
  278M1 heavy chain sequence (signal sequence underlined)
MKHLWFFLLLVAAPRWVLSQVQLQQSGAELMKPGASVKISCKASDYSFTSYYMHWVKQSHGKSLEWVGYVDPFNGGTSYNQKFKGKATLTVDKSSSTAYMHLSSLTSEDSGVYYCAFIAGFANWGQGTLVTVSAAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 55 - protein, 233 aa, artificial sequence
  278M1 light chain sequence (signal sequence underlined)
MKHLWFFLLLVAAPRWVLSDIVMTQSPSSMYASLGERVTITCKASQDIKSYLSWYQQKPWKSPKTLIYYATSLADGVPSRFSGSGSGQDFSLTISSLESDDTATYYCLQHGESPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 56 - protein, 257 aa, artificial sequence
  Human B7-H4 extracellular domain (aa 1-257 including signal sequence
MASLGQILFWSIISIIIILAGAIALIIGFGISGRHSITVTTVASAGNIGEDGILSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIKRRSHLQLLNSK

SEQ ID NO: 57 - DNA, 2814 bp, Artificial Sequence
  349B4 humanized anti-B7-H4/ hLT alpha-beta-beta heavy chain precursor DNA
atggactggacctggcggattctctttctcgtggctgctgccacaggcgcccactctcaggtccagctcgtgcagtctggcgctgaggtgaagaagcctggggcctccgtgaaggtttcctgcaaggcttctggatacaccttcacttcctactacatgcactgggtgcgccaggcccccggacaaaggctcgaatggatgggatatgttgatcctttcaatggcggaacatcctacaaccagaaattcaagggcagagtcaccattaccgttgacacatcctctagcacagcctacatggagctctccagcctgcggtctgaagacactgctgtgtattactgtgcctttattgctgggtttgctaactggggccagggcaccctggtcaccgtcagctcagccagcacaaagggcccctccgtgttccctctggccccttcctccaagtccacctccggcggcaccgccgctctgggctgcctggtgaaggactacttccctgagcctgtgaccgtgtcctggaactctggcgctctgacctctggcgtccacaccttcccagccgtgctgcagtcctccggcctgtactccctgtcctccgtggtgactgtgccttcctcctccctgggcacccagacctacatctgcaacgtgaaccacaagccttccaacaccaaggtggacaagcgggtggagcctaagtcctgcgacaagacccacacctgccctccctgccctgcccctgagctgctgggcggaccttccgtgttcctgttccctcctaagcctaaggacaccctgatgatctcccggacccctgaggtgacatgcgtggtggtggacgtgtcccacgaggaccctgaggtgaagttcaactggtatgtggacggcgtggaagtgcataacgctaagaccaagccaagggaggagcagtacgcctccacctaccgggtggtgtctgtgctgaccgtgctgcaccaggactggctgaacggcaaagaatacaagtgcaaggtctccaacaaggccctgcccgctcccatcgagaaaaccatctccaaggccaagggccagcctcgcgagcctcaggtgtacaccctgccacccagccgggaggagctgaccaagaaccaggtgtccctgacctgtctggtgaagggcttctacccttccgatattgccgtggagtgggagtctaacggccagcccgagaacaactacaagaccacccctcctgtgctggactccgacggctccttcttcctgtactccaagctgaccgtggacaagtcccggtggcagcagggcaacgtgttctcctgctccgtgatgcacgaggccctgcacaaccactacacccagaagtccctgtctctgtctcctggcaaggcccacagcaccctcaaacctgctgctcacctcattggcgaccccagcaagcaaaactcactgctctggcgggcaaacactgaccgggccttcctccaagatggtttctccctctctaacaattctctcctggtccccacctctggcatctacttcgtctactcccaagtggtcttctctgggaaagcctactctcccaaagccacctcctccccactctacctggcccatgaggtccagctcttctcctcccaataccccttccatgtgcctctcctctcttcccaaaaaatggtgtatccagggctgcaggaaccctggctgcactctatgtaccacggggctgctttccaactcacccagggagaccagctctccacccacactgatggcatcccccacctcgtcctctctccttctactgtcttctttggagccttcgctctgctctcacccgggctcccagctgcccacctcatcggcgctccactgaaagggcaggggctcggctgggagactactaaggaacaggcttttctgaccagcgggacccagttctccgacgccgaggggctggccctcccccaggacggcctctattacctctactgtctcgtcggctaccggggccgcgccccccctggcggcggggacccccagggccgctccgtcaccctgcgctcctctctgtaccgggccgggggcgcatacggccccggcactcccgagctgctgctcgaaggggccgagaccgtgactccagtgctggaccccgccaggagacaaggctacgggcctctctggtacaccagcgtggggttcggcggcctggtgcagctccggaggggcgagagggtgtacgtcaacatctcccaccccgatatggtggacttcgccagagggaagaccttctttggggccgtgatggtcggactgtctcctggcctgcctgccgcacatctgattggtgcccctctcaaaggacaaggactcggatgggaaacaacaaaagaacaagcattcctcacatccggaacacaattttctgatgcagaagggctcgcactgccacaagatgggctgtactatctgtattgcctggttgggtatcgcggtcgcgcacctcccggggggggcgatcctcaagggcggtcagttaccctccggagcagcctctatcgggcaggcggggcttatggacctggaacccctgaactcctcctggaaggggctgaaaccgtcacccccgtcctcgatcccgctcggcggcaaggctatggccccctgtggtatacctccgtcggctttggggggctcgtccaactgcgccggggggaacgggtctatgtgaatatttcccatcctgacatggtcgattttgcccggggcaaaacatttttcggcgctgtcatggtcggctga

SEQ ID NO: 58 - DNA, 705 bp, Artificial Sequence
  349B4 humanized anti-B7-H4/hLT alpha-beta-beta light chain precursor DNA
atggtgctccagacccaggtcttcatttccctgctgctctggatcagcggagcctacggggacatccagatgacccagtctccatcctccctgtctgcatctgttggagacagagtcaccatcacttgcaaggcatctcaggacattaaaagctatctcagctggtatcagcagaaaccagggaaagcccctaagaccctgatctattatgcaacaagcctcgcagatggggtcccatcaaggttctctggctctggatctgggacagatttcactctcaccatcagctctctgcaacctgaagattttgcaacttactactgtctgcagcatggcgagagcccttacacattcggcggagggaccaaggtggagatcaaacgcacggtggctgcaccatctgtcttcatcttccctccatctgatgagcagctcaaatctggaactgcctctgttgtgtgcctgctgaataacttctatcccagagaggccaaagtccagtggaaggtggataacgccctccaatccggcaactcccaggaatctgtcacagagcaggacagcaaggacagcacctacagcctcagctccaccctgacactgagcaaagcagactacgagaaacacaaagtctatgcctgcgaagtcacccatcagggcctgtcttcccccgtcacaaagagcttcaacaggggagagtgctaa

SEQ ID NO: 59 - protein, 234 aa, artificial sequence
  278M1-LC2 humanized light chain with signal sequence (signal sequence underlined)
MVLQTQVFISLLLWISGAYGDIQMTQSPSSLSASVGDRVTITCKASQDIKSYLSWYQQKPGKAPKTLIYYATSLADGVPSRFSGSGSGTDFTLTISSLQPEDFATYYCLQHGESPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 60 - protein, 214 aa, artificial sequence
  278M1-LC2 humanized light chain without signal sequence
DIQMTQSPSSLSASVGDRVTITCKASQDIKSYLSWYQQKPGKAPKTLIYYATSLADGVPSRFSGSGSGTDFTLTISSLQPEDFATYYCLQHGESPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 61 - protein, 128 aa, artificial sequence
  278M1-VL2 humanized light chain variable domain with the signal sequence (signal sequence underlined)
MVLQTQVFISLLLWISGAYGDIQMTQSPSSLSASVGDRVTITCKASQDIKSYLSWYQQKPGKAPKTLIYYATSLADGVPSRFSGSGSGTDFTLTISSLQPEDFATYYCLQHGESPYTFGGGTKVEIKR

SEQ ID NO: 62 - protein, 108 aa, artificial sequence
  278M1-VL2 humanized light chain variable domain without signal sequence
DIQMTQSPSSLSASVGDRVTITCKASQDIKSYLSWYQQKPGKAPKTLIYYATSLADGVPSRFSGSGSGTDFTLTISSLQPEDFATYYCLQHGESPYTFGGGTKVEIKR

SEQ ID NO: 63 - protein, 470 aa, artificial sequence
  278M1_VH2-IgG1 humanized heavy chain with signal sequence (signal sequence underlined)
MDWTWRILFLVAAATGAHSQVQLVQSGAEVKKPGASVKVSCKASGYTFTSYYMHWVRQAPGQRLEWMGYVDPFNGGTSYNQKFKGRVTITVDTSSSTAYMELSSLRSEDTAVYYCAFIAGFANWGQGTLVTVSSASTKGPASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 64 - protein, 451 aa, artificial sequence
  278M1_VH2-IgG1 humanized heavy chain without signal sequence
QVQLVQSGAEVKKPGASVKVSCKASGYTFTSYYMHWVRQAPGQRLEWMGYVDPFNGGTSYNQKFKGRVTITVDTSSSTAYMELSSLRSEDTAVYYCAFIAGFANWGQGTLVTVSSASTKGPASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 65 - protein, 134 aa, artificial sequence
  278M1-VH2 humanized heavy chain variable domain with the signal sequence (signal sequence underlined)
MDWTWRILFLVAAATGAHSQVQLVQSGAEVKKPGASVKVSCKASGYTFTSYYMHWVRQAPGQRLEWMGYVDPFNGGTSYNQKFKGRVTITVDTSSSTAYMELSSLRSEDTAVYYCAFIAGFANWGQGTLVTVSS

SEQ ID NO: 66 - protein, 121 aa, artificial sequence
  278M1-VH2 humanized heavy chain variable domain without signal sequence
QVQLVQSGAEVKKPGASVKVSCKASGYTFTSYYMHWVRQAPGQRLEWMGYVDPFNGGTSYNQKFKGRVTITVDTSSSTAYMELSSLRSEDTAVYYCAFIAGFANWGQGTLVTVSSASTKGP

SEQ ID NO: 67 - protein, 239 aa, artificial sequence
  173M36 LC1 humanized light chain with signal sequence (signal sequence underlined)
MVLQTQVFISLLLWISGAYGDIVMTQSPLSLPVTPGEPASISCRSSQSIVQSNGNTYLEWYLQKPGQSPQLLIYKVSNQFSGVPDRFSGSGSGTDFTLKISRVEAEDVGVYYCFQGSHVPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 68 - protein, 219 aa, artificial sequence
  173M36 LC1 humanized light chain without signal sequence
DIVMTQSPLSLPVTPGEPASISCRSSQSIVQSNGNTYLEWYLQKPGQSPQLLIYKVSNQFSGVPDRFSGSGSGTDFTLKISRVEAEDVGVYYCFQGSHVPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 69 - protein, 239 aa, artificial sequence
  173M36 LC3 humanized light chain with signal sequence (signal sequence underlined)
MVLQTQVFISLLLWISGAYGDIVMTQSPSSLPVTPGEPASISCRSSQSIVQSNGNTYLEWYLQKPGQSPQLLLYKVSNQFSGVPDRISGSGSGTDFTLKISRVEAEDVGVYYCFQGSHVPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 70 - protein, 219 aa, artificial sequence
  173M36 LC3 humanized light chain without signal sequence
DIVMTQSPSSLPVTPGEPASISCRSSQSIVQSNGNTYLEWYLQKPGQSPQLLLYKVSNQFSGVPDRISGSGSGTDFTLKISRVEAEDVGVYYCFQGSHVPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 71 - protein, 133 aa, artificial sequence
  173M36 VL1 light chain variable domain with the signal sequence (signal sequence underlined)
MVLQTQVFISLLLWISGAYGDIVMTQSPLSLPVTPGEPASISCRSSQSIVQSNGNTYLEWYLQKPGQSPQLLIYKVSNQFSGVPDRFSGSGSGTDFTLKISRVEAEDVGVYYCFQGSHVPLTFGQGTKVEIKR

SEQ ID NO: 72 - protein, 113 aa, artificial sequence
  173M36 VL1 light chain variable domain without signal sequence
DIVMTQSPLSLPVTPGEPASISCRSSQSIVQSNGNTYLEWYLQKPGQSPQLLIYKVSNQFSGVPDRFSGSGSGTDFTLKISRVEAEDVGVYYCFQGSHVPLTFGQGTKVEIKR

SEQ ID NO: 73 - protein, 16 aa, artificial sequence
  173M36 LC CDR1
RSSQSIVQSNGNTYLE

SEQ ID NO: 74 - protein, 7 aa, artificial sequence
  173M36 LC CDR2
KVSNQFS

SEQ ID NO: 75 - protein, 7 aa, artificial sequence
  173M36 LC CDR3
QGSHVPL

SEQ ID NO: 76 - protein, 469 aa, artificial sequence
  173M36_VH2-IgG1 humanized heavy chain with signal sequence (signal sequence underlined)
MDWTWRILFLVAAATGAHSEVQLVQSGGGLVKPGGSLRLSCAASGFTFSTYGMSWVRQAPGKGLEWVATISDGGSYTYYPDSVKGRFTISRDNAKNSLYLQMNSLRAEDTAVYYCARHYYGSDWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 77 - protein, 450 aa, artificial sequence
  173M36_VH2-IgG1 humanized heavy chain without signal sequence
EVQLVQSGGGLVKPGGSLRLSCAASGFTFSTYGMSWVRQAPGKGLEWVATISDGGSYTYYPDSVKGRFTISRDNAKNSLYLQMNSLRAEDTAVYYCARHYYGSDWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 78 - protein, 139 aa, artificial sequence
  173M36 VH2 humanized heavy chain variable domain with the signal sequence (signal sequence underlined)
MDWTWRILFLVAAATGAHSEVQLVQSGGGLVKPGGSLRLSCAASGFTFSTYGMSWVRQAPGKGLEWVATISDGGSYTYYPDSVKGRFTISRDNAKNSLYLQMNSLRAEDTAVYYCARHYYGSDWYFDVWGQGTTVTVSS

SEQ ID NO: 79 - protein, 120 aa, artificial sequence
  173M36 VH2 humanized heavy chain variable domain without signal sequence
EVQLVQSGGGLVKPGGSLRLSCAASGFTFSTYGMSWVRQAPGKGLEWVATISDGGSYTYYPDSVKGRFTISRDNAKNSLYLQMNSLRAEDTAVYYCARHYYGSDWYFDVWGQGTTVTVSS

SEQ ID NO: 80 - protein, 6 aa, artificial sequence
  173M36 VH CDR1
STYGMS

SEQ ID NO: 81 - protein, 19 aa, artificial sequence
  173M36 VH CDR2
ATISDGGSYTYYPDSVKGR

SEQ ID NO: 82 - protein, 13 aa, artificial sequence
  173M36 VH CDR3
ARHYYGSDWYFDV

SEQ ID NO: 83 - protein, 240 aa, artificial sequence
  Human LIGHT with signal sequence (signal sequence underlined)
MEESVVRPSVFVVDGQTDIPFTRLGRSHRRQSCSVARVGLGLLLLLMGAGLAVQGWFLLQLHWRLGEMVTRLPDGPAGSWEQLIQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMV

SEQ ID NO: 84 - protein, 182 aa, artificial sequence
  Human LIGHT extracellular domain (aa 59-240)
LQLHWRLGEMVTRLPDGPAGSWEQLIQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMV

SEQ ID NO: 85 - protein, 152 aa, artificial sequence
  Human LIGHT fragment (aa 89-240)
SHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMV

SEQ ID NO: 86 - protein, 456 aa, artificial sequence
  Single gene human LIGHT homotrimer
SHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMVSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMVSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMV

SEQ ID NO: 87 - protein, 282 aa, artificial sequence
  Human B7-H4 with signal sequence (signal sequence underlined)
MASLGQILFWSIISIIIILAGAIALIIGFGISGRHSITVTTVASAGNIGEDGILSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIKRRSHLQLLNSKASLCVSSFFAISWALLPLSPYLMLK

SEQ ID NO: 88 - protein, 228 aa, artificial sequence
  Human B7-H4 extracellular domain without signal sequence
GISGRHSITVTTVASAGNIGEDGILSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIKRRSHLQLLNSK

SEQ ID NO: 89 - protein, 829 aa, artificial sequence
  Human CDH3 (signal sequence underlined)
MGLPRGPLASLLLLQVCWLQCAASEPCRAVFREAEVTLEAGGAEQEPGQALGKVFMGCPGQEPALFSTDNDDFTVRNGETVQERRSLKERNPLKIFPSKRILRRHKRDWVVAPISVPENGKGPFPQRLNQLKSNKDRDTKIFYSITGPGADSPPEGVFAVEKETGWLLLNKPLDREEIAKYELFGHAVSENGASVEDPMNISIIVTDQNDHKPKFTQDTFRGSVLEGVLPGTSVMQVTATDEDDAIYTYNGVVAYSIHSQEPKDPHDLMFTIHRSTGTISVISSGLDREKVPEYTLTIQATDMDGDGSTTTAVAVVEILDANDNAPMFDPQKYEAHVPENAVGHEVQRLTVTDLDAPNSPAWRATYLIMGGDDGDHFTITTHPESNQGILTTRKGLDFEAKNQHTLYVEVTNEAPFVLKLPTSTATIVVHVEDVNEAPVFVPPSKVVEVQEGIPTGEPVCVYTAEDPDKENQKISYRILRDPAGWLAMDPDSGQVTAVGTLDREDEQFVRNNIYEVMVLAMDNGSPPTTGTGTLLLTLIDVNDHGPVPEPRQITICNQSPVRQVLNITDKDLSPHTSPFQAQLTDDSDIYWTAEVNEEGDTVVLSLKKFLKQDTYDVHLSLSDHGNKEQLTVIRATVCDCHGHVETCPGPWKGGFILPVLGAVLALLFLLLVLLLLVRKKRKIKEPLLLPEDDTRDNVFYYGEEGGGEEDQDYDITQLHRGLEARPEVVLRNDVAPTIIPTPMYRPRPANPDEIGNFIIENLKAANTDPTAPPYDTLLVFDYEGSGSDAASLSSLTSSASDQDQDYDYLNEWGSRFKKLADMYGGGEDD

SEQ ID NO: 90 - protein, 653 aa, artificial sequence
  Human CDH3 extracellular domain (signal sequence underlined)
MGLPRGPLASLLLLQVCWLQCAASEPCRAVFREAEVTLEAGGAEQEPGQALGKVFMGCPGQEPALFSTDNDDFTVRNGETVQERRSLKERNPLKIFPSKRILRRHKRDWVVAPISVPENGKGPFPQRLNQLKSNKDRDTKIFYSITGPGADSPPEGVFAVEKETGWLLLNKPLDREEIAKYELFGHAVSENGASVEDPMNISIIVTDQNDHKPKFTQDTFRGSVLEGVLPGTSVMQVTATDEDDAIYTYNGVVAYSIHSQEPKDPHDLMFTIHRSTGTISVISSGLDREKVPEYTLTIQATDMDGDGSTTTAVAVVEILDANDNAPMFDPQKYEAHVPENAVGHEVQRLTVTDLDAPNSPAWRATYLIMGGDDGDHFTITTHPESNQGILTTRKGLDFEAKNQHTLYVEVTNEAPFVLKLPTSTATIVVHVEDVNEAPVFVPPSKVVEVQEGIPTGEPVCVYTAEDPDKENQKISYRILRDPAGWLAMDPDSGQVTAVGTLDREDEQFVRNNIYEVMVLAMDNGSPPTTGTGTLLLTLIDVNDHGPVPEPRQITICNQSPVRQVLNITDKDLSPHTSPFQAQLTDDSDIYWTAEVNEEGDTVVLSLKKFLKQDTYDVHLSLSDHGNKEQLTVIRATVCDCHGHVETCPGPWKG

SEQ ID NO: 91 - protein, 546 aa, artificial sequence
  Mature human CDH3 extracellular domain
DWVVAPISVPENGKGPFPQRLNQLKSNKDRDTKIFYSITGPGADSPPEGVFAVEKETGWLLLNKPLDREEIAKYELFGHAVSENGASVEDPMNISIIVTDQNDHKPKFTQDTFRGSVLEGVLPGTSVMQVTATDEDDAIYTYNGVVAYSIHSQEPKDPHDLMFTIHRSTGTISVISSGLDREKVPEYTLTIQATDMDGDGSTTTAVAVVEILDANDNAPMFDPQKYEAHVPENAVGHEVQRLTVTDLDAPNSPAWRATYLIMGGDDGDHFTITTHPESNQGILTTRKGLDFEAKNQHTLYVEVTNEAPFVLKLPTSTATIVVHVEDVNEAPVFVPPSKVVEVQEGIPTGEPVCVYTAEDPDKENQKISYRILRDPAGWLAMDPDSGQVTAVGTLDREDEQFVRNNIYEVMVLAMDNGSPPTTGTGTLLLTLIDVNDHGPVPEPRQITICNQSPVRQVLNITDKDLSPHTSPFQAQLTDDSDIYWTAEVNEEGDTVVLSLKKFLKQDTYDVHLSLSDHGNKEQLTVIRATVCDCHGHVETCPGPWKG

SEQ ID NO: 92 - protein, 132 aa, artificial sequence
  173M36 VL3 light chain variable domain with the signal sequence (signal sequence underlined)
MVLQTQVFISLLLWISGAYDIVMTQSPSSLPVTPGEPASISCRSSQSIVQSNGNTYLEWYLQKPGQSPQLLLYKVSNQFSGVPDRISGSGSGTDFTLKISRVEAEDVGVYYCFQGSHVPLTFGQGTKVEIKR

SEQ ID NO: 93 - protein, 113 aa, artificial sequence
  173M36 VL3 light chain variable domain without signal sequence
DIVMTQSPSSLPVTPGEPASISCRSSQSIVQSNGNTYLEWYLQKPGQSPQLLLYKVSNQFSGVPDRISGSGSGTDFTLKISRVEAEDVGVYYCFQGSHVPLTFGQGTKVEIKR

SEQ ID NO: 94 - protein, 682 aa, artificial sequence
  363F1 mFc-mLTalpha-beta-beta
GCKPCICTVPEVSSVFIFPPKPKDVLTITLTPKVTCVVVDISKDDPEVQFSWFVDDVEVHTAQTQPREEQFASTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGKKPAAHLVGYPSKQNSLLWRASTDRAFLRHGFSLSNNSLLIPTSGLYFVYSQVVFSGESCSPRAIPTPIYLAHEVQLFSSQYPFHVPLLSAQKSVYPGLQGPWVRSMYQGAVFLLSKGDQLSTHTDGISHLHFSPSSVFFGAFALLNPELPAAHLIGAWMSGQGLSWEASQEEAFLRSGAQFSPTHGLALPQDGVYYLYCHVGYRGRTPPAGRSRARSLTLRSALYRAGGAYGRGSPELLLEGAETVTPVVDPIGYGSLWYTSVGFGGLAQLRSGERVYVNISHPDMVDYRRGKTFFGAVMVGLNPELPAAHLIGAWMSGQGLSWEASQEEAFLRSGAQFSPTHGLALPQDGVYYLYCHVGYRGRTPPAGRSRARSLTLRSALYRAGGAYGRGSPELLLEGAETVTPVVDPIGYGSLWYTSVGFGGLAQLRSGERVYVNISHPDMVDYRRGKTFFGAVMVG

SEQ ID NO: 95 - protein, 695 aa, artificial sequence
  363F2 hFc-hLT alpha-beta-beta
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKKPAAHLIGDPSKQNSLLWRANTDRAFLQDGFSLSNNSLLVPTSGIYFVYSQVVFSGKAYSPKATSSPLYLAHEVQLFSSQYPFHVPLLSSQKMVYPGLQEPWLHSMYHGAAFQLTQGDQLSTHTDGIPHLVLSPSTVFFGAFALLSPGLPAAHLIGAPLKGQGLGWETTKEQAFLTSGTQFSDAEGLALPQDGLYYLYCLVGYRGRAPPGGGDPQGRSVTLRSSLYRAGGAYGPGTPELLLEGAETVTPVLDPARRQGYGPLWYTSVGFGGLVQLRRGERVYVNISHPDMVDFARGKTFFGAVMVGLSPGLPAAHLIGAPLKGQGLGWETTKEQAFLTSGTQFSDAEGLALPQDGLYYLYCLVGYRGRAPPGGGDPQGRSVTLRSSLYRAGGAYGPGTPELLLEGAETVTPVLDPARRQGYGPLWYTSVGFGGLVQLRRGERVYVNISHPDMVDFARGKTFFGAVMVG

SEQ ID NO: 96 - protein, 695 aa, artificial sequence
  363F3 hFc-hLT beta-alpha-beta
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKLSPGLPAAHLIGAPLKGQGLGWETTKEQAFLTSGTQFSDAEGLALPQDGLYYLYCLVGYRGRAPPGGGDPQGRSVTLRSSLYRAGGAYGPGTPELLLEGAETVTPVLDPARRQGYGPLWYTSVGFGGLVQLRRGERVYVNISHPDMVDFARGKTFFGAVMVGKPAAHLIGDPSKQNSLLWRANTDRAFLQDGFSLSNNSLLVPTSGIYFVYSQVVFSGKAYSPKATSSPLYLAHEVQLFSSQYPFHVPLLSSQKMVYPGLQEPWLHSMYHGAAFQLTQGDQLSTHTDGIPHLVLSPSTVFFGAFALLSPGLPAAHLIGAPLKGQGLGWETTKEQAFLTSGTQFSDAEGLALPQDGLYYLYCLVGYRGRAPPGGGDPQGRSVTLRSSLYRAGGAYGPGTPELLLEGAETVTPVLDPARRQGYGPLWYTSVGFGGLVQLRRGERVYVNISHPDMVDFARGKTFFGAVMVG

SEQ ID NO: 97 - protein, 695 aa, artificial sequence
  363F4 hFc-hLT beta-beta-alpha
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKLSPGLPAAHLIGAPLKGQGLGWETTKEQAFLTSGTQFSDAEGLALPQDGLYYLYCLVGYRGRAPPGGGDPQGRSVTLRSSLYRAGGAYGPGTPELLLEGAETVTPVLDPARRQGYGPLWYTSVGFGGLVQLRRGERVYVNISHPDMVDFARGKTFFGAVMVGLSPGLPAAHLIGAPLKGQGLGWETTKEQAFLTSGTQFSDAEGLALPQDGLYYLYCLVGYRGRAPPGGGDPQGRSVTLRSSLYRAGGAYGPGTPELLLEGAETVTPVLDPARRQGYGPLWYTSVGFGGLVQLRRGERVYVNISHPDMVDFARGKTFFGAVMVGKPAAHLIGDPSKQNSLLWRANTDRAFLQDGFSLSNNSLLVPTSGIYFVYSQVVFSGKAYSPKATSSPLYLAHEVQLFSSQYPFHVPLLSSQKMVYPGLQEPWLHSMYHGAAFQLTQGDQLSTHTDGIPHLVLSPSTVFFGAFAL

SEQ ID NO: 98 - protein, 942 aa, artificial sequence
  364B4 humanized anti-CDH3/hLT alpha-beta-beta heavy chain precursor protein (signal sequence underlined)
MDWTWRILFLVAAATGAHSEVQLVQSGGGLVKPGGSLRLSCAASGFTFSTYGMSWVRQAPGKGLEWVATISDGGSYTYYPDSVKGRFTISRDNAKNSLYLQMNSLRAEDTAVYYCARHYYGSDWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREELTKNQVSLTCLVKGFYPSDIAVEWESNGQPENNYKTTPPVLDSDGSFFLYSKLTVDKSRWQQGNVFSCSVMHEALHNHYTQKSLSLSPGKAHSTLKPAAHLIGDPSKQNSLLWRANTDRAFLQDGFSLSNNSLLVPTSGIYFVYSQVVFSGKAYSPKATSSPLYLAHEVQLFSSQYPFHVPLLSSQKMVYPGLQEPWLHSMYHGAAFQLTQGDQLSTHTDGIPHLVLSPSTVFFGAFALLSPGLPAAHLIGAPLKGQGLGWETTKEQAFLTSGTQFSDAEGLALPQDGLYYLYCLVGYRGRAPPGGGDPQGRSVTLRSSLYRAGGAYGPGTPELLLEGAETVTPVLDPARRQGYGPLWYTSVGFGGLVQLRRGERVYVNISHPDMVDFARGKTFFGAVMVGLSPGLPAAHLIGAPLKGQGLGWETTKEQAFLTSGTQFSDAEGLALPQDGLYYLYCLVGYRGRAPPGGGDPQGRSVTLRSSLYRAGGAYGPGTPELLLEGAETVTPVLDPARRQGYGPLWYTSVGFGGLVQLRRGERVYVNISHPDMVDFARGKTFFGAVMVG

SEQ ID NO: 99 - protein, 923 aa, artificial sequence
  364B4 humanized anti-CDH3/hLT alpha-beta-beta heavy chain mature protein
EVQLVQSGGGLVKPGGSLRLSCAASGFTFSTYGMSWVRQAPGKGLEWVATISDGGSYTYYPDSVKGRFTISRDNAKNSLYLQMNSLRAEDTAVYYCARHYYGSDWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREELTKNQVSLTCLVKGFYPSDIAVEWESNGQPENNYKTTPPVLDSDGSFFLYSKLTVDKSRWQQGNVFSCSVMHEALHNHYTQKSLSLSPGKAHSTLKPAAHLIGDPSKQNSLLWRANTDRAFLQDGFSLSNNSLLVPTSGIYFVYSQVVFSGKAYSPKATSSPLYLAHEVQLFSSQYPFHVPLLSSQKMVYPGLQEPWLHSMYHGAAFQLTQGDQLSTHTDGIPHLVLSPSTVFFGAFALLSPGLPAAHLIGAPLKGQGLGWETTKEQAFLTSGTQFSDAEGLALPQDGLYYLYCLVGYRGRAPPGGGDPQGRSVTLRSSLYRAGGAYGPGTPELLLEGAETVTPVLDPARRQGYGPLWYTSVGFGGLVQLRRGERVYVNISHPDMVDFARGKTFFGAVMVGLSPGLPAAHLIGAPLKGQGLGWETTKEQAFLTSGTQFSDAEGLALPQDGLYYLYCLVGYRGRAPPGGGDPQGRSVTLRSSLYRAGGAYGPGTPELLLEGAETVTPVLDPARRQGYGPLWYTSVGFGGLVQLRRGERVYVNISHPDMVDFARGKTFFGAVMVG

SEQ ID NO: 100 - DNA, 2829 bp, Artificial Sequence
  364B4 humanized anti-CDH3/hLT alpha-beta-beta heavy chain precursor DNA
atggactggacctggaggatactctttctcgtggctgcagccacaggagcccactccgaggtgcagctggtgcaatctgggggaggactggtcaagcctggggggtccctgagactctcctgtgcagcctctggattcaccttctctacctatggcatgtcttgggtccgccaagctccagggaaggggctggagtgggtcgcaaccatttctgatggtggtagctacacctactatccagactccgtgaaggggcggttcaccatctccagagacaacgccaagaactcactgtatctgcaaatgaacagcctgagagccgaggacacagctgtgtattactgtgctagacattactacggttctgactggtacttcgatgtctgggggcaagggaccaccgtcaccgtcagctcagccagcacaaagggcccctccgtgttccctctggccccttcctccaagtccacctccggcggcaccgccgctctgggctgcctggtgaaggactacttccctgagcctgtgaccgtgtcctggaactctggcgctctgacctctggcgtccacaccttcccagccgtgctgcagtcctccggcctgtactccctgtcctccgtggtgactgtgccttcctcctccctgggcacccagacctacatctgcaacgtgaaccacaagccttccaacaccaaggtggacaagcgggtggagcctaagtcctgcgacaagacccacacctgccctccctgccctgcccctgagctgctgggcggaccttccgtgttcctgttccctcctaagcctaaggacaccctgatgatctcccggacccctgaggtgacatgcgtggtggtggacgtgtcccacgaggaccctgaggtgaagttcaactggtatgtggacggcgtggaagtgcataacgctaagaccaagccaagggaggagcagtacgcctccacctaccgggtggtgtctgtgctgaccgtgctgcaccaggactggctgaacggcaaagaatacaagtgcaaggtctccaacaaggccctgcccgctcccatcgagaaaaccatctccaaggccaagggccagcctcgcgagcctcaggtgtacaccctgccacccagccgggaggagctgaccaagaaccaggtgtccctgacctgtctggtgaagggcttctacccttccgatattgccgtggagtgggagtctaacggccagcccgagaacaactacaagaccacccctcctgtgctggactccgacggctccttcttcctgtactccaagctgaccgtggacaagtcccggtggcagcagggcaacgtgttctcctgctccgtgatgcacgaggccctgcacaaccactacacccagaagtccctgtctctgtctcctggcaaggcccacagcaccctcaaacctgctgctcacctcattggcgaccccagcaagcaaaactcactgctctggcgggcaaacactgaccgggccttcctccaagatggtttctccctctctaacaattctctcctggtccccacctctggcatctacttcgtctactcccaagtggtcttctctgggaaagcctactctcccaaagccacctcctccccactctacctggcccatgaggtccagctcttctcctcccaataccccttccatgtgcctctcctctcttcccaaaaaatggtgtatccagggctgcaggaaccctggctgcactctatgtaccacggggctgctttccaactcacccagggagaccagctctccacccacactgatggcatcccccacctcgtcctctctccttctactgtcttctttggagccttcgctctgctctcacccgggctcccagctgcccacctcatcggcgctccactgaaagggcaggggctcggctgggagactactaaggaacaggcttttctgaccagcgggacccagttctccgacgccgaggggctggccctcccccaggacggcctctattacctctactgtctcgtcggctaccggggccgcgccccccctggcggcggggacccccagggccgctccgtcaccctgcgctcctctctgtaccgggccgggggcgcatacggccccggcactcccgagctgctgctcgaaggggccgagaccgtgactccagtgctggaccccgccaggagacaaggctacgggcctctctggtacaccagcgtggggttcggcggcctggtgcagctccggaggggcgagagggtgtacgtcaacatctcccaccccgatatggtggacttcgccagagggaagaccttctttggggccgtgatggtcggactgtctcctggcctgcctgccgcacatctgattggtgcccctctcaaaggacaaggactcggatgggaaacaacaaaagaacaagcattcctcacatccggaacacaattttctgatgcagaagggctcgcactgccacaagatgggctgtactatctgtattgcctggttgggtatcgcggtcgcgcacctcccggggggggcgatcctcaagggcggtcagttaccctccggagcagcctctatcgggcaggcggggcttatggacctggaacccctgaactcctcctggaaggggctgaaaccgtcacccccgtcctcgatcccgctcggcggcaaggctatggccccctgtggtatacctccgtcggctttggggggctcgtccaactgcgccggggggaacgggtctatgtgaatatttcccatcctgacatggtcgattttgcccggggcaaaacatttttcggcgctgtcatggtcggctga

SEQ ID NO: 101 - protein, 239 aa, artificial sequence; identical to SEQ ID NO: 67
  364B4 humanized anti-CDH3/hLT alpha-beta-beta light chain precursor protein (signal sequence underlined)

SEQ ID NO: 102 - protein, 219 aa, artificial sequence; identical to SEQ ID NO: 68
  364B4 humanized anti-CDH3/hLT alpha-beta-beta light chain mature protein

SEQ ID NO: 103 - DNA, 720 bp, Artificial Sequence
  364B4 humanized anti-CDH3/hLT alpha-beta-beta light chain precursor
atggtgctccagacccaggtcttcatttccctgctgctctggatcagcggagcctacggggatattgtgatgactcagtctccactctccctgcccgtcacccctggagagcctgcctccatctcctgccggtcttcacaatccattgttcaatctaatggaaacacctatctcgaatggtatctgcagaagccagggcagtctccacagctcctgatctataaagtttccaaccaattttctggggtccctgatcggttctctggctctggatcaggcacagattttacactgaaaatcagcagagtggaggctgaggatgttggggtttattactgctttcaaggttcacatgttcctctcacattcggccaagggaccaaggtggaaatcaagcgtacggtggctgcaccatctgtcttcatcttccctccatctgatgagcagctcaaatctggaactgcctctgttgtgtgcctgctgaataacttctatcccagagaggccaaagtccagtggaaggtggataacgccctccaatccggcaactcccaggaatctgtcacagagcaggacagcaaggacagcacctacagcctcagctccaccctgacactgagcaaagcagactacgagaaacacaaagtctatgcctgcgaagtcacccatcagggcctgtcttcccccgtcacaaagagcttcaacaggggagagtgctaa

SEQ ID NO: 104 - protein, 937 aa, artificial sequence
  349B4 humanized anti-B7-H4/ hLT alpha-beta-beta heavy chain precursor protein (signal sequence underlined)
MDWTWRILFLVAAATGAHSQVQLVQSGAEVKKPGASVKVSCKASGYTFTSYYMHWVRQAPGQRLEWMGYVDPFNGGTSYNQKFKGRVTITVDTSSSTAYMELSSLRSEDTAVYYCAFIAGFANWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREELTKNQVSLTCLVKGFYPSDIAVEWESNGQPENNYKTTPPVLDSDGSFFLYSKLTVDKSRWQQGNVFSCSVMHEALHNHYTQKSLSLSPGKAHSTLKPAAHLIGDPSKQNSLLWRANTDRAFLQDGFSLSNNSLLVPTSGIYFVYSQVVFSGKAYSPKATSSPLYLAHEVQLFSSQYPFHVPLLSSQKMVYPGLQEPWLHSMYHGAAFQLTQGDQLSTHTDGIPHLVLSPSTVFFGAFALLSPGLPAAHLIGAPLKGQGLGWETTKEQAFLTSGTQFSDAEGLALPQDGLYYLYCLVGYRGRAPPGGGDPQGRSVTLRSSLYRAGGAYGPGTPELLLEGAETVTPVLDPARRQGYGPLWYTSVGFGGLVQLRRGERVYVNISHPDMVDFARGKTFFGAVMVGLSPGLPAAHLIGAPLKGQGLGWETTKEQAFLTSGTQFSDAEGLALPQDGLYYLYCLVGYRGRAPPGGGDPQGRSVTLRSSLYRAGGAYGPGTPELLLEGAETVTPVLDPARRQGYGPLWYTSVGFGGLVQLRRGERVYVNISHPDMVDFARGKTFFGAVMVG

SEQ ID NO: 105 - protein, 918 aa, artificial sequence
  349B4 humanized anti-B7-H4/hLT alpha-beta-beta mature chain mature protein
QVQLVQSGAEVKKPGASVKVSCKASGYTFTSYYMHWVRQAPGQRLEWMGYVDPFNGGTSYNQKFKGRVTITVDTSSSTAYMELSSLRSEDTAVYYCAFIAGFANWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREELTKNQVSLTCLVKGFYPSDIAVEWESNGQPENNYKTTPPVLDSDGSFFLYSKLTVDKSRWQQGNVFSCSVMHEALHNHYTQKSLSLSPGKAHSTLKPAAHLIGDPSKQNSLLWRANTDRAFLQDGFSLSNNSLLVPTSGIYFVYSQVVFSGKAYSPKATSSPLYLAHEVQLFSSQYPFHVPLLSSQKMVYPGLQEPWLHSMYHGAAFQLTQGDQLSTHTDGIPHLVLSPSTVFFGAFALLSPGLPAAHLIGAPLKGQGLGWETTKEQAFLTSGTQFSDAEGLALPQDGLYYLYCLVGYRGRAPPGGGDPQGRSVTLRSSLYRAGGAYGPGTPELLLEGAETVTPVLDPARRQGYGPLWYTSVGFGGLVQLRRGERVYVNISHPDMVDFARGKTFFGAVMVGLSPGLPAAHLIGAPLKGQGLGWETTKEQAFLTSGTQFSDAEGLALPQDGLYYLYCLVGYRGRAPPGGGDPQGRSVTLRSSLYRAGGAYGPGTPELLLEGAETVTPVLDPARRQGYGPLWYTSVGFGGLVQLRRGERVYVNISHPDMVDFARGKTFFGAVMVG

SEQ ID NO: 106 - protein, 234 aa, artificial sequence; identical to SEQ ID NO: 59
  349B4 humanized anti-B7-H4/hLT alpha-beta-beta light chain precursor protein (signal sequence underlined)

SEQ ID NO: 107 - protein, 214 aa, artificial sequence; identical to SEQ ID NO: 60
  349B4 humanized anti-B7-H4/hLT alpha-beta-beta light chain mature protein

SEQ ID NO: 108 - protein, 157 aa, artificial sequence
  Minimal LT Beta sequence (aa 87-243)
LPAAHLIGAPLKGQGLGWETTKEQAFLTSGTQFSDAEGLALPQDGLYYLYCLVGYRGRAPPGGGDPQGRSVTLRSSLYRAGGAYGPGTPELLLEGAETVTPVLDPARRQGYGPLWYTSVGFGGLVQLRRGERVYVNISHPDMVDFARGKTFFGAVMV

(19) United States
(12) Patent Application Publication
(10) Pub. No.: US 2018/0222958 A1
(43) Pub. Date: Aug. 9, 2018

(54) LYMPHOTOXIN-BETA RECEPTOR-BINDING AGENTS, TARGETING ANTIBODIES, AND USES THEREOF

(71) Applicant: OncoMed Pharmaceuticals, Inc., Redwood City, CA (US)

(72) Inventors: Austin L. GURNEY, San Francisco, CA (US); Inkyung Park, Palo Alto, CA (US); Fumiko Takada Axelrod, Palo Alto, CA (US)

(73) Assignee: OncoMed Pharmaceuticals, Inc.

(21) Appl. No.: 15/849,368

(22) Filed: Dec. 20, 2017

Related U.S. Application Data
(60) Provisional application No. 62/455,398, filed on Feb. 6, 2017.
(60) Provisional application No. 62/436,616, filed on Dec. 20, 2016.

(51) Int. Cl. C07K 14/525; C07K 16/28; A61K 38/19; A61P 35/00; A61K 45/06

(52) U.S. Cl. CPC C07K 14/5255; A61K 2039/505; C07K 16/28; A61K 38/191; A61P 35/00; A61K 45/06; C07K 2319/74; C07K 2317/622; C07K 2317/55; C07K 2317/565; C07K 2317/56; C07K 2317/21; C07K 2317/52; C07K 2319/30; C07K 16/2827

(57) ABSTRACT

Polypeptides, agents, and molecules that bind lymphotoxin-beta receptor (LTβR) and/or tumor-associated antigens are disclosed. The polypeptides, agents, or molecules may include, without limitation, fusion or single-chain lymphotoxin-αββ polypeptides and homodimer and heterodimer molecules comprising the lymphotoxin-αββ polypeptides. Antibodies that specifically bind B7-H4 and P-CADHERIN are also disclosed. Also disclosed are methods of using the polypeptides, agents, molecules, or antibodies for inducing and/or enhancing the immune response, as well as methods for the treatment of diseases such as cancer.

### CROSS-REFERENCE TO RELATED APPLICATIONS

This application claims the priority benefit of U.S. Provisional Application No. 62/455,398, filed Feb. 6, 2017, and U.S. Provisional Application No. 62/436,616, filed Dec. 20, 2016, each of which is hereby incorporated by reference herein in its entirety.

### FIELD OF THE INVENTION

The present invention generally relates to polypeptides, or agents or molecules comprising the polypeptides, that bind lymphotoxin-beta receptor and/or that bind tumor-associated antigens, including, without limitation, antibodies that bind B7-H4 or P-CADHERIN (CDH3). The invention also relates to methods of using the polypeptides, agents, molecules and antibodies for the modulation of immune responses and/or the treatment of diseases such as cancer.

### BACKGROUND OF THE INVENTION

The basis for immunotherapy is the manipulation and/or modulation of the immune system, including both innate immune responses and adaptive immune responses. The general aim of immunotherapy is to treat diseases by controlling the immune response to a “foreign agent,” for example a pathogen or a tumor cell. However, in some instances immunotherapy is used to treat autoimmune diseases which may arise from an abnormal immune response against proteins, molecules, and/or tissues normally present in the body. Immunotherapy may include methods to induce or enhance specific immune responses or to inhibit or reduce specific immune responses.

The immune system is a highly complex system made up of a great number of cell types, including but not limited to, T-cells, B-cells, natural killer cells, antigen-presenting cells, dendritic cells, monocytes, and macrophages. These cells possess complex and subtle systems for controlling their interactions and responses. The cells utilize both activating and inhibitory mechanisms and feedback loops to keep responses in check and not allow negative consequences of an uncontrolled immune response (e.g., autoimmune diseases or a cytokine storm).

The concept of cancer immunosurveillance is based on the theory that the immune system can recognize tumor cells, mount an immune response, and suppress the development and/or progression of a tumor. However, it is clear that many cancerous cells have developed mechanisms to evade the immune system which can allow for uninhibited growth of tumors. Cancer/tumor immunotherapy (immuno-oncology) focuses on the development of new and novel agents that can activate and/or boost the immune system to achieve a more effective attack against tumor cells resulting in the increased killing of tumor cells and/or inhibition of tumor growth.

### BRIEF SUMMARY OF THE INVENTION

The presence or absence of lymphocytes within a tumor (often referred to as “tumor infiltrating lymphocytes” or TILs) is believed to be a strong prognostic factor for patient survival and patient response to immunotherapeutic strategies. Given the strong correlation with patient benefit, it is important to identify strategies to increase the infiltration of TILs into tumors. Migration of leukocyte populations is directed by members of the chemokine family of cytokines. Gradients of specific chemokines enable chemotaxis of specific leukocyte populations. The generation of the adaptive immune response, in which T-cells and B-cells play central roles, may also be regulated by chemokine gradients. An important mechanism by which the adaptive response refines its response to specific antigens involves the creation of a germinal center, in which various immune cells types including dendritic cells, T-cells, and B-cells orchestrate the adaptive immune response. Germinal centers can be formed in the spleen, the lymph nodes, and other secondary lymphoid tissues, as well as in peripheral tissues. When formed in peripheral tissues, germinal centers are often referred to as “tertiary lymphoid structures.” It is noteworthy that the presence of tertiary lymphoid structures is sometimes observed within tumors and that the presence of such tertiary lymphoid structures has been strongly associated with favorable outcome. The inventors believe that a therapeutic strategy that promotes the formation of tertiary lymphoid structures within tumors might enable patient benefit, both by specifically facilitating the generation of an adaptive immune response against the tumor and by more generally eliciting the recruitment of TILs which might additionally enhance the impact of other immunotherapeutic agents.

Lymphotoxin-alpha/beta/beta is a central factor involved in the initiation of the germinal center formation. Lymphotoxin-αββ is a heterotrimeric species comprised of one subunit or copy of lymphotoxin-alpha and two subunits or copies of lymphotoxin-beta. Lymphotoxin-αββ binds to the lymphotoxin-beta receptor (LTβR). The activation of LTβR initiates a signaling event resulting in the expression of chemokines, including but not limited to, CXCL12, CXCL13, CCL19, and CCL21. These chemokines serve to induce the migration of dendritic cells, T-cells, and B-cells to establish the germinal center. The central role of lymphotoxin-αββ in the organization of lymphoid tissue is highlighted by the phenotype of mice deficient in either lymphotoxin-alpha or lymphotoxin-beta. These mice do not develop peripheral lymph nodes, Peyer's patches, or splenic germinal centers. A second ligand also exists for LTβR, termed LIGHT. LIGHT is also a member of the TNF family. In addition to binding LTβR, LIGHT also binds to a separate receptor, herpesvirus entry mediator (HVEM), as well as secreted decoy receptor DcR3. In contrast to the phenotypes of mice deficient in either lymphotoxin-alpha or lymphotoxin-beta, mice deficient in LIGHT have a normal complement of lymphoid organs. The binding of LIGHT to HVEM also contributes to a distinct biology involving co-stimulation of T-cells. It is noteworthy that constitutive expression of LIGHT leads to tissue destruction and autoimmune-like disease syndromes.

In order to increase the immune response to tumors, we sought to leverage the potential for lymphotoxin-αββ, which acts as a central factor involved in the initiation of germinal center formation, to provide an anti-tumor effect. To this end, we have developed means to deliver lymphotoxin-αββ to the tumor and/or the tumor microenvironment. In particular, one strategy to enable this is through the use of a bispecific agent wherein one component of the agent is selective for a tumor-associated antigen (TAA), and thereby directs the second component, lymphotoxin-αββ, to the tumor.

In certain aspects, the present invention, therefore, provides a variety of polypeptides, agents, and molecules that bind and/or activate human lymphotoxin-beta receptor (LTβR). In some embodiments, the polypeptides, agents, and molecules do not bind and/or do not activate HVEM. As used herein, the term “molecule” includes, but is not limited to, polypeptides, fusion proteins, homodimeric molecules, heterodimeric molecules, and other LTβR-binding agents. In certain embodiments, the polypeptide, agent, or molecule is an LTβR agonist. In some embodiments, the polypeptide or molecule that binds LTβR is a soluble polypeptide comprising a lymphotoxin-αββ heterotrimer. The invention provides methods of using the polypeptides and molecules described herein. In some embodiments, the invention provides methods of using the polypeptides and molecules for cancer immunotherapy or immuno-oncology. In some embodiments, the polypeptides and molecules are used in methods of inducing, activating, promoting, increasing, enhancing, or prolonging an immune response. In some embodiments, the polypeptides and molecules are used in methods of inducing, activating, promoting, increasing, enhancing, or prolonging an immune response to cancer, a tumor, and/or tumor cells. In some embodiments, the polypeptides and molecules are used in methods of inhibiting the growth of a tumor or tumor cells. In some embodiments, the polypeptides and molecules are used in methods for the treatment of cancer. In some embodiments, the methods comprise inhibiting the growth of cancer cells. The invention also provides compositions comprising the polypeptides and molecules described herein. In some embodiments, the compositions are pharmaceutical compositions comprising the polypeptides and molecules described herein. Polynucleotides encoding the polypeptides and molecules and methods of making the polypeptides and molecules are also provided.

In one aspect, the invention features a fusion polypeptide comprising (a) a first copy and a second copy of the extracellular domain of human lymphotoxin-beta or a fragment thereof, and (b) a copy of human lymphotoxin-alpha or a fragment thereof, where the fusion polypeptide is capable of binding the human lymphotoxin-beta receptor. The copies of lymphotoxin-beta and lymphotoxin-alpha may be directly linked to each other, or the copies of lymphotoxin-beta and lymphotoxin-alpha may be linked to each other with a peptide linker. In some embodiments, the polypeptide may further comprise a targeting moiety.

In another aspect, the invention features a single-chain polypeptide comprising (a) a first amino acid sequence comprising the extracellular domain of human lymphotoxin-beta, a variant thereof having at least 70%, 80%, 85%, 90%, 95%, 98%, or 99% sequence identity (e.g., 80%, 90%, or 95%) to the extracellular domain of human lymphotoxin-beta, or a fragment thereof; (b) a second amino acid sequence comprising the extracellular domain of human lymphotoxin-beta, a variant thereof having at least 70%, 80%, 85%, 90%, 95%, 98%, or 99% sequence identity (e.g., 80%, 90%, or 95%) to the extracellular domain of human lymphotoxin-beta, or a fragment thereof; and (c) a third amino acid sequence comprising human lymphotoxin-alpha, a variant thereof having at least 70%, 80%, 85%, 90%, 95%, 98%, or 99% sequence identity (e.g., 80%, 90%, or 95%) to human lymphotoxin-alpha, or a fragment thereof; wherein the polypeptide is capable of binding the human lymphotoxin-beta receptor, and the amino acid sequences are directly linked through a peptide bond. The polypeptide may, in certain embodiments, further comprise a targeting moiety. In certain embodiments, the targeting moiety is an antibody (e.g., an antibody that binds a tumor-associated antigen such as B7-H4 or P-CADHERIN).

In a further aspect, the invention provides a single-chain polypeptide comprising: (a) a first amino acid sequence comprising a sequence having at least about 90%, at least about 95%, or at least about 98% sequence identity to SEQ ID NO: 15 or SEQ ID NO: 108 (fragments of human lymphotoxin-beta); (b) a second amino acid sequence comprising a sequence having at least about 90%, at least about 95%, or at least about 98% sequence identity to SEQ ID NO: 15 or SEQ ID NO: 108; and (c) a third amino acid sequence comprising a sequence having at least about 90%, at least about 95%, or at least about 98% sequence identity to SEQ ID NO: 12 (a fragment of human lymphotoxin-alpha); wherein the polypeptide is capable of binding the human lymphotoxin-beta receptor. In some embodiments, the first amino acid sequence comprises a sequence having at least about 90% sequence identity to SEQ ID NO: 15 and the second amino acid sequence comprises a sequence having at least about 90% sequence identity to SEQ ID NO: 15. In certain embodiments, the amino acid sequences are directly linked through a peptide bond.

In certain embodiments of each of the aforementioned aspects, the polypeptide may be structured sequentially (from N to C terminal) as (a) lymphotoxin-alpha-lymphotoxin-beta-lymphotoxin-beta; (b) lymphotoxin-beta-lymphotoxin-alpha-lymphotoxin-beta; or (c) lymphotoxin-beta-lymphotoxin-beta-lymphotoxin-alpha. In some embodiments, the first copy or amino acid sequence and the second copy or amino acid sequence of human lymphotoxin-beta each comprise SEQ ID NO: 14 or a fragment thereof, and the copy or amino acid sequence of human lymphotoxin-alpha comprises SEQ ID NO: 11 or a fragment thereof. In some embodiments, the first copy or amino acid sequence and the second copy or amino acid sequence of human lymphotoxin-beta may each comprise amino acids 83-244 of SEQ ID NO: 13, and the copy or amino acid sequence of human lymphotoxin-alpha may comprise amino acids 62-205 of SEQ ID NO: 10. In some embodiments, the first copy or amino acid sequence and the second copy or amino acid sequence of human lymphotoxin-beta may each comprise SEQ ID NO: 15, and the copy or the amino acid sequence of human lymphotoxin-alpha may comprise SEQ ID NO: 12. In some embodiments, the polypeptide of comprises SEQ ID NO: 16, SEQ ID NO: 17, or SEQ ID NO: 18. The polypeptide may further comprise a targeting moiety (e.g., an antibody that specifically binds B7-H4 or P-CADHERIN).

In another aspect, the invention provides a single-chain polypeptide capable of binding human LTβR, wherein the single-chain polypeptide comprises a polypeptide having at least about 90%, at least about 95%, or at least about 98% sequence identity to SEQ ID NO: 16, SEQ ID NO: 17, or SEQ ID NO: 18. In certain embodiments, the polypeptide may be linked to a targeting moiety.

In another aspect, the invention features a poly peptide comprising (a) the polypeptide of any of the aforementioned aspects; and (b) a targeting moiety. In another aspect, the invention features an agent comprising (a) the polypeptide of any of the aforementioned aspects; and (b) a targeting moiety linked to the polypeptide. In some embodiments, the polypeptide (a) and targeting moiety (b) are linked by a peptide bond or by a linker peptide. In certain embodiments, the targeting moiety is an antibody (e.g., an antibody that binds a tumor-associated antigen such as B7-H4 or P-CADHERIN). In some embodiments, the targeting moiety is an antigen-binding fragment of an antibody (or a functional antigen-binding site from an antibody).

In a further aspect, the invention provides an agent comprising (a) a heterotrimer that is capable of binding the human lymphotoxin-beta receptor and (b) a targeting moiety linked to the heterotrimer. In certain embodiments, the heterotrimer is a lymphotoxin αββ heterotrimer. In certain embodiments, the heterotrimer comprises (i) a first amino acid sequence comprising a sequence having at least about 90%, at least about 95%, or at least about 98% sequence identity to SEQ ID NO: 15 or SEQ ID NO: 108; (ii) a second amino acid sequence comprising a sequence having at least about 90% sequence identity to SEQ ID NO:15 or SEQ ID NO: 108; and (iii) a third amino acid sequence comprising a sequence having at least about 90% sequence identity to SEQ ID NO: 12. In certain embodiments, the first and second amino acid sequences of the heterotrimer each comprise SEQ ID NO: 15 and the second amino acid sequence of the heterotrimer comprises SEQ ID NO: 12. In certain embodiments, the heterotrimer comprises SEQ ID NO: 16, SEQ ID NO: 17, or SEQ ID NO: 18. In certain embodiments, the heterotrimer may be a single-chain polypeptide.

In another aspect, the invention features an agent comprising (a) a heterotrimer comprising (i) a first amino acid sequence comprising the extracellular domain of human lymphotoxin-beta, a variant thereof having at least 70%, 80%, 85%, 90%, 95%, 98%, or 99% sequence identity (e.g., 80%, 90%, or 95%) to the extracellular domain of human lymphotoxin-beta, or a fragment thereof; (ii) a second amino acid sequence comprising the extracellular domain of lymphotoxin-beta, a variant thereof having at least 70%, 80%, 85%, 90%, 95%, 98%, or 99% sequence identity (e.g., 80%, 90%, or 95%) to the extracellular domain of human lymphotoxin-beta, or a fragment thereof; and (iii) a third amino acid sequence comprising lymphotoxin-alpha, a variant thereof having at least 70%, 80%, 85%, 90%, 95%, 98%, or 99% sequence identity (e.g., 80%, 90%, or 95%) to human lymphotoxin-alpha, or a fragment thereof; where the heterotrimer is capable of binding the human lymphotoxin-beta receptor; and (b) a targeting moiety linked to the heterotrimer. In some embodiments, the heterotrimer is a single-chain polypeptide.

In some embodiments, the polypeptides or agents described above may comprise a targeting moiety capable of binding a target cell (e.g., a tumor cell). In certain embodiments, the polypeptide or agents are capable of binding an antigen on the surface of the cell (e.g., a tumor-associated antigen).

In some embodiments, the polypeptide or agent described above may comprise a targeting moiety that comprises a non-lymphotoxin polypeptide. The polypeptide or agent may be directly linked to the non-lymphotoxin polypeptide or may be connected to the non-lymphotoxin polypeptide by a linker. In certain embodiments, the N-terminal end of the polypeptide described above is linked to the C-terminal end of the non-lymphotoxin polypeptide. In certain embodiments, the C-terminal end of the polypeptide described above is linked to the N-terminal end of the non-lymphotoxin polypeptide. In some embodiments, the non-lymphotoxin polypeptide comprises an immunoglobulin heavy chain (e.g., IgG1, IgG2, IgG3, and IgG4). The immunoglobulin heavy chain may be mutated at a glycosylation site (e.g., any described herein). The immunoglobulin heavy chain may be associated with an immunoglobulin light chain, for example, where the immunoglobulin heavy chain and immunoglobulin light chain form an antigen-binding site or an antibody (e.g., a monoclonal antibody), for example, an antigen-binding site or antibody that binds a tumor-associated antigen (e.g., any described herein). The non-lymphotoxin polypeptide may comprise a single-chain antibody or a Fab, for example, a single-chain antibody or Fab that binds a tumor-associated antigen (e.g., any described herein). The tumor-associated antigen may be selected from the group consisting of B7-H4, P-CADHERIN (CDH3), GABRP, ACPP, SLC45A3, STEAP1, STEAP2, GPA33, GUCY2C, GARP, B7-H3, PVRL4, mesothelin and CA9. In a particular embodiment, the tumor-associated antigen is B7-H4. In a particular embodiment, the tumor-associated antigen is CDH3 (P-CADHERIN).

In some embodiments, any of the polypeptides or agents described above may comprise a non-lymphotoxin polypeptide, e.g., directly linked to the non-lymphotoxin polypeptide or connected to the non-lymphotoxin polypeptide by a linker. The N-terminal end of the polypeptide described above may be linked to the C-terminal end of the non-lymphotoxin polypeptide, or the C-terminal end of the polypeptide described above may be linked to the N-terminal end of the non-lymphotoxin polypeptide. The non-lymphotoxin polypeptide may comprise a human Fc region (e.g., an Fc region from an IgG1, IgG2, IgG3, or IgG4 immunoglobulin). The human Fc region may be mutated at the glycosylation site or may contain a mutation that allows heterodimer formation (e.g., any described herein). The human Fc region may be selected from the group consisting of SEQ ID NO:27, SEQ ID NO:28, SEQ ID NO:29, SEQ ID NO:30, SEQ ID NO:31. SEQ ID NO:32. SEQ ID NO:33, SEQ ID NO:34, SEQ ID NO:38, SEQ ID NO:39, SEQ ID NO:40, and SEQ ID NO:41.

In another aspect, the invention features a homodimeric molecule, where each monomer comprises a polypeptide or agent described above. In some embodiments, the homodimeric molecule includes monomers that each comprise (a) a first polypeptide comprising a first copy and a second copy of the extracellular domain of human lymphotoxin-beta or a fragment thereof, and a copy of human lymphotoxin-alpha or a fragment thereof; and (b) a second polypeptide comprising a targeting moiety, e.g., that is capable of binding a target cell, such as a tumor cell. The targeting moiety may be an antibody (e.g., an IgG1, IgG2, IgG3, or IgG4 antibody). The antibody may be mutated at a glycosylation site within the immunoglobulin heavy chain (e.g., any described herein). The second polypeptide may comprise an antibody that specifically binds a tumor-associated antigen, for example, any described herein, such as B7-H4, P-CADHERIN (CDH3), GABRP, ACPP, SLC45A3, STEAP1, STEAP2, GPA33, GUCY2C, GARP, B7-H3, PVRL4, mesothelin or CA9. In one embodiment, the tumor-associated antigen is B7-H4. In one embodiment, the tumor-associated antigen is P-CADHERIN (CDH3). The first polypeptide of each monomer may be structured sequentially (from N to C terminal) as (a) lymphotoxin-alpha-lymphotoxin-beta-lymphotoxin-beta; (b) lymphotoxin-beta-lymphotoxin-alpha-lymphotoxin-beta; or (c) lymphotoxin-beta-lymphotoxin-beta-lymphotoxin-alpha. The copies of lymphotoxin-beta and lymphotoxin-alpha may be directly linked to each other, or the copies of lymphotoxin-beta and lymphotoxin-alpha may be linked to each other with a peptide linker. The first copy and the second copy of human lymphotoxin-beta each may comprise SEQ ID NO: 14 or a fragment thereof, and/or the copy of human lymphotoxin-alpha may comprise SEQ ID NO: 11 or a fragment thereof. The first copy and the second copy of human lymphotoxin-beta may each comprise amino acids 83-244 of SEQ ID NO: 13, and the copy of human lymphotoxin-alpha may comprises amino acids 62-205 of SEQ ID NO: 10. The first copy and the second copy of human lymphotoxin-beta may each comprise SEQ ID NO: 15, and the copy of human lymphotoxin-alpha may comprise SEQ ID NO: 12. Each monomer may comprise SEQ ID NO: 16, SEQ ID NO: 17, or SEQ ID NO: 18. The first polypeptide may be directly linked to the second polypeptide, or the first polypeptide may be connected to the second polypeptide by a linker. For example, the N-terminal end of the first polypeptide may be linked to the C-terminal end of the second polypeptide.

In another aspect, the invention features a heterodimeric molecule comprising (a) first monomer comprising a polypeptide described above; and (b) a second monomer comprising a targeting moiety. The invention also features a heterodimeric molecule, where a first monomer comprises a first copy and a second copy of the extracellular domain of human lymphotoxin-beta or a fragment thereof, and a copy of human lymphotoxin-alpha or a fragment thereof; and a second monomer comprises a targeting moiety. The targeting moiety may be capable of binding a target cell (e.g., a tumor cell). The targeting moiety may comprise an antigen-binding site or may be an antibody. The second monomer may comprise an antigen-binding site or antibody that specifically binds a tumor-associated antigen (e.g., B7-H4, P-CADHERIN (CDH3), GABRP, ACPP, SLC45A3. STEAP1, STEAP2, GPA33, GUCY2C. GARP, B7-H3, PVRL4, mesothelin or CA9). In one embodiment, the tumor-associated antigen is B7-H4. In one embodiment, the tumor-associated antigen is P-CADHERIN (CDH3). The first copy and the second copy of human lymphotoxin-beta may each comprise SEQ ID NO: 14 or a fragment thereof, and the copy of human lymphotoxin-alpha may comprise SEQ ID NO: 11 or a fragment thereof. The first copy and the second copy of human lymphotoxin-beta may each comprise amino acids 83-244 of SEQ ID NO: 13, and the copy of human lymphotoxin-alpha may comprises amino acids 62-205 of SEQ ID NO: 10. The first copy and the second copy of human lymphotoxin-beta may each comprise SEQ ID NO: 15, and the copy of human lymphotoxin-alpha may comprise SEQ ID NO: 12. The first monomer may comprise SEQ ID NO: 16, SEQ ID NO: 17, or SEQ ID NO: 18. In some embodiments, the first monomer further comprises a human Fc region (e.g., a human Fc region from an IgG1, IgG2. IgG3, or IgG4 immunoglobulin). In particular embodiments, the human Fc region is mutated or contains a mutation at the glycosylation site and/or is mutated or contains a mutation to allow heterodimer formation. The human Fc region may be selected from the group consisting of: SEQ ID NO:27, SEQ ID NO:28. SEQ ID NO:29, SEQ ID NO:30, SEQ ID NO:31, SEQ ID NO:32, SEQ ID NO:33, SEQ ID NO:34, SEQ ID NO:38, SEQ ID NO:39, SEQ ID NO:40, and SEQ ID NO:41. In other embodiments, the second monomer comprises an immunoglobulin heavy chain (e.g., an IgG1, IgG2, IgG3, or IgG4 heavy chain). The immunoglobulin heavy chain may be mutated or may contain a mutation at the glycosylation site. The immunoglobulin heavy chain may be mutated or may contain mutation to allow heterodimer formation. In some embodiments, the immunoglobulin heavy chain is associated with an immunoglobulin light chain.

In another aspect, the invention provides an agent comprising (a) an antibody that specifically binds a tumor-associated antigen; and (b) an LTβR-binding moiety, wherein the LTβR-binding moiety is linked to the antibody. In certain embodiments, the antibody specifically binds B7-H4, PP-CADHERIN (CDH3), GABRP, ACPP, SLC45A3. STEAP1, STEAP2, GPA33, GUCY2C, GARP, B7-H3, PVRL4, mesothelin and CA9. In some embodiments, the antibody specifically binds human B7-H4. In certain other embodiments, the antibody specifically binds human P-CADHERIN. In certain embodiments, the antibody is a full-length antibody. In certain other embodiments, the antibody is an antigen-binding antibody fragment. In certain embodiments, the LTβR-binding moiety comprises a lymphotoxin app heterotrimer. In certain alternative embodiments, the LTβR-binding moiety comprises a LIGHT homotrimer or an antibody that specifically binds LTβR (e.g., human LTβR).

In still another aspect, the invention provides an agent comprising (a) an antibody that specifically binds a cell-surface antigen (e.g., a tumor-associated antigen); and (b) a single-chain LTβR-binding moiety, wherein the LTβR-binding moiety is linked to the antibody. In certain embodiments, the LTβR-binding moiety is a single-chain lymphotoxin αββ heterotrimer. In certain embodiments, the LTβR-binding moiety is a single-chain polypeptide or fusion polypeptide described herein that binds human LTβR.

Any of the polypeptides, agents, homodimeric molecules, and heterodimeric molecules described above may activate the human lymphotoxin-beta receptor.

Any of the polypeptides, agents, homodimeric molecules, and heterodimeric molecules described above may induce human lymphotoxin-beta receptor signaling.

In any of the above aspects or embodiments where the tumor-associated antigen to be targeted is B7-H4, the targeting moiety, antigen-binding site, antibody, single-chain antibody, or Fab may bind B7-H4 and comprise (a) a heavy chain CDR1 comprising TSYYMH (SEQ ID NO:42), a heavy chain CDR2 comprising YVDPFNGGTSYNQKFKG (SEQ ID NO:43), and a heavy chain CDR3 comprising FIAGFAN (SEQ ID NO:44) or IAGFAN (SEQ ID NO:45); and (b) a light chain CDR1 comprising KASQDIKSYLS (SEQ ID NO:46), a light chain CDR2 comprising YATSLAD (SEQ ID NO:47), and a light chain CDR3 comprising LQHGESPYT (SEQ ID NO:48) or LQHGESPY (SEQ ID NO:49) for example, comprising (a) a heavy chain variable region comprising SEQ ID NO:50 and (b) a light chain variable region comprising SEQ ID NO:51. The antibody may be an antibody with CDRs and/or variable chain regions from the heavy chain sequence of SEQ ID NO:54 and/or the light chain sequence of SEQ ID NO:55. e.g., a humanized version thereof. An another non-limiting example, the targeting moiety, antigen-binding site, antibody, single-chain antibody, or Fab that binds B7-H4 may comprise (a) a heavy chain variable region having at least about 90% sequence identity to SEQ ID NO:66, and/or (b) a light chain variable region having at least about 90% sequence identity to SEQ ID NO:62. In certain embodiments, the targeting moiety, antigen-binding site, antibody, single-chain antibody, or Fab comprises (a) a heavy chain variable region comprising SEQ ID NO:66 and (b) a light chain variable region comprising SEQ ID NO:62.

In any of the above aspects or embodiments where the tumor-associated antigen is P-CADHERIN, the targeting moiety, antigen-binding site, antibody, single-chain antibody, or Fab may bind P-CADHERIN and comprise (a) a heavy chain CDR1 comprising STYGMS (SEQ ID NO:80), a heavy chain CDR2 comprising ATISDGGSYTYYPDSVKGR (SEQ ID NO:81), and a heavy chain CDR3 comprising ARHYYGSDWYFDV (SEQ ID NO:82); and (b) a light chain CDR1 comprising RSSQSIVQSNGNTYLE (SEQ ID NO:73), a light chain CDR2 comprising KVSNQFS (SEQ ID NO:74), and a light chain CDR3 comprising QGSHVPL (SEQ ID NO:75). In certain embodiments, the targeting moiety, antigen-binding site, antibody, single-chain antibody, or Fab comprises (a) a heavy chain variable region having at least about 90% sequence identity to SEQ ID NO:79; and/or (b) a light chain variable region having at least about 90% sequence identity to SEQ ID NO:72 or SEQ ID NO:93. In some embodiments, the targeting moiety, antigen-binding site, antibody, single-chain antibody, or Fab comprises (a) a heavy chain variable region comprising SEQ ID NO:79 and (b) a light chain variable region comprising SEQ ID NO:72 or SEQ ID NO:93.

In another aspect, the invention features a method of activating or enhancing LTβR signaling in a cell, comprising contacting the cell with an effective amount of the polypeptide, agent, or molecule described above.

In another aspect, the invention features a method of inducing, activating, promoting, increasing, enhancing, or prolonging an immune response in a subject, comprising administering a therapeutically effective amount of any of the polypeptides, agents, homodimeric molecules, and heterodimeric molecules described above. The immune response may be against a tumor or cancer.

In still another aspect, the invention features a method of inhibiting the growth of a tumor, comprising contacting a tumor or tumor cell with an effective amount of any of the polypeptides, agents, homodimeric molecules, and heterodimeric molecules described above.

In another aspect, the invention features a method of inhibiting the growth of a tumor in a subject, comprising administering to the subject a therapeutically effective amount of any of the polypeptides, agents, homodimeric molecules, and heterodimeric molecules described above.

In yet another aspect, a method of increasing the responsiveness of a tumor to treatment with a second therapeutic agent is provided. The method comprises administering to the subject with the tumor a therapeutically effective amount of the polypeptide, agent, molecule, or antibody described above or elsewhere herein. In certain embodiments, the second therapeutic agent is an immunotherapeutic agent, such as a checkpoint inhibitor.

In the methods described above, the tumor may be selected from the group consisting of colorectal tumor, colon tumor, ovarian tumor, pancreatic tumor, lung tumor, liver tumor, breast tumor, kidney tumor, prostate tumor, gastrointestinal tumor, melanoma, cervical tumor, bladder tumor, glioblastoma, and head and neck tumor.

In another aspect, the invention features a method of treating cancer in a subject, comprising administering to the subject a therapeutically effective amount of any of the polypeptides, agents, homodimeric molecules, and heterodimeric molecules described above. Cancer may be selected from the group consisting of colorectal cancer, colon cancer, ovarian cancer, pancreatic cancer, lung cancer, liver cancer, breast cancer, kidney cancer, prostate cancer, gastrointestinal cancer, melanoma, cervical cancer, bladder cancer, glioblastoma, head and neck cancer, lymphoma and leukemia.

In another aspect, the invention features a method of increasing T-cell activity in a subject, comprising administering to the subject a therapeutically effective amount of any of the polypeptides, agents, homodimeric molecules, and heterodimeric molecules described above.

In another aspect, the invention features a method of recruiting tumor-infiltrating lymphocytes to a tumor in a subject, comprising administering to the subject a therapeutically effective amount of any of the polypeptides, agents, homodimeric molecules, and heterodimeric molecules described above. The tumor may be selected from the group consisting of colorectal tumor, colon tumor, ovarian tumor, pancreatic tumor, lung tumor, liver tumor, breast tumor, kidney tumor, prostate tumor, gastrointestinal tumor, melanoma, cervical tumor, bladder tumor, glioblastoma, and head and neck tumor.

A method of promoting the formation of lymphoid structures within a tumor or tumor microenvironment, comprising administering to the subject a therapeutically effective amount of any of the polypeptides, agents, homodimeric molecules, and heterodimeric molecules described above. The tumor may be selected from the group consisting of colorectal tumor, colon tumor, ovarian tumor, pancreatic tumor, lung tumor, liver tumor, breast tumor, kidney tumor, prostate tumor, gastrointestinal tumor, melanoma, cervical tumor, bladder tumor, glioblastoma, and head and neck tumor.

A method of increasing cytolytic T-cell (CTL) activity in a subject, comprising administering to the subject a therapeutically effective amount of any of the polypeptides, agents, homodimeric molecules, and heterodimeric molecules described above.

In another aspect, the invention features a pharmaceutical composition comprising (a) any of the polypeptides, agents, homodimeric molecules, or heterodimeric molecules described above; and (b) a pharmaceutically acceptable carrier.

In certain other aspects, the invention provides novel antibodies, which, in certain embodiments, are useful for targeting tumors. Thus, in one aspect, the invention provides antibodies that specifically bind B7-H4. In some embodiments, the antibody that specifically binds B7-H4 comprises: (a) a heavy chain CDR1 comprising TSYYMH (SEQ ID NO:42), a heavy chain CDR2 comprising YVDPFNGGTSYNQKFKG (SEQ ID NO:43), and a heavy chain CDR3 comprising FIAGFAN (SEQ ID NO:44) or IAGFAN (SEQ ID NO:45); and (b) a light chain CDR1 comprising KASQDIKSYLS (SEQ ID NO:46), a light chain CDR2 comprising YATSLAD (SEQ ID NO:47), and a light chain CDR3 comprising LQHGESPYT (SEQ ID NO:48) or LQHGESPY (SEQ ID NO:49). The antibody may comprise (a) a heavy chain variable region having at least 90% sequence identity to SEQ ID NO:50; and (b) a light chain variable region having at least 90% sequence identity to SEQ ID NO:51. The antibody may comprise (a) a heavy chain variable region having at least 95% sequence identity to SEQ ID NO:50; and (b) a light chain variable region having at least 95% sequence identity to SEQ ID NO:51. The antibody may comprise a heavy chain variable region comprising SEQ ID NO:50 and a light chain variable region comprising SEQ ID NO:51. The antibody may be an antibody with CDRs and/or variable chain regions from the heavy chain sequence of SEQ ID NO:54 and/or the light chain sequence of SEQ ID NO:55, e.g., a humanized version thereof. The antibody may comprise (a) a heavy chain variable region having at least about 90%, at least about 95%, or at least about 98% sequence identity to SEQ ID NO:66, and (b) a light chain variable region having at least about 90%, at least about 95%, or at least about 98% sequence identity to SEQ ID NO:62. In certain embodiments, the antibody comprises (a) a heavy chain variable region comprising SEQ ID NO:66 and (b) a light chain variable region comprising SEQ ID NO:62. In certain embodiments, the antibody is linked to an LTβR-binding moiety or a polypeptide that binds human LTβR.

In another aspect, the invention features antibodies that compete with each of the above-indicated antibodies for binding to human B7-H4. In a further aspect, an antibody that binds the same epitope, or substantially the same epitope, on B7-H4 as any of the above-indicated B7-H4 antibodies is also provided. In a still further aspect, the invention provides an antibody that binds an epitope on B7-H4 that overlaps with the epitope of any of the other B7-H4 antibodies described herein.

In certain embodiments, the antibodies that specifically bind B7-H4 may be useful for targeting a tumor, e.g., a breast or ovarian tumor, in a subject. Other methods of use of the B7-H4 antibodies are also provided. For instance, the invention features a method of inhibiting the growth of a tumor (e.g., a breast or ovarian tumor) in a subject, comprising administering a therapeutically effective amount of a B7-H4 antibody described herein (or a polypeptide, agent or molecule comprising such antibody) to the subject. Methods of treating cancer (e.g., breast or ovarian cancer) in a subject comprising administering a therapeutically effective amount of a B7-H4 antibody described herein (or a polypeptide, agent or molecule comprising such antibody) are also provided.

In another aspect, the invention provides antibodies that specifically bind P-CADHERIN (CDH3). In certain embodiments, the antibody that specifically binds P-CADHERIN comprises (a) a heavy chain CDR1 comprising STYGMS (SEQ ID NO:80), a heavy chain CDR2 comprising ATISDGGSYTYYPDSVKGR (SEQ ID NO:81), and a heavy chain CDR3 comprising ARHYYGSDWYFDV (SEQ ID NO:82); and (b) a light chain CDR1 comprising RSSQSIVQSNGNTYLE (SEQ ID NO:73), a light chain CDR2 comprising KVSNQFS (SEQ ID NO:74), and a light chain CDR3 comprising QGSHVPL (SEQ ID NO:75). In certain embodiments, the antibody comprises (a) a heavy chain variable region having at least about 90%, at least about 95%, or at least about 98% sequence identity to SEQ ID NO:79, and (b) a light chain variable region having at least about 90%, at least about 95%, or at least about 98% sequence identity to SEQ ID NO:72 or SEQ ID NO:93. In some embodiments, the antibody comprises (a) a heavy chain variable region comprising SEQ ID NO:79 and (b) a light chain variable region comprising SEQ ID NO:72 or SEQ ID NO:93. In certain embodiments, the antibody is linked to an LTβR-binding moiety or a polypeptide that binds human LTβR.

In another aspect, the invention features antibodies that compete with each of the above-indicated antibodies for binding to human P-CADHERIN. In a further aspect, an antibody that binds the same epitope, or substantially the same epitope, on P-CADHERIN as any of the above-indicated P-CADHERIN antibodies is also provided. In a still further aspect, the invention provides an antibody that binds an epitope on P-CADHERIN that overlaps with the epitope of any of the other P-CADHERIN antibodies described herein.

In certain embodiments, the antibodies that specifically bind P-CADHERIN may be useful for targeting a tumor, e.g., a bladder, breast, colon, lung, melanoma, ovarian, pancreatic, or stomach tumor, in a subject. Other methods of use of the P-CADHERIN antibodies are also provided. For instance, the invention features a method of inhibiting the growth of a tumor (e.g., a bladder, breast, colon, lung, melanoma, ovarian, pancreatic, or stomach tumor) in a subject, comprising administering a therapeutically effective amount of a P-CADHERIN antibody described herein (or a polypeptide, agent or molecule comprising such antibody) to the subject. Methods of treating cancer (e.g., bladder, breast, colon, lung, melanoma, ovarian, pancreatic, or stomach cancer) in a subject comprising administering a therapeutically effective amount of a B7-H4 antibody described herein (or a polypeptide, agent or molecule comprising such antibody) are also provided.

Each antibody of the invention (e.g., an anti-B7H4 or anti-P-CADHERIN antibody) may be a recombinant antibody, a monoclonal antibody, a chimeric antibody, a bispecific antibody, a humanized antibody, a human antibody, an IgG antibody, an IgG2 antibody, or an antibody fragment comprising an antigen-binding site. The antibodies (e.g., antibodies that specifically bind B7-H4 or P-CADHERIN) may be detectably labeled (e.g., an affinity label, an enzymatic label, a fluorescent label, a radioisotope label, and a magnetic label). In some embodiments, the antibodies may be conjugated to cytotoxic agents.

The invention also features an isolated polynucleotide comprising a polynucleotide that encodes the antibody of each of the aforementioned aspects, a vector comprising the isolated polynucleotide, or a cell comprising the isolated polynucleotide or vector. The invention also features a cell or hybridoma that produces or is capable of producing the antibodies described herein. The invention also features a cell comprising the antibodies described herein.

In another aspect, the invention provides compositions comprising a polypeptide, agent, molecule or antibody described herein. Methods of using a composition comprising a polypeptide, molecule, agent or antibody described herein are also provided.

In another aspect, the invention provides pharmaceutical compositions comprising a polypeptide, molecule, agent or antibody described herein and a pharmaceutically acceptable carrier. Methods of treating cancer and/or inhibiting tumor growth in a subject (e.g., a human) comprising administering to the subject an effective amount of a composition comprising a polypeptide, molecule, agent, or antibody described herein are also provided. Methods of treating a viral infection in a subject (e.g., a human) comprising administering to the subject an effective amount of a composition comprising a polypeptide, molecule, or agent described herein are also provided.

In certain embodiments of each of the aforementioned aspects, as well as other aspects and/or embodiments described elsewhere herein, the polypeptide, molecule, agent, or antibody is isolated. In certain embodiments, the polypeptide, molecule, antibody or agent is substantially pure.

In another aspect, the invention provides polynucleotides comprising a polynucleotide that encodes a polypeptide, molecule, antibody or agent described herein. In some embodiments, the polynucleotide is isolated. In some embodiments, the invention provides vectors that comprise the polynucleotides, as well as cells that comprise the vectors and/or the polynucleotides. In some embodiments, the invention also provides cells comprising or producing a polypeptide, molecule, antibody or agent described herein. In some embodiments, the cell is a monoclonal cell line.

In another aspect, the invention provides methods of modulating the immune response of a subject. In some embodiments, the invention provides a method of inducing an immune response in a subject comprising administering a polypeptide, molecule, or agent described herein. In some embodiments, the invention provides a method of activating an immune response in a subject comprising administering a polypeptide, molecule, or agent described herein. In some embodiments, the invention provides a method of promoting an immune response in a subject comprising administering a polypeptide, molecule, or agent described herein. In some embodiments, the invention provides a method of increasing an immune response in a subject comprising administering a polypeptide, molecule, or agent described herein. In some embodiments, the invention provides a method of enhancing an immune response in a subject comprising administering a polypeptide, molecule, or agent described herein. In some embodiments, the invention provides a method of prolonging an immune response in a subject comprising administering a polypeptide, molecule, or agent described herein. In some embodiments, the immune response is to antigenic stimulation. In some embodiments, the antigenic stimulation is a tumor or a tumor cell. In some embodiments, the antigenic stimulation is a pathogen. In some embodiments, the antigenic stimulation is a virus. In some embodiments, the antigenic stimulation is a virally-infected cell.

In some embodiments, the invention provides a method of increasing the activity of immune cells. In some embodiments, the invention provides a method of increasing the activity of immune cells comprising contacting the cells with an effective amount of a polypeptide, molecule, or agent described herein. In some embodiments, the immune cells are T-cells, NK cells, monocytes, macrophages, antigen-presenting cells (APCs), and/or B-cells. In some embodiments, the invention provides a method of increasing the activity of NK cells in a subject comprising administering to the subject a therapeutically effective amount of a polypeptide, molecule, or agent described herein. In some embodiments, the invention provides a method of increasing the activity of T-cells in a subject comprising administering to the subject a therapeutically effective amount of a polypeptide, molecule, or agent described herein. In some embodiments, the invention provides a method of increasing the activity of CD4+ and/or CD8+ T-cells in a subject comprising administering to the subject a therapeutically effective amount of a polypeptide, molecule, or agent described herein. In some embodiments, the invention provides a method of increasing the activity of CTLs in a subject comprising administering to the subject a therapeutically effective amount of a polypeptide, molecule, or agent described herein. In some embodiments, the invention provides a method of increasing the activation of T-cells, CTLs, and/or NK cells in a subject comprising administering to the subject a therapeutically effective amount of a polypeptide, molecule, or agent described herein. In some embodiments, the invention provides a method of increasing the T-cell response in a subject comprising administering to the subject a therapeutically effective amount of a polypeptide, molecule, or agent described herein. In some embodiments, the invention provides a method of inhibiting the activity of Tregs in a subject comprising administering to the subject a therapeutically effective amount of a polypeptide, molecule, or agent described herein. In some embodiments, the invention provides a method of inhibiting the suppressive activity of Tregs in a subject comprising administering to the subject a therapeutically effective amount of a polypeptide, molecule, or agent described herein. In some embodiments, the invention provides a method of inhibiting the activity of MDSCs in a subject comprising administering to the subject a therapeutically effective amount of a polypeptide, molecule, or agent described herein. In some embodiments, the invention provides a method of inhibiting the suppressive activity of MDSCs in a subject comprising administering to the subject a therapeutically effective amount of a polypeptide, molecule, or agent described herein. In some embodiments, the invention provides a method of inducing an immune response in a subject without causing substantial side effects and/or immune-based toxicities comprising administering to the subject a therapeutically effective amount of a polypeptide, molecule, or agent described herein. In some embodiments, the invention provides a method of inducing an immune response in a subject without causing cytokine release syndrome or a cytokine storm comprising administering to the subject a therapeutically effective amount of a polypeptide, molecule, or agent described herein.

In another aspect, the invention provides methods of inducing, activating, promoting, increasing, enhancing, or prolonging an immune response in a subject. In some embodiments, the invention provides methods of inducing, activating, promoting, increasing, enhancing, or prolonging an immune response in a subject, comprising administering to the subject a therapeutically effective amount of a polypeptide, molecule, or agent described herein. In some embodiments, the immune response is against a tumor cell, a tumor or cancer. In some embodiments, the immune response is against a viral infection, a viral antigen, or a virally-infected cell.

In some embodiments, the invention provides a method of increasing T-cell activity in a subject, comprising administering to the subject a therapeutically effective amount of a polypeptide, molecule, or agent described herein. In some embodiments, the invention provides a method of increasing NK activity in a subject, comprising administering to the subject a therapeutically effective amount of a polypeptide, molecule, or agent described herein. In some embodiments, the invention provides a method of decreasing or inhibiting Treg activity in a subject, comprising administering to the subject a therapeutically effective amount of a polypeptide, molecule, or agent described herein. In some embodiments, the invention provides a method of decreasing or inhibiting MDSC activity in a subject, comprising administering to the subject a therapeutically effective amount of a polypeptide, molecule, or agent described herein. In some embodiments of the methods described herein, the subject has cancer.

In some embodiments, the invention provides a method of increasing T-cell activity in a subject, comprising administering to the subject a therapeutically effective amount of any of the polypeptides, molecules, or agents, and/or bispecific agents described herein. In some embodiments, the invention provides a method of increasing CTL activity in a subject, comprising administering to the subject a therapeutically effective amount of the polypeptide of any of the polypeptides, molecules, agents, and/or bispecific agents described herein. In some embodiments, the invention provides a method of increasing NK activity in a subject, comprising administering to the subject a therapeutically effective amount of the polypeptide of any of the polypeptides, molecules, agents, and/or bispecific agents described herein. In some embodiments, the invention provides a method of decreasing or inhibiting Treg activity in a subject, comprising administering to the subject a therapeutically effective amount of any of the polypeptides, molecules, agents, and/or bispecific agents described herein. In some embodiments, the invention provides a method of decreasing or inhibiting MDSC activity in a subject, comprising administering to the subject a therapeutically effective amount of the polypeptide of any of the polypeptides, molecules, agents, and/or bispecific agents described herein. In some embodiments of the methods described herein, the subject has cancer.

In another aspect, the invention provides a method of enhancing the antigen-specific memory response to a tumor. In some embodiments, a method of enhancing the antigen-specific memory response to a tumor comprises administering to a subject a therapeutically effective amount of any of the polypeptides, molecules, agents, and/or bispecific agents described herein.

In another aspect, the invention provides a method of activating or enhancing a persistent or long-term immune response to a tumor. In some embodiments, a method of activating or enhancing a persistent immune response to a tumor comprises administering to a subject a therapeutically effective amount of any of the polypeptides, molecules, agents, and/or bispecific agents described herein.

In another aspect, the invention provides a method of inducing a persistent or long-term immunity which inhibits tumor relapse or tumor regrowth. In some embodiments, a method of inducing a persistent immunity which inhibits tumor relapse or tumor regrowth comprises administering to a subject a therapeutically effective amount of any of the polypeptides, molecules, agents, and/or bispecific agents described herein.

In another aspect, the invention provides methods of inhibiting tumor growth comprising contacting a tumor or tumor cell with an effective amount of a polypeptide, molecule, or agent described herein.

In another aspect, the invention provides methods of inhibiting tumor growth in a subject comprising administering to the subject a therapeutically effective amount of a polypeptide, molecule, or agent described herein.

In another aspect, the invention provides methods of treating cancer in a subject comprising administering to the subject a therapeutically effective amount of a polypeptide, molecule, or agent described herein.

In another aspect, the invention provides methods of stimulating a protective response in a subject comprising administering to the subject a therapeutically effective amount of a polypeptide or agent described herein in combination with an antigen of interest. In some embodiments, the antigen of interest is a tumor antigen or tumor-associated antigen (TAA), for example, any described herein, such as B7-H4 or P-CADHERIN (CDH3). In some embodiments, the antigen of interest is a cancer cell biomarker. In some embodiments, the antigen of interest is a cancer stem cell marker.

In some embodiments of each of the aforementioned aspects and embodiments, as well as other aspects and embodiments described herein, the methods further comprise administering to the subject at least one additional therapeutic agent. In some embodiments, the at least one additional therapeutic agent is a chemotherapeutic agent. In some embodiments, the at least one additional therapeutic agent is an immunotherapeutic agent.

Where aspects or embodiments of the invention are described in terms of a Markush group or another grouping of alternatives, the present invention encompasses not only the entire group listed as a whole, but also each member of the group individually and all possible subgroups of the main group, and also the main group absent one or more of the group members. The present invention also envisages the explicit exclusion of one or more of any of the group members in the claimed invention.

### BRIEF DESCRIPTION OF THE FIGURES

FIGS. 1A and 1B. FIG. 1A. Shown is a representative drawing depicting a homodimeric molecule comprising an anti-tumor associated antigen antibody and a lymphotoxin-αββ polypeptide. FIG. 1B. Shown is a representative drawing depicting a heterodimeric molecule that comprises a lymphotoxin-αββ polypeptide linked to a Fe region as part of a first monomer and an anti-tumor associated antigen-binding site as part of a second monomer.

FIG. 2. FACS analysis of anti-mB7-H4/lymphotoxin-αββ binding to mB7-H4 and lymphotoxin 3 receptor. Human HEK-293T cells were transiently transfected with expression vectors encoding a membrane-bound extracellular domain of mouse B7-H4 (mB7-H4-CD4TM-GFP) or a membrane-bound extracellular domain of mouse LTβR (mLTβR-CD4TM-GFP). The transfected cells were incubated in the presence of 349B1 (anti-mB7-H4/lymphotoxin-αββ) for 30 minutes and stained with an APC-conjugated anti-mouse Fc secondary antibody to detect cells bound by 349B1. Cells were incubated with an anti-APC antibody as a negative control. The cells were analyzed on a FACS Canto instrument (BD Biosciences), and the data were processed using FlowJo software.

FIG. 3. Activation of LTβR signaling by anti-mB7-H4/lymphotoxin-αββ. A HEK-293 cell line was co-transfected with an expression vector encoding full-length mouse LTβR and an expression vector encoding an NF-kB-luciferase reporter construct. A stably transfected cell line was identified and selected. For the assay, cells were plated into a 96 well plate and incubated overnight. 349B1 (anti-mB7-H4/lymphotoxin-αββ), anti-mB7-H4/LIGHT trimer 351B1, and anti-mB7-H4/LIGHT trimer mutant 351B2 were tested over a range of concentrations (5-fold dilutions 50 μg/ml to 0.016 μg/ml). Luciferase activity was determined using a Steady-Glo assay kit (Promega) according to the manufacturer's instructions.

FIGS. 4A and 4B. Inhibition of tumor growth by anti-mB7-H4/lymphotoxin-αββ. The murine colon tumor line CT26.WT-B7H4 was implanted subcutaneously into Balb/c mice (n=10 mice/group). Mice were administered anti-mB7-H4/lymphotoxin-up 349B1, anti-mPD-L1 antibody, a combination of 349B1 and anti-mPD-L1 antibody, or a control antibody. Tumor growth was monitored, and tumor volumes were measured with electronic calipers at the indicated time points. Data are shown as tumor volume (mm³) over days post-injection. FIG. 4A. The mean values±SEM for anti-mB7-H4/lymphotoxin-αββ 349B1. FIG. 4B. The mean values±SEM for anti-mB7-H4/lymphotoxin-αββ P 349B1 in combination with an anti-mPD-L1 antibody.

FIGS. 5A and 5B. Expression of chemokines CXCL13 and CCL19. FIG. 5A shows expression of CXCL13 in mice receiving anti-mB7-H4/lymphotoxin-αββ 349B1, anti-mPD-L1 antibody, a combination of 349B1 and anti-mPD-L1 antibody, or a control antibody. FIG. 5B shows expression of CXCL13 in mice receiving anti-mB7-H4/lymphotoxin-αββ 349B1, anti-mPD-L1 antibody, a combination of 349B1 and anti-mPD-L1 antibody, or a control antibody.

FIGS. 6A-6D. Expression of immune cell markers in tumors. These graphs show expression of CD45 (FIG. 6A), CD3e (FIG. 6B), CD4 (FIG. 6C), and CD8a (FIG. 6D) in tumors of mice receiving anti-mB7-H4/lymphotoxin-αββ 349B1, anti-mPD-L1 antibody, a combination of 349B1 and anti-mPD-L1 antibody, or a control antibody.

FIGS. 7A and 7B. Levels and expression of interferon-γ. These graphs show protein levels (FIG. 7A) and mRNA expression levels (FIG. 7B) of interferon-γ in tumors of mice receiving anti-mB7-H4/lymphotoxin-αββ 349B1, anti-mPD-L1 antibody, a combination of 349B1 and anti-mPD-L1 antibody, or a control antibody.

FIGS. 8A and 8B. Binding of murine monoclonal antibody 278M1 to mouse and human B7-H4. These graphs show FACS binding data of antibody 278M1 to mouse (FIG. 8A) and human (FIG. 8B) B7-H4.

FIG. 9. Expression of B7-H4 in tumors. These are graphs (Left panel) and images (right panels) showing expression of B7-H4 in ovarian and breast tumors.

FIG. 10. Table is showing that P-CADHERIN/CDH3 is expressed in many tumor types, as well as in normal breast, ovarian, and prostate tissue.

FIG. 11. Levels of interleukin 6 (IL-6). The graph shows protein levels of IL-6 in tumors of mice receiving 349B1, anti-mPD-L1 antibody, a combination of 349B1 and anti-mPD-L1 antibody, or a control antibody.

FIGS. 12A-12D. Tumor targeting is required for anti-tumor activity of lymphotoxin-αββ in vivo. Cells from a murine colon tumor line (MC38) or an MC38 tumor line overexpressing mouse B7-H4 (MC38-B7H4) were implanted subcutaneously into C57BL/6N mice. Mice were administered 349B1, anti-mPD-L1 antibody, a combination of 349B1 and anti-mPD-L1 antibody, or a control antibody. Tumor growth was monitored, and tumor volumes were measured with electronic calipers at the indicated time points. Data are shown as tumor volume (mm³) over days post-injection (FIGS. 12A-12C) or as percent tumor growth inhibition over 349B1 dose (FIG. 12D). FIG. 12A. Mean tumor volumes in mice implanted with MC38-B7H4 cells and treated with 349B1 or control antibody. FIG. 12B. Mean tumor volumes in mice implanted with MC38-B7H4 tumor cells and treated with 349B1 in combination with an anti-mPD-L1 antibody or with a control antibody. FIG. 12C. Mean tumor volumes in mice implanted with MC38 tumor cells and treated with 349B1 or control antibody. FIG. 12D. Comparison of the percent inhibition of growth of MC38-B7H4 tumors and MC38 tumors at different doses of 349B1 in vivo.

FIG. 13. Tumor targeting is required for anti-tumor activity of lymphotoxin-αββ in vivo. MC38-B7H4 tumor cells were implanted subcutaneously into C57BL/6N mice. Mice were administered 363F1 (lymphotoxin-αββ), 278M24 (anti-mB7-H4 antibody), 349B1 (anti-mB7-H4/lymphotoxin-αββ), an anti-mouse PD-L1 antibody (332M9), a combination of 332M9 with either 363F1 or 349B1, or a control antibody. Tumor growth was monitored, and tumor volumes were measured with electronic calipers at the indicated time points. Data are shown as mean tumor volume (mm³) over days post-injection.

FIGS. 14A and 14B. Anti-mB7-H4/lymphotoxin-αββ 349B1 increases T-cell infiltration in tumors. FIG. 14A. Percentage of CD3+ T cells in tumor tissue cells in mice treated with 349B1 or control. FIG. 14B. Percentage of CD3⁺ T cells within the total immune cells (CD45⁺) from tumor tissue in mice treated with 349B1 or control.

FIGS. 15A-15E. Immune cell infiltration and inhibition of tumor growth upon single dose administration of anti-mB7-H4/lymphotoxin-αββ. CT26WT-B7H4 cells were implanted into BALB/c mice and dosed once with 349B1 on the seventh day after cell implantation at the indicated dosage level. FIG. 15A. Anti-tumor activity of single doses of 349B1 in mice. Mean tumor volumes are shown over days post-injection. FIG. 15B. Percent granulocytic myeloid-derived suppressor cells (G-MDSCs; Cd45⁺Cd11b⁺Gr1 high cells) in Cd45⁺Cd11b⁺ cells from the tumors at the indicated days post-injection. FIG. 15C. Percent Cd11b⁻ cells in Cd45⁺ cells from the tumors. FIG. 15D. The ratio of CD8 cytotoxic T cells to G-MDSCs. FIG. 15E. The ratio of CD4 effector T-cells to G-MDSCs.

FIGS. 16A-16B. Induction of tertiary lymphoid structure induced by anti-mB7-H4/lymphotoxin-αββ. FIG. 16A. Immunohistochemistry with the anti-Pax5 antibody. FIG. 16B. Immunohistochemistry with anti-CD8 (darker stain) and anti-CD45 (light stain) antibodies.

FIG. 17. Anti-tumor activity of anti-mB7-H4/lymphotoxin-αββ on TC1-mB7-H4 tumors. Mean tumor volumes are shown over days post-injection.

FIGS. 18A-18B. Flow cytometry analysis of cells from TC1-mB7-H4 tumors treated with anti-mB7-H4/lymphotoxin-αββ. FIG. 18A. Analysis of CD4, CD8 and NK cell populations. FIG. 18B. Analysis of IFN gamma production in CD4, CD8 and NK cells.

FIG. 19. Anti-tumor activity of anti-mB7-H4/lymphotoxin-αββ on EMT6-mB7-H4 tumors. Mean tumor volumes are shown over days post-injection.

FIG. 20. Flow cytometry analysis of cells from EMT6-mB7-H4 tumors treated with anti-mB7-H4/lymphotoxin-up.

FIGS. 21A-21E. Tumor-specific antigen expression in patient-derived tumors. FIG. 21A. P-CADHERIN (CDH3) expression levels. FIG. 21B. PVRL4 expression levels. FIG. 21C. CD276 (B7H3) expression levels. FIG. 21D. Mesothelin expression levels. FIG. 21E. CA9 expression levels.

FIG. 22. FACS analysis of humanized B7-H4 antibody 278M1 L2H2 binding to human B7-H4 (top) and mouse B7-H4 (bottom).

FIG. 23. FACS analysis of binding by humanized P-CADHERIN (CDH3) antibodies 173M36 L1H2 (left) and 173M36 L3H2 (right) to human P-CADHERIN (top row) and mouse P-CADHERIN (bottom row).

FIG. 24. FACS analysis of binding by single-chain human lymphotoxin (hLT) heterotrimers. The ability of hLTαββ (363F2), hLTβαβ (363F3), hLTββα (363F4), and mouse LTαββ (363F1) Fc fusion constructs to bind human LTβR (top row) and mouse LTβR (bottom row) was measured by FACS.

FIG. 25. Agonist activity of hLT heterotrimers in NFκB reporter assay. The ability of 363F1, 363F2, 363F3, and 363F4 Fc fusion constructs and 349B1 to activate NFκB signaling was measured in an in vitro luciferase reporter assay.

FIG. 26. 364B4, humanized anti-CDH3/humanLT-αββ p, binds to human and mouse CDH3 and LTβR.

### DETAILED DESCRIPTION OF THE INVENTION

In one aspect, the present invention provides novel molecules/agents, including, but not limited to, polypeptides, soluble proteins, fusion proteins, homodimeric molecules, and heterodimeric molecules that comprise lymphotoxin-αββ or other moieties that bind lymphotoxin-beta receptor (LTβR). The molecules/agents include agonists of LTβR that is involved in development and organization of lymphoid tissue. LTβR is also involved in the development of secondary and tertiary lymphoid tissue and in chemokine secretion/release. Related polypeptides and polynucleotides, compositions comprising the molecules/agents, and methods of making the molecules/agents are also provided. Methods of screening the molecules/agents described herein are provided. Methods of using the novel molecules/agents, such as methods of activating/enhancing LTβR signaling, activating an immune response, methods of stimulating an immune response, methods of promoting an immune response, methods of increasing an immune response, methods of recruiting immune cells to a target, methods of recruiting tumor-infiltrating lymphocytes to a tumor, methods of activating T-cells, including CTLs, methods of increasing the activity of T-cells, including CTLs, methods of promoting the activity of T-cells, including CTLs, methods of inhibiting tumor growth, and/or methods of treating cancer are provided.

In another aspect, the invention provides novel molecules/agents that comprise both an LTβR-binding moiety (e.g., lymphotoxin αββ heterotrimer) and a targeting moiety (e.g., an antibody that specifically binds a cell-surface antigen such as B7-H4 or P-CADHERIN). Related polypeptides and polynucleotides, compositions comprising the molecules/agents, and methods of making the molecules/agents are also provided. Methods of screening the molecules/agents described herein are provided. Methods of using the novel molecules/agents, such as methods of increasing the responsiveness of a tumor to treatment with a second therapeutic agent (e.g., a second immunotherapeutic agent), methods of activating/enhancing LTβR signaling, methods of recruiting immune cells to a target, methods of recruiting tumor-infiltrating lymphocytes to a tumor, methods of inhibiting tumor growth, and/or methods of treating cancer are provided.

In a further aspect, the invention provides novel antibodies that bind B7-H4 or P-CADHERIN, as well as polypeptides and agents comprising such antibodies. Related polypeptides and polynucleotides, compositions comprising the antibodies, and methods of making or screening the antibodies are also provided. Methods of using the antibodies, such as methods of targeting a tumor, methods of inhibiting tumor growth, and/or methods of treating cancer are further provided.

### I. Definitions

To facilitate an understanding of the present invention, a number of terms and phrases are defined below.

The terms “agonist” and “agonistic” as used herein refer to or describe a polypeptide or molecule that is capable of, directly or indirectly, substantially inducing, activating, promoting, increasing, or enhancing the biological activity of a target and/or a pathway. The term “agonist” is used herein to include any agent that partially or fully induces, activates, promotes, increases, or enhances the activity of a protein or other target of interest.

The terms “antagonist” and “antagonistic” as used herein refer to or describe a polypeptide or molecule that is capable of, directly or indirectly, partially or fully blocking, inhibiting, reducing, or neutralizing a biological activity of a target and/or pathway. The term “antagonist” is used herein to include any agent that partially or fully blocks, inhibits, reduces, or neutralizes the activity of a protein or other target of interest.

The terms “modulation” and “modulate” as used herein refer to a change or an alteration in a biological activity. Modulation includes, but is not limited to, stimulating an activity or inhibiting an activity. Modulation may be an increase in activity or a decrease in activity, a change in binding characteristics, or any other change in the biological, functional, or immunological properties associated with the activity of a protein, a pathway, a system, or other biological targets of interest.

The term “soluble protein” as used herein refers to a protein or a fragment thereof that can be secreted from a cell in soluble form.

The term “fusion protein” or “fusion polypeptide” as used herein refers to a hybrid protein expressed by a nucleic acid molecule comprising nucleotide sequences of at least two genes.

The term “linker” or “linker region” as used herein refers to a linker inserted between a first polypeptide (e.g., copies of lymphotoxin-beta extracellular domain or fragments thereof) and a second polypeptide (e.g., lymphotoxin-alpha). In some embodiments, the linker is a peptide linker. Linkers should not adversely affect the expression, secretion, or bioactivity of the polypeptides. Preferably, linkers are not antigenic and do not elicit an immune response.

The term “antibody” as used herein refers to an immunoglobulin molecule that recognizes and specifically binds a target, such as a protein, polypeptide, peptide, carbohydrate, polynucleotide, lipid, or a combination of any of the foregoing, through at least one antigen-binding site wherein the antigen-binding site is usually within the variable region of the immunoglobulin molecule. As used herein, the term encompasses intact polyclonal antibodies, intact monoclonal antibodies, antibody fragments (such as Fab. Fab′, F(ab′)2, and Fv fragments), single-chain Fv (scFv) antibodies, multispecific antibodies, bispecific antibodies, monospecific antibodies, monovalent antibodies, chimeric antibodies, humanized antibodies, human antibodies, fusion proteins comprising an antigen-binding site of an antibody, and any other modified immunoglobulin molecule comprising an antigen-binding site (e.g., dual variable domain immunoglobulin molecules) as long as the antibodies exhibit the desired biological activity. An antibody can be any of the five major classes of immunoglobulins: IgA, IgD, IgE. IgG, and IgM, or subclasses (isotypes) thereof (e.g., IgG1, IgG2, IgG3, IgG4, IgA1 and lgA2), based on the identity of their heavy-chain constant domains referred to as alpha, delta, epsilon, gamma, and mu, respectively. The different classes of immunoglobulins have different and well-known subunit structures and three-dimensional configurations. Antibodies can be naked or conjugated to other molecules, including but not limited to, toxins and radioisotopes.

The term “antibody fragment” refers to a portion of an intact antibody and refers to the antigenic determining variable regions of an intact antibody. Examples of antibody fragments include, but are not limited to, Fab, Fab′, F(ab′)2, and Fv fragments, linear antibodies, single-chain antibodies, and multispecific antibodies formed from antibody fragments. “Antibody fragment” as used herein comprises an antigen-binding site or epitope-binding site.

The term “variable region” of an antibody refers to the variable region of an antibody light chain, or the variable region of an antibody heavy chain, either alone or in combination. Generally, the variable region of heavy and light chains each consist of four framework regions (FR) and three complementarity determining regions (CDRs), also known as “hypervariable regions”. The CDRs in each chain are held together in close proximity by the framework regions and, with the CDRs from the other chain, contribute to the formation of the antigen-binding sites of the antibody. There are at least two techniques for determining CDRs: (1) an approach based on cross-species sequence variability (i.e., Kabat et al., 1991, Sequences of Proteins of Immunological Interest, 5th Edition, National Institutes of Health, Bethesda Md.), and (2) an approach based on crystallographic studies of antigen-antibody complexes (Al Lazikani et al., 1997, J. Mol. Biol., 273:927-948). In addition, combinations of these two approaches are sometimes used in the art to determine CDRs.

The term “monoclonal antibody” as used herein refers to a homogenous antibody population involved in the highly specific recognition and binding of a single antigenic determinant or epitope. This is in contrast to polyclonal antibodies that typically include a mixture of different antibodies directed against different antigenic determinants. The term “monoclonal antibody” encompasses both intact and full-length monoclonal antibodies as well as antibody fragments (e.g., Fab, Fab′, F(ab′)2, Fv), single-chain (scFv) antibodies, fusion proteins comprising an antibody fragment, and any other modified immunoglobulin molecule comprising an antigen-binding site. Furthermore, “monoclonal antibody” refers to such antibodies made by any number of techniques, including but not limited to, hybridoma production, phage selection, recombinant expression, and transgenic animals.

The term “humanized antibody” as used herein refers to forms of non-human (e.g., murine) antibodies that are specific immunoglobulin chains, chimeric immunoglobulins, or fragments thereof that contain minimal non-human sequences. Typically, humanized antibodies are human immunoglobulins in which residues of the CDRs are replaced by residues from the CDRs of a non-human species (e.g., mouse, rat, rαββit, or hamster) that have the desired specificity, affinity, and/or binding capability. In some instances, the Fv framework region residues of a human immunoglobulin are replaced with the corresponding residues in an antibody from a non-human species. The humanized antibody can be further modified by the substitution of additional residues either in the Fv framework region and/or within the replaced non-human residues to refine and optimize antibody specificity, affinity, and/or binding capability. The humanized antibody may comprise variable domains containing all or substantially all of the CDRs that correspond to the non-human immunoglobulin whereas all or substantially all of the framework regions are those of a human immunoglobulin sequence. In some embodiments, the variable domains comprise the framework regions of a human immunoglobulin sequence. In some embodiments, the variable domains comprise the framework regions of a human immunoglobulin consensus sequence. The humanized antibody can also comprise at least a portion of an immunoglobulin constant region or domain (Fc), typically that of a human immunoglobulin. A humanized antibody is usually considered distinct from a chimeric antibody.

The term “human antibody” as used herein refers to an antibody produced by a human or an antibody having an amino acid sequence corresponding to an antibody produced by a human made using any of the techniques known in the art.

The term “chimeric antibody” as used herein refers to an antibody wherein the amino acid sequence of the immunoglobulin molecule is derived from two or more species. Typically, the variable region of both light and heavy chains corresponds to the variable region of antibodies derived from one species of mammals (e.g., mouse, rat, rαββit, etc.) with the desired specificity, affinity, and/or binding capability, while the constant regions are homologous to the sequences in antibodies derived from another species (usually human) to avoid eliciting an immune response in that species.

The terms “epitope” and “antigenic determinant” are used interchangeably herein and refer to that portion of an antigen capable of being recognized and specifically bound by a particular antibody. When the antigen is a polypeptide, epitopes can be formed both from contiguous amino acids and noncontiguous amino acids juxtaposed by tertiary folding of a protein. Epitopes formed from contiguous amino acids (also referred to as linear epitopes) are typically retained upon protein denaturing, whereas epitopes formed by tertiary folding (also referred to as conformational epitopes) are typically lost upon protein denaturing. An epitope typically includes at least 3, and more usually, at least 5, 6, 7, or 8-10 amino acids in a unique spatial conformation.

The terms “selectively binds” or “specifically binds” mean that a polypeptide or molecule interacts more frequently, more rapidly, with greater duration, with greater affinity, or with some combination of the above to the epitope, protein, or target molecule than with alternative substances, including related and unrelated proteins. In certain embodiments “specifically binds” means, for instance, that a polypeptide or molecule binds a protein or target with a K_{D} of about 0.1 mM or less, but more usually less than about 1 μM. In certain embodiments, “specifically binds” means that a polypeptide or molecule binds a target with a Ku of at least about 0.1 μM or less, at least about 0.01 μM or less, or at least about 1 nM or less. Because of the sequence identity between homologous proteins in different species, specific binding can include a polypeptide or molecule that recognizes a protein or target in more than one species. Likewise, because of homology within certain regions of polypeptide sequences of different proteins, specific binding can include a polypeptide or molecule that recognizes more than one protein or target. It is understood that, in certain embodiments, a polypeptide or molecule that specifically binds a first target may or may not specifically bind a second target. As such, “specific binding” does not necessarily require (although it can include) exclusive binding, i.e. binding to a single target. Thus, a polypeptide or molecule may, in certain embodiments, specifically bind more than one target. In certain embodiments, multiple targets may be bound by the same antigen-binding site on the polypeptide or molecule. For example, an antibody may, in certain instances, comprise two identical antigen-binding sites, each of which specifically binds the same epitope on two or more proteins. In certain alternative embodiments, an antibody may be bispecific and comprise at least two antigen-binding sites with differing specificities. Generally, but not necessarily, reference to “binding” means “specific binding”.

The terms “polypeptide” and “peptide” and “protein” are used interchangeably herein and refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids), as well as other modifications known in the art. It is understood that, because the polypeptides of this invention may be based upon antibodies or other members of the immunoglobulin superfamily, in certain embodiments, the polypeptides can occur as single chains or as associated chains.

The terms “polynucleotide” and “nucleic acid” and “nucleic acid molecule” are used interchangeably herein and refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase.

The terms “identical” or percent “identity” in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity may be measured using sequence comparison software or algorithms or by visual inspection. Various algorithms and software that may be used to obtain alignments of amino acid or nucleotide sequences are well-known in the art. These include, but are not limited to, BLAST, ALIGN, Megalign, BestFit, GCG Wisconsin Package, and variants thereof. In some embodiments, two nucleic acids or polypeptides of the invention are substantially identical, meaning they have at least 70%, at least 75%, at least 80%, at least 85%, at least 90%, and in some embodiments at least 95%, 96%, 97%, 98%, 99% nucleotide or amino acid residue identity, when compared and aligned for maximum correspondence, as measured using a sequence comparison algorithm or by visual inspection. In some embodiments, identity exists over a region of the amino acid sequences that is at least about 10 residues, at least about 20 residues, at least about 40-60 residues, at least about 60-80 residues in length or any integral value there between. In some embodiments, identity exists over a longer region than 60-80 residues, such as at least about 80-100 residues, and in some embodiments the sequences are substantially identical over the full length of the sequences being compared, such as the coding region of a target protein or an antibody. In some embodiments, identity exists over a region of the nucleotide sequences that is at least about 10 bases, at least about 20 bases, at least about 40-60 bases, at least about 60-80 bases in length or any integral value there between. In some embodiments, identity exists over a longer region than 60-80 bases, such as at least about 80-1000 bases or more, and in some embodiments the sequences are substantially identical over the full length of the sequences being compared, such as a nucleotide sequence encoding a protein of interest.

A “conservative amino acid substitution” is one in which one amino acid residue is replaced with another amino acid residue having a similar side chain. Families of amino acid residues having similar side chains have been generally defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is a conservative substitution. Generally, conservative substitutions in the sequences of the polypeptides, soluble proteins, and/or antibodies of the invention do not abrogate the binding of the polypeptide, soluble protein, or antibody containing the amino acid sequence, to the target binding site. Methods of identifying amino acid conservative substitutions which do not eliminate binding are well-known in the art.

The term “vector” as used herein means a construct, which is capable of delivering, and usually expressing, one or more gene(s) or sequence(s) of interest in a host cell. Examples of vectors include, but are not limited to, viral vectors, naked DNA or RNA expression vectors, plasmid, cosmid, or phage vectors, DNA or RNA expression vectors associated with cationic condensing agents, and DNA or RNA expression vectors encapsulated in liposomes.

A polypeptide, soluble protein, antibody, polynucleotide, vector, cell, or composition which is “isolated” is a polypeptide, soluble protein, antibody, polynucleotide, vector, cell, or composition which is in a form not found in nature. Isolated polypeptides, soluble proteins, antibodies, polynucleotides, vectors, cells, or compositions include those which have been purified to a degree that they are no longer in a form in which they are found in nature. In some embodiments, a polypeptide, soluble protein, antibody, polynucleotide, vector, cell, or composition which is isolated is substantially pure.

The term “substantially pure” as used herein refers to material which is at least 50% pure (i.e., free from contaminants), at least 90% pure, at least 95% pure, at least 98% pure, or at least 99% pure.

The term “immune response” as used herein includes responses from both the innate immune system and the adaptive immune system. It includes both cell-mediated and/or humoral immune responses. It includes both T-cell and B-cell responses, as well as responses from other cells of the immune system such as natural killer (NK) cells, monocytes, macrophages, etc.

The terms “cancer” and “cancerous” as used herein refer to or describe the physiological condition in mammals in which a population of cells are characterized by unregulated cell growth. Examples of cancer include, but are not limited to, carcinoma, blastoma, sarcoma, and hematologic cancers such as lymphoma and leukemia.

The terms “tumor” and “neoplasm” as used herein refer to any mass of tissue that results from excessive cell growth or proliferation, either benign (noncancerous) or malignant (cancerous) including pre-cancerous lesions.

The term “metastasis” as used herein refers to the process by which a cancer spreads or transfers from the site of origin to other regions of the body with the development of a similar cancerous lesion at the new location. A “metastatic” or “metastasizing” cell is one that loses adhesive contacts with neighboring cells and migrates via the bloodstream or lymph from the primary site of disease to invade neighboring body structures.

The terms “cancer cell” and “tumor cell” refer to the total population of cells derived from a cancer or tumor or pre-cancerous lesion, including both non-tumorigenic cells, which comprise the bulk of the cancer cell population, and tumorigenic stem cells (cancer stem cells). As used herein, the terms “cancer cell” or “tumor cell” will be modified by the term “non-tumorigenic” when referring solely to those cells lacking the capacity to renew and differentiate to distinguish those tumor cells from cancer stem cells.

The term “subject” refers to any animal (e.g., a mammal), including, but not limited to, humans, non-human primates, canines, felines, rodents, and the like, which is to be the recipient of a particular treatment. Typically, the terms “subject” and “patient” are used interchangeably herein in reference to a human subject.

The term “pharmaceutically acceptable” refers to a substance approved or approvable by a regulatory agency of the Federal government or a state government or listed in the U.S. Pharmacopeia or other generally recognized pharmacopeia for use in animals, including humans.

The terms “pharmaceutically acceptable excipient, carrier or adjuvant” or “acceptable pharmaceutical carrier” refer to an excipient, carrier or adjuvant that can be administered to a subject, together with at least one agent of the present disclosure, and which does not destroy the pharmacological activity thereof and is nontoxic when administered in doses sufficient to deliver a therapeutic effect. In general, those of skill in the art and the U.S. FDA consider a pharmaceutically acceptable excipient, carrier, or adjuvant to be an inactive ingredient of any formulation.

The terms “effective amount” or “therapeutically effective amount” or “therapeutic effect” refer to an amount of a polypeptide or molecule described herein (e.g., a fusion protein, a soluble ligand, an antibody, a polypeptide, a polynucleotide, a small organic molecule, or other drug) effective to “treat” a disease or disorder in a subject such as, a mammal. In the case of cancer or a tumor, the therapeutically effective amount of a polypeptide or molecule (e.g., polypeptide, soluble protein, or antibody) has a therapeutic effect and as such can boost the immune response, boost the anti-tumor response, increase cytolytic activity of immune cells, increase killing of tumor cells by immune cells, reduce the number of tumor cells; decrease tumorigenicity, tumorigenic frequency or tumorigenic capacity: reduce the number or frequency of cancer stem cells; reduce the tumor size; reduce the cancer cell population; inhibit or stop cancer cell infiltration into peripheral organs including, for example, the spread of cancer into soft tissue and bone; inhibit and stop tumor or cancer cell metastasis; inhibit and stop tumor or cancer cell growth; relieve to some extent one or more of the symptoms associated with the cancer; reduce morbidity and mortality; improve quality of life; or a combination of such effects.

The terms “treating” or “treatment” or “to treat” or “alleviating” or “to alleviate” refer to both (1) therapeutic measures that cure, slow down, lessen symptoms of, and/or halt progression of a diagnosed pathologic condition or disorder and (2) prophylactic or preventative measures that prevent or slow the development of a targeted pathologic condition or disorder. Thus those in need of treatment include those already with the disorder, those prone to have the disorder; and those in whom the disorder is to be prevented. In the case of cancer or a tumor, a subject is successfully “treated” according to the methods of the present invention if the patient shows one or more of the following: an increased immune response, an increased anti-tumor response, increased cytolytic activity of immune cells, increased killing of tumor cells by immune cells, a reduction in the number of or complete absence of cancer cells; a reduction in the tumor size; inhibition of or an absence of cancer cell infiltration into peripheral organs including the spread of cancer cells into soft tissue and bone; inhibition of or an absence of tumor or cancer cell metastasis; inhibition or an absence of cancer growth; relief of one or more symptoms associated with the specific cancer; reduced morbidity and mortality; improvement in quality of life; reduction in tumorigenicity; reduction in the number or frequency of cancer stem cells; or some combination of effects.

As used in the present disclosure and claims, the singular forms “a”, “an” and “the” include plural forms unless the context clearly dictates otherwise.

It is understood that wherever embodiments are described herein with the language “comprising” otherwise analogous embodiments described in terms of“consisting of” and/or “consisting essentially of” are also provided. It is also understood that wherever embodiments are described herein with the language “consisting essentially of” otherwise analogous embodiments described in terms of “consisting of” are also provided.

As used herein, reference to “about” or “approximately” a value or parameter includes (and describes) embodiments that are directed to that value or parameter. For example, description referring to “about X” includes description of “X”.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B. and C; A, B, or C; A or C; A or B; B or C; A and C; A and B; B and C; A (alone); B (alone); and C (alone).

### II. Molecules/Agents Comprising Lymphotoxin-αββ

In one aspect, the present invention provides molecules/agents that comprise lymphotoxin-αββ p (or a lymphotoxin αββ heterotrimer). In some embodiments, the molecule or agent binds the lymphotoxin-beta receptor (LTβR). These molecules/agents, as well as other molecules/agents described herein which bind LTβR, may be referred to herein as “LTβR-binding agents”. In certain embodiments, the molecule or agent is an LTβR agonist. In certain embodiments, the molecule or agent activates LTβR and/or induces LTβR signaling. Assays for measuring activation of LTβR and induction of LTβR signaling are known in the art and certain specific examples of such assays are provided in the Examples below. In some embodiments, the molecules and agents consist essentially of or consist of lymphotoxin-αββ.

In certain embodiments, the molecule/agent is a polypeptide. In certain embodiments, the molecule/agent is a soluble protein. In some embodiments, the molecule/agent is a fusion polypeptide. In certain embodiments, the molecule/agent is a single-chain polypeptide. In some embodiments, the single-chain polypeptide may be a fully human. In some embodiments, the molecule/agent is a soluble ligand. In some embodiments, the molecule/agent is a homodimeric molecule. In some embodiments, the molecule/agent is a heterodimeric molecule. In some embodiments, the molecule/agent is a homodimeric bispecific molecule. In some embodiments, the molecule/agent is a heterodimeric bispecific molecule.

In some embodiments, a polypeptide, molecule, or agent comprises a first copy and a second copy of the extracellular domain of lymphotoxin-beta or a fragment thereof, and a copy of lymphotoxin-alpha or a fragment thereof. In some embodiments, the fragment of lymphotoxin-beta and/or lymphotoxin-alpha is a functional fragment, e.g., is able to form a lymphotoxin αββ heterotrimer and able to bind LTβR.

The full-length amino acid (aa) sequences of mouse lymphotoxin-alpha and mouse lymphotoxin-beta are known in the art (UniProt No. P09225 and P41155, respectively) and are provided herein as SEQ ID NO:1 and SEQ ID NO:4. The full-length amino acid sequences of human lymphotoxin-alpha and human lymphotoxin-beta are known in the art (UniProt No. P01374 and Q06643, respectively) and are provided herein as SEQ ID NO: 10 and SEQ ID NO: 13. Lymphotoxin-alpha is a soluble protein and lymphotoxin-beta is a membrane-bound protein. The extracellular domain of human lymphotoxin-beta is generally considered to be approximately amino acids 49-244 of SEQ ID NO: 13. Those of skill in the art may differ in their understanding of the exact amino acids corresponding to the extracellular domain of human lymphotoxin-beta. Thus, the N-terminus and/or C-terminus of the extracellular domain described herein may extend or be shortened by 1, 2, 3, 4, 5, 6, 7, 8, 9, 10 or more amino acids.

In some embodiments, a fusion polypeptide comprises a first copy and a second copy of the extracellular domain of human lymphotoxin-beta or a fragment thereof, and a copy of human lymphotoxin-alpha or a fragment thereof. In some embodiments, the fusion polypeptide is capable of binding an LTβR. In some embodiments, the fusion polypeptide is capable of binding the human LTβR. In some embodiments, the fusion polypeptide is structured sequentially as (a) lymphotoxin-alpha-lymphotoxin-beta-lymphotoxin-beta; (b) lymphotoxin-beta-lymphotoxin-alpha-lymphotoxin-beta or (c) lymphotoxin-beta-lymphotoxin-beta-lymphotoxin-alpha. In some embodiments, the fusion polypeptide is structured sequentially as lymphotoxin-alpha-lymphotoxin-beta-lymphotoxin-beta. In some embodiments, the copies of lymphotoxin-beta and lymphotoxin-alpha are all directly linked to each other. In some embodiments, the copies of lymphotoxin-beta and lymphotoxin-alpha are linked to each other with a linker. In some embodiments, the copies of lymphotoxin-beta and lymphotoxin-alpha are linked to each other with a peptide linker.

As used herein, the term “linker” refers to a linker inserted between a first polypeptide (e.g., a lymphotoxin-alpha or a fragment thereof) and a second polypeptide (e.g., an extracellular domain of lymphotoxin-beta or a fragment thereof). In some embodiments, the linker is a peptide linker. Linkers should not adversely affect the expression, secretion, or bioactivity of the fusion protein. Linkers should not be antigenic and should not elicit an immune response. Suitable linkers are known to those of skill in the art and often include mixtures of glycine and serine residues and often include amino acids that are sterically unhindered. Other amino acids that can be incorporated into useful linkers include threonine and alanine residues. Linkers can range in length, for example from 1-50 amino acids in length, 1-22 amino acids in length, 1-10 amino acids in length, 1-5 amino acids in length, or 1-3 amino acids in length. As used herein, a linker is an intervening peptide sequence that does not include amino acid residues from either the C-terminus of the first polypeptide or the N-terminus of the second polypeptide.

In some embodiments, a polypeptide comprises a first copy of human lymphotoxin-beta comprising SEQ ID NO: 14 or a fragment thereof, a second copy of human lymphotoxin-beta comprising SEQ ID NO: 14 or a fragment thereof, and a copy of human lymphotoxin-alpha comprising SEQ ID NO: 11 or a fragment thereof. In some embodiments, a polypeptide comprises a first copy of human lymphotoxin-beta comprising amino acids 49-244 of SEQ ID NO: 13 or a fragment thereof, a second copy of human lymphotoxin-beta comprising amino acids 49-244 of SEQ ID NO: 13 or a fragment thereof, and a copy of human lymphotoxin-alpha comprising amino acids 35-205 of SEQ ID NO: SEQ ID NO: 10 or a fragment thereof. In some embodiments, a polypeptide comprises a first copy and a second copy of human lymphotoxin-beta comprising amino acids 83-244 of SEQ ID NO: 13, and a copy of human lymphotoxin-alpha comprising amino acids 62-205 of SEQ ID NO: 10. In some embodiments, a polypeptide comprises a first copy and a second copy of human lymphotoxin-beta each comprising SEQ ID NO: 15 or SEQ ID NO: 108, and a copy of human lymphotoxin-alpha comprising SEQ ID NO: 12. In some embodiments, a polypeptide comprises a first copy and a second copy of human lymphotoxin-beta each comprising SEQ ID NO:15, and a copy of human lymphotoxin-alpha comprising SEQ ID NO: 12. In some embodiments, a polypeptide comprises SEQ ID NO: 16, SEQ ID NO: 17, or SEQ ID NO: 18.

In certain embodiments, a polypeptide, an agent or a molecule comprising the polypeptide (e.g., a homodimeric molecule, a heterodimeric molecule, or a binding agent) comprises a first copy and a second copy of the extracellular domain of human lymphotoxin-beta or a fragment thereof, and a copy of human lymphotoxin-alpha or a fragment thereof. In certain embodiments, a polypeptide, or a molecule comprising the polypeptide, comprises SEQ ID NO: 16. In certain embodiments, a polypeptide, or a molecule comprising the polypeptide, comprises a polypeptide having at least about 90% sequence identity to SEQ ID NO: 16. In certain embodiments, a polypeptide, or a molecule comprising the polypeptide, comprises a polypeptide having at least about 95% sequence identity to SEQ ID NO: 16. In certain embodiments, a polypeptide, or a molecule comprising the polypeptide, comprises a polypeptide having at least 96%, 97%, 98%, 99% sequence identity to SEQ ID NO: 16. In some embodiments, a polypeptide, or a molecule comprising the polypeptide, comprises a polypeptide consisting essentially of SEQ ID NO: 16. In some embodiments, a polypeptide, or a molecule comprising the polypeptide, comprises a polypeptide consisting of SEQ ID NO: 16. In certain embodiments, a polypeptide, or a molecule comprising the polypeptide, comprises SEQ ID NO: 17. In certain embodiments, a polypeptide, or a molecule comprising the polypeptide, comprises a polypeptide having at least about 90% sequence identity to SEQ ID NO: 17. In certain embodiments, a polypeptide, or a molecule comprising the polypeptide, comprises a polypeptide having at least about 95% sequence identity to SEQ ID NO: 17. In certain embodiments, a polypeptide, or a molecule comprising the polypeptide, comprises a polypeptide having at least 96%, 97%, 98%, 99% sequence identity to SEQ ID NO: 17. In some embodiments, a polypeptide, or a molecule comprising the polypeptide, comprises a polypeptide consisting essentially of SEQ ID NO: 17. In some embodiments, a polypeptide, or a molecule comprising the polypeptide, comprises a polypeptide consisting of SEQ ID NO: 17. In certain embodiments, a polypeptide, or a molecule comprising the polypeptide, comprises SEQ ID NO: 18. In certain embodiments, a polypeptide, or a molecule comprising the polypeptide, comprises a polypeptide having at least about 90% sequence identity to SEQ ID NO: 18. In certain embodiments, a polypeptide, or a molecule comprising the polypeptide, comprises a polypeptide having at least about 95% sequence identity to SEQ ID NO: 18. In certain embodiments, a polypeptide, or a molecule comprising the polypeptide, comprises a polypeptide having at least 96%, 97%, 98%, 99% sequence identity to SEQ ID NO: 18. In some embodiments, a polypeptide, or a molecule comprising the polypeptide, comprises a polypeptide consisting essentially of SEQ ID NO: 18. In some embodiments, a polypeptide, or a molecule comprising the polypeptide, comprises a polypeptide consisting of SEQ ID NO: 18.

In certain embodiments, a polypeptide, or a molecule or agent comprising the polypeptide, comprises a variant of the extracellular domain lymphotoxin-beta amino acid sequence or a fragment thereof that comprises one or more (e.g., one, two, three, four, five, six, seven, eight, nine, ten, etc.) conservative substitutions. In certain embodiments, a polypeptide, or a molecule comprising the polypeptide, comprises a variant of lymphotoxin-alpha amino acid sequence or a fragment thereof that comprises one or more (e.g., one, two, three, four, five, six, seven, eight, nine, ten, etc.) conservative substitutions. In some embodiments, the copies of the extracellular domain of lymphotoxin-beta comprise substitutions, deletions, and/or additions to the amino acid sequence of human lymphotoxin-beta as compared to the wild-type sequence. In some embodiments, the copy of lymphotoxin-alpha comprises substitutions, deletions, and/or additions to the amino acid sequence of human lymphotoxin-alpha as compared to the wild-type sequence. A fusion polypeptide comprising these lymphotoxin variants still maintains the ability to bind human LTβR.

In one aspect, the invention provides a single-chain polypeptide (and polypeptides, molecules and agents comprising the single-chain polypeptide) that is capable of binding human LTβR. In certain embodiments, the single-chain polypeptide comprises (a) a first amino acid sequence comprising the extracellular domain of human lymphotoxin-beta, a variant thereof having at least about 70%, 80%, 85%, 90%, 95%, 98%, or 99% sequence identity (e.g., 80%, 90%, or 95%) to the extracellular domain of human lymphotoxin-beta, or a fragment thereof, (b) a second amino acid sequence comprising the extracellular domain of human lymphotoxin-beta, a variant thereof having at least about 70%, 80%, 85%, 90%, 95%, 98%, or 99% sequence identity (e.g., 80%, 90%, or 95%) to the extracellular domain of human lymphotoxin-beta, or a fragment thereof; and (c) a third amino acid sequence comprising human lymphotoxin-alpha, a variant thereof having at least about 70%, 80%, 85%, 90%, 95%, 98%, or 99% sequence identity (e.g., 80%, 90%, or 95%) to human lymphotoxin-alpha, or a fragment thereof, wherein the polypeptide is capable of binding the human lymphotoxin-beta receptor. In some embodiments, the single-chain polypeptide comprises (a) a first amino acid sequence comprising the extracellular domain of human lymphotoxin-beta, a variant thereof having at least about 80% sequence identity to the extracellular domain of human lymphotoxin-beta, or a fragment thereof; (b) a second amino acid sequence comprising the extracellular domain of human lymphotoxin-beta, a variant thereof having at least about 80% sequence identity to the extracellular domain of human lymphotoxin-beta, or a fragment thereof; and/or (c) a third amino acid sequence comprising human lymphotoxin-alpha, a variant thereof having at least about 80% sequence identity to human lymphotoxin-alpha, or a fragment thereof. In further embodiments, the single-chain polypeptide comprises (a) a first amino acid sequence comprising the extracellular domain of human lymphotoxin-beta, a variant thereof having at least about 90% sequence identity to the extracellular domain of human lymphotoxin-beta, or a fragment thereof; (b) a second amino acid sequence comprising the extracellular domain of human lymphotoxin-beta, a variant thereof having at least about 90% sequence identity to the extracellular domain of human lymphotoxin-beta, or a fragment thereof; and/or (c) a third amino acid sequence comprising human lymphotoxin-alpha, a variant thereof having at least about 90% sequence identity to human lymphotoxin-alpha, or a fragment thereof. In certain embodiments, the single-chain polypeptide comprises (a) a first amino acid sequence comprising the extracellular domain of human lymphotoxin-beta, a variant thereof having at least about 95% sequence identity to the extracellular domain of human lymphotoxin-beta, or a fragment thereof; (b) a second amino acid sequence comprising the extracellular domain of human lymphotoxin-beta, a variant thereof having at least about 95% sequence identity to the extracellular domain of human lymphotoxin-beta, or a fragment thereof; and/or (c) a third amino acid sequence comprising human lymphotoxin-alpha, a variant thereof having at least about 95% sequence identity to human lymphotoxin-alpha, or a fragment thereof. In certain embodiments, the single-chain polypeptide comprises (a) a first amino acid sequence comprising the extracellular domain of human lymphotoxin-beta, or a fragment thereof; (b) a second amino acid sequence comprising the extracellular domain of human lymphotoxin-beta, or a fragment thereof; and (c) a third amino acid sequence comprising human lymphotoxin-alpha, or a fragment thereof. The first, second, and third amino acid sequences of the single-chain polypeptide may be in any order. Thus, in some embodiments, the single-chain polypeptide is structured sequentially (from the N to C terminal ends) as lymphotoxin-alpha-lymphotoxin-beta-lymphotoxin-beta. In other embodiments, the single-chain polypeptide is structured sequentially as lymphotoxin-beta-lymphotoxin-alpha-lymphotoxin-beta or lymphotoxin-beta-lymphotoxin-beta-lymphotoxin-alpha. In certain embodiments, the amino acid sequences are directly linked to each other (e.g., through a peptide bond). In certain alternative embodiments, the amino acid sequences are linked to each other with a peptide linker (e.g., a non-lymphotoxin peptide linker).

In another aspect, the invention features a single-chain polypeptide that comprises (a) a first amino acid sequence comprising a sequence having at least about 90%, at least about 95%, or at least about 98% sequence identity to SEQ ID NO: 15 or SEQ ID NO: 108; (b) a second amino acid sequence comprising a sequence having at least about 90%, at least about 95%, or at least about 98% sequence identity to SEQ ID NO: 15 or SEQ ID NO: 108; and/or (c) a third amino acid sequence comprising a sequence having at least about 90%, at least about 95%, or at least about 98% sequence identity to SEQ ID NO:12, wherein the single-chain polypeptide is capable of binding the human lymphotoxin-beta receptor. In some embodiments, the single-chain polypeptide comprises; (a) a first amino acid sequence comprising a sequence having at least about 90% sequence identity to SEQ ID NO: 15; (b) a second amino acid sequence comprising a sequence having at least about 90% sequence identity to SEQ ID NO: 15; and (c) a third amino acid sequence comprising a sequence having at least about 90% sequence identity to SEQ ID NO: 12. In some embodiments, the single-chain polypeptide comprises; (a) a first amino acid sequence comprising a sequence having at least about 95% sequence identity to SEQ ID NO: 15; (b) a second amino acid sequence comprising a sequence having at least about 95% sequence identity to SEQ ID NO:15; and (c) a third amino acid sequence comprising a sequence having at least about 95% sequence identity to SEQ ID NO: 12. In further embodiments, the single-chain polypeptide comprises; (a) a first amino acid sequence comprising a sequence having at least about 98% sequence identity to SEQ ID NO: 15; (b) a second amino acid sequence comprising a sequence having at least about 98% sequence identity to SEQ ID NO: 15 and (c) a third amino acid sequence comprising a sequence having at least about 98% sequence identity to SEQ ID NO: 12. In still further embodiments, the single-chain polypeptide comprises (a) a first amino acid sequence comprising SEQ ID NO: 15; (b) a second amino acid sequence comprising SEQ ID NO: 15; and (c) a third amino acid sequence comprising SEQ ID NO: 12. In certain embodiments, the amino acid sequences are directly linked through a peptide bond. In certain alternative embodiments, the amino acid sequences are linked to each other with a peptide linker (e.g., a non-lymphotoxin peptide linker). In certain embodiments, the single-chain polypeptide is structured sequentially (from N to C terminal) as lymphotoxin-alpha-lymphotoxin-beta-lymphotoxin-beta. In certain other embodiments, the single-chain polypeptide is structured sequentially as lymphotoxin-beta-lymphotoxin-alpha-lymphotoxin-beta or lymphotoxin-beta-lymphotoxin-beta-lymphotoxin-alpha. In certain embodiments, the single-chain polypeptide further comprises a targeting moiety.

The invention further provides a single-chain polypeptide that is capable of binding human LTβR and comprises (a) a first amino acid sequence comprising SEQ ID NO: 15 or SEQ ID NO: 108. (b) a second amino acid sequence of comprising SEQ ID NO: 15 or SEQ ID NO: 108, and (c) an amino acid sequence comprising SEQ ID NO: 12. In certain embodiments, the first and second amino acid sequence each comprise SEQ ID NO: 15. In certain embodiments, the amino acid sequences are directly linked to each other (e.g., through a peptide bond). In certain alternative embodiments, the amino acid sequences are linked to each other with a peptide linker (e.g., a non-lymphotoxin peptide linker). In some embodiments, the single-chain polypeptide is structured sequentially (from N to C terminal) as lymphotoxin-alpha-lymphotoxin-beta-lymphotoxin-beta. In certain other embodiments, the single-chain polypeptide is structured sequentially as lymphotoxin-beta-lymphotoxin-alpha-lymphotoxin-beta or lymphotoxin-beta-lymphotoxin-beta-lymphotoxin-alpha. In some embodiments, the single-chain polypeptide comprises SEQ ID NO: 16, SEQ ID NO: 17, or SEQ ID NO: 18. For example, in certain embodiments, the single-chain polypeptide comprises SEQ ID NO: 16. In certain other embodiments, the single-chain polypeptide comprises SEQ ID NO: 17. Alternatively, the single-chain polypeptide may comprise SEQ ID NO: 18. In certain embodiments, the single-chain polypeptide further comprises a targeting moiety (e.g., a full-length antibody or an antigen-binding antibody fragment that specifically binds a tumor-associated antigen).

In another aspect, the invention provides a single-chain polypeptide capable of binding LTβR, wherein the single-chain polypeptide comprises a polypeptide having at least about 90%, at least about 95%, or at least about 98% sequence identity to SEQ ID NO: 16, SEQ ID NO: 17, or SEQ ID NO:18. In certain embodiments, the single-chain polypeptide comprises a polypeptide having at least about 95% sequence identity to SEQ ID NO: 16, SEQ ID NO: 17, or SEQ ID NO: 18. Thus, in some embodiments, the single-chain polypeptide comprises a polypeptide having at least about 95% sequence identity to SEQ ID NO:16. In certain embodiments, the single-chain polypeptide comprises a polypeptide having at least about 95% sequence identity to SEQ ID NO: 17. In certain embodiments, the single-chain polypeptide comprises a polypeptide having at least about 95% sequence identity to SEQ ID NO: 18. Single-chain polypeptides comprising a polypeptide having at least about 98% sequence identity to SEQ ID NO: 16, SEQ ID NO: 17, or SEQ ID NO: 18 are also provided. In certain embodiments, the single-chain polypeptide comprises a polypeptide having at least about 98% sequence identity to SEQ ID NO: 16. In some embodiments, the single-chain polypeptide comprises, consists essentially of, or consists of SEQ ID NO: 16. SEQ ID NO: 17, or SEQ ID NO:18. In certain embodiments, the single-chain polypeptide further comprises a targeting moiety (e.g., a full-length antibody or an antigen-binding antibody fragment that specifically binds a tumor-associated antigen).

The invention further provides agents, molecules, and polypeptides comprising each of the single-chain polypeptides described above and elsewhere herein. In certain embodiments, such agents, molecules, and polypeptides further comprise a targeting moiety linked to the single-chain polypeptides described herein. In certain embodiments, the targeting moiety binds a cell-surface antigen, such as a tumor-associated antigen (e.g., B7-H4 or P-CADHERIN). The targeting moiety may be linked to the single-chain polypeptide directly (e.g., by a peptide bond) or by a linker peptide. In certain embodiments, the agents, molecules, and polypeptides comprising the single-chain polypeptide may further comprise a human Fc region. In certain embodiments, targeting moiety of the agents, molecules, and polypeptides may be a full-length antibody (e.g., a monoclonal antibody) that binds a tumor-associated antigen and the LTβR-binding single-chain polypeptide may be linked to the C-terminus of the heavy chain of the antibody.

In some embodiments, a molecule, agent or polypeptide comprising lymphotoxin-αββ (e.g., a fusion polypeptide or single-chain polypeptide described herein) further comprises a targeting moiety. Active targeting generally takes advantage of ligand-receptor, antigen-antibody, and other forms of molecular recognition to deliver an agent, molecule, protein, drug, etc. to a specific location. For cancer treatment, active targeting moieties may be particularly beneficial because they reduce or eliminate the delivery of potentially toxic drugs to healthy tissue. In some embodiments, a polypeptide comprises a polypeptide comprising lymphotoxin-αββ described herein and a targeting moiety. In some embodiments, the targeting moiety is capable of binding a target cell. In some embodiments, the target cell is a tumor cell. In some embodiments, targeting moiety comprises a non-lymphotoxin polypeptide. In some embodiments, the targeting moiety comprises an antigen-binding site. In some embodiments, the targeting moiety comprises an antibody. In some embodiments, the targeting moiety comprises an antibody that specifically binds a tumor-associated antigen (TAA). Non-limiting examples of tumor-associated antigens are provided below in the section entitled “III. Targeting to specific cell types.”

In some embodiments, the polypeptide comprising lymphotoxin αββ (e.g., fusion polypeptide or single-chain polypeptide) described herein is directly linked to a non-lymphotoxin polypeptide. In some embodiments, the polypeptide comprising lymphotoxin αββ described herein is connected to a non-lymphotoxin polypeptide by a linker. In some embodiments, the N-terminal end of the polypeptide comprising lymphotoxin αββ described herein is linked to the C-terminal end of a non-lymphotoxin polypeptide. In some embodiments, the C-terminal end of the polypeptide comprising lymphotoxin αββ described herein is linked to the N-terminal end of a non-lymphotoxin polypeptide. In some embodiments, the non-lymphotoxin polypeptide comprises an immunoglobulin heavy chain. In some embodiments, the immunoglobulin heavy chain is IgG1, IgG2, IgG3, or IgG4. In some embodiments, the immunoglobulin heavy chain is mutated at the glycosylation site. In some embodiments, the immunoglobulin heavy chain is associated with an immunoglobulin light chain. In some embodiments, the non-lymphotoxin polypeptide comprises a Fc region, one or more protein tags (e.g., myc, FLAG, GST), other endogenous proteins or protein fragments, or any other useful protein sequence. In some embodiments, the non-lymphotoxin polypeptide comprises a human Fc region. The Fc region can be obtained from any of the classes of immunoglobulin, IgG, IgA, IgM, IgD and IgE. In some embodiments, the Fc region is a human IgG1 Fe region. In some embodiments, the Fc region is a human IgG2 Fc region. In some embodiments, the Fc region is a wild-type Fc region. In some embodiments, the Fc region is a natural variant of a wild-type Fc region. In some embodiments, the Fc region is a mutated Fc region. In some embodiments, the Fc region is truncated at the N-terminal end by 1, 2, 3, 4, 5, 6, 7, 8, 9, or 10 amino acids, (e.g., in the hinge domain). In some embodiments, the Fc region is truncated at the C-terminal end (e.g., lysine is absent). In some embodiments, an amino acid in the hinge domain is changed to hinder undesirable disulfide bond formation. In some embodiments, a cysteine is replaced with a different amino acid to hinder undesirable disulfide bond formation. In some embodiments, a cysteine is replaced with a serine to hinder undesirable disulfide bond formation. In some embodiments, the Fc region is mutated at the glycosylation site. In some embodiments, the Fc region is mutated to allow heterodimer formation. In some embodiments, the Fc region is selected from the group consisting of: SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:38, SEQ ID NO:39, SEQ ID NO:40, and SEQ ID NO:41. In some embodiments, the Fc region comprises SEQ ID NO:38, SEQ ID NO:39, SEQ ID NO:40, or SEQ ID NO:41.

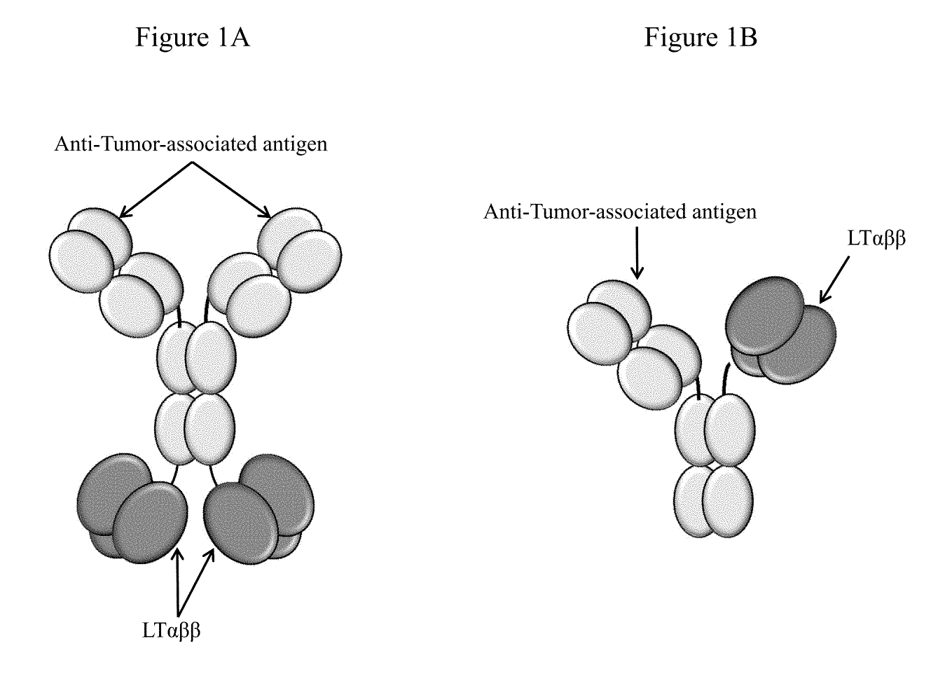

In another aspect of the invention, a homodimeric molecule comprises a lymphotoxin fusion polypeptide or single-chain polypeptide described herein. In some embodiments, a homodimeric molecule comprises two monomers, wherein each monomer comprises (a) a first polypeptide comprising a first copy and a second copy of the extracellular domain of human lymphotoxin-beta or a fragment thereof, and a copy of human lymphotoxin-alpha or a fragment thereof; and (b) a second polypeptide comprising a targeting moiety. For a representative example of this homodimeric format, see FIG. 1A. In some embodiments, the first polypeptide is structured sequentially as; (a) lymphotoxin-alpha-lymphotoxin-beta-lymphotoxin-beta; (b) lymphotoxin-beta-lymphotoxin-alpha-lymphotoxin-beta; or (c) lymphotoxin-beta-lymphotoxin-beta-lymphotoxin-alpha. In some embodiments, the copies of lymphotoxin-beta and lymphotoxin-alpha are all directly linked to each other. In some embodiments, the copies of lymphotoxin-beta and lymphotoxin-alpha are linked to each other with a peptide linker. In some embodiments, the first copy and the second copy of human lymphotoxin-beta each comprise SEQ ID NO:14 or a fragment thereof, and the copy of human lymphotoxin-alpha comprises SEQ ID NO: 11 or a fragment thereof. In some embodiments, a polypeptide comprises a first copy of human lymphotoxin-beta comprising amino acids 49-244 of SEQ ID NO: 13 or a fragment thereof, a second copy of human lymphotoxin-beta comprising amino acids 49-244 of SEQ ID NO: 13 or a fragment thereof, and a copy of human lymphotoxin-alpha comprising amino acids 35-205 of SEQ ID NO: SEQ ID NO: 10 or a fragment thereof. In some embodiments, the first copy and the second copy of human lymphotoxin-beta each comprise amino acids 83-244 of SEQ ID NO: 13, and the copy of human lymphotoxin-alpha comprises amino acids 62-205 of SEQ ID NO: 10. In some embodiments, the first copy and the second copy of human lymphotoxin-beta each comprise SEQ ID NO: 15, and the copy of human lymphotoxin-alpha comprises SEQ ID NO: 12. In some embodiments, each monomer of the homodimeric molecule comprises SEQ ID NO: 16, SEQ ID NO: 17, or SEQ ID NO: 18. In some embodiments, each monomer of the homodimeric molecule comprises SEQ ID NO: 16. In some embodiments, the first polypeptide is directly linked to the second polypeptide. In some embodiments, the first polypeptide is connected to the second polypeptide by a linker. In some embodiments, the N-terminal end of the first polypeptide is linked to the C-terminal end of the second polypeptide. In some embodiments, the N-terminal end of the lymphotoxin αββ polypeptide is directly linked to the C-terminal end of an antibody immunoglobulin heavy chain.

In some embodiments, each monomer of the homodimeric molecule comprises a targeting moiety. In some embodiments, the targeting moiety is capable of binding a target cell. In some embodiments, the target cell is a tumor cell. In some embodiments, the targeting moiety is an antibody. In some embodiments, the second polypeptide is an antibody. In some embodiments, the antibody is an IgG1, IgG2, IgG3, or IgG4 antibody. In some embodiments, the antibody is mutated at the glycosylation site within the immunoglobulin heavy chain. In some embodiments, the antibody specifically binds a tumor cell. In some embodiments, the antibody specifically binds a tumor-associated antigen. In some embodiments, the tumor-associated antigen is B7-H4. In some embodiments, the tumor-associated antigen is P-CADHERIN. It will be understood by one of skill in the art that this homodimeric molecule platform allows for almost any antibody or antigen-binding site to be used as part of each monomer.

In certain embodiments, the polypeptide, molecule, or agent comprises SEQ ID NO:95, SEQ ID NO:96, or SEQ ID NO:97. For example, a monomer of a homodimeric molecule may comprise SEQ ID NO:95, SEQ ID NO:96, or SEQ ID NO:97. In certain embodiments, the polypeptide (e.g., fusion polypeptide or single-chain polypeptide), molecule, or agent comprises, consists essentially of, or consists of SEQ ID NO:95. In certain embodiments, the polypeptide, molecule, or agent comprises, consists essentially of, or consists of SEQ ID NO:96. In certain embodiments, the polypeptide, molecule, or agent comprises, consists essentially of, or consists of SEQ ID NO:97. In certain embodiments, the polypeptide molecule comprises SEQ ID NO:95, SEQ ID NO:96, or SEQ ID NO:97 and further comprises a targeting moiety (e.g., an antibody that specifically binds a tumor-associated antigen).

In another aspect of the invention, a heterodimeric molecule comprises a lymphotoxin fusion polypeptide or single-chain polypeptide described herein. In some embodiments, a heterodimeric molecule comprises two monomers, wherein a first monomer comprises a first copy and a second copy of the extracellular domain of human lymphotoxin-beta or a fragment thereof, and a copy of human lymphotoxin-alpha or a fragment thereof, and a second monomer comprises a targeting moiety. For a representative example of the heterodimeric format, see FIG. 1B. In some embodiments, the first monomer comprises a first copy and a second copy of human lymphotoxin-beta each comprising SEQ ID NO: 14 or a fragment thereof, and a copy of human lymphotoxin-alpha comprising SEQ ID NO: 11 or a fragment thereof. In some embodiments, the first monomer comprises a first copy of human lymphotoxin-beta comprising amino acids 49-244 of SEQ ID NO: 13 or a fragment thereof, a second copy of human lymphotoxin-beta comprising amino acids 49-244 of SEQ ID NO: 13 or a fragment thereof, and a copy of human lymphotoxin-alpha comprising amino acids 35-205 of SEQ ID NO: 10 or a fragment thereof. In some embodiments, the first monomer comprises a first copy and a second copy of human lymphotoxin-beta each comprising amino acids 83-244 of SEQ ID NO: 13, and a copy of human lymphotoxin-alpha comprising amino acids 62-205 of SEQ ID NO: 10. In some embodiments, the first monomer comprises a first copy and a second copy of human lymphotoxin-beta each comprising SEQ ID NO: 15, and a copy of human lymphotoxin-alpha comprising SEQ ID NO:12. In some embodiments, the first monomer comprises SEQ ID NO: 16, SEQ ID NO: 17, or SEQ ID NO: 18. In some embodiments, the first monomer comprises SEQ ID NO: 16. In some embodiments, the first monomer further comprises a human Fc region. In some embodiments, the human Fc region is from an IgG1, IgG2, IgG3, or IgG4 immunoglobulin. In some embodiments, the human Fc region is mutated at the glycosylation site. In some embodiments, the human Fc region is mutated to allow heterodimer formation. In some embodiments, the human Fc region is selected from the group consisting of: SEQ ID NO:27, SEQ ID NO:28, SEQ ID NO:29, SEQ ID NO:30, SEQ ID NO:31. SEQ ID NO:32. SEQ ID NO:33, SEQ ID NO:34, SEQ ID NO:38, SEQ ID NO:39, SEQ ID NO:40, and SEQ ID NO:41. In some embodiments, the second monomer of the heterodimeric molecule comprises an immunoglobulin heavy chain. In some embodiments, the immunoglobulin heavy chain is IgG1, IgG2, IgG3, or IgG4. In some embodiments, the immunoglobulin heavy chain is mutated at the glycosylation site. In some embodiments, the immunoglobulin heavy chain is mutated to allow heterodimer formation. In some embodiments, the immunoglobulin heavy chain is associated with an immunoglobulin light chain. In some embodiments, the immunoglobulin heavy chain is associated with an immunoglobulin light chain to form an antigen-binding site. In some embodiments, the second monomer comprises an antigen-binding site that specifically binds a tumor-associated antigen. In some embodiments, the second monomer comprises an antibody that specifically binds a tumor-associated antigen. Additional tumor-associated antigens are described in the section below. In particular embodiments, the tumor antigen is selected from the group consisting of B7-H4, P-CADHERIN (CDH3), GABRP, ACPP, SLC45A3, STEAP1, STEAP2, GPA33, GUCY2C, PVRL4, and GARP. In some embodiments, the tumor-associated antigen is B7-H4. In some embodiments, the tumor-associated antigen is P-CADHERIN. It will be understood by one of skill in the art that this heterodimeric molecule platform allows for almost any antibody or antigen-binding site to be used as part of the second monomer.

In another aspect, the invention provides molecules and agents that comprise (a) an LTβR-binding moiety, wherein the LTβR-binding moiety comprises a lymphotoxin αββ heterotrimer, and (b) a targeting moiety (e.g., an antibody). In some embodiments, the molecules or agents may be polypeptides. In certain embodiments, the LTβR-binding moiety may activate LTβR and/or induce LTβR signaling. The LTβR-binding moiety may be a single-chain polypeptide. The LTβR-binding moiety may be a fully human single-chain polypeptide. Thus, in certain embodiments, the molecule or agent provided herein comprises a single-chain lymphotoxin αββ heterotrimer as the LTβR-binding moiety. In certain embodiments, the LTβR-binding moiety comprises any lymphotoxin-αββ (e.g., fusion polypeptide or single-chain polypeptide) described herein that binds LTβR. In some embodiments, the LTβR-binding moiety comprises any of the fusion polypeptides or single-chain polypeptides described herein that bind LTβR. In certain embodiments, the LTβR-binding moiety comprises a polypeptide (e.g., single-chain polypeptide) comprising (a) a first amino acid sequence comprising the extracellular domain of human lymphotoxin-beta, a variant thereof having at least about 80%, at least about 90%, at least about 95%, or at least about 98% sequence identity to the extracellular domain of human lymphotoxin-beta, or a fragment thereof; (b) a second amino acid sequence comprising the extracellular domain of human lymphotoxin-beta, a variant thereof having at least about 80%, at least about 90%, at least about 95%, or at least about 98% sequence identity to the extracellular domain of human lymphotoxin-beta, or a fragment thereof; and/or (c) a third amino acid sequence comprising human lymphotoxin-alpha, a variant thereof having at least about 80%, at least about 90%, at least about 95%, or at least about 98% sequence identity to human lymphotoxin-alpha, or a fragment thereof. In certain alternative embodiments, the LTβR-binding moiety comprises a polypeptide comprising (a) a first amino acid sequence comprising a sequence having at least about 90%, at least about 95%, or at least about 98% sequence identity to SEQ ID NO: 15 or SEQ ID NO: 108. (b) a second amino acid sequence comprising a sequence having at least about 90%, at least about 95%, or at least about 98% sequence identity to SEQ ID NO: 15 or SEQ ID NO: 108; and/or (c) a third amino acid sequence comprising a sequence having at least about 90%, at least about 95%, or at least about 98% sequence identity to SEQ ID NO: 12. For example, the LTβR-binding moiety may comprises a polypeptide comprising (a) a first amino acid sequence comprising SEQ ID NO: 15; (b) a second amino acid sequence comprising SEQ ID NO: 15; and/or (c) a third amino acid sequence comprising SEQ ID NO: 12. In another embodiment, the LTβR-binding moiety may comprise a polypeptide having at least about 95% sequence identity to SEQ ID NO: 16, SEQ ID NO: 17, or SEQ ID NO: 18. In another embodiment, the LTβR-binding moiety may comprise SEQ ID NO: 16, SEQ ID NO: 17, or SEQ ID NO: 18. In some embodiments, the molecule or agent comprises SEQ ID NO:95, SEQ ID NO:96 or SEQ ID NO:97.

In certain embodiments, the targeting moiety of the molecule or agent comprising both an LTβR-binding moiety and a targeting moiety is capable of binding a cell-surface antigen. In certain embodiments, the targeting moiety selectively targets the target cell (and/or the tissue containing the target cell). In certain embodiments, the targeting moiety specifically binds a tumor-associated antigen (e.g., a tumor-associated antigen listed in the section below entitled “III. Targeting to specific cell types”). In certain embodiments, the tumor-associated antigen is selected from the group consisting of B7-H4, B7-H3. P-CADHERIN (CDH3), GABRP, ACPP, SLC45A3, STEAP1, STEAP2, GPA33, GUCY2C, GARP, PVRL4, mesothelin and CA9. In some embodiments, the targeting moiety is an antibody (e.g., a full-length antibody or an antigen-binding antibody fragment). By way of non-limiting example, the targeting moiety of the molecule or agent may be an antibody that specifically binds the extracellular domain of human B7-H4. In certain other embodiments, the targeting moiety of the molecule or agent may be an antibody that specifically binds the extracellular domain of human P-CADHERIN. The B7-H4 or P-CADHERIN antibody may, in some embodiments, be selected from the B7-H4 or P-CADHERIN antibodies described in the section below entitled “IV. Antibodies to cell-surface antigens” or described elsewhere herein.

Thus, in some embodiments, the molecule or agent comprises (a) an antibody that specifically binds a cell surface antigen (e.g., a tumor-associated antigen such as B7-H4 or P-CADHERIN); and (b) a single-chain lymphotoxin αββ heterotrimer.

In another aspect, the invention provides a polypeptide, agent or molecule that binds both the extracellular domain of human B7-H4 and human LTβR. In certain embodiments, the polypeptide, agent, or molecule also activates human LTβR and/or induces LTβR signaling. In some embodiments, the polypeptide, agent or molecule comprises SEQ ID NO: 105 and/or SEQ ID NO: 107. In certain embodiments, the polypeptide, agent or molecule comprises SEQ ID NO: 105 and SEQ ID NO: 107

In a further aspect, the invention provides a polypeptide, agent or molecule that binds both the extracellular domain of mature human P-CADHERIN and human LTβR. In certain embodiments, the polypeptide, agent, or molecule also activates human LTβR and/or induces LTβR signaling. In some embodiments, the polypeptide, agent or molecule comprises SEQ ID NO:99 and/or SEQ ID NO: 102. In certain embodiments, the polypeptide, agent or molecule comprises SEQ ID NO:99 and SEQ ID NO: 102

In certain embodiments, a molecule, polypeptide, agent, single-chain polypeptide or fusion polypeptide described herein comprises the Fc region of an immunoglobulin. Those skilled in the art will appreciate that some of the molecules, polypeptides or agents of this invention will comprise fusion proteins or other polypeptides in which at least a portion of the Fc region has been deleted or otherwise altered so as to provide desired biochemical characteristics, such as increased cancer cell localization, increased tumor penetration, reduced serum half-life, or increased serum half-life, when compared with a fusion protein of approximately the same immunogenicity comprising a native or unaltered Fc region. Modifications to the Fc region may include additions, deletions, or substitutions of one or more amino acids in one or more domains. The modified fusion proteins or other polypeptides disclosed herein may comprise alterations or modifications to one or more of the two heavy chain constant domains (CH2 or CH3) or to the hinge region. In other embodiments, the entire CH2 domain may be removed (ΔCH2 constructs). In some embodiments, the omitted constant region domain is replaced by a short amino acid spacer (e.g., 10 aa residues) that provides some of the molecular flexibility typically imparted by the absent constant region domain.

In some embodiments, the modified fusion proteins or other modified polypeptides are engineered to link the CH3 domain directly to the hinge region or to the first polypeptide. In other embodiments, a peptide spacer or linker is inserted between the hinge region or the first polypeptide and the modified CH2 and/or CH3 domains. For example, constructs may be expressed wherein the CH2 domain has been deleted and the remaining CH3 domain (modified or unmodified) is joined to the hinge region or first polypeptide with a 5-20 amino acid spacer. Such a spacer may be added to ensure that the regulatory elements of the constant domain remain free and accessible or that the hinge region remains flexible. However, it should be noted that amino acid spacers may, in some cases, prove to be immunogenic and elicit an unwanted immune response against the construct. Accordingly, in certain embodiments, any spacer added to the construct will be relatively non-immunogenic so as to maintain the desired biological qualities of the fusion protein or other polypeptide.

In some embodiments, the modified fusion proteins or other polypeptides may have only a partial deletion of a constant domain or substitution of a few or even a single amino acid. For example, the mutation of a single amino acid in selected areas of the CH2 domain may be enough to substantially reduce Fc binding and thereby increase cancer cell localization and/or tumor penetration. Similarly, it may be desirable to simply delete that part of one or more constant region domains that control a specific effector function (e.g., complement C1q binding). Such partial deletions of the constant regions may improve selected characteristics of the polypeptide or molecule (e.g., serum half-life) while leaving other desirable functions associated with the subject constant region domain intact. Moreover, as alluded to above, the constant regions of the disclosed fusion proteins may be modified through the mutation or substitution of one or more amino acids that enhances the profile of the resulting construct. In this respect it may be possible to disrupt the activity provided by a conserved binding site (e.g., Fc binding) while substantially maintaining the configuration and immunogenic profile of the modified fusion protein. In certain embodiments, the modified fusion proteins or other polypeptides comprise the addition of one or more amino acids to the constant region to enhance desirable characteristics such as decreasing or increasing effector function, or provide for more cytotoxin or carbohydrate attachment sites.

It is known in the art that the constant region mediates several effector functions. For example, binding of the C1 component of complement to the Fc region of IgG or IgM antibodies (bound to antigen) activates the complement system. Activation of complement is important in the opsonization and lysis of cell pathogens. The activation of complement also stimulates the inflammatory response and can also be involved in autoimmune hypersensitivity. In addition, the Fc region can bind to a cell expressing a Fc receptor (FcR). There are a number of Fc receptors which are specific for different classes of antibody, including IgG (gamma receptors), IgE (epsilon receptors), IgA (alpha receptors) and IgM (mu receptors).

In some embodiments, the modified fusion proteins or other polypeptides provide for altered effector functions that, in turn, affect the biological profile of the polypeptide or molecule. For example, in some embodiments, the deletion or inactivation (through point mutations or other means) of a constant region domain may reduce Fc receptor binding of the circulating modified agent, thereby increasing cancer cell localization and/or tumor penetration. In other embodiments, the constant region modifications increase or reduce the serum half-life of the polypeptide or molecule. In some embodiments, the constant region is modified to eliminate disulfide linkages or oligosaccharide moiety attachment sites.

In certain embodiments, a modified fusion protein or other polypeptide does not have one or more effector functions normally associated with an Fc region. In some embodiments, the polypeptide or molecule has no antibody-dependent cell-mediated cytotoxicity (ADCC) activity, and/or no complement-dependent cytotoxicity (CDC) activity. In certain embodiments, the polypeptide or molecule does not bind to the Fc receptor and/or complement factors. In certain embodiments, the polypeptide or molecule has no effector function normally associated with an Fc region.

In some embodiments, a polypeptide, agent, or molecule described herein specifically binds human LTβR with a dissociation constant (K_{D}) of about 1 μM or less, about 100 nM or less, about 40 nM or less, about 20 nM or less, about 10 nM or less, about 1 nM or less, or about 0.1 nM or less. In some embodiments, a polypeptide or molecule described herein specifically binds human LTβR with a K₀ of about 10 nM or less. In some embodiments, a polypeptide or molecule described herein specifically binds human LTβR with a K_{D} of about 1 nM or less. In some embodiments, a polypeptide or molecule described herein specifically binds human LTβR with a K_{D} of about 0.1 nM or less.

In some embodiments, the dissociation constant of the polypeptide, agent, or molecule described herein specifically is the dissociation constant determined using an LTβR fusion protein comprising at least a portion of the LTβR extracellular domain immobilized on a Biacore chip.

In some embodiments, a polypeptide, agent or molecule binds a human LTβR with a half maximal effective concentration (ECs) of about 1 μM or less, about 100 nM or less, about 40 nM or less, about 20 nM or less, about 10 nM or less, about 1 nM or less, or about 0.1 nM or less.

In certain embodiments, fusion polypeptides, single-chain polypeptides, and other polypeptides are made using recombinant DNA techniques as known to one skilled in the art. In some embodiments, polynucleotides encoding a specific protein or a fragment thereof are isolated from mammalian cells, such as by RT-PCR using oligonucleotide primers that specifically amplify the gene encoding the protein, and the nucleotide sequence is determined using conventional techniques. The isolated polynucleotides encoding the protein may be cloned into suitable expression vectors which produce the polypeptide when transfected into host cells such as E. coli, simian COS cells, or Chinese hamster ovary (CHO) cells. In other embodiments, recombinant proteins, or fragments thereof, can be isolated from phage display libraries or using other cell surface display techniques.

The polynucleotide(s) encoding a protein can be modified in a number of different manners using recombinant DNA technology to generate alternative or variant proteins. Site-directed or high-density mutagenesis of a protein can be used to optimize specificity, affinity, stability, etc. of a recombinant protein.

Proteins generally contain a signal sequence that directs the transport of the proteins. Signal sequences (also referred to as signal peptides or leader sequences) are located at the N-terminus of nascent polypeptides. They target the polypeptide to the endoplasmic reticulum, and the proteins are sorted to their destinations, for example, to the inner space of an organelle, to an interior membrane, to the cell outer membrane, or to the cell exterior via secretion. Most signal sequences are cleaved from the protein by a signal peptidase after the proteins are transported to the endoplasmic reticulum. The cleavage of the signal sequence from the polypeptide usually occurs at a specific site in the amino acid sequence and is dependent upon amino acid residues within the signal sequence. Although there is usually one specific cleavage site, more than one cleavage site may be recognized and/or used by a signal peptidase resulting in a non-homogenous N-terminus of the polypeptide. For example, the use of different cleavage sites within a signal sequence can result in a polypeptide expressed with different N-terminal amino acids. Accordingly, in some embodiments, the polypeptides as described herein may comprise a mixture of polypeptides with different N-termini. In some embodiments, the N-termini differ in length by 1, 2, 3, 4, 5, 6, 7, 8, 9, 10, or more amino acids. In some embodiments, the N-termini differ in length by 1, 2, 3, 4, or 5 amino acids. In some embodiments, the polypeptide is substantially homogeneous, i.e., the polypeptides have the same N-terminus. In some embodiments, the signal sequence of the polypeptide comprises one or more (e.g., one, two, three, four, five, six, seven, eight, nine, ten, etc.) amino acid substitutions and/or deletions as compared to the native sequence of the protein. In some embodiments, the signal sequence of the polypeptide comprises amino acid substitutions and/or deletions that allow one cleavage site to be dominant, thereby resulting in a substantially homogeneous polypeptide with one N-terminus. In some embodiments, the signal sequence of a fusion polypeptide or other polypeptide is not the native signal sequence of the protein(s) contained within the fusion polypeptide.

In some embodiments, a heterodimer molecule described herein comprises a first CH3 domain and a second CH3 domain, each of which is modified to promote the formation of heteromultimers or heterodimers. In some embodiments, the first and second CH3 domains are modified using a knobs-into-holes technique. In some embodiments, the first and second CH3 domains comprise changes in amino acids that result in altered electrostatic interactions. In some embodiments, the first and second CH3 domains comprise changes in amino acids that result in altered hydrophobic/hydrophilic interactions (see, for example, U.S. Patent App. Publication No. 2011/0123532).

In some embodiments, the heterodimeric molecule comprises heavy chain constant regions selected from the group consisting of; (a) a first human IgG1 constant region, wherein the amino acids at positions corresponding to positions 253 and 292 of SEQ ID NO: 19 or SEQ ID NO:20 are replaced with glutamate or aspartate, and a second human IgG constant region, wherein the amino acids at positions corresponding to 240 and 282 of SEQ ID NO: 19 or SEQ ID NO:20 are replaced with lysine; (b) a first human IgG2 constant region, wherein the amino acids at positions corresponding to positions 249 and 288 of SEQ ID NO:21 or SEQ ID NO:22 are replaced with glutamate or aspartate, and a second human IgG2 constant region wherein the amino acids at positions corresponding to positions 236 and 278 of SEQ ID NO:21 or SEQ ID NO:22 are replaced with lysine; (c) a first human IgG3 constant region, wherein the amino acids at positions corresponding to positions 300 and 339 of SEQ ID NO:23 or SEQ ID NO:24 are replaced with glutamate or aspartate, and a second human IgG3 constant region wherein the amino acids at positions corresponding to positions 287 and 329 of SEQ ID NO:23 or SEQ ID NO:24 are replaced with lysine; and (d) a first human IgG4 constant region, wherein the amino acids at positions corresponding to positions 250 and 289 of SEQ ID NO:25 or SEQ ID NO:26 are replaced with glutamate or aspartate, and a second IgG4 constant region wherein the amino acids at positions corresponding to positions 237 and 279 of SEQ ID NO:25 or SEQ ID NO:26 are replaced with lysine.

In some embodiments, the heterodimeric molecule (e.g., a bispecific agent) comprises heavy chain CH2 and CH3 domains selected from the group consisting of; (a) a first human IgG1 CH2 and CH3 domain, wherein the amino acids at positions corresponding to positions 253 and 292 of SEQ ID NO: 19 or SEQ ID NO:20 are replaced with glutamate or aspartate, and a second human IgG1 CH2 and CH3 domain, wherein the amino acids at positions corresponding to 240 and 282 of SEQ ID NO: 19 or SEQ ID NO:20 are replaced with lysine; (b) a first human IgG2 CH2 and CH3 domain, wherein the amino acids at positions corresponding to positions 249 and 288 of SEQ ID NO:21 or SEQ ID NO:22 are replaced with glutamate or aspartate, and a second human IgG2 CH2 and CH3 domain, wherein the amino acids at positions corresponding to positions 236 and 278 of SEQ ID NO:21 or SEQ ID NO:22 are replaced with lysine; (c) a first human IgG3 CH2 and CH3 domain, wherein the amino acids at positions corresponding to positions 300 and 339 of SEQ ID NO:23 or SEQ ID NO:24 are replaced with glutamate or aspartate, and a second human IgG3 CH2 and CH3 domain, wherein the amino acids at positions corresponding to positions 287 and 329 of SEQ ID NO:23 or SEQ ID NO:24 are replaced with lysine; and (d) a first human IgG4 CH2 and CH3 domain, wherein the amino acids at positions corresponding to positions 250 and 289 of SEQ ID NO:25 or SEQ ID NO:26 are replaced with glutamate or aspartate, and a second IgG4 CH2 and CH3 domain, wherein the amino acids at positions corresponding to positions 237 and 279 of SEQ ID NO:25 or SEQ ID NO:26 are replaced with lysine.

In some embodiments, the polypeptides, molecules, agent, or binding agents are monovalent. In some embodiments, the polypeptide, molecule, or binding agent is a soluble protein that is monovalent. In some embodiments, the polypeptides, molecules, or binding agents described herein are bivalent. In some embodiments, the polypeptides, molecules, or binding agents described herein are trivalent. In some embodiments, the polypeptides, molecules, or binding agents described herein are monospecific. In some embodiments, the polypeptides, molecules, or binding agents described herein are bispecific. In some embodiments, the polypeptides, molecules, or binding agents described herein are multispecific. In some embodiments, the molecule or binding agent is a heterodimeric molecule that comprises two arms wherein at least one arm is monovalent. In some embodiments, the molecule or binding agent is a heterodimeric molecule that comprises two arms wherein at least one arm is bivalent. In some embodiments, the molecule or binding agent is a heterodimeric protein that comprises two arms wherein at least one arm is trivalent (i.e., binds three target molecules).

In some embodiments, a polypeptide, molecule, agent, or binding agent comprises polypeptides that are substantially homologous to the fusion proteins, polypeptides, and/or molecules described herein. These polypeptides, molecules, agents, or binding agents can contain, for example, conservative substitution mutations, i.e. the substitution of one or more amino acids by similar amino acids.

The polypeptides, molecules, and agents of the present invention can be assayed for specific binding to a target by any method known in the art. The immunoassays which can be used include, but are not limited to, competitive and non-competitive assay systems using techniques such as Biacore analyses, FACS analyses, immunofluorescence, immunocytochemistry, Western blot analyses, radioimmunoassays, ELISAs, “sandwich” immunoassays, immunoprecipitation assays, precipitation reactions, gel diffusion precipitin reactions, immunodiffusion assays, agglutination assays, complement-fixation assays, immunoradiometric assays, fluorescent immunoassays, and protein A immunoassays. Such assays are routine and well-known in the art.

For example, the specific binding of a test agent (e.g., a polypeptide) to human LTβR may be determined using ELISA. An ELISA assay comprises preparing human LTβR, coating wells of a 96-well microtiter plate with the LTβR protein, adding the test agent conjugated to a detectable compound such as an enzymatic substrate (e.g. horseradish peroxidase or alkaline phosphatase) to the well, incubating for a period of time and detecting the presence of the agent bound to LTβR. In some embodiments, the test agent is not conjugated to a detectable compound, but instead, a labeled secondary antibody that recognizes the agent is added to the well. In some embodiments, instead of coating the well with LTβR, the test agent can be coated to the well, LTβR is added, and a second antibody conjugated to a detectable compound that recognizes LTβR can be used to detect binding. One of skill in the art would be knowledgeable as to the parameters that can be modified to increase the signal detected as well as other variations of ELISAs known in the art.

In another example, the specific binding of a test agent, e.g., a polypeptide, to human LTβR may be determined using FACS. A FACS screening assay may comprise generating a cDNA construct that expresses LTβR, transfecting the construct into cells, expressing LTβR on the surface of the cells, mixing the test agent with the transfected cells, and incubating for a period of time. The cells bound by the test agent may be identified by using a secondary antibody conjugated to a detectable compound (e.g., PE-conjugated anti-Fe antibody) and a flow cytometer. One of skill in the art would be knowledgeable as to the parameters that can be modified to optimize the signal detected as well as other variations of FACS that may enhance screening (e.g., screening for blocking antibodies).

The binding affinity of a test agent to a target (e.g., human LTβR) and the off-rate of an agent-target interaction can be determined by competitive binding assays. One example of a competitive binding assay is a radioimmunoassay comprising the incubation of labeled target (e.g., ³H or ¹²⁵I-labeled LTβR), or fragment or variant thereof, with the agent of interest in the presence of increasing amounts of unlabeled target followed by the detection of the agent bound to the labeled target. The affinity of the agent for a target (e.g., human LTβR) and the binding off-rates can be determined from the data by Scatchard plot analysis. In some embodiments, the Biacore kinetic analysis is used to determine the binding on and off rates of agents that bind a target (e.g., human LTβR). The biacore kinetic analysis comprises analyzing the binding and dissociation of agents from chips with the immobilized target (e.g., human LTβR) on the chip surface.

In some embodiments, a polypeptide, agent, or molecule described herein specifically binds LTβR and acts as an LTβR agonist. In certain embodiments, a polypeptide, agent or molecule described herein is an agonist (either directly or indirectly) of human LTβR. In some embodiments, a polypeptide or molecule is an agonist of LTβR and activates and/or increases an immune response. In some embodiments, a polypeptide, agent or molecule is an agonist of LTβR and activates and/or increases the activity of T-cells (e.g., cytolytic activity or cytokine production). In some embodiments, a polypeptide, agent or molecule is an agonist of LTβR and activates and/or increases chemokine production. In certain embodiments, a polypeptide, agent or molecule increases the activity by at least about 10%, at least about 20%, at least about 30%, at least about 50%, at least about 75%, at least about 90%, or about 100%.

In some embodiments, a polypeptide, agent, or molecule described herein specifically binds LTβR and activates LTβR signaling. In some embodiments, a polypeptide, agent or molecule described herein specifically binds LTβR and induces, activates, promotes, increases, enhances, or prolongs LTβR activity.

In some embodiments, a polypeptide, agent or molecule described herein specifically binds LTβR and modulates an immune response. In some embodiments, a polypeptide, agent or molecule described herein specifically binds LTβR and induces, augments, increases, and/or prolongs an immune response. In some embodiments, a polypeptide, agent or molecule described herein specifically binds LTβR and recruits T-cells to a specific target. In some embodiments, a polypeptide, agent or molecule described herein specifically binds LTβR and facilitates T-cell infiltration into a tumor and/or tumor microenvironment. In some embodiments, a polypeptide, agent or molecule described herein specifically binds LTβR and facilitates the formation of lymphoid structures in tumor tissues and/or within the tumor microenvironment. In some embodiments, a polypeptide, agent or molecule described herein specifically binds LTβR and regulates the expression of various chemokines and adhesion molecules in tumor tissues and/or within the tumor microenvironment.

In certain embodiments, a polypeptide, agent or molecule described herein binds LTβR and modulates an immune response. In some embodiments, a polypeptide, agent or molecule described herein activates and/or increases an immune response. In some embodiments, a polypeptide, agent or molecule described herein increases, promotes, or enhances cell-mediated immunity. In some embodiments, a polypeptide, agent or molecule described herein increases, promotes, or enhances innate cell-mediated immunity. In some embodiments, a polypeptide, agent or molecule described herein increases, promotes, or enhances adaptive cell-mediated immunity. In some embodiments, a polypeptide, agent or molecule described herein increases, promotes, or enhances T-cell activity. In some embodiments, a polypeptide, agent or molecule described herein increases, promotes, or enhances CD4+ T-cell activity. In some embodiments, a polypeptide, agent or molecule described herein increases, promotes, or enhances CD8+ T-cell activity. In some embodiments, a polypeptide, agent or molecule described herein increases, promotes, or enhances CTL activity. In some embodiments, a polypeptide, agent or molecule described herein increases, promotes, or enhances NK cell activity. In some embodiments, a polypeptide, agent or molecule described herein increases, promotes, or enhances lymphokine-activated killer cell (LAK) activity. In some embodiments, a polypeptide, agent or molecule described herein increases, promotes, or enhances tumor-infiltrating lymphocyte (TIL) activity. In some embodiments, a polypeptide, agent or molecule described herein increases, promotes, or enhances tumor cell killing. In some embodiments, a polypeptide, agent or molecule described herein increases, promotes, or enhances the inhibition of tumor growth. In some embodiments, a polypeptide, agent or molecule described herein increases or enhances an effective immune response without causing substantial side effects and/or immune-based toxicities. In some embodiments, a polypeptide, agent or molecule described herein increases or enhances an effective immune response without causing cytokine release syndrome (CRS) or a cytokine storm.

In certain embodiments, a polypeptide, agent, or molecule increases activation of a T-cell. In certain embodiments, the activation of a T-cell by a polypeptide or molecule results in an increase in the level of activation of a T-cell of at least about 10%, at least about 25%, at least about 50%, at least about 75%, at least about 90%, or at least about 95%.

In vivo and in vitro assays for determining whether a polypeptide, agent, or molecule (or candidate binding agent) modulates an immune response are known in the art or are being developed. In some embodiments, a functional assay that detects T-cell activation can be used. In some embodiments, a functional assay that detects NK cell activity can be used. In some embodiments, a functional assay that detects cytolytic T-cell activity can be used. In some embodiments, an assay that detects cytokine production can be used. In some embodiments, an assay that detects cytokine-producing cells can be used. In some embodiments, an assay that detects chemokine-producing cells can be used.

In certain embodiments, a polypeptide, agent, or molecule described herein is capable of inhibiting tumor growth. In certain embodiments, the polypeptide, agent, or molecule is capable of inhibiting tumor growth in vivo (e.g., in a mouse model and/or in a human having cancer).

In certain embodiments, a polypeptide, agent, or molecule described herein has one or more of the following effects: inhibits proliferation of tumor cells, inhibits tumor growth, reduces the tumorigenicity of a tumor, triggers cell death of tumor cells, increases cell contact-dependent growth inhibition, increases tumor cell apoptosis, or decreases survival of tumor cells.

In certain embodiments, a polypeptide, agent, or molecule described herein has a circulating half-life in mice, rats, cynomolgus monkeys, or humans of at least about 5 hours, at least about 10 hours, at least about 24 hours, at least about 2 days, at least about 3 days, at least about 1 week, at least about 2 weeks, or at least 3 weeks. In certain embodiments, the polypeptide or molecule is an IgG (e.g., IgG1 or IgG2) fusion protein that has a circulating half-life in mice, rats, cynomolgus monkeys, or humans of at least about 5 hours, at least about 10 hours, at least about 24 hours, at least about 3 days, at least about 1 week, at least about 2 weeks, or at least 3 weeks. Methods of increasing (or decreasing) the half-life of agents such as polypeptides and soluble proteins are known in the art. For example, known methods of increasing the circulating half-life of IgG fusion proteins include the introduction of mutations in the Fc region which increase the pH-dependent binding of the antibody to the neonatal Fc receptor (FcRn) at pH 6.0. Known methods of increasing the circulating half-life of soluble receptors/ligands lacking a Fc region include such techniques as PEGylation.

In some embodiments of the present invention, the molecule, agent, or binding agent is a polypeptide. The polypeptide can be a recombinant polypeptide, a natural polypeptide, or a synthetic polypeptide that binds LTβR. It will be recognized in the art that some amino acid sequences of the invention can be varied without significant effect of the structure or function of the protein. Thus, the invention further includes variations of the polypeptides which show substantial binding activity to LTβR. In some embodiments, amino acid sequence variations of the polypeptides include deletions, insertions, inversions, repeats, and/or other types of substitutions.

The polypeptides, analogs, and variants thereof, can be further modified to contain additional chemical moieties not normally part of the polypeptide. The derivatized moieties can improve the solubility, the biological half-life, and/or absorption of the polypeptide. The moieties can also reduce or eliminate undesirable side effects of the polypeptides and variants. An overview for chemical moieties can be found in Remington: The Science and Practice of Pharmacy, 22^{nd} Edition, 2012, Pharmaceutical Press. London.

The polypeptides described herein can be produced by any suitable method known in the art. Such methods range from direct protein synthesis methods to constructing a DNA sequence encoding polypeptide sequences and expressing those sequences in a suitable host. In some embodiments, a DNA sequence is constructed using recombinant technology by isolating or synthesizing a DNA sequence encoding a wild-type protein of interest. Optionally, the sequence can be mutagenized by site-specific mutagenesis to provide functional analogs thereof.

In some embodiments, a DNA sequence encoding a polypeptide of interest may be constructed by chemical synthesis using an oligonucleotide synthesizer. Oligonucleotides can be designed based on the amino acid sequence of the desired polypeptide and selecting those codons that are favored in the host cell in which the recombinant polypeptide of interest will be produced. Standard methods can be applied to synthesize a polynucleotide sequence encoding an isolated polypeptide of interest. For example, a complete amino acid sequence can be used to construct a back-translated gene. Further, a DNA oligomer containing a nucleotide sequence coding for the particular isolated polypeptide can be synthesized. For example, several small oligonucleotides coding for portions of the desired polypeptide can be synthesized and then ligated. The individual oligonucleotides typically contain 5′ or 3′ overhangs for complementary assembly.

Once assembled (by synthesis, site-directed mutagenesis, or another method), the polynucleotide sequences encoding a particular polypeptide of interest can be inserted into an expression vector and operatively linked to an expression control sequence appropriate for expression of the protein in the desired host. The proper assembly can be confirmed by nucleotide sequencing, restriction enzyme mapping, and/or expression of a biologically active polypeptide in a suitable host. As is well-known in the art, in order to obtain high expression levels of a transfected gene in a host, the gene must be operatively linked to transcriptional and translational expression control sequences that are functional in the chosen expression host.

In certain embodiments, a recombinant expression vector is used to amplify and express DNA encoding a polypeptide or molecule described herein. For example, a recombinant expression vector can be a replicable DNA construct which has synthetic or cDNA-derived DNA fragments encoding a polypeptide chain of an agent operatively linked to suitable transcriptional and/or translational regulatory elements derived from mammalian, microbial, viral or insect genes. A transcriptional unit generally comprises an assembly of (1) a genetic element or elements having a regulatory role in gene expression, for example, transcriptional promoters or enhancers, (2) a structural or coding sequence which is transcribed into mRNA and translated into protein, and (3) appropriate transcription and translation initiation and termination sequences. Regulatory elements can include an operator sequence to control transcription. The ability to replicate in a host, usually conferred by an origin of replication, and a selection gene to facilitate recognition of transformants can additionally be incorporated. DNA regions are “operatively linked” when they are functionally related to each other. For example, DNA for a signal peptide (secretory leader) is operatively linked to DNA for a polypeptide if it is expressed as a precursor which participates in the secretion of the polypeptide; a promoter is operatively linked to a coding sequence if it controls the transcription of the sequence; or a ribosome binding site is operatively linked to a coding sequence if it is positioned so as to permit translation. In some embodiments, structural elements intended for use in yeast expression systems include a leader sequence enabling extracellular secretion of translated protein by a host cell. In other embodiments, where recombinant protein is expressed without a leader or transport sequence, it can include an N-terminal methionine residue. This residue can optionally be subsequently cleaved from the expressed recombinant protein to provide a final product.

The choice of an expression control sequence and an expression vector depends upon the choice of host. A wide variety of expression host/vector combinations can be employed. Useful expression vectors for eukaryotic hosts include, for example, vectors comprising expression control sequences from SV40, bovine papilloma virus, adenovirus, and cytomegalovirus. Useful expression vectors for bacterial hosts include known bacterial plasmids, such as plasmids from E. coli, including pCR1, pBR322, pMB9 and their derivatives, and wider host range plasmids, such as M13 and other filamentous single-stranded DNA phages.

Suitable host cells for expression of a polypeptide (or a protein to use as a target) include prokaryotes, yeast cells, insect cells, or higher eukaryotic cells under the control of appropriate promoters. Prokaryotes include gram-negative or gram-positive organisms, for example, E. coli or Bacillus. Higher eukaryotic cells include established cell lines of mammalian origin as described below. Cell-free translation systems may also be employed. Appropriate cloning and expression vectors for use with bacterial, fungal, yeast, and mammalian cellular hosts are well known by those skilled in the art.

Various mammalian cell culture systems are used to express recombinant polypeptides. Expression of recombinant proteins in mammalian cells can be preferred because such proteins are generally correctly folded, appropriately modified, and biologically functional. Examples of suitable mammalian host cell lines include COS-7 (monkey kidney-derived), L-929 (murine fibroblast-derived), C127 (murine mammary tumor-derived), 3T3 (murine fibroblast-derived), CHO (Chinese hamster ovary-derived), HeLa (human cervical cancer-derived), BHK (hamster kidney fibroblast-derived), and HEK-293 (human embryonic kidney-derived) cell lines and variants thereof. Mammalian expression vectors can comprise non-transcribed elements such as an origin of replication, a suitable promoter and enhancer linked to the gene to be expressed, and other 5′ or 3′ flanking non-transcribed sequences, and 5′ or 3′ non-translated sequences, such as necessary ribosome binding sites, a polyadenylation site, splice donor and acceptor sites, and transcriptional termination sequences.

Expression of recombinant proteins in insect cell culture systems (e.g., baculovirus) also offers a robust method for producing correctly folded and biologically functional proteins. Baculovirus systems for production of heterologous proteins in insect cells are well-known to those of skill in the art.

Thus, the present invention provides cells comprising the polypeptides and agents described herein. In some embodiments, the cells produce the polypeptides and agents described herein. In certain embodiments, the cells produce a fusion protein. In some embodiments, the cells produce a soluble receptor/ligand. In some embodiments, the cells produce an antibody. In some embodiments, the cells produce a bispecific agent. In some embodiments, the cells produce a bispecific antibody. In some embodiments, the cells produce a homodimeric bispecific agent. In some embodiments, the cells produce a heterodimeric bispecific agent.

The proteins produced by a transformed host can be purified according to any suitable method. Standard methods include chromatography (e.g., ion exchange, affinity, and sizing column chromatography), centrifugation, differential solubility, or by any other standard technique for protein purification. Affinity tags such as hexa-histidine, maltose binding domain, influenza coat sequence, and glutathione-S-transferase can be attached to the protein to allow easy purification by passage over an appropriate affinity column. Isolated proteins can also be physically characterized using such techniques as proteolysis, mass spectrometry (MS), nuclear magnetic resonance (NMR), high performance liquid chromatography (HPLC), and x-ray crystallography.

In some embodiments, supernatants from expression systems which secrete recombinant protein into culture media can be first concentrated using a commercially available protein concentration filter, for example, an Amicon or Millipore Pellicon ultrafiltration unit. Following the concentration step, the concentrate can be applied to a suitable purification matrix. In some embodiments, an anion exchange resin can be employed, for example, a matrix or substrate having pendant dicthylaminoethyl (DEAE) groups. The matrices can be acrylamide, agarose, dextran, cellulose, or other types commonly employed in protein purification. In some embodiments, a cation exchange step can be employed. Suitable cation exchangers include various insoluble matrices comprising sulfopropyl or carboxymethyl groups. In some embodiments, a hydroxyapatite media can be employed, including but not limited to, ceramic hydroxyapatite (CHT). In certain embodiments, one or more reverse-phase HPLC steps employing hydrophobic RP-HPLC media, e.g., silica gel having pendant methyl or other aliphatic groups, can be employed to further purify a polypeptide or molecule. Some or all of the foregoing purification steps, in various combinations, can also be employed to provide a homogeneous recombinant protein.

In some embodiments, recombinant protein produced in bacterial culture can be isolated, for example, by initial extraction from cell pellets, followed by one or more concentration, salting-out, aqueous ion exchange, or size exclusion chromatography steps. HPLC can be employed for final purification steps. Microbial cells employed in expression of a recombinant protein can be disrupted by any convenient method, including freeze-thaw cycling, sonication, mechanical disruption, or use of cell lysing agents.

In certain embodiments, a polypeptide or molecule described herein is a polypeptide that does not comprise an immunoglobulin Fc region. In certain embodiments, the polypeptide comprises a protein scaffold of a type selected from the group consisting of protein A, protein G, a lipocalin, a fibronectin domain, an ankyrin consensus repeat domain, and thioredoxin. A variety of methods for identifying and producing non-antibody polypeptides that bind with high affinity to a protein target are known in the art.

In certain embodiments, phage display technology may be used to produce and/or identify a binding polypeptide. In certain embodiments, mammalian cell display technology may be used to produce and/or identify a binding polypeptide.

It can further be desirable to modify a polypeptide in order to increase (or decrease) its serum half-life. This can be achieved, for example, by incorporation of a salvage receptor binding epitope into the polypeptide by mutation of the appropriate region in the polypeptide or by incorporating the epitope into a peptide tag that is then fused to the polypeptide at either end or in the middle (e.g., by DNA or peptide synthesis).

Heteroconjugate molecules are also within the scope of the present invention. Heteroconjugate molecules are composed of two covalently joined polypeptides. Such molecules have, for example, been proposed to target immune cells to unwanted cells, such as tumor cells. It is also contemplated that the heteroconjugate molecules can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate.

In certain embodiments, a polypeptide, agent, or molecule described herein can be used in any one of a number of conjugated (i.e. an immunoconjugate or radioconjugate) or non-conjugated forms. In certain embodiments, the polypeptides or agents can be used in a non-conjugated form to harness the subject's natural defense mechanisms including CDC and ADCC to eliminate malignant or cancer cells.

In certain embodiments, a molecule or binding agent described herein is a small molecule. The term “small molecule” generally refers to a low molecular weight organic compound which is by definition not a peptide/protein.

In some embodiments, a polypeptide, agent, or molecule described herein is conjugated to a cytotoxic agent. In some embodiments, the cytotoxic agent is a chemotherapeutic agent including, but not limited to, methotrexate, adriamycin, doxorubicin, melphalan, mitomycin C, chlorambucil, daunorubicin or other intercalating agents. In some embodiments, the cytotoxic agent is an enzymatically active toxin of bacterial, fungal, plant, or animal origin, or fragments thereof, including, but not limited to, diphtheria A chain, nonbinding active fragments of diphtheria toxin, exotoxin A chain, ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes. In some embodiments, the cytotoxic agent is a radioisotope to produce a radioconjugate or a radioconjugated agent. A variety of radionuclides are available for the production of radioconjugated agents including, but not limited to, ⁹⁰Y, ¹²⁵I, ¹³¹I, ¹²³I, ¹¹¹In, ¹³¹In, ¹⁰⁵Rh, ¹⁵³Sm, ⁶⁷Cu, ⁶⁷Ga, ¹⁶⁶Ho, ¹⁷⁷Lu, ¹⁸⁶Re, ¹⁸⁸Re, and ²¹²Bi. Conjugates of a polypeptide or molecule and one or more small molecule toxins, such as a calicheamicin, maytansinoids, a trichothene, and CC1065, and the derivatives of these toxins that have toxin activity, can also be used. Conjugates of a polypeptide or molecule and cytotoxic agent are made using a variety of bifunctional protein-coupling agents such as N-succinimidyl-3-(2-pyridyidithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis(p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene).

### III. Targeting to Specific Cell Types

The polypeptides, molecules, agents, homotrimers, and heterotrimers described herein may be targeted to specific cell types. The polypeptides, molecules, agents, homotrimers, and heterotrimers described herein may comprise or be linked to a targeting moiety that binds a cell-surface antigen on a particular cell type. In certain embodiments, the cell-surface antigen may be expressed by the targeted cell type. In certain embodiments, the cell-surface antigen may be overexpressed by the targeted cell type.

In particular, the polypeptides, molecules, agents, homotrimers, and heterotrimers described herein may be targeted to tumor cells. One approach for targeting to tumor cells involves the use of tumor-associated antigens. These antigens allow for selective delivery of the polypeptide, molecule, agent, homotrimer or heterotrimer to tumor cells rather than normal tissue, and any appropriate tumor-associated antigen can be used for this purpose. In certain embodiments, the targeted tumors are solid tumors. In certain embodiments, the targeted tumors express or overexpress the targeted tumor-associated antigens.

Particular examples of tumor-associated antigens include those described in the TANTIGEN database available from Bioinformatics Core at Cancer Vaccine Center, Dana-Farber Cancer Institute, which include 707AP, ANKRD30A, DCT, GPRI43, KLK3, KLK4, MC1R, MLANA, OCA2, RAB38, SCGB2A2, SILV, SOX2, TYR, TYRP1, XAGE1, ABCC3, ACPP, ADAM17, ADFP, AFP, AIM2, ALDH1A1, ALK, AML1, ART4, BCL-2, BCL2L1, BIRC5, BIRC7, BST2, CA9, CCN1, CCNB1, CCND1, CEL, CEACAM5, CLCA2, CPSF1, CSPG4, CSF1, CYP1B1, DDR1, DEK, DKK1, EGFR, ENAH, EPHA2, EPHA3, ERBB2, ETV5, EZH2, FGF5, F4.2, FMNL1, FOLH1, GPC3, HSPA1A, IL13RA2, KAAG1, MCL1, MDM2, MMP2, MRPL28, MSLN, MUC1, MUC2, NPM1, PAX3, PPIB, PRAME, RAGE, RGS5, RHAMM, RNF43, SARTI, SART3, SCRNI, SFMBTI, SOX10, SOX11, SOX4, STEAP1, SYND1, TACSTD1, TERT, TOP2A, TOP2B, TP53, TPBG, TRG, TRIM68, TRPM8, TSPYL1, WDR46, WT1, XBP1, ZNF395, ANXA2, BATE, CCDC110, CDAG2, CTAG1, CTAG2, CXORF61, GAGE1, GAGE2, GAGE3, GAGE4, GAGE5, GAGE6, GAGE7, HERV-K-MEL, GAGE8, MAGEA1, MAGEA10, MAGEA12, MAGEA2, MAGEA3, MAGEA4, MAGEA6, MAGEA9, MAGEB1, MAGEB2, MAGEC2, MGAT5, SAGE1, SPA17, SSX2, SSX4, SYCP1, TGFBR2, VENTXP1, ABI2, ABL1, ACRBP, AKAP13, APC ARTC1, ATIC, BAAT, BCAP31, BCR, BTBD2, CALR3, CAN, CDC2, CDKNIA, COTL1, CTSH, DNAJC8, EIF4EBP1, ETV6, FMOD, FOXO1, FUTI, H3F3A, HSMD, HMHA 1, HMOX1, HSPE, HNRPL, IER3, IGF2BP3, ITGB8, ITPR2, JUP, LCK, LDLR, LGALS3BP, LRPI, LY6K, MAGED4, MET, MGFE8, MFI2, MMP14, OAS3, PA2G4, PAGE4, PAK2, PARP12, PGK1, PML 1, PRTN3, PSCA, PTHLH, PXDNL, RARA, RCVRN, RPA 1, RPL 10A, RPS2, RPSA, SDCBP, SEPT2, SLPB, SLC35A4, SLC45A3, SSX1, STATI, SUPT7L, SYT, TAPBP, TOR3A, TPM4, TRGC2, TTK, TYMS, UBE2A, UBE2V1, WHSC2, and WNK2.

In particular embodiments, the tumor antigen is selected from the group consisting of B7-H4, P-CADHERIN (CDH3), GABRP, ACPP, SLC45A3, STEAP1, STEAP2, GPA33, GUCY2C, and GARP (See Table 1). Alternatively, the tumor-associated antigen may be PVRL4, B7-H3, mesothelin or CA9. Examples of tumors that can be treated using antibodies targeted to certain tumor-associated antigens may be as indicated below in Table 1.

TABLE 1

| Tumor or cell type | Antigen |
|---|---|
| Breast | B7-H4 |
| | CDH3 |
| | GABRP |
| Ovarian | B7-H4 |
| | CDH3 |
| Colon | GPA33 |
| | GUCY2C |
| Prostate | ACPP |
| | SLC45A3 |
| | STEAP1 |
| | STEAP2 |
| T-regulatory cells | GARP/LRC32 |

In addition, as reported below in Example 15 below, expression data indicate that the tumor-associated antigens B7-H4, P-CADHERIN, PVRL4, B7-H3, mesothelin, and CA9 may be used to target the polypeptides, molecules, agents, homotrimers, and heterotrimers described herein to the tumor tissues indicated below in Table 2.

TABLE 2

| Tumor antigens | Tumor Tissues of origin |
|---|---|
| B7-H4 | breast, ovarian |
| P-CADHERIN (CDH3) | bladder, breast, colon, lung, melanoma, |
| | ovarian, pancreas, and stomach |
| PVRL4 | bladder, breast, lung, ovarian, pancreas, |
| | stomach, colon |
| B7-H3 (CD276) | brain, bladder, breast, lung, melanoma, |
| | ovarian, colon, pancreas, prostate, stomach |
| MESOTHELIN (MSLN) | mesothelioma, ovarian, pancreatic |
| CA9 | bladder, brain, breast, colon, lung, ovarian, |
| | pancreas, stomach |

The present invention provides a variety of targeting moieties. In certain embodiments, a targeting moiety is capable of binding an antigen on the surface of a target cell. In certain embodiments, the targeting moiety selectively targets the target cell (and/or the tissue containing the target cell). In some instances, the target cell may be a tumor cell. Thus, in certain embodiments, the targeting moieties bind tumor-associated antigens.

Tumor-associated antigens that may be bound by a targeting moiety described herein include without limitation the following: B7-H4, B7-H3, P-CADHERIN (CDH3). GABRP. ACPP. SLC45A3, STEAP1, STEAP2. GPA33. GUCY2C, GARP, PVRL4, mesothelin, and CA9. Thus, in certain embodiments, the targeting moiety specifically binds B7-H4, P-CADHERIN (CDH3), GABRP, ACPP, SLC45A3, STEAP1, STEAP2, GPA33, GUCY2C, or GARP. In some other embodiments, the targeting moiety specifically binds PVRL4, B7-H3, mesothelin or CA9. In certain embodiments, the targeting moiety specifically binds human B7-H4. In certain alternative embodiments, the targeting moiety specifically binds human P-CADHERIN. In certain embodiments, the targeting moiety specifically binds GPA33. In certain embodiments, the targeting moiety specifically binds B7-H3.

In certain embodiments, the targeting moiety is an antibody. In certain embodiments, the antibody is an antibody that specifically binds a cell-surface antigen, such as a tumor-associated antigen. The tumor-associated antigen may be a human protein. The targeting moiety used in the agents, molecules, and polypeptides described herein may, in some embodiments, be an antibody that binds B7-H4, B7-H3, P-CADHERIN (CDH3), GABRP, ACPP, SLC45A3, STEAP1, STEAP2, GPA33, GUCY2C, GARP, PVRL4, mesothelin, or CA9. Thus, in certain embodiments, the targeting moiety is an antibody that specifically binds B7-H4, P-CADHERIN (CDH3), GABRP, ACPP, SLC45A3, STEAP1, STEAP2, GPA33, GUCY2C, or GARP. In some other embodiments, the targeting moiety is an antibody that specifically binds PVRL4, B7-H3, mesothelin or CA9. In certain embodiments, the targeting moiety is an antibody that specifically binds human B7-H4. In certain alternative embodiments, the targeting moiety is an antibody that specifically binds the extracellular domain of mature human P-CADHERIN. In certain embodiments, the targeting moiety is an antibody that specifically binds GPA33. In certain embodiments, the targeting moiety is an antibody that specifically binds B7-H3.

In certain embodiments where the targeting moiety (e.g., antibody) binds B7-H4, the targeted tumor may be breast or ovarian. In certain other embodiments where the targeting moiety binds P-CADHERIN, the targeted tumors may be bladder, breast, colon, lung, melanoma, ovarian, pancreas, or stomach tumors. Alternatively, where the targeting moiety binds GPA33, the targeted tumor may, in certain embodiments, be colon tumor. In some embodiments, the targeting moiety binds B7-H3 and brain, bladder, breast, lung, melanoma, ovarian, colon, pancreas, prostate, or stomach tumors are targeted. In certain other embodiments, the targeting moiety binds PVRL4 and the tumors targeted may be bladder, breast, lung, ovarian, pancreas, stomach, or colon cancer. If the targeting moiety binds mesothelin, on the other hand, examples, without limitation, of tumor types that may be targeted include mesothelioma, ovarian tumors, and pancreatic tumors. In certain alternative embodiments, the targeting moiety binds CA9 and the targeted tumor type is bladder, brain, breast, colon, lung, ovarian, pancreatic, or stomach tumor. Also, if the targeted tumor type is prostate cancer, targeting moieties that bind ACPP, SLC45A3, STEAP1, or STEAP2 may be useful.

### IV. Antibodies to Cell-Surface Antigens

The present invention provides antibodies that bind cell-surface antigens, including, without limitation, tumor-associated antigens. In certain embodiments, the antibody binds the extracellular domain of a cell surface antigen (e.g., a tumor-associated antigen). In certain embodiments, the antibody binds a tumor-associated antigen selected from the group consisting of the following: B7-H4, B7-H3, P-CADHERIN (CDH3), GABRP, ACPP, SLC45A3, STEAP1, STEAP2, GPA33, GUCY2C, GARP, PVRL4, mesothelin and CA9. In certain embodiments, the antibody specifically binds B7-H4, P-CADHERIN. GABRP, ACPP, SLC45A3, STEAP1, STEAP2, GPA33, or GUCY2C. In certain embodiments, the antibody specifically binds B7-H3, GARP, PVRL4, mesothelin or CA9. In certain embodiments the antibody specifically binds human B7-H4. In certain alternative embodiments, the antibody specifically binds human P-CADHERIN. In certain embodiments, the antibody specifically binds GPA33. In certain embodiments, the antibody specifically binds B7-H3.

In certain embodiments, the antibody specifically binds human B7-H4. The full-length amino acid (aa) sequence for human B7-H4 is known in the art and is provided herein as SEQ ID NO:87. The sequence of the extracellular domain of mature human B7-H4 is also known in the art and is provided herein as SEQ ID NO:88. In certain embodiments, the antibody also specifically binds mouse B7-H4.

In certain embodiments, the antibody specifically binds B7-H4 and comprises; (a) a heavy chain CDR1 comprising TSYYMH (SEQ ID NO:42), or a variant thereof comprising 1, 2, 3 or 4 amino acid substitutions; a heavy chain CDR2 comprising YVDPFNGGTSYNQKFKG (SEQ ID NO:43), or a variant thereof comprising 1, 2, 3, or 4 amino acid substitutions; and a heavy chain CDR3 comprising FIAGFAN (SEQ ID NO:44), or a variant thereof comprising 1, 2, 3 or 4 amino acid substitutions, or IAGFAN (SEQ ID NO:45), or a variant thereof comprising 1, 2, 3 or 4 amino acid substitutions; and/or (b) a light chain CDR1 comprising KASQDIKSYLS (SEQ ID NO:46), or a variant thereof comprising 1, 2, 3 or 4 amino acid substitutions; a light chain CDR2 comprising YATSLAD (SEQ ID NO:47), or a variant thereof comprising 1, 2, 3 or 4 amino acid substitutions; and a light chain CDR3 comprising LQHGESPYT (SEQ ID NO:48), or a variant thereof comprising 1, 2, 3 or 4 amino acid substitutions, or LQHGESPY (SEQ ID NO:49), or a variant thereof comprising 1, 2, 3 or 4 amino acid substitutions. In certain embodiments, the amino acid substitutions are conservative substitutions. In some embodiments, the substitutions are made as part of a humanization process. In some embodiments, the substitutions are made as part of a germline humanization process. In certain embodiments, the antibody may comprise a heavy chain variable region having at least about 90% (e.g., at least about 95%) sequence identity to SEQ ID NO:50 and a light chain variable region having at least about 90% sequence identity (e.g., at least about 95%) to SEQ ID NO:51. In certain alternative embodiments, the antibody may comprise a heavy chain variable region having at least about 90% (e.g., at least about 95%) sequence identity to SEQ ID NO:66 and a light chain variable region having at least about 90% (e.g., at least about 95%) sequence identity to SEQ ID NO:62.

In certain embodiments, the antibody that specifically binds B7-H4 comprises (a) a heavy chain CDR1 comprising TSYYMH (SEQ ID NO:42), a heavy chain CDR2 comprising YVDPFNGGTSYNQKFKG (SEQ ID NO:43), and a heavy chain CDR3 comprising FIAGFAN (SEQ ID NO:44) or IAGFAN (SEQ ID NO:45) or (b) a light chain CDR1 comprising KASQDIKSYLS (SEQ ID NO:46), a light chain CDR2 comprising YATSLAD (SEQ ID NO:47), and a light chain CDR3 comprising LQHGESPYT (SEQ ID NO:48) or LQHGESPY (SEQ ID NO:49). In certain embodiments, the antibody comprises (a) a heavy chain CDR1 comprising TSYYMH (SEQ ID NO:42), a heavy chain CDR2 comprising YVDPFNGGTSYNQKFKG (SEQ ID NO:43), and a heavy chain CDR3 comprising FIAGFAN (SEQ ID NO:44) or IAGFAN (SEQ ID NO:45) and (b) a light chain CDR1 comprising KASQDIKSYLS (SEQ ID NO:46), a light chain CDR2 comprising YATSLAD (SEQ ID NO:47), and a light chain CDR3 comprising LQHGESPYT (SEQ ID NO:48) or LQHGESPY (SEQ ID NO:49). In certain embodiments, the antibody may comprise a heavy chain variable region having at least about 90% (e.g., at least about 95%) sequence identity to SEQ ID NO:50 and a light chain variable region having at least about 90% sequence identity (e.g., at least about 95%) to SEQ ID NO:51. In certain embodiments, the antibody may comprise a heavy chain variable region having at least about 90% (e.g., at least about 95%) sequence identity to SEQ ID NO:66 and a light chain variable region having at least about 90% (e.g., at least about 95%) sequence identity to SEQ ID NO:62. In certain other embodiments, the antibody may comprise a heavy chain variable region having at least about 98% sequence identity to SEQ ID NO:66 and a light chain variable region having at least about 98% sequence identity to SEQ ID NO:62. In some embodiments, the heavy chain variable region comprises SEQ ID NO:66 and the light chain variable region comprises SEQ ID NO:62.

In certain embodiments, the invention provides an antibody that specifically binds B7-H4, wherein the antibody comprises a heavy chain variable region having at least about 900%, at least about 95%, or at least about 98% sequence identity to SEQ ID NO:50 or SEQ ID NO:66. In certain embodiments, the antibody may comprise a heavy chain variable region having at least about 90% sequence identity to SEQ ID NO:50. In certain other embodiments, the antibody may comprise a heavy chain variable having at least about 90% sequence identity to SEQ ID NO:66. In certain embodiments, the antibody may comprise a heavy chain variable region having at least about 95% sequence identity to SEQ ID NO:50. In alternative embodiments, the antibody may comprises a heavy chain variable region having at least about 95% sequence identity to SEQ ID NO:66. In further embodiments, the antibody may comprise a heavy chain variable region having at least about 98% sequence identity to SEQ ID NO:66. The heavy chain variable region may comprise (a) a heavy chain CDR1 comprising TSYYMH (SEQ ID NO:42), a heavy chain CDR2 comprising YVDPFNGGTSYNQKFKG (SEQ ID NO:43), and a heavy chain CDR3 comprising FIAGFAN (SEQ ID NO:44) or IAGFAN (SEQ ID NO:45). In certain embodiments, the antibody may comprise a heavy chain variable region comprising SEQ ID NO:50. In certain further embodiments, the antibody may comprise a heavy chain variable region comprising SEQ ID NO:66.

The invention further provides an antibody that specifically binds B7-H4, wherein the antibody comprises a light chain variable region having at least about 90% (e.g., at least about 95% or at least about 98%) sequence identity to SEQ ID NO:51 or SEQ ID NO:62. In certain embodiments, the antibody comprises a light chain variable region having at least about 95% sequence identity to SEQ ID NO:51 or SEQ ID NO:62. The antibody may comprise a light chain variable region having at least about 98% sequence identity to SEQ ID NO:51 or SEQ ID NO:62. Optionally, the antibody may comprise a light chain CDR1 comprising KASQDIKSYLS (SEQ ID NO:46), a light chain CDR2 comprising YATSLAD (SEQ ID NO:47), and a light chain CDR3 comprising LQHGESPYT (SEQ ID NO:48) or LQHGESPY (SEQ ID NO:49). In certain embodiments, the antibody comprises a light chain variable region comprising SEQ ID NO:51. In certain alternative embodiments, the antibody comprises a light chain variable region comprising SEQ ID NO:62.

In certain embodiments, the antibody may comprise both a heavy chain variable region having at least about 90% sequence identity to SEQ ID NO:50 and a light chain variable region having at least about 90% sequence identity to SEQ ID NO:51. In certain embodiments, the antibody may comprise a heavy chain variable region having at least about 95% sequence identity to SEQ ID NO:50 and a light chain variable region having at least about 95% sequence identity to SEQ ID NO:51. In further embodiments, the antibody may comprise a heavy chain variable region having at least about 98% sequence identity to SEQ ID NO:50 and a light chain variable region having at least about 98% sequence identity to SEQ ID NO:51.

The invention further provides an antibody that specifically binds B7-H4 and comprises (a) a heavy chain variable region having at least about 90%, at least about 95% or at least about 98% sequence identity to SEQ ID NO:66, and (b) a light chain variable region having at least about 90%, at least about 95%, or at least about 98% sequence identity to SEQ ID NO:62. In certain embodiments, the antibody comprises (a) a heavy chain variable region having at least about 90% sequence identity to SEQ ID NO:66, and (b) a light chain variable region having at least about 90% sequence identity to SEQ ID NO:62. In some embodiments, the antibody comprises (a) a heavy chain variable region having at least about 95% sequence identity to SEQ ID NO:66, and (b) a light chain variable region having at least about 95% sequence identity to SEQ ID NO:62. In some embodiments, the antibody comprises (a) a heavy chain variable region having at least about 98% sequence identity to SEQ ID NO:66, and (b) a light chain variable region having at least about 98% sequence identity to SEQ ID NO:62.

The invention also provides an antibody that specifically binds B7-H4 and comprises (a) a heavy chain variable region comprising SEQ ID NO:66, and (b) a light chain variable region comprising SEQ ID NO:62.

In certain embodiments, the invention provides an antibody that specifically binds human B7-H4, wherein the antibody comprises one, two, three, four, five, and/or six of the CDRs of antibody 278M1 (see Table 4 in Example 16 below).

In certain embodiments, the antibody that specifically binds B7-H4 comprises the heavy chain variable region and light chain variable region of the 278M1 antibody (see Table 4 in Example 16 below). In certain embodiments, the antibody that specifically binds B7-H4 comprises, consists essentially of, or consists of, the antibody 278M1. Humanized and chimeric versions of the 278M1 antibody are also provided.

In certain embodiments, the antibody that specifically binds B7-H4 comprises the heavy chain variable region and light chain variable region of the 278M1 L2H2 antibody (see Table 4 in Example 16 below). In certain embodiments, the antibody that specifically binds B7-H4 comprises, consists essentially of, or consists of, the antibody 278M1 L2H2.

Antibodies comprising (a) a heavy chain variable region derived from the variable region of 278M1 or 278M1 L2H2 and (b) a light chain variable region derived from the variable region of 278M1 or 278M1 L2H2 are also provided. In some embodiments, the derived antibody is affinity matured.

The invention further provides antibodies which bind to the same epitope, or substantially the same epitope, on human B7-H4 as an antibody described herein. In certain embodiments, the antibodies bind to the same epitope, or substantially the same epitope, on human B7-H4 as an antibody comprising a heavy chain variable region comprising SEQ ID NO:50 and a light chain variable region comprising SEQ ID NO:51. In some exemplary embodiments, the antibodies bind to the same epitope, or substantially the same epitope, on human B7-H4 as an antibody comprising a heavy chain variable region comprising SEQ ID NO:66 and a light chain variable region comprising SEQ ID NO:62. In certain embodiments, the antibodies bind to the same epitope, or substantially the same epitope, on human B7-H4 as 278M1 or 278M1 L2H2. In some instances, antibodies that bind the same epitope, or substantially the same epitope, on B7-H4 may compete for binding on B7-H4 in a competition assay.

Antibodies which bind an epitope on B7-H4 that overlaps with the epitope of a B7-H4-binding antibody described herein are also provided. For example, an antibody which binds an epitope on B7-H4 that overlaps with the epitope of an antibody comprising a heavy chain variable region comprising SEQ ID NO:50 and a light chain variable region comprising SEQ ID NO:51 is provided. Likewise, an antibody that binds an epitope on B7-H4 that overlaps with the epitope of an antibody comprising a heavy chain variable region comprising SEQ ID NO:66 and a light chain variable region comprising SEQ ID NO:62 is also provided, as are antibodies which bind an epitope on B7-H4 that overlaps with the epitope of 278M1 or 278M1 L2H2. In some instances, antibodies that bind overlapping epitopes on B7-H4 may compete for binding on B7-H4 in a competition assay.

Antibodies that compete for binding on B7-H4 with the antibodies of the invention are also provided.

In certain embodiments, the antibody specifically binds P-CADHERIN (CDH3). In some embodiments, the antibody specifically binds human P-CADHERIN. In some embodiments, the antibody also specifically binds mouse P-CADHERIN. The full-length amino acid (aa) sequence for human P-CADHERIN and its extracellular domain are known in the art and are provided herein as SEQ ID NO:89 and SEQ ID NO:90. The extracellular domain of the mature P-CADHERIN protein is also known in the art and is provided herein as SEQ ID NO:91. In certain embodiments, the P-CADHERIN is mature human P-CADHERIN. Thus, in certain embodiments, the antibody that binds the extracellular domain of P-CADHERIN is an antibody that binds the extracellular domain of the mature human P-CADHERIN protein.

In certain embodiments, the antibody specifically binds the extracellular domain of mature human P-CADHERIN and comprises; (a) a heavy chain CDR1 comprising STYGMS (SEQ ID NO:80), or a variant thereof comprising 1, 2, 3 or 4 amino acid substitutions; a heavy chain CDR2 comprising ATISDGGSYTYYPDSVKGR (SEQ ID NO:81), or a variant thereof comprising 1, 2, 3, or 4 amino acid substitutions; and a heavy chain CDR3 comprising ARHYYGSDWYFDV (SEQ ID NO:82), or a variant thereof comprising 1, 2, 3 or 4 amino acid substitutions; and/or (b) a light chain CDR1 comprising RSSQSIVQSNGNTYLE (SEQ ID NO:73), or a variant thereof comprising 1, 2, 3 or 4 amino acid substitutions; a light chain CDR2 comprising KVSNQFS (SEQ ID NO:74), or a variant thereof comprising 1, 2, 3 or 4 amino acid substitutions; and a light chain CDR3 comprising QGSHVPL (SEQ ID NO:75), or a variant thereof comprising 1, 2, 3 or 4 amino acid substitutions. In certain embodiments, one or more of the amino acid substitutions are conservative amino acid substitutions. In some embodiments, the substitutions are made as part of a humanization process. In some embodiments, the substitutions are made as part of a germline humanization process. In still further embodiments, the substitutions are made as part of affinity maturation. In certain embodiments, the antibody may comprise a heavy chain variable region having at least about 90% (e.g., at least about 95%) sequence identity to SEQ ID NO:79 and a light chain variable region having at least about 90% sequence identity (e.g., at least about 95%) to SEQ ID NO:72 or SEQ ID NO:93.

In certain embodiments, the antibody that specifically binds P-CADHERIN comprises (a) a heavy chain CDR1 comprising STYGMS (SEQ ID NO:80), a heavy chain CDR2 comprising ATISDGGSYTYYPDSVKGR (SEQ ID NO:81), and a heavy chain CDR3 comprising ARHYYGSDWYFDV (SEQ ID NO:82) or (b) a light chain CDR1 comprising RSSQSIVQSNGNTYLE (SEQ ID NO:73), a light chain CDR2 comprising KVSNQFS (SEQ ID NO:74), and a light chain CDR3 comprising QGSHVPL (SEQ ID NO:75). In certain embodiments, the antibody comprises (a) a heavy chain CDR1 comprising STYGMS (SEQ ID NO:80), a heavy chain CDR2 comprising ATISDGGSYTYYPDSVKGR (SEQ ID NO:81), and a heavy chain CDR3 comprising ARHYYGSDWYFDV (SEQ ID NO:82) and (b) a light chain CDR1 comprising RSSQSIVQSNGNTYLE (SEQ ID NO:73), a light chain CDR2 comprising KVSNQFS (SEQ ID NO:74), and a light chain CDR3 comprising QGSHVPL (SEQ ID NO:75). In certain embodiments, the antibody may comprise a heavy chain variable region having at least about 90% (e.g., at least about 95%) sequence identity to SEQ ID NO:79 and a light chain variable region having at least about 90% sequence identity (e.g., at least about 95%) to SEQ ID NO:72 or SEQ ID NO:93. In certain alternative embodiments, the antibody may comprise a heavy chain variable region having at least about 90% (e.g., at least about 95%) sequence identity to SEQ ID NO:79 and a light chain variable region having at least about 90% (e.g., at least about 95%) sequence identity to SEQ ID NO:72 or SEQ ID NO:93.

In certain embodiments, the invention provides an antibody that specifically binds P-CADHERIN, wherein the antibody comprises a heavy chain variable region having at least about 90%, at least about 95%, or at least about 98% sequence identity to SEQ ID NO:79. In certain embodiments, the antibody may comprise a heavy chain variable region having at least about 90% sequence identity to SEQ ID NO:79. In certain embodiments, the antibody may comprise a heavy chain variable region having at least about 95% sequence identity to SEQ ID NO:79. In further embodiments, the antibody may comprise a heavy chain variable region having at least about 98% sequence identity to SEQ ID NO:79. The heavy chain variable region may comprise a heavy chain CDR1 comprising STYGMS (SEQ ID NO:80), a heavy chain CDR2 comprising ATISDGGSYTYYPDSVKGR (SEQ ID NO:81), and a heavy chain CDR3 comprising ARHYYGSDWYFDV (SEQ ID NO:82). In certain embodiments, the antibody may comprise a heavy chain variable region comprising SEQ ID NO:79.

The invention further provides an antibody that specifically binds P-CADHERIN, wherein the antibody comprises a light chain variable region having at least about 90% (e.g., at least about 95% or at least about 98%) sequence identity to SEQ ID NO:72 or SEQ ID NO:93. In certain embodiments, the antibody comprises a light chain variable region having at least about 95% sequence identity to SEQ ID NO:72 or SEQ ID NO:93. The antibody may comprise a light chain variable region having at least about 98% sequence identity to SEQ ID NO:72 or SEQ ID NO:93. Optionally, the antibody may comprise a light chain CDR1 comprising RSSQSIVQSNGNTYLE (SEQ ID NO:73), a light chain CDR2 comprising KVSNQFS (SEQ ID NO:74), and a light chain CDR3 comprising QGSHVPL (SEQ ID NO:75). In certain embodiments, the antibody comprises a light chain variable region comprising SEQ ID NO:72. In certain alternative embodiments, the antibody comprises a light chain variable region comprising SEQ ID NO:93.

The invention further provides an antibody that specifically binds P-CADHERIN and comprises (a) a heavy chain variable region having at least about 90%, at least about 95% or at least about 98% sequence identity to SEQ ID NO:79, and (b) a light chain variable region having at least about 90%, at least about 95%, or at least about 98% sequence identity to SEQ ID NO:72 or SEQ ID NO:93. In certain embodiments, the antibody comprises (a) a heavy chain variable region having at least about 90% sequence identity to SEQ ID NO:79, and (b) a light chain variable region having at least about 90% sequence identity to SEQ ID NO:72 or SEQ ID NO:93. In some embodiments, the antibody comprises (a) a heavy chain variable region having at least about 95% sequence identity to SEQ ID NO:79, and (b) a light chain variable region having at least about 95% sequence identity to SEQ ID NO:72 or SEQ ID NO:93. In some embodiments, the antibody comprises (a) a heavy chain variable region having at least about 98% sequence identity to SEQ ID NO:79, and (b) a light chain variable region having at least about 98% sequence identity to SEQ ID NO:72 or SEQ ID NO:93. The antibody may comprise (a) a heavy chain variable region comprising SEQ ID NO:79, and (b) a light chain variable region comprising SEQ ID NO:72. In certain alternative embodiments, the antibody may comprise (a) a heavy chain variable region comprising SEQ ID NO:79, and (b) a light chain variable region comprising SEQ ID NO:93

The invention also provides an antibody that specifically binds P-CADHERIN and comprises (a) a heavy chain variable region comprising SEQ ID NO:79, and (b) a light chain variable region comprising SEQ ID NO:72. An antibody that specifically binds P-CADHERIN and comprises (a) a heavy chain variable region comprising SEQ ID NO:79, and (b) a light chain variable region comprising SEQ ID NO:93 is also provided.

In certain embodiments, the invention provides an antibody that specifically binds human P-CADHERIN, wherein the antibody comprises one, two, three, four, five, and/or six of the CDRs of antibody 173M36 L1H2 (or 173M36 L3H2) (see Table 5 in Example 17 below).

In certain embodiments, the antibody that specifically binds P-CADHERIN comprises the heavy chain variable region and light chain variable region of the 173M36 L1H2 antibody (see Table 5 in Example 17 below). In certain embodiments, the antibody that specifically binds P-CADHERIN comprises, consists essentially of, or consists of, the antibody 173M36 L1H2.

In certain embodiments, the antibody that specifically binds P-CADHERIN comprises the heavy chain variable region and light chain variable region of the 173M36 L3H2 antibody (see Table 5 in Example 17 below). In certain embodiments, the antibody that specifically binds P-CADHERIN comprises, consists essentially of, or consists of, the antibody 173M36 L3H2.

Antibodies comprising (a) a heavy chain variable region derived from the variable region of 173M36 L1H2 or 173M36 L3H2 and (b) a light chain variable region derived from the variable region of 173M36 L1H2 or 173M36 L3H2 are also provided. In some embodiments, the derived antibody is affinity matured.

The invention further provides antibodies which bind to the same epitope, or substantially the same epitope, on human P-CADHERIN as an antibody described herein. In certain embodiments, the antibodies bind to the same epitope, or substantially the same epitope, on human P-CADHERIN as an antibody comprising a heavy chain variable region comprising SEQ ID NO:79 and a light chain variable region comprising SEQ ID NO:72. In some exemplary embodiments, the antibodies bind to the same epitope, or substantially the same epitope, on human P-CADHERIN as an antibody comprising a heavy chain variable region comprising SEQ ID NO:79 and a light chain variable region comprising SEQ ID NO:93. In certain embodiments, the antibodies bind to the same epitope, or substantially the same epitope, on human P-CADHERIN as 173M36 L1H2 or 173M36 L3H2. In some instances, antibodies that bind the same epitope, or substantially the same epitope, on P-CADHERIN may compete for binding on P-CADHERIN in a competitive binding assay.

Antibodies which bind an epitope on P-CADHERIN that overlaps with the epitope of a P-CADHERIN-binding antibody described herein are also provided. For example, an antibody which binds an epitope on P-CADHERIN that overlaps with the epitope of an antibody comprising a heavy chain variable region comprising SEQ ID NO:79 and a light chain variable region comprising SEQ ID NO:72 is provided. Likewise, an antibody which binds an epitope on P-CADHERIN that overlaps with the epitope of an antibody comprising a heavy chain variable region comprising SEQ ID NO:79 and a light chain variable region comprising SEQ ID NO:93 is also provided, as are antibodies which bind an epitope on P-CADHERIN that overlaps with the epitope of 173M36 L1H2 or 173M36 L3H2. In some instances, antibodies that bind overlapping epitopes on P-CADHERIN may compete for binding on P-CADHERIN in a competitive binding assay.

Antibodies that compete for binding on P-CADHERIN with the antibodies of the invention are also provided.

A variety of different versions of the antibodies described above and elsewhere herein that specifically bind a cell-surface antigen (e.g., a tumor-associated antigen such as B7-H4 or P-CADHERIN) are provided. In some embodiments, for example, the antibody is a recombinant antibody. In some embodiments, the antibody is a monoclonal antibody. In some embodiments, the antibody is a chimeric antibody. In some embodiments, the antibody is a humanized antibody. In some embodiments, the antibody is a human antibody. In some embodiments, the antibody is an IgA, IgD, IgE, IgG, or IgM antibody. In certain embodiments, the antibody is an IgG1 antibody. In certain embodiments, the antibody is an IgG2 antibody. In some embodiments, the antibody is an IgG4 antibody. In certain embodiments, the antibody that specifically binds the cell-surface antigen is an antibody fragment comprising an antigen-binding site. In some embodiments, the antibody is a bispecific antibody or a multispecific antibody. In some embodiments, the antibody is a monovalent antibody. In some embodiments, the antibody is a monospecific antibody. In some embodiments, the antibody is a bivalent antibody. In some embodiments, the antibody is conjugated to a cytotoxic moiety. In some embodiments, the antibody is isolated. In some embodiments, the antibody is substantially pure.

In certain embodiments, the antibody binds a cell-surface antigen with a dissociation constant (K_{D}) of about 100 nM or less, about 40 nM or less, about 20 nM or less, about 10 nM or less, about 1 nM or less, or about 0.1 nM or less. In some embodiments, the antibody binds the antigen with a K_{D} of about 20 nM or less. In some embodiments, the antibody binds the antigen with a K_{D} of about 10 nM or less. In some embodiments, the antibody specifically binds the antigen a K_{D} of about 1 nM or less. In some embodiments, the antibody binds both the human and mouse homologs of the antigen with a K_{D} of about 10 nM or less. In some embodiments, the dissociation constant of the antibody to the antigen is the dissociation constant determined using a fusion protein comprising at least a portion of the extracellular domain of antigen immobilized on a Biacore chip. In some embodiments, the dissociation constant of the antibody binding the antigen is the dissociation constant determined using the binding agent captured by an anti-human IgG antibody on a Biacore chip and a soluble form of the antigen.

In certain embodiments, the antibody binds the cell-surface antigen with a half maximal effective concentration (EC₅₀) of about 1 μM or less, about 100 nM or less, about 40 nM or less, about 200 nM or less, about 10 nM or less, about 1 nM or less, or about 0.1 nM or less. In certain embodiments, the antibody binds to the antigen with a half maximal effective concentration (EC₅₀) of about 40 nM or less. In certain embodiments, the antibody binds both mouse and/or human homologs of the antigen with an EC₅₀ of about 40 nM or less.

In some embodiments, the antibodies are polyclonal antibodies. Polyclonal antibodies can be prepared by any known method. In some embodiments, polyclonal antibodies are produced by immunizing an animal (e.g., a rabbit, rat, mouse, goat, donkey) with an antigen of interest (e.g., a purified peptide fragment, full-length recombinant protein, or fusion protein) using multiple subcutaneous or intraperitoneal injections. The antigen can be optionally conjugated to a carrier such as keyhole limpet hemocyanin (KLH) or serum albumin. The antigen (with or without a carrier protein) is diluted in sterile saline and usually combined with an adjuvant (e.g., Complete or Incomplete Freund's Adjuvant) to form a stable emulsion. After a sufficient period of time, polyclonal antibodies are recovered from the immunized animal, usually from blood or ascites. The polyclonal antibodies can be purified from serum or ascites according to standard methods in the art including, but not limited to, affinity chromatography, ion-exchange chromatography, gel electrophoresis, and dialysis.

Alternatively, in some embodiments, the antibody is a monoclonal antibody. Monoclonal antibodies can be prepared using hybridoma methods known to one of skill in the art. In some embodiments, using the hybridoma method, a mouse, rat, rabbit, hamster, or other appropriate host animal, is immunized as described above to elicit the production of antibodies that specifically bind the immunizing antigen. In some embodiments, lymphocytes can be immunized in vitro. In some embodiments, the immunizing antigen can be a human protein or a fragment thereof. In some embodiments, the immunizing antigen can be a mouse protein or a fragment thereof.

Following immunization, lymphocytes are isolated and fused with a suitable myeloma cell line using, for example, polyethylene glycol. The hybridoma cells are selected using specialized media as known in the art and unfused lymphocytes and myeloma cells do not survive the selection process. Hybridomas that produce monoclonal antibodies directed specifically against a chosen antigen may be identified by a variety of methods including, but not limited to, immunoprecipitation, immunoblotting, and in vitro binding assays (e.g., flow cytometry, FACS, ELISA, and radioimmunoassay). The hybridomas can be propagated either in in vitro culture using standard methods or in vivo as ascites tumors in an animal. The monoclonal antibodies can be purified from the culture medium or ascites fluid according to standard methods in the art including, but not limited to, affinity chromatography, ion-exchange chromatography, gel electrophoresis, and dialysis.

In certain embodiments, monoclonal antibodies can be made using recombinant DNA techniques as known to one skilled in the art. The polynucleotides encoding a monoclonal antibody are isolated from mature B-cells or hybridoma cells, such as by RT-PCR using oligonucleotide primers that specifically amplify the genes encoding the heavy and light chains of the antibody, and their sequence is determined using standard techniques. The isolated polynucleotides encoding the heavy and light chains are then cloned into suitable expression vectors which produce the monoclonal antibodies when transfected into host cells such as E. coli, simian COS cells, Chinese hamster ovary (CHO) cells, or myeloma cells that do not otherwise produce immunoglobulin proteins.

In one aspect, the invention features a cell or hybridoma that produces or is capable of producing the antibodies described herein. The invention also features a cell comprising the antibodies described herein.

In certain other embodiments, recombinant monoclonal antibodies, or fragments thereof, can be isolated from phage display libraries expressing variable domains or CDRs of a desired species.

The polynucleotide(s) encoding a monoclonal antibody can be modified, for example, by using recombinant DNA technology to generate alternative antibodies. In some embodiments, the constant domains of the light chain and heavy chain of, for example, a mouse monoclonal antibody can be substituted for constant regions of, for example, a human antibody to generate a chimeric antibody, or for a non-immunoglobulin polypeptide to generate a fusion antibody. In some embodiments, the constant regions are truncated or removed to generate a desired antibody fragment of a monoclonal antibody. Site-directed or high-density mutagenesis of the variable region(s) can be used to optimize specificity, affinity, etc. of a monoclonal antibody.

In some embodiments, the antibody is a humanized antibody. Typically, humanized antibodies are human immunoglobulins in which the amino acid residues of the CDRs are replaced by amino acid residues from CDRs of a non-human species (e.g., mouse, rat, rabbit, hamster, etc.) that have the desired specificity, affinity, and/or binding capability using methods known to one skilled in the art. In some embodiments, some of the framework variable region amino acid residues of a human immunoglobulin are replaced with corresponding amino acid residues in an antibody from a non-human species. In some embodiments, a humanized antibody can be further modified by the substitution of additional residues either in the framework variable region and/or within the replaced non-human residues to further refine and optimize antibody specificity, affinity, and/or capability. In general, a humanized antibody will comprise variable domain regions containing all, or substantially all, of the CDRs that correspond to the non-human immunoglobulin whereas all, or substantially all, of the framework regions are those of a human immunoglobulin sequence. In some embodiments, the framework regions are those of a human consensus immunoglobulin sequence. In some embodiments, a humanized antibody can also comprise at least a portion of an immunoglobulin constant region or domain (Fc), typically that of a human immunoglobulin. In certain embodiments, such humanized antibodies are used therapeutically because they may reduce antigenicity and HAMA (human anti-mouse antibody) responses when administered to a human subject.

In certain embodiments, the antibody is a human antibody. Human antibodies can be directly prepared using various techniques known in the art. In some embodiments, human antibodies may be generated from immortalized human B lymphocytes immunized in vitro or from lymphocytes isolated from an immunized individual. In either case, cells that produce an antibody directed against a target antigen can be generated and isolated. In some embodiments, the human antibody can be selected from a phage library, where that phage library expresses human antibodies. Alternatively, phage display technology can be used to produce human antibodies and antibody fragments in vitro, from immunoglobulin variable domain gene repertoires from unimmunized donors. Techniques for the generation and use of antibody phage libraries are well known in the art. Once antibodies are identified, affinity maturation strategies known in the art, including but not limited to, chain shuffling and site-directed mutagenesis, may be employed to generate higher affinity human antibodies.

In some embodiments, human antibodies can be made in transgenic mice that contain human immunoglobulin loci. Upon immunization, these mice are capable of producing the full repertoire of human antibodies in the absence of endogenous immunoglobulin production.

In some embodiments, the antibody is a bispecific antibody. Thus, this invention encompasses bispecific antibodies that specifically recognize a cell surface antigen on a target cell and at least one additional target. Bispecific antibodies are capable of specifically recognizing and binding at least two different antigens or epitopes. The different epitopes can either be within the same molecule (e.g., two epitopes on the cell-surface antigen) or on different molecules (e.g., one epitope on a cell-surface antigen and one epitope on a different protein). In some embodiments, a bispecific antibody has enhanced potency as compared to an individual antibody or to a combination of more than one antibody. It is known to those of skill in the art that any therapeutic agent may have unique pharmacokinetics (PK) (e.g., circulating half-life). In some embodiments, a bispecific antibody has the ability to synchronize the PK of two active binding agents wherein the two individual binding agents have different PK profiles. In some embodiments, a bispecific antibody has the ability to concentrate the actions of two agents in a common area (e.g., a tumor and/or tumor microenvironment). In some embodiments, a bispecific antibody has the ability to concentrate the actions of two agents to a common target (e.g., a tumor or a tumor cell). In some embodiments, a bispecific antibody has the ability to target the actions of two agents to more than one biological pathway or function. In some embodiments, a bispecific antibody has the ability to target two different cells and bring them closer together (e.g., an immune cell and a tumor cell).

In some embodiments, the antibodies can specifically recognize and bind a first antigen target, (e as well as a second antigen target, such as an effector molecule on an immune cell (e.g., CD2, CD3, CD28, CTLA4, PD-1, PD-L1, CD80, or CD86) or a Fc receptor (e.g., CD64, CD32, or CD16) so as to focus cellular defense mechanisms to the cell expressing and/or producing the first antigen target. In some embodiments, the antibodies can be used to direct cytotoxic agents to cells which express a particular target antigen. In certain embodiments, these antibodies possess an antigen-binding arm and an arm which binds a cytotoxic agent or a radionuclide chelator, such as EOTUBE, DPTA, DOTA, or TETA.

Techniques for making bispecific antibodies are known by those skilled in the art. In some embodiments, the bispecific antibodies comprise heavy chain constant regions with modifications in the amino acids which are part of the interface between the two heavy chains. In some embodiments, the bispecific antibodies can be generated using a “knobs-into-holes” strategy. In some cases, the “knobs” and “holes” terminology is replaced with the terms “protuberances” and “cavities.” In some embodiments, the bispecific antibodies may comprise variant hinge regions incapable of forming disulfide linkages between the heavy chains. In some embodiments, the modifications may comprise changes in amino acids that result in altered electrostatic interactions. In some embodiments, the modifications may comprise changes in amino acids that result in altered hydrophobic/hydrophilic interactions.

Bispecific antibodies can be intact antibodies or antibody fragments comprising antigen-binding sites. Antibodies with more than two valencies are also contemplated. For example, trispecific antibodies can be prepared. Thus, in certain embodiments the antibodies described herein are multispecific.

In certain embodiments, the antibodies (or other polypeptides) described herein may be monospecific.

In certain embodiments, the antibody is an antigen-binding antibody fragment. Antibody fragments may have different functions or capabilities than intact antibodies, for example, antibody fragments can have increased tumor penetration. Various techniques are known for the production of antibody fragments including, but not limited to, proteolytic digestion of intact antibodies. In some embodiments, antibody fragments include a F(ab′)2 fragment produced by pepsin digestion of an antibody molecule. In some embodiments, antibody fragments include a Fab fragment generated by reducing the disulfide bridges of an F(ab′)2 fragment. In other embodiments, antibody fragments include a Fab fragment generated by the treatment of the antibody molecule with papain and a reducing agent. In certain embodiments, antibody fragments are produced by recombinant methods. In some embodiments, antibody fragments include Fv or single-chain Fv (scFv) fragments. Fab. Fv, and scFv antibody fragments can be expressed in and secreted from E. coli or other host cells, allowing for the production of large amounts of these fragments. In some embodiments, antibody fragments are isolated from antibody phage libraries as discussed herein. For example, methods can be used for the construction of Fab expression libraries to allow rapid and effective identification of monoclonal Fab fragments with the desired specificity for the antigen or derivatives, fragments, analogs or homologs thereof. In some embodiments, antibody fragments are linear antibody fragments. In certain embodiments, antibody fragments are monospecific or bispecific. In certain embodiments, the antibody is a scFv. Various techniques can be used for the production of single-chain antibodies specific to the antigen.

In some embodiments, especially in the case of antibody fragments, an antibody is modified in order to alter (e.g., increase or decrease) its serum half-life. This can be achieved, for example, by incorporation of a salvage receptor binding epitope into the antibody fragment by mutation of the appropriate region in the antibody fragment or by incorporating the epitope into a peptide tag that is then fused to the antibody fragment at either end or in the middle (e.g., by DNA or peptide synthesis).

Heteroconjugate antibodies are also within the scope of the present invention. Heteroconjugate antibodies are composed of two covalently joined antibodies. Such antibodies have, for example, been proposed to target immune cells to unwanted cells. It is also contemplated that the heteroconjugate antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate.

For the purposes of the present invention, it should be appreciated that modified antibodies can comprise any type of variable region that provides for the association of the antibody with the target antigen. In this regard, the variable region may comprise or be derived from any type of mammal that can be induced to mount a humoral response and generate immunoglobulins against the desired antigen. As such, the variable region of the modified antibodies can be, for example, of human, murine, rat, rabbit, non-human primate (e.g., cynomolgus monkeys, macaques, etc.), or rabbit origin. In some embodiments, both the variable and constant regions of the modified immunoglobulins are human. In other embodiments, the variable regions of compatible antibodies (usually derived from a non-human source) can be engineered or specifically tailored to improve the binding properties or reduce the immunogenicity of the molecule. In this respect, variable regions useful in the present invention can be humanized or otherwise altered through the inclusion of imported amino acid sequences.

In certain embodiments, the variable domains in both the heavy and light chains are altered by at least partial replacement of one or more CDRs and, if necessary, by partial framework region replacement and sequence modification and/or alteration. Although the CDRs may be derived from an antibody of the same class or even subclass as the antibody from which the framework regions are derived, it is envisaged that the CDRs may be derived from an antibody of different class and often from an antibody from a different species. It may not be necessary to replace all of the CDRs with all of the CDRs from the donor variable region to transfer the antigen-binding capacity of one variable domain to another. Rather, it may only be necessary to transfer those residues that are required to maintain the activity of the antigen-binding site.

Alterations to the variable region notwithstanding, those skilled in the art will appreciate that the modified antibodies of this invention will comprise antibodies (e.g., full-length antibodies or immunoreactive fragments thereof) in which at least a fraction of one or more of the constant region domains has been deleted or otherwise altered so as to provide desired biochemical characteristics such as increased tumor localization or increased serum half-life when compared with an antibody of approximately the same immunogenicity comprising a native or unaltered constant region. In some embodiments, the constant region of the modified antibodies will comprise a human constant region. Modifications to the constant region compatible with this invention comprise additions, deletions or substitutions of one or more amino acids in one or more domains. The modified antibodies disclosed herein may comprise alterations or modifications to one or more of the three heavy chain constant domains (CH1, CH2 or CH3) and/or to the light chain constant domain (CL). In some embodiments, one or more domains are partially or entirely deleted from the constant regions of the modified antibodies. In some embodiments, the modified antibodies will comprise domain deleted constructs or variants wherein the entire CH2 domain has been removed (ΔCH2 constructs). In some embodiments, the omitted constant region domain is replaced by a short amino acid spacer (e.g., 10 amino acid residues) that provides some of the molecular flexibility typically imparted by the absent constant region.

In some embodiments, the modified antibodies are engineered to fuse the CH3 domain directly to the hinge region of the antibody. In other embodiments, a peptide spacer is inserted between the hinge region and the modified CH2 and/or CH3 domains. For example, constructs may be expressed wherein the CH2 domain has been deleted, and the remaining CH3 domain (modified or unmodified) is joined to the hinge region with a 5-20 amino acid spacer. Such a spacer may be added to ensure that the regulatory elements of the constant domain remain free and accessible or that the hinge region remains flexible. However, it should be noted that amino acid spacers may, in some cases, prove to be immunogenic and elicit an unwanted immune response against the construct. Accordingly, in certain embodiments, any spacer added to the construct will be relatively non-immunogenic so as to maintain the desired biological qualities of the modified antibodies.

In some embodiments, the modified antibodies may have only a partial deletion of a constant domain or substitution of a few or even a single amino acid. For example, the mutation of a single amino acid in selected areas of the CH2 domain may be enough to substantially reduce Fc binding. In some embodiments, the mutation of a single amino acid in selected areas of the CH2 domain may be enough to substantially reduce Fc binding and increase cancer cell localization and/or tumor penetration. Similarly, it may be desirable to simply delete the part of one or more constant region domains that control a specific effector function (e.g., complement C1q binding) to be modulated. Such partial deletions of the constant regions may improve selected characteristics of the antibody (serum half-life) while leaving other desirable functions associated with the subject constant region domain intact. Moreover, as alluded to above, the constant regions of the disclosed antibodies may be modified through the mutation or substitution of one or more amino acids that enhances the profile of the resulting construct. In this respect, it may be possible to disrupt the activity provided by a conserved binding site (e.g., Fc binding) while substantially maintaining the configuration and immunogenic profile of the modified antibody. In certain embodiments, the modified antibodies comprise the addition of one or more amino acids to the constant region to enhance desirable characteristics such as decreasing or increasing effector function or provide for more cytotoxin or carbohydrate attachment sites.

It is known in the art that the constant region mediates several effector functions. For example, binding of the C1 component of complement to the Fc region of IgG or IgM antibodies (bound to antigen) activates the complement system. Activation of complement is important in the opsonization and lysis of cell pathogens. The activation of complement also stimulates the inflammatory response and can also be involved in autoimmune hypersensitivity. In addition, the Fc region of an antibody can bind a cell expressing a Fc receptor (FcR). There are a number of Fc receptors which are specific for different classes of antibody, including IgG (gamma receptors), IgE (epsilon receptors), IgA (alpha receptors) and IgM (mu receptors). Binding of antibody to Fc receptors on cell surfaces triggers a number of important and diverse biological responses including engulfment and destruction of antibody-coated particles, clearance of immune complexes, lysis of antibody-coated target cells by killer cells (called antibody-dependent cell cytotoxicity or ADCC), release of inflammatory mediators, placental transfer, and control of immunoglobulin production.

In certain embodiments, the modified antibodies provide for altered effector functions that, in turn, affect the biological profile of the administered antibody. For example, in some embodiments, the deletion or inactivation (through point mutations or other means) of a constant region domain may reduce Fc receptor binding of the circulating modified antibody. In some embodiments, the deletion or inactivation (through point mutations or other means) of a constant region domain may reduce Fc receptor binding of the circulating modified antibody thereby increasing cancer cell localization and/or tumor penetration. In other embodiments, the constant region modifications increase the serum half-life of the antibody. In other embodiments, the constant region modifications reduce the serum half-life of the antibody. In some embodiments, the constant region is modified to eliminate disulfide linkages or oligosaccharide moieties. Modifications to the constant region in accordance with this invention may easily be made using well known biochemical or molecular engineering techniques.

In certain embodiments, the antibody described herein does not have one or more effector functions. For instance, in some embodiments, the antibody has no ADCC activity, and/or no complement-dependent cytotoxicity (CDC) activity. In certain embodiments, the antibody does not bind an Fc receptor and/or complement factors. In certain embodiments, the antibody has no effector function(s).

The present invention further embraces variants and equivalents which are substantially homologous to the recombinant, monoclonal, chimeric, humanized, and human antibodies, or antibody fragments thereof, described herein. These variants can contain, for example, conservative substitution mutations, i.e. the substitution of one or more amino acids by similar amino acids.

The present invention provides methods for producing an antibody that binds a target antigen (e.g., B7-H4 or P-CADHERIN), including bispecific antibodies that specifically bind both the first target antigen and a second target. In some embodiments, the method for producing the antibody comprises using hybridoma techniques. In some embodiments, a method for producing an antibody that binds human B7-H4 or human P-CADHERIN is provided. In some embodiments, the method comprises using a polypeptide comprising the extracellular domain of human B7-H4 or human P-CADHERIN, or a fragment thereof, as an antigen. In some embodiments, the method for producing an antibody that binds human B7-H4 comprises using a polypeptide of SEQ ID NO:88, or a fragment thereof, as an antigen. In some embodiments, the method for producing an antibody that binds the extracellular domain of human P-CADHERIN comprises using a polypeptide of SEQ ID NO:91, or a fragment thereof, as an antigen. In some embodiments, the method of generating an antibody that binds B7-H4 or P-CADHERIN comprises screening a phage library, for example, a human phage library. The present invention further provides methods of identifying an antibody that binds B7-H4 or P-CADHERIN. In some embodiments, the antibody is identified by FACS screening for binding to B7-H4 or P-CADHERIN or a fragment thereof. In some embodiments, the antibody is identified by screening using ELISA for binding to B7-H4 or P-CADHERIN, or a fragment thereof.

The antibodies of the present invention can be assayed for specific binding by any method known in the art. The immunoassays which can be used include, but are not limited to, competitive and non-competitive assay systems using techniques such as Biacore analysis, FACS analysis, immunofluorescence, immunocytochemistry, Western blot analysis, radioimmunoassay, ELISA, “sandwich” immunoassay, immunoprecipitation assay, precipitation reaction, gel diffusion precipitin reaction, immunodiffusion assay, agglutination assay, complement-fixation assay, immunoradiometric assay, fluorescent immunoassay, and protein A immunoassay. Such assays are routine and well-known in the art (see, e.g., Ausubel et al., Editors, 1994-present, Current Protocols in Molecular Biology, John Wiley & Sons, Inc., New York, N.Y.).

In a non-limiting example, screening for specific binding of an antibody to its target antigen may be determined using ELISA. An ELISA comprises preparing antigen, coating wells of a 96-well microtiter plate with antigen, adding the test antibodies conjugated to a detectable compound such as an enzymatic substrate (e.g. horseradish peroxidase or alkaline phosphatase) to the well, incubating for a period of time, and detecting the presence of an antibody bound to the antigen. In some embodiments, the test antibodies are not conjugated to a detectable compound, but instead, a secondary antibody that recognizes the antibody (e.g., an anti-Fc antibody) and is conjugated to a detectable compound is added to the wells. In some embodiments, instead of coating the well with the antigen, the test antibodies can be coated to the wells, the antigen is added to the wells, followed by a secondary antibody conjugated to a detectable compound. One of skill in the art would be knowledgeable as to the parameters that can be modified to increase the signal detected as well as other variations of ELISAs known in the art.

In another non-limiting example, the specific binding of an antibody may be determined using FACS. A FACS screening assay may comprise generating a cDNA construct that expresses an antigen as a full-length protein or a fusion protein (e.g., an antigen-CD4TM fusion protein), transfecting the construct into cells, expressing the antigen on the surface of the cells, mixing the test antibodies with the transfected cells, and incubating for a period of time. The cells bound by the test antibodies may be identified using a secondary antibody conjugated to a detectable compound (e.g., PE-conjugated anti-Fc antibody) and a flow cytometer. One of skill in the art would be knowledgeable as to the parameters that can be modified to optimize the signal detected as well as other variations of FACS that may enhance screening (e.g., screening for blocking antibodies).

The binding affinity of an antibody or other binding agent to an antigen and the off-rate of an antibody-antigen interaction can be determined by competitive binding assays. One example of a competitive binding assay is a radioimmunoassay comprising the incubation of labeled antigen (e.g., ³H or ¹²⁵I-antigen), or fragment or variant thereof, with the antibody of interest in the presence of increasing amounts of unlabeled antigen followed by the detection of the antibody bound to the labeled antigen. The affinity of the antibody for the antigen and the binding off-rates can be determined from the data by Scatchard plot analysis. In some embodiments, the Biacore kinetic analysis is used to determine the binding on and off rates of antibodies or agents that bind an antigen. In some embodiments, the Biacore kinetic analysis comprises analyzing the binding and dissociation of antibodies from chips with immobilized antigen on their surface. In some embodiments, the Biacore kinetic analysis comprises analyzing the binding and dissociation of antigen from chips with the immobilized antibody on their surface.

In certain embodiments, the antibodies can be used in any one of a number of conjugated (i.e., an immunoconjugate or radioconjugate) or non-conjugated forms. In certain embodiments, the antibodies can be used in a non-conjugated form to harness the subject's natural defense mechanisms including complement-dependent cytotoxicity (CDC) and antibody-dependent cellular cytotoxicity (ADCC) to eliminate malignant or cancer cells.

In some embodiments, the antibody is conjugated to a cytotoxic agent. In some embodiments, the antibody is conjugated to a cytotoxic agent as an ADC (antibody-drug conjugate). In some embodiments, the cytotoxic agent is a chemotherapeutic agent including, but not limited to, methotrexate, adriamycin/doxorubicin, melphalan, mitomycin C, chlorambucil, daunorubicin, pyrrolobenzodiazepines (PBDs), or other intercalating agents. In some embodiments, the cytotoxic agent is an enzymatically active toxin of bacterial, fungal, plant, or animal origin, or fragments thereof, including, but not limited to, diphtheria A chain, non-binding active fragments of diphtheria toxin, exotoxin A chain, ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes. In some embodiments, the cytotoxic agent is a radioisotope to produce a radioconjugate or a radioconjugated antibody. A variety of radionuclides are available for the production of radioconjugated antibodies including, but not limited to, ⁹⁰Y, ¹²⁵I, ¹³¹I, ¹²³I, ¹¹¹In, ¹³¹In, ¹⁰⁵Rh, ¹⁵³Sm, ⁶⁷Cu, ⁶⁷Ga, ¹⁶⁶Ho, ¹⁷⁷Lu, ¹⁸⁶Re, ¹⁸⁸Re, and ²¹²Bi. Conjugates of an antibody and one or more small molecule toxins, such as calicheamicins, maytansinoids, trichothenes, and CC1065, and the derivatives of these toxins that have toxin activity, can also be used. Conjugates of an antibody and cytotoxic agent may be made using a variety of bifunctional protein-coupling agents such as N-succinimidyl-3-(2-pyridyidithiol) propionate (SPDP), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis(p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene).

In certain embodiments, the antibody is detectably labeled. By way of non-limiting example, the antibody may be labeled with an affinity label, an enzymatic label, a fluorescent label, a radioisotope label, or a magnetic label.

In certain embodiments, the antibody is linked to a LTβR-binding moiety or a polypeptide that binds human LTβR. By way of non-limiting example, the antibodies of the invention may be linked to any of the fusion or single-chain polypeptides described herein including, without limitation, those described in the section above entitled “II. Molecules/agents comprising lymphotoxin-αββ.”

In certain alternative embodiments, the antibody is linked to a therapeutic agent.

Agents comprising each of the antibodies described herein are also provided. In some embodiments, the agents are polypeptides. Polypeptides and agents comprising each of the antibodies described herein as a targeting moiety are also provided. In certain embodiments, the polypeptides or agents further comprise an LTβR-binding moiety. In certain embodiments, the polypeptides or agents comprise an antibody fragment or antigen-binding site that specifically binds to human B7-H4 or human P-CADHERIN.

In another aspect, the invention provides polypeptides comprising a polypeptide having a sequence selected from the group consisting of the following: SEQ ID NO:50, SEQ ID NO:51, SEQ ID NOs:59-66, and SEQ ID NOs: 104-107. In an alternative aspect, the invention provides a polypeptide comprising a polypeptide having a sequence selected from the group consisting of SEQ ID NOs:67-72, SEQ ID NOs:76-79, SEQ ID NO:92, SEQ ID NO:93, SEQ ID NO:98, SEQ ID NO:99, SEQ ID NO: 101, and SEQ ID NO: 102. In certain embodiments, the polypeptide comprises a polypeptide having the sequence of SEQ ID NO:62. In other embodiments, the polypeptide comprises a polypeptide having the sequence of SEQ ID NO:66. In some embodiments, the polypeptide comprises a polypeptide having the sequence of SEQ ID NO:79. In some alternative embodiments, the polypeptide comprises a polypeptide having the sequence of SEQ ID NO:72 or SEQ ID NO:93. In some embodiments, the polypeptide comprises SEQ ID NO: 105 and/or SEQ ID NO: 107. In some other embodiments, the polypeptide comprises SEQ ID NO:99 and/or SEQ ID NO: 102. Cells producing or comprising one or more of the polypeptides are also provided.

### V. Polynucleotides

In certain embodiments, the invention encompasses polynucleotides comprising polynucleotides that encode a polypeptide, agent, antibody, or molecule described herein. The term “polynucleotides that encode a polypeptide” encompasses a polynucleotide which includes only coding sequences for the polypeptide as well as a polynucleotide which includes additional coding and/or non-coding sequences. The polynucleotides of the invention can be in the form of RNA or in the form of DNA. DNA includes cDNA, genomic DNA, and synthetic DNA; and can be double-stranded or single-stranded, and if single stranded can be the coding strand or non-coding (anti-sense) strand.

In certain embodiments, the polynucleotide comprises a polynucleotide encoding a polypeptide comprising an amino acid sequence selected from the group consisting of: SEQ ID NO:2, SEQ ID NO:3, SEQ ID NO:5, SEQ ID NO:6, SEQ ID NO:7, SEQ ID NO:8, and SEQ ID NO:9. In certain embodiments, the polynucleotide comprises a polynucleotide encoding a polypeptide comprising an amino acid sequence selected from the group consisting of: SEQ ID NO: 11, SEQ ID NO: 12, SEQ ID NO: 14. SEQ ID NO: 15, SEQ ID NO: 16, SEQ ID NO: 17, and SEQ ID NO: 18. In certain embodiments, the polynucleotide comprises a polynucleotide encoding a polypeptide having an amino acid sequence selected from the group consisting of: SEQ ID NO:95, SEQ ID NO:96, and SEQ ID NO:97. In some embodiments, a polynucleotide comprises a polynucleotide that encodes a polypeptide comprising any of the lymphotoxin αββ proteins described herein. In some embodiments, a polynucleotide comprises a polynucleotide that encodes a polypeptide of any of the lymphotoxin αββ polypeptides described herein and a signal sequence. In some embodiments, a vector comprises the polynucleotide. In some embodiments, a cell comprises the polynucleotide. In some embodiments, a cell comprises the vector. In some embodiments, the cell is isolated.

In certain other embodiments, the polynucleotide comprises a polynucleotide encoding a polypeptide comprising SEQ ID NO:86. In certain embodiments, the polynucleotide comprises a polynucleotide encoding a polypeptide comprising SEQ ID NO:86. In some embodiments, a vector comprises the polynucleotide. In some embodiments, a cell comprises the polynucleotide. In some embodiments, a cell comprises the vector. In some embodiments, the cell is isolated.

In certain other embodiments, the polynucleotide comprises a polynucleotide encoding a polypeptide comprising an amino acid sequence selected from the group consisting of: SEQ ID NO:50, SEQ ID NO:51, SEQ ID NOs:59-66, and SEQ ID NOs:104-107. In certain embodiments, the polynucleotide comprises a polynucleotide encoding a polypeptide comprising an amino acid sequence selected from the group consisting of: SEQ ID NO:50, SEQ ID NO:51, SEQ ID NOs:59-66, and SEQ ID NOs:104-107. Vectors comprising the polynucleotides are also provided. In some embodiments, a cell comprises the polynucleotide. In some embodiments, a cell comprises the vector. In some embodiments, the cell is isolated.

In certain further embodiments, the polynucleotide comprises a polynucleotide encoding a polypeptide comprising an amino acid sequence selected from the group consisting of: SEQ ID NOs:67-72, SEQ ID NOs:76-79, SEQ ID NO:92, SEQ ID NO:93, SEQ ID NO:98, SEQ ID NO:99, SEQ ID NO: 101, and SEQ ID NO:102. In certain embodiments, the polynucleotide comprises a polynucleotide encoding a polypeptide comprising an amino acid sequence selected from the group consisting of: SEQ ID NOs:67-72, SEQ ID NOs:76-79, SEQ ID NO:92, SEQ ID NO:93, SEQ ID NO:98, SEQ ID NO:99, SEQ ID NO: 101, and SEQ ID NO: 102. Vectors comprising the polynucleotides are also provided. In some embodiments, a cell comprises the polynucleotide. In some embodiments, a cell comprises the vector. In some embodiments, the cell is isolated.

In one aspect, a polynucleotide comprising SEQ ID NO:57 and/or SEQ ID NO:58 is provided. In another aspect, a polynucleotide comprising SEQ ID NO: 100 and/or SEQ ID NO: 103 is provided.

In certain embodiments, a polynucleotide comprises a polynucleotide having a nucleotide sequence at least 80% identical, at least 85% identical, at least 90% identical, at least 95% identical, and in some embodiments, at least 96%, 97%, 98% or 99% identical to a polynucleotide encoding an amino acid sequence selected from the group consisting of: SEQ ID NO:2, SEQ ID NO:3. SEQ ID NO:5, SEQ ID NO:6. SEQ ID NO:7, SEQ ID NO:8, SEQ ID NO:9, SEQ ID NO: 11. SEQ ID NO: 12, SEQ ID NO: 14, SEQ ID NO: 15, SEQ ID NO: 16, SEQ ID NO:17, and SEQ ID NO: 18. In certain embodiments, the polynucleotide comprises a polynucleotide having a nucleotide sequence at least 80% identical, at least 85% identical, at least 90% identical, at least 95% identical, and in some embodiments, at least 96%, 97%, 98% or 99% identical to a polynucleotide encoding an amino acid sequence selected from the group consisting of: SEQ ID NO:95, SEQ ID NO:96, and SEQ ID NO:97.

In certain embodiments, a polynucleotide comprises a polynucleotide having a nucleotide sequence at least 80% identical, at least 85% identical, at least 90% identical, at least 95% identical, and in some embodiments, at least 96%, 97%, 98% or 99% identical to a polynucleotide encoding SEQ ID NO:86.

In some embodiments, a polynucleotide comprises a polynucleotide having a nucleotide sequence at least 80% identical, at least 85% identical, at least 90% identical, at least 95% identical, and in some embodiments, at least 96%, 97%, 98% or 99% identical to a polynucleotide encoding an amino acid sequence selected from the group consisting of SEQ ID NO:50, SEQ ID NO:51, SEQ ID NOs:59-66, and SEQ ID NOs: 104-107. In certain other embodiments, the polynucleotide comprises a polynucleotide having a nucleotide sequence at least 80% identical, at least 85% identical, at least 90% identical, at least 95% identical, and in some embodiments, at least 96%, 97%, 98% or 99% identical to a polynucleotide encoding an amino acid sequence selected from the group consisting of SEQ ID NOs:67-72, SEQ ID NOs:76-79, SEQ ID NO:92, SEQ ID NO:93, SEQ ID NO:98, SEQ ID NO:99, SEQ ID NO: 101, and SEQ ID NO: 102.

Also provided is a polynucleotide that comprises a polynucleotide that hybridizes to a polynucleotide encoding an amino acid sequence selected from the group consisting of: SEQ ID NO:2, SEQ ID NO:3, SEQ ID NO:5, SEQ ID NO:6, SEQ ID NO:7, SEQ ID NO:8, SEQ ID NO:9, SEQ ID NO: 11, SEQ ID NO: 12, SEQ ID NO: 14, SEQ ID NO: 15, SEQ ID NO: 16, SEQ ID NO: 17, and SEQ ID NO:18.

Polynucleotides that hybridize to a polynucleotide encoding SEQ ID NO:95, SEQ ID NO:96, or SEQ ID NO:97 are also provided.

In another aspect, the invention provides a polynucleotide that comprises a polynucleotide that hybridizes to a polynucleotide encoding SEQ ID NO:86.

In another aspect, the invention provides a polynucleotide that comprises a polynucleotide that hybridizes to a polynucleotide encoding an amino acid sequence selected from the group consisting of: SEQ ID NO:50, SEQ ID NO:51, SEQ ID NOs:59-66, and SEQ ID NOs:104-107. In certain alternative embodiments, the polynucleotide comprises a polynucleotide that hybridizes to a polynucleotide encoding an amino acid sequence selected from the group consisting of: SEQ ID NOs:67-72, SEQ ID NOs:76-79, SEQ ID NO:92, SEQ ID NO:93, SEQ ID NO:98, SEQ ID NO:99, SEQ ID NO: 101, and SEQ ID NO: 102.

In a further aspect, the invention provides a polynucleotide comprising a polynucleotide that hybridizes to SEQ ID NO:57, SEQ ID NO:58. SEQ ID NO: 100, or SEQ ID NO: 103.

In certain embodiments, the hybridization is under conditions of high stringency. Conditions of high stringency are known to those of skill in the art and may include but are not limited to, (1) employ low ionic strength and high temperature for washing, for example, 15 mM sodium chloride/1.5 mM sodium citrate (1×SSC) with 0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 in 5×SSC (0.75M NaCl, 75 mM sodium citrate) at 42° C.; or (3) employ 50% formamide, 5×SSC, 50 mM sodium phosphate (pH 6.8), 0.1% sodium pyrophosphate, 5×Denhardt's solution, sonicated salmon sperm DNA (50 g/ml), 0.1% SDS, and 10% dextran sulfate at 42° C., with washes in 0.2×SSC containing 50% formamide at 55° C., followed by a high-stringency wash consisting of 0.1×SSC containing EDTA at 55° C.

The invention further relates to variants of the hereinabove described polynucleotides encoding, for example, fragments, analogs, and/or derivatives.

In certain embodiments, the present invention provides a polynucleotide comprising a polynucleotide having a nucleotide sequence at least about 80% identical, at least about 85% identical, at least about 90% identical, at least about 95% identical, and in some embodiments, at least about 96%, 97%, 98% or 99% identical to a polynucleotide encoding a polypeptide comprising a polypeptide or molecule described herein.

As used herein, the phrase a polynucleotide having a nucleotide sequence at least, for example, 95% “identical” to a reference nucleotide sequence is intended to mean that the nucleotide sequence of the polynucleotide is identical to the reference sequence except that the polynucleotide sequence can include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence can be deleted or substituted with another nucleotide, or a number of nucleotides up to 5% of the total nucleotides in the reference sequence can be inserted into the reference sequence. These mutations of the reference sequence can occur at the 5′ or 3′ terminal positions of the reference nucleotide sequence or anywhere between those terminal positions, interspersed either individually among nucleotides in the reference sequence or in one or more contiguous groups within the reference sequence.

The polynucleotide variants can contain alterations in the coding regions, non-coding regions, or both. In some embodiments, a polynucleotide variant contains alterations which produce silent substitutions, additions, or deletions, but does not alter the properties or activities of the encoded polypeptide. In some embodiments, a polynucleotide variant comprises silent substitutions that results in no change to the amino acid sequence of the polypeptide (due to the degeneracy of the genetic code). Polynucleotide variants can be produced for a variety of reasons, for example, to optimize codon expression for a particular host (i.e., change codons in the human mRNA to those preferred by a bacterial host such as E. coli). In some embodiments, a polynucleotide variant comprises at least one silent mutation in a non-coding or a coding region of the sequence.

In some embodiments, a polynucleotide variant is produced to modulate or alter expression (or expression levels) of the encoded polypeptide. In some embodiments, a polynucleotide variant is produced to increase expression of the encoded polypeptide. In some embodiments, a polynucleotide variant is produced to decrease expression of the encoded polypeptide. In some embodiments, a polynucleotide variant has increased expression of the encoded polypeptide as compared to a parental polynucleotide sequence. In some embodiments, a polynucleotide variant has decreased expression of the encoded polypeptide as compared to a parental polynucleotide sequence.

In some embodiments, at least one polynucleotide variant is produced (without changing the amino acid sequence of the encoded polypeptide) to increase production of a heterodimeric molecule. In some embodiments, at least one polynucleotide variant is produced (without changing the amino acid sequence of the encoded polypeptide) to increase production of a bispecific agent, a bispecific antibody, or a heterodimeric agent.

In certain embodiments, the polynucleotides are isolated. In certain embodiments, the polynucleotides are substantially pure.

Vectors and cells comprising the polynucleotides described herein are also provided. In some embodiments, an expression vector comprises a polynucleotide molecule. In some embodiments, a host cell comprises an expression vector comprising the polynucleotide molecule. In some embodiments, a host cell comprises a polynucleotide molecule.

### VI. Molecules/Agents Comprising Alternative LTβR-Binding Moieties

In a further aspect, the invention provides molecules and/or agents that comprise an alternative LTβR-binding moiety. In certain embodiments, the molecules or agents further comprise a targeting moiety. In some embodiments, the molecules or agents may be polypeptides. In certain embodiments, the LTβR-binding moiety may activate LTβR and/or induce LTβR signaling. The LTβR-binding moiety may be a single-chain polypeptide. The LTβR-binding moiety may be a fully human single-chain polypeptide.

In certain embodiments, the alternative LTβR-binding moiety comprises LIGHT (e.g., human LIGHT) or a fragment thereof. As a non-limiting example, the LTβR-binding moiety may comprise the extracellular domain of LIGHT or a fragment thereof. In certain embodiments, the LTβR-binding moiety comprises a LIGHT homotrimer (e.g., a single-chain LIGHT homotrimer). For instance, the LTβR-binding moiety may comprise the extracellular domain of human LIGHT, a variant thereof having at least 80% sequence identity to the extracellular domain of human LIGHT, or a fragment thereof. In certain embodiments, the LTβR-binding moiety may comprise a polypeptide (e.g., a LIGHT homotrimer) having at least about 80%, at least about 90%, at least about 95%, at least about 98%, or 100% sequence identity to SEQ ID NO:85. In some embodiments, the LTβR-binding moiety is a single-chain polypeptide. In certain embodiments, the LTβR-binding moiety comprises a polypeptide having at least about 90%, at least about 95%, or at least about 98% sequence identity to SEQ ID NO:86. For example, the LTβR-binding moiety may comprise SEQ ID NO:86. In some embodiments, the LTβR-binding moiety comprises a mutant LIGHT homotrimer that has reduced the ability to bind to or activate HVEM.

In certain other embodiments, the LTβR-binding moiety comprises an antibody that specifically binds LTβR. The antibody may, in some embodiments, be an agonist of LTβR. Agonist antibodies that specifically bind LTβR are known in the art. For example, see WO 2006/114284, US 2006/0104971, and U.S. Pat. No. 7,429,644, each of which is hereby incorporated by reference herein. In certain embodiments, the antibody is an antigen-binding antibody fragment.

In certain embodiments, the targeting moiety of the molecule or agent comprising both an LTβR-binding moiety and a targeting moiety is capable of binding an antigen on the surface of a target cell. In certain embodiments, the targeting moiety selectively targets the target cell (and/or the tissue containing the target cell). In certain embodiments, the targeting moiety specifically binds a tumor-associated antigen. In certain embodiments, the tumor-associated antigen is selected from the group consisting of B7-H4, B7-H3, P-CADHERIN (CDH3), GABRP, ACPP, SLC45A3, STEAP1, STEAP2, GPA33, GUCY2C, GARP, PVRL4, mesothelin, and CA9. In some embodiments, the targeting moiety is an antibody (e.g., a full-length antibody or an antigen-binding antibody fragment). By way of non-limiting example, the targeting moiety of the molecule or agent may be an antibody that specifically binds the extracellular domain of human B7-H4. In certain other embodiments, the targeting moiety of the molecule or agent may be an antibody that specifically binds the extracellular domain of human P-CADHERIN. The B7-H4 or P-CADHERIN antibody may, in some embodiments, be selected from the B7-H4 or P-CADHERIN antibodies described in the section above entitled “IV. Antibodies to cell-surface antigens” or described elsewhere herein.

The invention further provides polypeptides that comprise SEQ ID NO:86. Cells producing the polypeptide are also provided.

### VII. Methods of Use and Pharmaceutical Compositions

The polypeptides, agents, antibodies, and molecules of the invention are useful in a variety of applications including, but not limited to, therapeutic treatment methods, such as immunotherapy for cancer. In certain embodiments, a polypeptide, agent, antibody, or molecule described herein is useful for activating, promoting, increasing, and/or enhancing an immune response, recruiting TILs to a tumor, promoting and/or enhancing the formation of lymphoid structure within a tumor or tumor microenvironment, increasing CTL activity, inhibiting tumor growth, reducing tumor volume, inducing tumor regression, increasing tumor cell apoptosis, and/or reducing the tumorigenicity of a tumor. The methods of use may be in vitro, ex vivo, or in vivo methods.

The present invention provides methods for activating an immune response in a subject using a polypeptide, agent, antibody, or molecule described herein. In some embodiments, the invention provides methods for promoting an immune response in a subject using a polypeptide, agent, antibody, or molecule described herein. In some embodiments, the invention provides methods for increasing an immune response in a subject using a polypeptide, agent, antibody, or molecule described herein. In some embodiments, the invention provides methods for enhancing an immune response in a subject using a polypeptide, agent, antibody, or molecule described herein. In some embodiments, the activating, promoting, increasing, and/or enhancing of an immune response comprises increasing cell-mediated immunity. In some embodiments, the activating, promoting, increasing, and/or enhancing of an immune response comprises increasing Th1-type responses. In some embodiments, the activating, promoting, increasing, and/or enhancing of an immune response comprises increasing T-cell activity. In some embodiments, the activating, promoting, increasing, and/or enhancing of an immune response comprises increasing CD4+ T-cell activity. In some embodiments, the activating, promoting, increasing, and/or enhancing of an immune response comprises increasing CD8+ T-cell activity. In some embodiments, the activating, promoting, increasing, and/or enhancing of an immune response comprises increasing CTL activity. In some embodiments, the activating, promoting, increasing, and/or enhancing of an immune response comprises increasing NK cell activity. In some embodiments, the activating, promoting, increasing, and/or enhancing of an immune response comprises increasing T-cell activity and increasing NK cell activity. In some embodiments, the activating, promoting, increasing, and/or enhancing of an immune response comprises increasing CTL activity and increasing NK cell activity. In some embodiments, the activating, promoting, increasing, and/or enhancing of an immune response comprises increasing the number of the percentage of memory T-cells. In some embodiments, the activating, promoting, increasing, and/or enhancing of an immune response comprises increasing long-term immune memory function. In some embodiments, the activating, promoting, increasing, and/or enhancing of an immune response comprises increasing long-term memory. In some embodiments, the activating, promoting, increasing, and/or enhancing of an immune response comprises no evidence of substantial side effects and/or immune-based toxicities. In some embodiments, the activating, promoting, increasing, and/or enhancing of an immune response comprises no evidence of cytokine release syndrome (CRS) or a cytokine storm. In some embodiments, the immune response is a result of antigenic stimulation. In some embodiments, the antigenic stimulation is a tumor cell. In some embodiments, the antigenic stimulation is cancer.

In vivo and in vitro assays for determining whether a polypeptide, molecule, antibody, or agent modulates, activates, or inhibits an immune response are known in the art or are being developed.

In some embodiments, a method of increasing an immune response in a subject comprises administering to the subject a therapeutically effective amount of a polypeptide, agent, or molecule described herein, wherein the polypeptide, agent, or molecule binds human LTβR. In some embodiments, a method of increasing an immune response in a subject comprises administering to the subject a therapeutically effective amount of a polypeptide, agent, or molecule described herein, wherein the polypeptide, agent, or molecule comprises a fusion polypeptide comprising lymphotoxin-αββ that specifically binds to LTβR. In some embodiments, a method of increasing an immune response in a subject comprises administering to the subject a therapeutically effective amount of a polypeptide, agent, or molecule described herein, wherein the polypeptide, agent, or molecule comprises a fusion polypeptide comprising lymphotoxin-αββ. In certain embodiments, the methods described herein comprise administering a therapeutically effective amount of a polypeptide, agent, or molecule comprising a single-chain polypeptide described herein that binds human LTβR. In some embodiments, the methods may comprise administering a therapeutically effective amount of a polypeptide, agent, or molecule comprising another LTβR-binding moiety described herein.

In certain embodiments of the methods described herein, a method of activating or enhancing a persistent or long-term immune response to a tumor comprises administering to a subject a therapeutically effective amount of a polypeptide, agent, or molecule that binds human LTβR. In some embodiments, a method of activating or enhancing a persistent immune response to a tumor comprises administering to a subject a therapeutically effective amount of a polypeptide, agent, or molecule described herein, wherein the polypeptide, agent, or molecule is a fusion polypeptide comprising lymphotoxin-αββ that specifically binds to LTβR. In some embodiments, a method of activating or enhancing a persistent immune response to a tumor comprises administering to a subject a therapeutically effective amount of a polypeptide, agent, or molecule described herein, wherein the polypeptide, agent, or molecule comprises a fusion polypeptide comprising lymphotoxin-αββ. In some embodiments, the polypeptide comprises a fusion polypeptide comprising lymphotoxin-αββ and a targeting moiety. In some embodiments, the polypeptide comprises a fusion polypeptide comprising lymphotoxin-αββ and an antibody. In some embodiments, the polypeptide comprises a fusion polypeptide comprising lymphotoxin-αββ and an antibody that specifically binds a tumor-associated antigen. In some embodiments, the polypeptide comprises a comprises a fusion polypeptide comprising lymphotoxin-αββ and a Fc region. In certain embodiments, the methods described herein comprise administering a therapeutically effective amount of a polypeptide, agent, or molecule comprising a single-chain polypeptide described herein that binds human LTβR. In some embodiments, the methods may comprise administering a therapeutically effective amount of a polypeptide, agent, or molecule comprising another LTβR-binding moiety described herein.

In certain embodiments of the methods described herein, a method of inducing a persistent or long-term immunity which inhibits tumor relapse or tumor regrowth comprises administering to a subject a therapeutically effective amount of a polypeptide, agent, or molecule which binds human LTβR. In some embodiments, a method of inducing a persistent immunity which inhibits tumor relapse or tumor regrowth comprises administering to a subject a therapeutically effective amount of a polypeptide, agent, or molecule described herein, wherein the polypeptide, agent, or molecule comprises a fusion polypeptide comprising lymphotoxin-αββ. In some embodiments, the polypeptide comprises a fusion polypeptide comprising lymphotoxin-αββ and a targeting moiety. In some embodiments, the polypeptide comprises a fusion polypeptide comprising lymphotoxin-αββ and an antibody. In some embodiments, the polypeptide comprises a fusion polypeptide comprising lymphotoxin-αββ and an antibody that specifically binds a tumor-associated antigen. In some embodiments, the polypeptide comprises a comprises a fusion polypeptide comprising lymphotoxin-αββ and a Fc region. In certain embodiments, the methods described herein comprise administering a therapeutically effective amount of a polypeptide, agent, or molecule comprising a single-chain polypeptide described herein that binds human LTβR. In some embodiments, the methods may comprise administering to the subject a therapeutically effective amount of a polypeptide, agent, or molecule comprising another LTβR-binding moiety described herein.

In certain embodiments of the methods described herein, a method of inhibiting tumor relapse or tumor regrowth comprises administering to a subject a therapeutically effective amount of a polypeptide, agent, or molecule which binds human LTβR. In some embodiments, a method of inhibiting tumor relapse or tumor regrowth comprises administering to a subject a therapeutically effective amount of a polypeptide, agent, or molecule described herein, wherein the polypeptide, agent, or molecule is a single-chain fusion polypeptide that specifically binds to LTβR. In some embodiments, a method of inhibiting tumor relapse or tumor regrowth comprises administering to a subject a therapeutically effective amount of a polypeptide, agent, or molecule described herein, wherein the polypeptide, agent, or molecule comprises a fusion polypeptide comprising lyvmphotoxin-αββ. In some embodiments, the polypeptide comprises a fusion polypeptide comprising lymphotoxin-αββ and a targeting moiety. In some embodiments, the polypeptide comprises a fusion polypeptide comprising lymphotoxin-αββ and an antibody. In some embodiments, the polypeptide comprises a fusion polypeptide comprising lymphotoxin-αββ and an antibody that specifically binds a tumor-associated antigen. In some embodiments, the polypeptide comprises a comprises a fusion polypeptide comprising lymphotoxin-αββ and a Fc region. In certain embodiments, the methods described herein comprise administering to the subject a therapeutically effective amount of a polypeptide, agent, or molecule comprising a single-chain polypeptide described herein that binds human LTβR. In some embodiments, the methods may comprise administering to the subject a therapeutically effective amount of a polypeptide, agent, or molecule comprising another LTβR-binding moiety described herein.

In certain embodiments of the methods described herein, a method of increasing T-cell activity in a subject comprises administering to the subject a therapeutically effective amount of a polypeptide, agent, or molecule which binds human LTβR. In certain embodiments of the methods described herein, a method of increasing T-cell activity in a subject comprises administering to the subject a therapeutically effective amount of a polypeptide, agent, or molecule, wherein the polypeptide, agent, or molecule is a single-chain fusion polypeptide that specifically binds to LTβR. In some embodiments, a method of increasing T-cell activity in a subject comprises administering to the subject a therapeutically effective amount of a polypeptide, agent, or molecule described herein, wherein the polypeptide, agent, or molecule comprises a fusion polypeptide comprising lymphotoxin-αββ. In some embodiments, the polypeptide comprises a fusion polypeptide comprising lymphotoxin-αββ and a targeting moiety. In some embodiments, the polypeptide comprises a fusion polypeptide comprising lymphotoxin-αββ and an antibody. In some embodiments, the polypeptide comprises a fusion polypeptide comprising lymphotoxin-αββ and an antibody that specifically binds a tumor-associated antigen. In some embodiments, the polypeptide comprises a comprises a fusion polypeptide comprising lymphotoxin-αββ p and a Fe region. In certain embodiments, the methods described herein comprise administering to the subject a therapeutically effective amount of a polypeptide, agent, or molecule comprising a single-chain polypeptide described herein that binds human LTβR. In some embodiments, the methods may comprise administering to the subject a therapeutically effective amount of a polypeptide, agent, or molecule comprising another LTβR-binding moiety described herein.

In certain embodiments of the methods described herein, a method of recruiting tumor-infiltrating lymphocytes to a tumor in a subject comprises administering polypeptide to a subject a therapeutically effective amount of a polypeptide, agent, or molecule which binds human LTβR. In certain embodiments of the methods described herein, a method of recruiting tumor-infiltrating lymphocytes to a tumor in a subject comprises administering to the subject a therapeutically effective amount of a polypeptide, agent, or molecule, wherein the polypeptide, agent, or molecule is a single-chain fusion polypeptide that specifically binds to LTβR. In some embodiments, a method of recruiting tumor-infiltrating lymphocytes to a tumor in a subject comprises administering to the subject a therapeutically effective amount of a polypeptide, agent, or molecule described herein, wherein the polypeptide, agent, or molecule comprises a fusion polypeptide comprising lymphotoxin-αββ. In some embodiments, the polypeptide comprises a fusion polypeptide comprising lymphotoxin-αββ and a targeting moiety. In some embodiments, the polypeptide comprises a fusion polypeptide comprising lymphotoxin-αββ and an antibody. In some embodiments, the polypeptide comprises a fusion polypeptide comprising lymphotoxin-αββ 4 and an antibody that specifically binds a tumor-associated antigen. In some embodiments, the polypeptide comprises a comprises a fusion polypeptide comprising lymphotoxin-αββ and a Fc region. In certain embodiments, the methods described herein comprise administering to the subject a therapeutically effective amount of a polypeptide, agent, or molecule comprising a single-chain polypeptide described herein that binds human LTβR. In some embodiments, the methods may comprise administering to the subject a therapeutically effective amount of a polypeptide, agent, or molecule comprising another LTβR-binding moiety described herein.

In certain embodiments of the methods described herein, a method of promoting the formation of lymphoid structures within a tumor or tumor microenvironment comprises administering to the subject a therapeutically effective amount of a polypeptide, agent, or molecule which binds human LTβR. In certain embodiments of the methods described herein, a method of promoting the formation of lymphoid structures within a tumor or tumor microenvironment in a subject comprises administering to the subject a therapeutically effective amount of a polypeptide, agent, or molecule, wherein the polypeptide, agent, or molecule is a single-chain fusion polypeptide that specifically binds to LTβR. In some embodiments, a method of promoting the formation of lymphoid structures within a tumor or tumor microenvironment in a subject comprises administering to the subject a therapeutically effective amount of a polypeptide, agent, or molecule described herein, wherein the polypeptide, agent, or molecule comprises a fusion polypeptide comprising lymphotoxin-αββ. In some embodiments, the polypeptide comprises a fusion polypeptide comprising lymphotoxin-αββ and a targeting moiety. In some embodiments, the polypeptide comprises a fusion polypeptide comprising lymphotoxin-αββ and an antibody. In some embodiments, the polypeptide comprises a fusion polypeptide comprising lymphotoxin-αββ and an antibody that specifically binds a tumor-associated antigen. In some embodiments, the polypeptide comprises a comprises a fusion polypeptide comprising lymphotoxin-αββ and a Fc region. In certain embodiments, the methods described herein comprise administering to the subject a therapeutically effective amount of a polypeptide, agent, or molecule comprising a single-chain polypeptide described herein that binds human LTβR. In some embodiments, the methods may comprise administering to the subject a therapeutically effective amount of a polypeptide, agent, or molecule comprising another LTβR-binding moiety described herein.

In some embodiments, a method of reducing the number of myeloid-derived suppressor cells (MDSCs) in a tumor in a subject is provided. The method may comprise administering to the subject a therapeutically effective amount of a polypeptide, agent, or molecule described herein.

In certain embodiments of the methods described herein, a method of increasing cytolytic T-cell (CTL) activity in a subject comprises administering to the subject a therapeutically effective amount of a polypeptide, agent, or molecule which binds human LTβR. In certain embodiments of the methods described herein, a method of increasing cytolytic T-cell (CTL) activity in a subject comprises administering to the subject a therapeutically effective amount of a polypeptide, agent, or molecule, wherein the polypeptide, agent, or molecule is a single-chain fusion polypeptide that specifically binds to LTβR. In some embodiments, a method of increasing cytolytic T-cell (CTL) activity increasing cytolytic T-cell (CTL) activity in a subject comprises administering to the subject a therapeutically effective amount of a polypeptide, agent, or molecule described herein, wherein the polypeptide, agent, or molecule comprises a fusion polypeptide comprising lymphotoxin-αββ. In some embodiments, the polypeptide comprises a fusion polypeptide comprising lymphotoxin-αββ and a targeting moiety. In some embodiments, the polypeptide comprises a fusion polypeptide comprising lymphotoxin-αββ and an antibody. In some embodiments, the polypeptide comprises a fusion polypeptide comprising lymphotoxin-αββ and an antibody that specifically binds a tumor-associated antigen. In some embodiments, the polypeptide comprises a comprises a fusion polypeptide comprising lymphotoxin-αββ and a Fc region. In certain embodiments, the methods described herein comprise administering to the subject a therapeutically effective amount of a polypeptide, agent, or molecule comprising a single-chain polypeptide described herein that binds human LTβR. In some embodiments, the methods may comprise administering to the subject a therapeutically effective amount of a polypeptide, agent, or molecule comprising another LTβR-binding moiety described herein.

A method of increasing the responsiveness of a tumor in a subject to treatment with a second therapeutic agent is also provided. In certain embodiments, the method comprises administering to the subject a therapeutically effective amount of a polypeptide, agent, or molecule described herein. In certain embodiments, the polypeptide, agent, or molecule specifically binds human LTβR. In some embodiments, the polypeptide agent or molecule comprises a fusion polypeptide, single-chain polypeptide or other LTβR-binding moiety described herein. In certain embodiments, the tumor is resistant to a checkpoint inhibitor (e.g., an anti-PD1 or anti-PDL1 antibody). In some embodiments, the subject has previously failed (or progressed on) therapy with a checkpoint inhibitor. In certain embodiments the second therapeutic agent is further administered to the subject. As a non-limiting example, the second therapeutic agent may be an immunotherapeutic agent such as a checkpoint inhibitor (e.g., anti-PD1 or anti-PDL1 antibody).

The present invention also provides methods for inhibiting the growth of a tumor using a polypeptide, agent, antibody, or molecule described herein. In certain embodiments, the method of inhibiting the growth of a tumor comprises contacting a cell mixture with a polypeptide, agent, antibody, or molecule in vitro. For example, an immortalized cell line or a cancer cell line mixed with immune cells (e.g., T-cells, cytolytic T-cells, or NK cells) is cultured in medium to which is added a test agent. In some embodiments, tumor cells are isolated from a patient sample such as, for example, a tissue biopsy, pleural effusion, or blood sample, mixed with immune cells (e.g., T-cells, cytolytic T-cell, and/or NK cells), and cultured in medium to which is added a test agent. In some embodiments, the polypeptide, agent, antibody, or molecule increases, promotes, and/or enhances the activity of the immune cells. In some embodiments, the polypeptide, agent, antibody, or molecule inhibits tumor cell growth.

In some embodiments, the method of inhibiting the growth of a tumor comprises contacting the tumor or tumor cells with a polypeptide, agent, antibody, or molecule described herein in vivo. In certain embodiments, contacting a tumor or tumor cell with a polypeptide, agent, antibody, or molecule is undertaken in an animal model. For example, a test agent may be administered to mice which have tumors. In some embodiments, the polypeptide, agent, antibody, or molecule increases, promotes, and/or enhances the activity of immune cells in the mice. In some embodiments, the polypeptide, agent, antibody, or molecule inhibits tumor growth. In some embodiments, the polypeptide, agent, antibody, or molecule is administered at the same time or shortly after the introduction of tumor cells into the animal to prevent tumor growth (“preventative model”). In some embodiments, the polypeptide, agent, antibody, or molecule is administered as a therapeutic after tumors have grown to a specified size (“therapeutic model”).

In certain embodiments, the method of inhibiting the growth of a tumor comprises administering to a subject a therapeutically effective amount of a polypeptide, agent, antibody, or molecule described herein. In certain embodiments, the polypeptide, agent, antibody, or molecule specifically binds human LTβR. In some embodiments, the polypeptide, agent, antibody, or molecule comprises a fusion polypeptide, single-chain polypeptide or other LTβR-binding moiety described herein. In certain embodiments, the subject is a human. In certain embodiments, the subject has a tumor, or the subject had a tumor which was removed or at least partially removed.

In addition, the invention provides a method of inhibiting the growth of a tumor in a subject, comprising administering to the subject a therapeutically effective amount of a polypeptide, agent, antibody, or molecule described herein. In certain embodiments, the tumor comprises cancer stem cells.

In addition, the invention provides a method of reducing the tumorigenicity of a tumor in a subject, comprising administering to the subject a therapeutically effective amount of a polypeptide, agent, antibody, or molecule described herein. In certain embodiments, the tumor comprises cancer stem cells.

In some embodiments of the methods described herein, the tumor is a solid tumor. In certain embodiments, the tumor is a tumor selected from the group consisting of: colorectal tumor, pancreatic tumor, lung tumor, ovarian tumor, liver tumor, breast tumor, kidney tumor, prostate tumor, neuroendocrine tumor, gastrointestinal tumor, melanoma, cervical tumor, bladder tumor, glioblastoma, and head and neck tumor. In certain embodiments, the tumor is a colorectal tumor. In certain embodiments, the tumor is an ovarian tumor. In some embodiments, the tumor is a lung tumor. In certain embodiments, the tumor is a pancreatic tumor. In certain embodiments, the tumor is a melanoma tumor. In some embodiments, the tumor is a bladder tumor.

In some embodiments, the tumor (e.g., a solid tumor) expresses or overexpresses a tumor antigen targeted by the polypeptide, agent, antibody, or molecule, such as a homodimeric or heterodimeric molecule which comprises an antigen-binding site that specifically binds the tumor-associated antigen. As a non-limiting example, the tumor may express (or overexpress) B7-H4, B7-H3, P-CADHERIN (CDH3), GABRP, ACPP, SLC45A3, STEAP1, STEAP2, GPA33, GUCY2C, GARP, PVRL4, mesothelin or CA9. In certain embodiments, the tumor expresses B7-H4. For example, in certain embodiments, the tumor may be a breast or ovarian tumor that expresses B7-H4. Such a tumor might be treated or targeted with a polypeptide, agent, or molecule comprising an antibody (e.g., a full-length antibody, an antibody fragment or an antigen-binding site from an antibody) or targeting moiety that specifically binds B7-H4. In certain embodiments, the tumor expresses P-CADHERIN. As another non-limiting example, if a subject is administered an antibody that specifically binds P-CADHERIN, or a polypeptide, agent, or molecule comprising such an antibody or targeting moiety, then the tumor that is treated or targeted may be a tumor that expresses P-CADHERIN, such as a bladder, breast, colon, lung, melanoma, ovarian, stomach, or pancreatic tumor.

The present invention also provides a method of targeting a breast or ovarian tumor in a subject comprising administering a polypeptide, agent, antibody or molecule described herein that specifically binds B7-H4 to the subject. In certain embodiments, the breast or ovarian tumor is selectively targeted. In certain embodiments, any of the antibodies described herein that specifically bind human B7-H4, or polypeptides, agents, or molecules comprising such antibodies, may be used to target breast or ovarian tumors. For non-limiting examples of such antibodies, see the section above entitled “IV. Antibodies to cell-surface antigens.” In certain embodiments, the polypeptide, agent, antibody, or molecule also binds LTβR. In certain embodiments, the polypeptide, agent, antibody, or molecule activates LTβR. In an alternative aspect, the invention provides a method of targeting a bladder, breast, colon, lung, melanoma, ovarian, stomach, or pancreatic tumor in a subject comprising administering a polypeptide, agent, antibody or molecule described herein that specifically binds P-CADHERIN to the subject. In certain embodiments, the bladder, breast, colon, lung, melanoma, ovarian, stomach, or pancreatic tumor is selectively targeted. In certain embodiments, any of the antibodies described herein that specifically bind human P-CADHERIN, or polypeptides, agents, or molecules comprising such antibodies, may be used to target or treat breast or ovarian tumors. For non-limiting examples of such antibodies, see the section above entitled “IV. Antibodies to cell-surface antigens.” In certain embodiments, the polypeptide, agent, antibody, or molecule also binds LTβR. In certain embodiments, the polypeptide, agent, antibody, or molecule activates LTβR.

The present invention further provides methods for treating cancer in a subject comprising administering to the subject a therapeutically effective amount of a polypeptide, agent, antibody, or molecule described herein. In certain embodiments, the polypeptide, agent, antibody, or molecule specifically binds human LTβR. In some embodiments, the polypeptide, agent, antibody or molecule comprises a fusion polypeptide, single-chain polypeptide or other LTβR-binding moiety described herein. In some embodiments, the polypeptide, agent, antibody, or molecule binds LTβR and inhibits or reduces growth of the cancer.

The present invention provides methods of treating cancer comprising administering to a subject (e.g., a subject in need of treatment) a therapeutically effective amount of a polypeptide, agent, antibody, or molecule described herein. In certain embodiments, the subject is a human. In certain embodiments, the subject has a cancerous tumor. In certain embodiments, the subject has had a tumor removed. In certain embodiments, the subject has previously failed therapy with a checkpoint inhibitor (e.g., anti-PD or anti-PDL1).

In certain embodiments, the cancer is a cancer selected from the group consisting of colorectal cancer, pancreatic cancer, lung cancer, ovarian cancer, liver cancer, breast cancer, kidney cancer, prostate cancer, gastrointestinal cancer, melanoma, cervical cancer, neuroendocrine cancer, bladder cancer, brain cancer, glioblastoma, and head and neck cancer. In certain embodiments, the cancer is pancreatic cancer. In certain embodiments, the cancer is ovarian cancer. In certain embodiments, the cancer is colorectal cancer. In certain embodiments, the cancer is breast cancer. In certain embodiments, the cancer is prostate cancer. In certain embodiments, the cancer is lung cancer. In certain embodiments, the cancer is melanoma. In some embodiments, the cancer is bladder cancer.

In certain embodiments, the cancer is ovarian cancer or breast cancer and the polypeptide, agent, antibody or molecule that is administered to the subject comprises an antibody or other targeting moiety that specifically binds human B7-H4.

In certain embodiments, the cancer is bladder, breast, colon, lung, melanoma, ovarian, pancreatic, or stomach cancer and the polypeptide, agent, antibody or molecule that is administered to the subject comprises an antibody or other targeting moiety that specifically binds human P-CADHERIN. In certain embodiments, the polypeptide, agent, antibody or molecule further comprises a single-chain and/or fusion polypeptide described herein that binds LTβR or another LTβR-binding moiety. In certain embodiments, the polypeptide, agent, antibody or molecule comprises a lymphotoxin αββ heterotrimer (e.g., a single-chain lymphotoxin αββ heterotrimer)

In some embodiments, the cancer is a hematologic cancer. In some embodiment, the cancer is selected from the group consisting of: acute myelogenous leukemia (AML), Hodgkin lymphoma, multiple myeloma, T-cell acute lymphoblastic leukemia (T-ALL), chronic lymphocytic leukemia (CLL), hairy cell leukemia, chronic myelogenous leukemia (CML), non-Hodgkin lymphoma, diffuse large B-cell lymphoma (DLBCL), mantle cell lymphoma (MCL), and cutaneous T-cell lymphoma (CTCL).

The invention also provides a method of activating or enhancing LTβR signaling in a cell comprising contacting the cell with an effective amount of a polypeptide, agent, or molecule described herein (e.g., a single-chain or fusion polypeptide that forms a heterotrimer and binds LTβR). In some embodiments, a method of activating or enhancing LTβR signaling in a cell comprises contacting the cell with an effective amount of a polypeptide, agent, or molecule described herein. In certain embodiments, the polypeptide, agent, or molecule comprises a targeting moiety that binds to an antigen on the surface of the cell (e.g., a tumor-associated antigen). In certain embodiments, the method is an in vivo method wherein the step of contacting the cell with the polypeptide, agent, or molecule comprises administering a therapeutically effective amount of the polypeptide, agent, or molecule to the subject. In some embodiments, the method is an in vitro or ex vivo method. In certain embodiments, the cell is a tumor cell.

The present invention provides methods of determining the level of expression of a target, i.e., a tumor-associated antigen (TAA). In some embodiments, the level of expression of a TAA is determined. Methods for determining the level of nucleic acid expression in a cell, tumor, or cancer are known by those of skill in the art. These methods include, but are not limited to, PCR-based assays, microarray analyses, and nucleotide sequencing (e.g., NextGen sequencing). Methods for determining the level of protein expression in a cell, tumor, or cancer include, but are not limited to, Western blot analyses, protein arrays, ELISAs, immunohistochemistry (IHC), and FACS.

Methods for determining whether a tumor or cancer has an elevated level of expression of a nucleic acid or protein can use a variety of samples. In some embodiments, the sample is taken from a subject having a tumor or cancer. In some embodiments, the sample is a fresh tumor/cancer sample. In some embodiments, the sample is a frozen tumor/cancer sample. In some embodiments, the sample is a formalin-fixed paraffin-embedded sample. In some embodiments, the sample is a blood sample. In some embodiments, the sample is a plasma sample. In some embodiments, the sample is processed to a cell lysate. In some embodiments, the sample is processed to DNA or RNA.

The present invention provides compositions (e.g., pharmaceutical compositions) comprising a polypeptide, agent, antibody, or molecule described herein. The present invention also provides pharmaceutical compositions comprising a polypeptide, agent, antibody, or molecule described herein and a pharmaceutically acceptable carrier or vehicle. In some embodiments, the pharmaceutical compositions find use in immunotherapy. In some embodiments, the pharmaceutical compositions find use in immuno-oncology. In some embodiments, the compositions find use in inhibiting tumor growth. In some embodiments, the pharmaceutical compositions find use in inhibiting tumor growth in a subject (e.g., a human patient). In some embodiments, the compositions find use in treating cancer. In some embodiments, the pharmaceutical compositions find use in treating cancer in a subject (e.g., a human patient).

Formulations are prepared for storage and use by combining a purified agent of the present invention with a pharmaceutically acceptable vehicle (e.g., a carrier or excipient). Those of skill in the art generally consider pharmaceutically acceptable carriers, excipients, and/or stabilizers to be inactive ingredients of a formulation or pharmaceutical composition.

In some embodiments, the polypeptides, agents, antibodies, or molecules described herein are formulated in a buffer comprising of 20 mM histidine, 40 mM NaCl, 5% sucrose, and 0.01% polysorbate 20. In some embodiments, the polypeptides, molecules, antibodies, or agents described herein are formulated in a buffer comprising of 20 mM histidine, 40 mM NaCl, 5% sucrose, and 0.01% polysorbate 20 at pH 5.5. In some embodiments, the polypeptides, molecules, antibodies, or agents described herein are formulated in a buffer comprising of 20 mM histidine, 40 mM NaCl, 5% sucrose, and 0.01% polysorbate 20 at pH 6.0. In some embodiments, the polypeptides, molecules, antibodies, or agents described herein are formulated in a buffer comprising of 20 mM histidine, 40 mM NaCl, 5% sucrose, and 0.01% polysorbate 20 at pH 6.5. In some embodiments, the polypeptides, molecules, antibodies, or agents described herein are formulated in a buffer comprising of 20 mM histidine, 100 mM NaCl, 150 mM sucrose, and 0.01% polysorbate 20 at pH 6.0. In some embodiments, the polypeptides, molecules, antibodies, or agents described herein are formulated in a buffer comprising of 10 mM potassium phosphate and 0.04% polysorbate 20 at pH 7.5.

Thus, in some embodiments the invention provides compositions or pharmaceutical compositions comprising a polypeptide, agent, antibody, or molecule described herein and further comprising about 20 mM histidine, about 40 mM NaCl, about 5% sucrose, and about 0.01% polysorbate 20. In some embodiments the pH of the composition is about pH 5.5, about pH 6.0, or about pH 6.5.

In some embodiments, a polypeptide, agent, antibody, or molecule described herein is lyophilized and/or stored in a lyophilized form. In some embodiments, a formulation comprising a polypeptide, agent, antibody, or molecule described herein is lyophilized.

Suitable pharmaceutically acceptable vehicles include, but are not limited to, nontoxic buffers such as phosphate, citrate, and other organic acids; salts such as sodium chloride; antioxidants including ascorbic acid and methionine; preservatives such as octadecyldimethylbenzyl ammonium chloride, hexamethonium chloride, benzalkonium chloride, benzethonium chloride, phenol, butyl or benzyl alcohol, alkyl parabens, such as methyl or propyl paraben, catechol, resorcinol, cyclohexanol, 3-pentanol, and m-cresol; low molecular weight polypeptides (e.g., less than about 10 amino acid residues); proteins such as serum albumin, gelatin, or immunoglobulins; hydrophilic polymers such as polyvinylpyrrolidone; amino acids such as glycine, glutamine, asparagine, histidine, arginine, or lysine; carbohydrates such as monosaccharides, disaccharides, glucose, mannose, or dextrins; chelating agents such as EDTA; sugars such as sucrose, mannitol, trehalose or sorbitol; salt-forming counter-ions such as sodium; metal complexes such as Zn-protein complexes; and non-ionic surfactants such as TWEEN or polyethylene glycol (PEG). (Remington. The Science and Practice of Pharmacy. 22^{nd} Edition, 2012, Pharmaceutical Press, London.).

The pharmaceutical compositions of the present invention can be administered in any number of ways for either local or systemic treatment. Administration can be topical by epidermal or transdermal patches, ointments, lotions, creams, gels, drops, suppositories, sprays, liquids and powders; pulmonary by inhalation or insufflation of powders or aerosols, including by nebulizer, intratracheal, and intranasal; oral; or parenteral including intravenous, intraarterial, intratumoral, subcutaneous, intraperitoneal, intramuscular (e.g., injection or infusion), or intracranial (e.g., intrathecal or intraventricular).

The therapeutic formulation can be in unit dosage form. Such formulations include tablets, pills, capsules, powders, granules, solutions or suspensions in water or non-aqueous media, or suppositories. In solid compositions such as tablets the principal active ingredient is mixed with a pharmaceutical carrier. Conventional tableting ingredients include corn starch, lactose, sucrose, sorbitol, talc, stearic acid, magnesium stearate, dicalcium phosphate or gums, and diluents (e.g., water). These can be used to form a solid preformulation composition containing a homogeneous mixture of a compound of the present invention, or a non-toxic pharmaceutically acceptable salt thereof. The solid preformulation composition is then subdivided into unit dosage forms of a type described above. The tablets, pills, etc. of the formulation or composition can be coated or otherwise compounded to provide a dosage form affording the advantage of prolonged action. For example, the tablet or pill can comprise an inner composition covered by an outer component. Furthermore, the two components can be separated by an enteric layer that serves to resist disintegration and permits the inner component to pass intact through the stomach or to be delayed in release. A variety of materials can be used for such enteric layers or coatings, such materials include a number of polymeric acids and mixtures of polymeric acids with such materials as shellac, cetyl alcohol and cellulose acetate.

The polypeptides, agents, antibodies, or molecules described herein can also be entrapped in microcapsules. Such microcapsules are prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nanoparticles and nanocapsules) or in macroemulsions as described in Remington: The Science and Practice of Pharmacy, 22^{nd} Edition, 2012, Pharmaceutical Press, London.

In certain embodiments, pharmaceutical formulations include a polypeptide, agent, antibody, or molecule of the present invention complexed with liposomes. Methods to produce liposomes are known to those of skill in the art. For example, some liposomes can be generated by reverse phase evaporation with a lipid composition comprising phosphatidylcholine, cholesterol, and PEG-derivatized phosphatidylethanolamine (PEG-PE). Liposomes can be extruded through filters of defined pore size to yield liposomes with the desired diameter.

In certain embodiments, sustained-release preparations comprising the polypeptides, agents, antibodies, or molecules described herein can be produced. Suitable examples of sustained-release preparations include semi-permeable matrices of solid hydrophobic polymers containing a polypeptide, agent, antibody, or molecule, where the matrices are in the form of shaped articles (e.g., films or microcapsules). Examples of sustained-release matrices include polyesters, hydrogels such as poly(2-hydroxyethyl-methacrylate) or poly(vinyl alcohol), polylactides, copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(−)-3-hydroxybutyric acid.

In certain embodiments, in addition to administering a polypeptide, agent, antibody, or molecule described herein, the method or treatment further comprises administering at least one additional therapeutic agent. An additional therapeutic agent can be administered prior to, concurrently with, and/or subsequently to, administration of the polypeptide, agent, antibody, or molecule. Pharmaceutical compositions comprising a polypeptide, agent, antibody, or molecule and the additional therapeutic agent(s) are also provided. In some embodiments, the at least one additional therapeutic agent comprises 1, 2, 3, or more additional therapeutic agents.

Combination therapy with two or more therapeutic agents often uses agents that work by different mechanisms of action, although this is not required. Combination therapy using agents with different mechanisms of action may result in additive or synergetic effects. Combination therapy may allow for a lower dose of each agent than is used in monotherapy, thereby reducing toxic side effects and/or increasing the therapeutic index of the polypeptide, agent, antibody, or molecule(s). Combination therapy may decrease the likelihood that resistant cancer cells will develop. In some embodiments, combination therapy comprises a therapeutic agent that affects the immune response (e.g., enhances or activates the response) and a therapeutic agent that affects (e.g., inhibits or kills) the tumor/cancer cells.

In some embodiments of the methods described herein, the combination of a polypeptide, agent, antibody, or molecule described herein and at least one additional therapeutic agent results in additive or synergistic results. In some embodiments, the combination therapy results in an increase in the therapeutic index of the polypeptide, agent, antibody, or molecule. In some embodiments, the combination therapy results in an increase in the therapeutic index of the additional therapeutic agent(s). In some embodiments, the combination therapy results in a decrease in the toxicity and/or side effects of the polypeptide, agent, antibody, or molecule. In some embodiments, the combination therapy results in a decrease in the toxicity and/or side effects of the additional therapeutic agent(s).

Useful classes of therapeutic agents include, for example, anti-tubulin agents, auristatins, DNA minor groove binders, DNA replication inhibitors, alkylating agents (e.g., platinum complexes such as cisplatin, mono(platinum), bis(platinum) and tri-nuclear platinum complexes and carboplatin), anthracyclines, antibiotics, anti-folates, anti-metabolites, chemotherapy sensitizers, duocarmycins, etoposides, fluorinated pyrimidines, ionophores, lexitropsins, nitrosoureas, platinols, purine antimetabolites, puromycins, radiation sensitizers, steroids, taxanes, topoisomerase inhibitors, vinca alkaloids, or the like. In certain embodiments, the second therapeutic agent is an alkylating agent, an antimetabolite, an antimitotic, a topoisomerase inhibitor, or an angiogenesis inhibitor.

Therapeutic agents that may be administered in combination with the polypeptides or agents described herein include chemotherapeutic agents. Thus, in some embodiments, the method or treatment involves the administration of a polypeptide, agent, antibody, or molecule of the present invention in combination with a chemotherapeutic agent or in combination with a cocktail of chemotherapeutic agents. Treatment with a polypeptide, agent, antibody, or molecule can occur prior to, concurrently with, or subsequent to administration of chemotherapies. Combined administration can include co-administration, either in a single pharmaceutical formulation or using separate formulations, or consecutive administration in either order but generally within a time period such that all active agents can exert their biological activities simultaneously. Preparation and dosing schedules for such chemotherapeutic agents can be used according to manufacturers' instructions or as determined empirically by the skilled practitioner. Preparation and dosing schedules for such chemotherapy are also described in The Chemotherapy Source Book, 4^{th} Edition, 2008, M. C. Perry, Editor, Lippincott, Williams & Wilkins, Philadelphia, Pa.

Chemotherapeutic agents useful in the present invention include, but are not limited to, alkylating agents such as thiotepa and cyclosphosphamide (CYTOXAN); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamime; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytosine arabinoside, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenishers such as folinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrinc; pentostatin; phenamet; pirarubicin; podophyllinic acid; 2-ethylhydrazide; procarbazine; PSK; razoxane; sizofuran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (Ara-C); taxoids, e.g. paclitaxel (TAXOL) and docetaxel (TAXOTERE); chlorambucil; gemcitabine; 6-thioguanine; mercaptopurine; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; ibandronate; CPT11; topoisomerase inhibitor RFS 2000; difluoromethylornithine (DMFO); retinoic acid; esperamicins; capecitabine (XELODA); and pharmaceutically acceptable salts, acids or derivatives of any of the above. Chemotherapeutic agents also include anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (FARESTON); and anti-androgens such as flutamide, nilutamide, bicalutamide, leuprolide, and goserelin; and pharmaceutically acceptable salts, acids or derivatives of any of the above.

In certain embodiments of the methods described herein, the chemotherapeutic agent is a topoisomerase inhibitor. Topoisomerase inhibitors are chemotherapy agents that interfere with the action of a topoisomerase enzyme (e.g., topoisomerase I or II). Topoisomerase inhibitors include, but are not limited to, doxorubicin HCl, daunorubicin citrate, mitoxantrone HCl, actinomycin D, etoposide, topotecan HCl, teniposide (VM-26), and irinotecan, as well as pharmaceutically acceptable salts, acids, or derivatives of any of these. In some embodiments, the additional therapeutic agent is irinotecan.

In certain embodiments, the chemotherapeutic agent is an anti-metabolite. An anti-metabolite is a chemical with a structure that is similar to a metabolite required for normal biochemical reactions, yet different enough to interfere with one or more normal functions of cells, such as cell division. Anti-metabolites include, but are not limited to, gemcitabine, fluorouracil, capecitabine, methotrexate sodium, ralitrexed, pemetrexed, tegafur, cytosine arabinoside, thioguanine, 5-azacytidine, 6-mercaptopurine, azathioprine, 6-thioguanine, pentostatin, fludarabine phosphate, and cladribine, as well as pharmaceutically acceptable salts, acids, or derivatives of any of these. In certain embodiments, the additional therapeutic agent is gemcitabine.

In certain embodiments of the methods described herein, the chemotherapeutic agent is an antimitotic agent, including, but not limited to, agents that bind tubulin. In some embodiments, the agent is a taxane. In certain embodiments, the agent is paclitaxel or docetaxel, or a pharmaceutically acceptable salt, acid, or derivative of paclitaxel or docetaxel. In certain embodiments, the agent is paclitaxel (TAXOL), docetaxel (TAXOTERE), albumin-bound paclitaxel (nab-paclitaxel; ABRAXANE), DHA-paclitaxel, or PG-paclitaxel. In certain alternative embodiments, the antimitotic agent comprises a vinca alkaloid, such as vincristine, vinblastine, vinorelbine, or vindesine, or pharmaceutically acceptable salts, acids, or derivatives thereof. In some embodiments, the antimitotic agent is an inhibitor of kinesin Eg5 or an inhibitor of a mitotic kinase such as Aurora A or Plk 1. In certain embodiments, the additional therapeutic agent is paclitaxel. In certain embodiments, the additional therapeutic agent is nab-paclitaxel.

In some embodiments of the methods described herein, an additional therapeutic agent comprises an agent such as a small molecule. For example, treatment can involve the combined administration of a polypeptide, agent, antibody, or molecule of the present invention with a small molecule that acts as an inhibitor against tumor-associated antigens including, but not limited to, EGFR, HER2 (ErbB2), and/or VEGF. In some embodiments, a polypeptide, agent, antibody, or molecule of the present invention is administered in combination with a protein kinase inhibitor selected from the group consisting of: gefitinib (IRESSA), erlotinib (TARCEVA), sunitinib (SUTENT), lapatanib, vandetanib (ZACTIMA), AEE788, CI-1033, cediranib (RECENTIN), sorafenib (NEXAVAR), and pazopanib (GW786034B). In some embodiments, an additional therapeutic agent comprises an mTOR inhibitor.

In certain embodiments of the methods described herein, the additional therapeutic agent is a small molecule that inhibits a cancer stem cell pathway. In some embodiments, the additional therapeutic agent is an inhibitor of the Notch pathway. In some embodiments, the additional therapeutic agent is an inhibitor of the Wnt pathway. In some embodiments, the additional therapeutic agent is an inhibitor of the BMP pathway. In some embodiments, the additional therapeutic agent is an inhibitor of the Hippo pathway. In some embodiments, the additional therapeutic agent is an inhibitor of the mTOR/AKR pathway. In some embodiments, the additional therapeutic agent is an inhibitor of the RSPO/LGR pathway.

In some embodiments of the methods described herein, an additional therapeutic agent comprises a biological molecule, such as an antibody. For example, treatment can involve the combined administration of a polypeptide, agent, antibody, or molecule of the present invention with antibodies against tumor-associated antigens including, but not limited to, antibodies that bind EGFR. HER2/ErbB2, and/or VEGF. In certain embodiments, the additional therapeutic agent is an antibody specific for a cancer stem cell marker. In some embodiments, the additional therapeutic agent is an antibody that binds a component of the Notch pathway. In some embodiments, the additional therapeutic agent is an antibody that binds a component of the Wnt pathway. In certain embodiments, the additional therapeutic agent is an antibody that inhibits a cancer stem cell pathway. In some embodiments, the additional therapeutic agent is an inhibitor of the Notch pathway. In some embodiments, the additional therapeutic agent is an inhibitor of the Wnt pathway. In some embodiments, the additional therapeutic agent is an inhibitor of the BMP pathway. In some embodiments, the additional therapeutic agent is an antibody that inhibits-catenin signaling. In certain embodiments, the additional therapeutic agent is an antibody that is an angiogenesis inhibitor (e.g., an anti-VEGF or VEGF receptor antibody). In certain embodiments, the additional therapeutic agent is bevacizumab (AVASTIN), ramucirumab, trastuzumab (HERCEPTIN), pertuzumab (OMNITARG), panitumumab (VECTIBIX), nimotuzumab, zalutumumab, or cetuximab (ERBITUX).

In some embodiments of the methods described herein, the additional therapeutic agent is an antibody that modulates the immune response. In some embodiments, the additional therapeutic agent is an anti-PD-1 antibody, an anti-PD-L1 antibody, an anti-CTLA-4 antibody, or an anti-TIGIT antibody.

Furthermore, treatment with a polypeptide, agent, antibody, or molecule described herein can include combination treatment with other biologic molecules, such as one or more cytokines (e.g., lymphokines, interleukins, tumor necrosis factors, and/or growth factors) or can be accompanied by surgical removal of tumors, removal of cancer cells, or any other therapy deemed necessary by a treating physician. In some embodiments, the additional therapeutic agent is an immune response stimulating agent.

In some embodiments of the methods described herein, the polypeptide, agent, antibody, or molecule can be combined with a growth factor selected from the group consisting of: adrenomedullin (AM), angiopoietin (Ang), BMPs, BDNF, EGF, erythropoietin (EPO). FGF, GDNF, G-CSF, GM-CSF. GDF9, HGF, HDGF, IGF, migration-stimulating factor, myostatin (GDF-8), NGF, neurotrophins, PDGF, thrombopoietin, TGF-α, TGF-β, TNF-α, VEGF, PIGF, IL-1, IL-2, IL-3, IL-4, IL-5, IL-6, IL-7, IL-12, IL-15, and IL-18.

In some embodiments of the methods described herein, the additional therapeutic agent is an immunotherapeutic agent. In some embodiments, the immunotherapeutic agent is selected from the group consisting of granulocyte-macrophage colony stimulating factor (GM-CSF), macrophage colony stimulating factor (M-CSF), granulocyte colony stimulating factor (G-CSF), interleukin 3 (IL-3), interleukin 12 (IL-12), interleukin 1 (IL-1), interleukin 2 (IL-2), B7-1 (CD80), B7-2 (CD86), 4-1BB ligand, anti-CD3 antibody, anti-CTLA-4 antibody, anti-TIGIT antibody, anti-PD-1 antibody, anti-PD-L1 antibody, anti-LAG-3 antibody, and anti-TIM-3 antibody.

In some embodiments of the methods described herein, an immunotherapeutic agent is selected from the group consisting of: a modulator of PD-1 activity, a modulator of PD-L1 activity, a modulator of PD-L2 activity, a modulator of CTLA-4 activity, a modulator of CD28 activity, a modulator of CD80 activity, a modulator of CD86 activity, a modulator of 4-1BB activity, an modulator of OX40 activity, a modulator of KIR activity, a modulator of Tim-3 activity, a modulator of LAG3 activity, a modulator of CD27 activity, a modulator of CD40 activity, a modulator of GITR activity, a modulator of TIGIT activity, a modulator of CD20 activity, a modulator of CD96 activity, a modulator of IDO1 activity, a cytokine, a chemokine, an interferon, an interleukin, a lymphokine, a member of the tumor necrosis factor (TNF) family, and an immunostimulatory oligonucleotide.

In some embodiments of the methods described herein, an immunotherapeutic agent is selected from the group consisting of: a PD-1 antagonist, a PD-L1 antagonist, a PD-L2 antagonist, a CTLA-4 antagonist, a CD80 antagonist, a CD86 antagonist, a KIR antagonist, a Tim-3 antagonist, a LAG3 antagonist, a TIGIT antagonist, a CD20 antagonist, a CD96 antagonist, and/or an IDO1 antagonist.

In some embodiments of the methods described herein, the PD-1 antagonist is an antibody that specifically binds PD-1. In some embodiments, the antibody that binds PD-1 is KEYTRUDA (MK-3475), pidilizumab (CT-O 11), nivolumab (OPDIVO, BMS-936558, MDX-1106), MEDI0680 (AMP-514), REGN2810, BGB-A317. PDR-001, or STI-A1110. In some embodiments, the antibody that binds PD-1 is described in PCT Publication WO 2014/179664, for example, an antibody identified as APE2058, APE1922, APE1923, APE1924, APE 1950, or APE1963, or an antibody containing the CDR regions of any of these antibodies. In other embodiments, the PD-1 antagonist is a fusion protein that includes PD-L2, for example, AMP-224. In other embodiments, the PD-1 antagonist is a peptide inhibitor, for example, AUNP-12.

In some embodiments, the PD-L antagonist is an antibody that specifically binds PD-L. In some embodiments, the antibody that binds PD-L1 is atezolizumab (RG7446, MPDL3280A), MEDI4736, BMS-936559 (MDX-1105), avelumab (MSB0010718C), KD033, the antibody portion of KD033, or STI-A1014. In some embodiments, the antibody that binds PD-L1 is described in PCT Publication WO 2014/055897, for example. Ab-14, Ab-16, Ab-30, Ab-31, Ab-42, Ab-50, Ab-52, or Ab-55, or an antibody that contains the CDR regions of any of these antibodies.

In some embodiments, the CTLA-4 antagonist is an antibody that specifically binds CTLA-4. In some embodiments, the antibody that binds CTLA-4 is ipilimumab (YERVOY) or tremelimumab (CP-675,206). In some embodiments, the CTLA-4 antagonist a CTLA-4 fusion protein, for example, KAHR-102.

In some embodiments, the LAG3 antagonist is an antibody that specifically binds LAG3. In some embodiments, the antibody that binds LAG3 is IMP701, IMP731. BMS-986016, LAG525, and GSK2831781. In some embodiments, the LAG3 antagonist includes a soluble LAG3 receptor, for example, IMP321.

In some embodiments, the KIR antagonist is an antibody that specifically binds KIR. In some embodiments, the antibody that binds KIR is lirilumab.

In some embodiments, an immunotherapeutic agent is selected from the group consisting of: a CD28 agonist, a 4-1BB agonist, an OX40 agonist, a CD27 agonist, a CD80 agonist, a CD86 agonist, a CD40 agonist, and a GITR agonist.

In some embodiments, the OX40 agonist includes OX40 ligand, or an OX40-binding portion thereof. For example, the OX40 agonist may be MEDI6383. In some embodiments, the OX40 agonist is an antibody that specifically binds OX40. In some embodiments, the antibody that binds OX40 is MEDI6469, MEDI0562, or MOXR0916 (RG7888). In some embodiments, the OX40 agonist is a vector (e.g., an expression vector or virus, such as an adenovirus) capable of expressing OX40 ligand. In some embodiments the OX40-expressing vector is Delta-24-RGDOX or DNX2401.

In some embodiments, the 4-1BB (CD137) agonist is a binding molecule, such as an anticalin. In some embodiments, the anticalin is PRS-343. In some embodiments, the 4-1BB agonist is an antibody that specifically binds 4-1BB. In some embodiments, antibody that binds 4-1BB is PF-2566 (PF-05082566) or urelumab (BMS-663513).

In some embodiments, the CD27 agonist is an antibody that specifically binds CD27. In some embodiments, the antibody that binds CD27 is varlilumab (CDX-1127).

In some embodiments, the GITR agonist comprises GITR ligand or a GITR-binding portion thereof. In a one embodiment, the GITR agonist comprises any of those described in U.S. Patent Publication 2016/0256527, hereby incorporated by reference, for example, the molecule designated 336B11. In some embodiments, the GITR agonist is an antibody that specifically binds GITR. In some embodiments, the antibody that binds GITR is TRX518. MK-4166, or INBRX-110.

In some embodiments, immunotherapeutic agent includes, but is not limited to, cytokines such as chemokines, interferons, interleukins, lymphokines, and members of the tumor necrosis factor (TNF) family. In some embodiments, immunotherapeutic agents include immunostimulatory oligonucleotides, such as CpG dinucleotides.

In some embodiments, an immunotherapeutic agent includes, but is not limited to, anti-PD-1 antibodies, anti-PD-L1 antibodies, anti-PD-L2 antibodies, anti-CTLA-4 antibodies, anti-CD28 antibodies, anti-CD80 antibodies, anti-CD86 antibodies, anti-4-1BB antibodies, anti-OX40 antibodies, anti-KIR antibodies, anti-Tim-3 antibodies, anti-LAG3 antibodies, anti-CD27 antibodies, anti-CD40 antibodies, anti-GITR antibodies, anti-TIGIT antibodies, anti-CD20 antibodies, anti-CD96 antibodies, or anti-IDO1 antibodies.

In some embodiments, a method of treating cancer in a subject comprises administering to the subject a therapeutically effective amount of a polypeptide, agent, or molecule described herein in combination with a checkpoint inhibitor. In some embodiments, the checkpoint inhibitor is an anti-PD-1 antibody. In some embodiments, the checkpoint inhibitor is an anti-PD-1 antibody and the cancer is melanoma. In some embodiments, the checkpoint inhibitor is an anti-PD-1 antibody and the cancer is lung cancer. In some embodiments, the checkpoint inhibitor is an anti-PD-1 antibody and the cancer is bladder cancer. In some embodiments, the checkpoint inhibitor is an anti-PD-1 antibody and the cancer is a hematologic cancer. In some embodiments, the checkpoint inhibitor is an anti-PD-L1 antibody. In some embodiments, the checkpoint inhibitor is an anti-PD-L1 antibody and the cancer is melanoma. In some embodiments, the checkpoint inhibitor is an anti-PD-L1 antibody and the cancer is lung cancer. In some embodiments, the checkpoint inhibitor is an anti-PD-L1 antibody and the cancer is bladder cancer. In some embodiments, the checkpoint inhibitor is an anti-PD-L1 antibody and the cancer is breast cancer. In some embodiments, the checkpoint inhibitor is an anti-PD-L1 antibody and the cancer is a hematologic cancer.

In certain embodiments of the methods described herein, the treatment involves the administration of a polypeptide, agent, antibody, or molecule of the present invention in combination with radiation therapy. Treatment with a polypeptide, agent, antibody, or molecule can occur prior to, concurrently with, or subsequent to administration of radiation therapy. Dosing schedules for such radiation therapy can be determined by the skilled medical practitioner.

Combined administration can include co-administration, either in a single pharmaceutical formulation or using separate formulations, or consecutive administration in either order but generally within a time period such that all active agents can exert their biological activities simultaneously.

It will be appreciated that the combination of a polypeptide, agent, antibody, or molecule described herein and at least one additional therapeutic agent may be administered in any order or concurrently. In some embodiments, the polypeptide, agent, antibody, or molecule will be administered to patients that have previously undergone treatment with a second therapeutic agent. In certain other embodiments, the polypeptide, agent, antibody, or molecule and a second therapeutic agent will be administered substantially simultaneously or concurrently. For example, a subject may be given a polypeptide, agent, antibody, or molecule while undergoing a course of treatment with a second therapeutic agent (e.g., chemotherapy). In certain embodiments, a polypeptide, agent, antibody, or molecule will be administered within 1 year of the treatment with a second therapeutic agent. In certain alternative embodiments, a polypeptide, agent, antibody, or molecule will be administered within 10, 8, 6, 4, or 2 months of any treatment with a second therapeutic agent. In certain other embodiments, a polypeptide, agent, antibody, or molecule will be administered within 4, 3, 2, or 1 weeks of any treatment with a second therapeutic agent. In some embodiments, a polypeptide, agent, antibody, or molecule will be administered within 5, 4, 3, 2, or 1 days of any treatment with a second therapeutic agent. It will further be appreciated that the two (or more) agents or treatments may be administered to the subject within a matter of hours or minutes (i.e., substantially simultaneously).

The polypeptide, agent, antibody, or molecule can be administered one time or over a series of treatments lasting from several days to several months, or until a cure is effected or a diminution of the disease state is achieved (e.g., reduction in tumor size). In certain embodiments, dosage is from 0.01 μg to 100 mg/kg of body weight, from 0.1 μg to 100 mg/kg of body weight, from ling to 100 mg/kg of body weight, from 1 mg to 100 mg/kg of body weight, 1 mg to 80 mg/kg of body weight from 10 mg to 100 mg/kg of body weight, from 10 mg to 75 mg/kg of body weight, or from 10 mg to 50 mg/kg of body weight. In certain embodiments, the dosage of the polypeptide, agent, antibody, or molecule is from about 0.1 mg to about 20 mg/kg of body weight. In some embodiments, the dosage of the polypeptide, agent, antibody, or molecule is about 0.1 mg/kg of body weight. In some embodiments, the dosage of the polypeptide, agent, antibody, or molecule is about 0.25 mg/kg of body weight. In some embodiments, the dosage of the polypeptide, agent, antibody, or molecule is about 0.5 mg/kg of body weight. In some embodiments, the dosage of the polypeptide, agent, antibody, or molecule is about 1 mg/kg of body weight. In some embodiments, the dosage of the polypeptide, agent, antibody or molecule is about 1.5 mg/kg of body weight. In some embodiments, the dosage of the polypeptide, agent, antibody, or molecule is about 2 mg/kg of body weight. In some embodiments, the dosage of the polypeptide, agent, antibody, or molecule is about 2.5 mg/kg of body weight. In some embodiments, the dosage of the polypeptide, agent, antibody, or molecule is about 5 mg/kg of body weight. In some embodiments, the dosage of the polypeptide, agent, antibody, or molecule is about 7.5 mg/kg of body weight. In some embodiments, the dosage of the polypeptide, agent, antibody, or molecule is about 10 mg/kg of body weight. In some embodiments, the dosage of the polypeptide, agent, antibody, or molecule is about 12.5 mg/kg of body weight. In some embodiments, the dosage of the polypeptide, agent, antibody, or molecule is about 15 mg/kg of body weight. In certain embodiments, the dosage can be given once or more daily, weekly, monthly, or yearly. In certain embodiments, the polypeptide, agent, antibody, or molecule is given once every week, once every two weeks, once every three weeks, or once every four weeks.

In some embodiments, a polypeptide, agent, antibody, or molecule may be administered at an initial higher “loading” dose, followed by one or more lower doses. In some embodiments, the frequency of administration may also change. In some embodiments, a dosing regimen may comprise administering an initial dose, followed by additional doses (or “maintenance” doses) once a week, once every two weeks, once ever) three weeks, or once every month. For example, a dosing regimen may comprise administering an initial loading dose, followed by a weekly maintenance dose of, for example, one-half of the initial dose. Or a dosing regimen may comprise administering an initial loading dose, followed by maintenance doses of, for example one-half of the initial dose every other week. Or a dosing regimen may comprise administering three initial doses for 3 weeks, followed by maintenance doses of, for example, the same amount every other week.

As is known to those of skill in the art, administration of any therapeutic agent may lead to side effects and/or toxicities. In some cases, the side effects and/or toxicities are so severe as to preclude administration of the particular agent at a therapeutically effective dose. In some cases, drug therapy must be discontinued, and other agents may be tried. However, many agents in the same therapeutic class often display similar side effects and/or toxicities, meaning that the patient either has to stop therapy, or if possible, suffer from the unpleasant side effects associated with the therapeutic agent.

In some embodiments, the dosing schedule may be limited to a specific number of administrations or “cycles”. In some embodiments, the polypeptide, agent, antibody, or molecule is administered for 3, 4, 5, 6, 7, 8, or more cycles. For example, the polypeptide, agent, antibody, or molecule is administered every 2 weeks for 6 cycles, the polypeptide, agent, antibody, or molecule is administered every 3 weeks for 6 cycles, the polypeptide, agent, antibody, or molecule is administered every 2 weeks for 4 cycles, the polypeptide, agent, antibody, or molecule is administered every 3 weeks for 4 cycles, etc. Dosing schedules can be decided upon and subsequently modified by those skilled in the art.

Thus, the present invention provides methods of administering to a subject the polypeptides, agents, or molecules described herein comprising using an intermittent dosing strategy for administering one or more agents, which may reduce side effects and/or toxicities associated with administration of a polypeptide, agent, antibody, or molecule, chemotherapeutic agent, etc. In some embodiments, a method for treating cancer in a human subject comprises administering to the subject a therapeutically effective dose of a polypeptide, agent, antibody, or molecule in combination with a therapeutically effective dose of a chemotherapeutic agent, wherein one or both of the agents are administered according to an intermittent dosing strategy. In some embodiments, the intermittent dosing strategy comprises administering an initial dose of a polypeptide, agent, antibody, or molecule to the subject, and administering subsequent doses of the polypeptide, agent, antibody, or molecule about once every 2 weeks. In some embodiments, the intermittent dosing strategy comprises administering an initial dose of a polypeptide, agent, antibody, or molecule to the subject, and administering subsequent doses of the polypeptide, agent, antibody, or molecule about once every 3 weeks. In some embodiments, the intermittent dosing strategy comprises administering an initial dose of a polypeptide, agent, antibody, or molecule to the subject, and administering subsequent doses of the polypeptide, agent, antibody, or molecule about once every 4 weeks. In some embodiments, the polypeptide, agent, antibody, or molecule is administered using an intermittent dosing strategy and the chemotherapeutic agent is administered weekly.

### VIII. Screening

The present invention provides screening methods to identify agents that modulate the immune response. In some embodiments, the present invention provides methods for screening candidate agents, including but not limited to, proteins, antibodies, peptides, peptidomimetics, small molecules, compounds, or other drugs, which modulate the immune response.

In some embodiments, a method of screening for a candidate agent that modulates the immune response comprises determining if the polypeptide, agent, antibody, or molecule described herein has an effect on immune response cells. In some embodiments, a method of screening for a candidate agent that modulates the immune response comprises determining if the polypeptide, agent, antibody, or molecule is capable of increasing the activity of immune cells. In some embodiments, a method of screening for a candidate agent that modulates the immune response comprises determining if the polypeptide, agent, antibody, or molecule is capable of increasing the activity of cytolytic cells, such as CTLs and/or NK cells. In some embodiments, a method of screening for a candidate agent that modulates the immune response comprises determining if the polypeptide, agent, antibody, or molecule is capable of inhibiting the activity of suppressor cells, such as Tregs and/or MDSCs.

### IX. Kits Comprising Agents Described Herein

The present invention provides kits that comprise the polypeptides, molecules, antibodies, or agents described herein and that can be used to perform the methods described herein. In certain embodiments, a kit comprises at least one purified agent in one or more containers. In some embodiments, the kits contain all of the components necessary and/or sufficient to perform a detection assay, including all controls, directions for performing assays, and any necessary software for analysis and presentation of results. One skilled in the art will readily recognize that the disclosed polypeptides, molecules, antibodies, polynucleotides, and agents of the present invention can be readily incorporated into one of the established kit formats which are well known in the art.

Further provided are kits that comprise a polypeptide, agent, antibody, or molecule as well as at least one additional therapeutic agent. In certain embodiments, the second (or more) therapeutic agent is a chemotherapeutic agent. In certain embodiments, the second (or more) therapeutic agent is an angiogenesis inhibitor.

Embodiments of the present disclosure can be further defined by reference to the following non-limiting examples, which describe in detail preparation of certain antibodies of the present disclosure and methods for using antibodies of the present disclosure. It will be apparent to those skilled in the art that many modifications, both to materials and methods, may be practiced without departing from the scope of the present disclosure.

### EXAMPLES

### Example 1

### Generation of a Homodimeric Molecule Comprising Lymphotoxin and an Anti-TAA Antibody

A single-chain fusion polypeptide comprising one copy of mouse lymphotoxin-alpha and two copies of mouse lymphotoxin-beta linked to an anti-mouse B7-H4 antibody was generated. A representative diagram of the molecule format is shown in FIG. 1A. This molecule is referred to herein as 349B1 (SEQ ID NO:36 with a signal sequence and SEQ ID NO:37 without signal sequence).

The basic format can be used to generate a wide variety of molecules comprising the lymphotoxin-αββ trimer. The lymphotoxin-αββ trimer may be linked to any number of TAA-specific antibodies to target different tumors. A single-chain fusion polypeptide comprising the human lymphotoxin αββ trimer has also been generated.

### Example 2

FACS Analysis of Anti-mB7-H4/Lymphotoxin-αββ Binding to mB7-H4 and Lymphotoxin β Receptor

To test the binding ability of the 349B1 molecule to bind B7-H4 and LTβR, cell-based FACS studies were conducted. Human HEK-293T cells were transiently transfected with expression vectors encoding a membrane-bound extracellular domain of mouse B7-H4 (mB7-H4-CD4TM-GFP) or a membrane-bound extracellular domain of mouse LTβR (mLTβR-CD4TM-GFP). Forty-eight hours post-transfection, the cells were suspended in ice-cold HBSS containing 2% FBS and incubated in the presence of 349B1 for 30 minutes. The cells were stained with an APC-conjugated anti-mouse Fc secondary antibody to detect cells bound by the 349B1 molecule. Cells were incubated with an anti-APC antibody as a negative control. The cells were analyzed on a FACS Canto instrument (BD Biosciences), and the data were processed using FlowJo software.

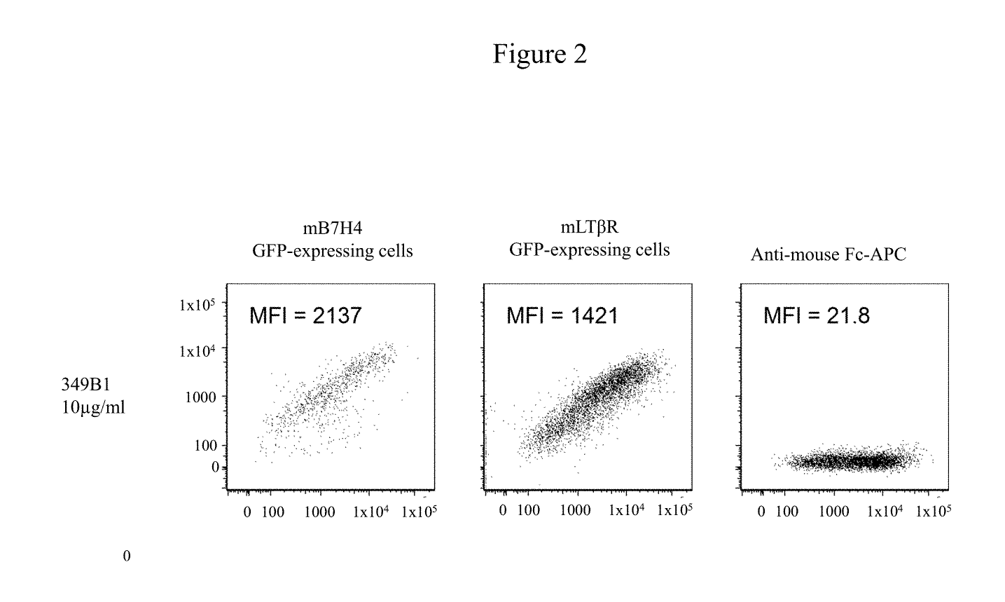

As shown in FIG. 2, 349B1 was able to bind mB7-H4 at the surface of B7-H4-expressing cells. Also, 349B1 was able to bind LTβR at the surface of mLTβR-expressing cells. These results demonstrated that both moieties of 349B1, the anti-B7-H4 antibody and the lymphotoxin-αββ trimer, formed into biologically functional structures.

### Example 3

### Activation of LTβR Signaling by Anti-mB7-H4/Lymphotoxin-αββ

To determine whether the anti-mB7-H4/lymphotoxin-αββ molecule would induce signaling through LTβR, luciferase reporter assays were conducted. A HEK-293 cell line was co-transfected with an expression vector encoding full-length mouse LTβR and an expression vector encoding a NF-kB-luciferase reporter construct. A stably transfected cell line was identified and selected. For the assay, cells were plated into a 96 well plate and incubated overnight. 349B1 (anti-mB7-H4/lymphotoxin-αββ) was tested over a range of concentrations (5-fold dilutions 50 μg/ml to 0.016 μg/ml). Two other molecules were evaluated, anti-mB7-H4/LIGHT trimer 351B1 and anti-mB7-H4/LIGHT trimer mutant 351B2. The LIGHT mutant has an amino acid substitution that is believed to reduce/eliminate the binding of LIGHT to the HVEM receptor but does not affect binding to LTβR. The test agents were added to the cells and incubated overnight. Luciferase activity was determined using a Steady-Glo assay kit (Promega) according to the manufacturer's instructions.

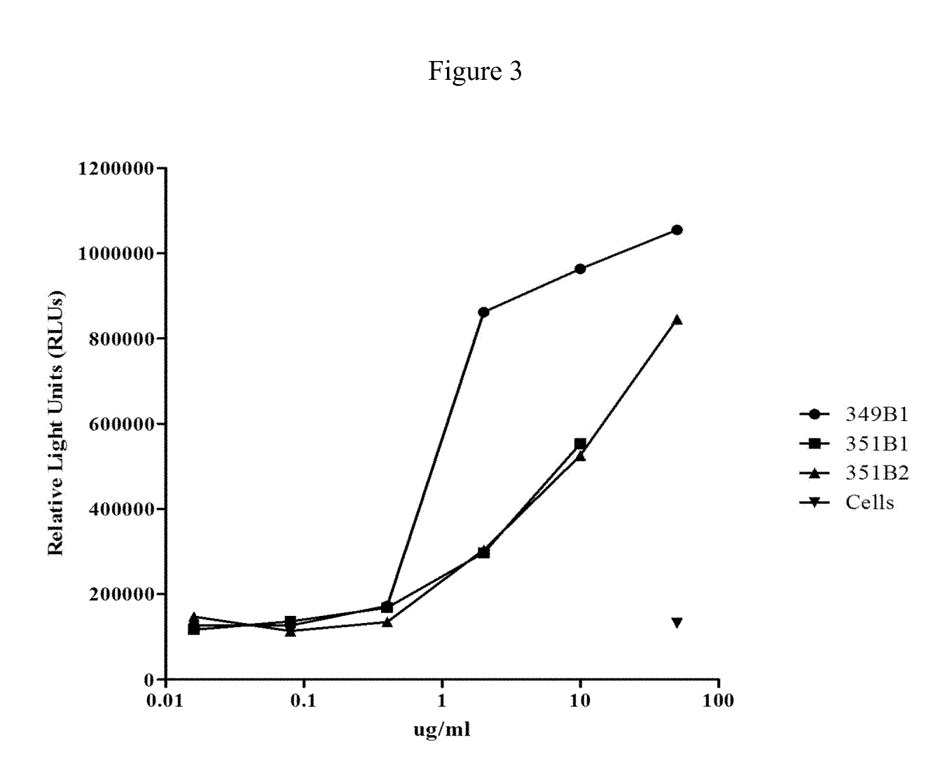

As shown in FIG. 3, anti-mB7-H4/lymphotoxin-αββ molecule 349B1 was able to robustly stimulate the NF-kB-luciferase reporter. The anti-mB7-H4/LIGHT molecules 351B1 and 351B2 were also capable of stimulating the luciferase reporter but to a lesser extent. These results suggest that the lymphotoxin-αββ moiety of 349B1 is able to strongly induce LTβR signaling in a biologically relevant manner.

### Example 4

### In Vivo Tumor Growth Inhibition by Anti-mB7-H4/Lymphotoxin-αββ Molecule

The murine colon tumor line CT26.WT was stably transfected with a full-length mouse B7-H4 construct and a cell line over-expressing mB7-H4 was established (CT26WT-B7-H4). CT26WT-B7-H4 cells were implanted subcutaneously (3×10⁵ cells/mouse) in Balb/c mice. Mice were treated with 349B1, an anti-mouse PD-L1 antibody (332M9), a combination of 349B1 and 332M9, or a control antibody (n=10 per group). 349B1 was administered at 1.25, 2.5, or 5 mg/kg 3 times a week for 2 weeks (6 total doses). 332M9 was administered at 10 mg/kg two times a week for 3 weeks. Mice were dosed by intraperitoneal injection. Tumor growth was monitored, and tumor volumes were measured with electronic calipers at the indicated time points.

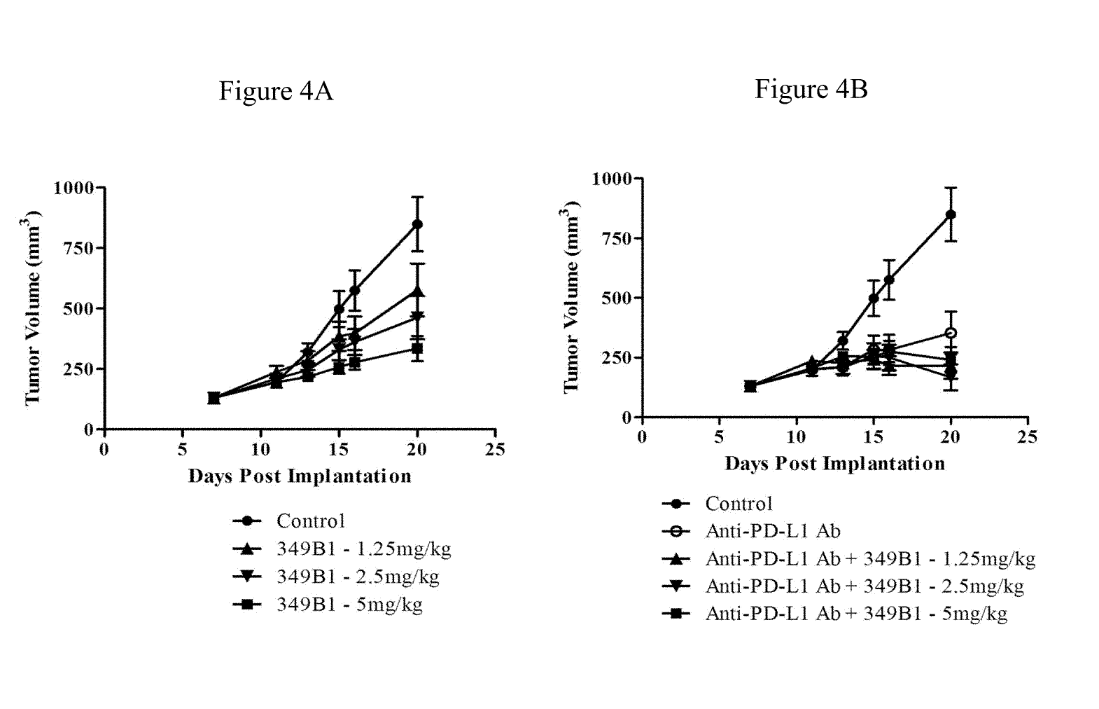

As is shown in FIG. 4A, treatment with anti-mB7-H4/lymphotoxin-αββ 349B1 inhibited the growth of the CT26.WT-B7H4 tumor in a dose-dependent manner. Tumor growth inhibition (TGI) for 349B1 was calculated to be 32% at 1.25 mg/kg, 45% at 2.5 mg/kg, and 61% at 5 mg/kg. Treatment with 349B1 in combination with an anti-PD-L1 antibody also inhibited tumor growth and at a greater level than either agent alone (FIG. 4B). TGI for anti-PD-L1 Ab as single agent was calculated to be 58%; combination with 349B1 to be 75% at 1.25 mg/kg, 80% at 2.5 mg/kg, and 72% at 5 mg/kg. These results indicate that anti-mB7-H4/lymphotoxin-αββ 349B1 is active as an immunotherapeutic agent and has the potential to be more effective in combination with additional therapeutic agents, such as checkpoint inhibitors.

### Example 5

### Anti-mB7-H4/Lymphotoxin-αββ Increases Immune Response in Tumors

Expression levels of a number of gene in tumors were measured as follows. Total RNAs was isolated from snap-frozen tumors using the RNeasy Fibrous Tissue Mini kit with DNase I treatment according to the manufacturer's protocols (Qiagen, Valencia, Calif.). All RNA samples were verified to be intact by Bioanalyzer and RNA 6000 Nano chips (Agilent Technologies, Santa Clara, Calif.). Quantitative gene expression analysis was performed with the QuantStudio 7 Flex (Thermo Fisher Scientific. Waltham, Mass.) on template generated from pre-amplified RNA using the SuperScript III One-Step RT-PCR with Platinum Taq kit (Invitrogen/Thermo Fisher Scientific, Waltham, Mass.). Calculations were performed using the comparative threshold method (ddCt). Tumor gene expression levels were normalized to GusB. One-way ANOVA tests followed by Dunnett's multiple comparison post-test were performed using GraphPad Prism. Gene expression assays were purchased directly from ThermoFisher, and are listed in Table 3.

TABLE 3

| | Gene | | | |
|---|---|---|---|---|
| Gene | Symbol | Description | Species | Assay |
| Ccl19 | Ccl19 | chemokine | Mouse | Mm00839967_g1 |
| | | (C-C motif) | | |
| | | ligand 19 | | |
| Cxcl13 | Cxcl13 | chemokine | Mouse | Mm04214185_s1 |
| | | (C-X-C motif) | | |
| | | ligand 13 | | |
| Ifng | Ifng | interferon gamma | Mouse | Mm01168134_m1 |
| Cd45 | Ptprc | protein tyrosine | Mouse | Mm01293577_m1 |
| | | phosphatase, | | |
| | | receptor type, C | | |
| Cd8 | Cd8a | CD8 antigen, | Mouse | Mm01182107_g1 |
| | | alpha chain | | |
| Cd4 | Cd4 | CD4 antigen | Mouse | Mm00442754_m1 |
| Cd3 | Cd3e | CD3 antigen, | Mouse | Mm01179194_m1 |
| | | epsilon polypeptide | | |
| Gusb | Gusb | Gusb | Mouse | Mm01197698_m1 |

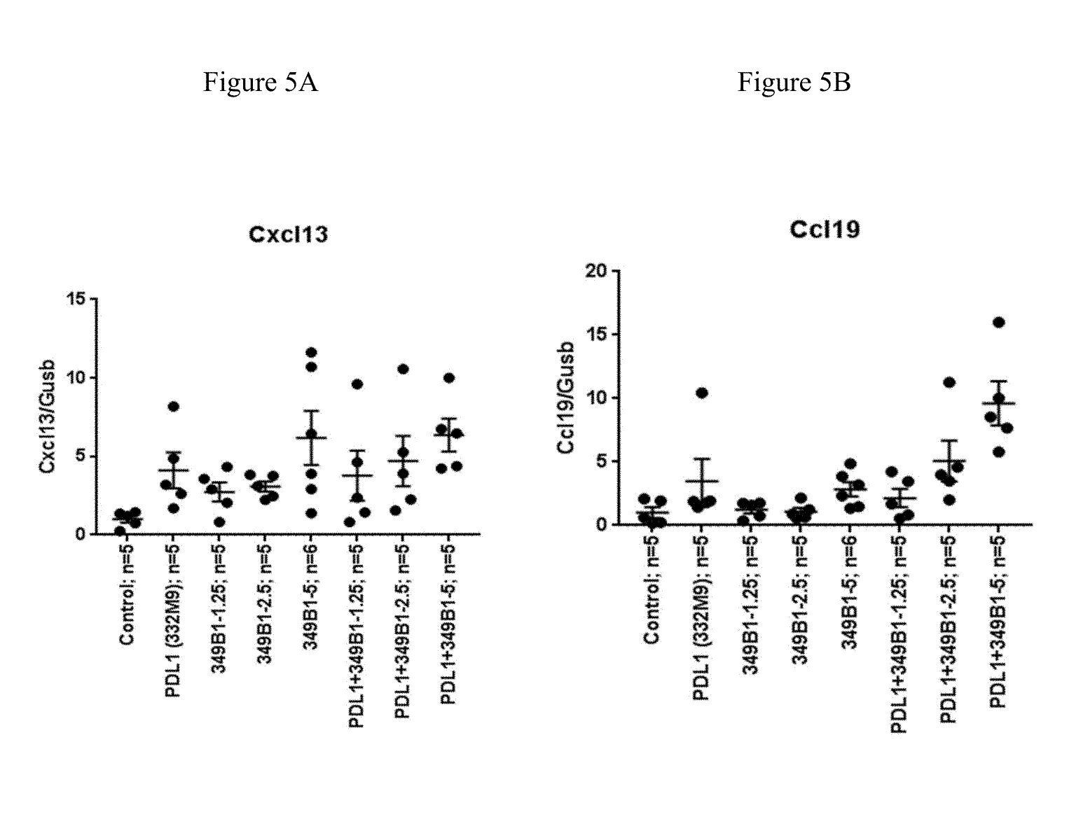

We measured expression levels of two chemokines, CXCL13, and CCL19, which are involved in recruiting immune response. As shown in FIGS. 5A and 5B, these chemokines were increased in mice receiving 349B1 as compared to controls in a dose-dependent manner, both as a single agent and in combination with an anti-PD-L1 Ab. These results demonstrate that 349B1 drives production of key chemokines involved in recruiting immune response.

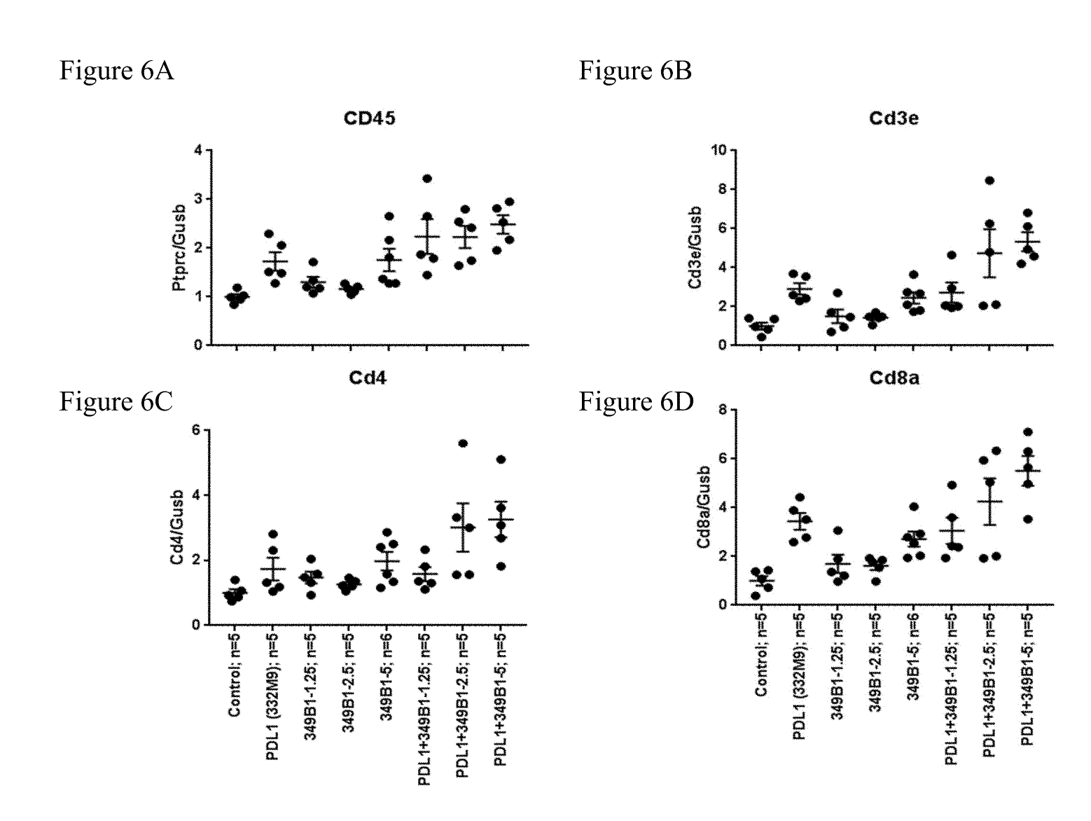

We also measured expression levels of cell surface markers that are associated with the presence of immune cells in tumors using TaqMan qPCR: CD45, CD3e, CD4, and CD8a. As shown in FIGS. 6A-6D, expression of these markers was increased in mice receiving 349B1 as compared to controls in a dose-dependent manner, both as a single agent and in combination with anti-PD-L1 Ab. These results demonstrate that 349B1 recruits immune cells to the tumor.

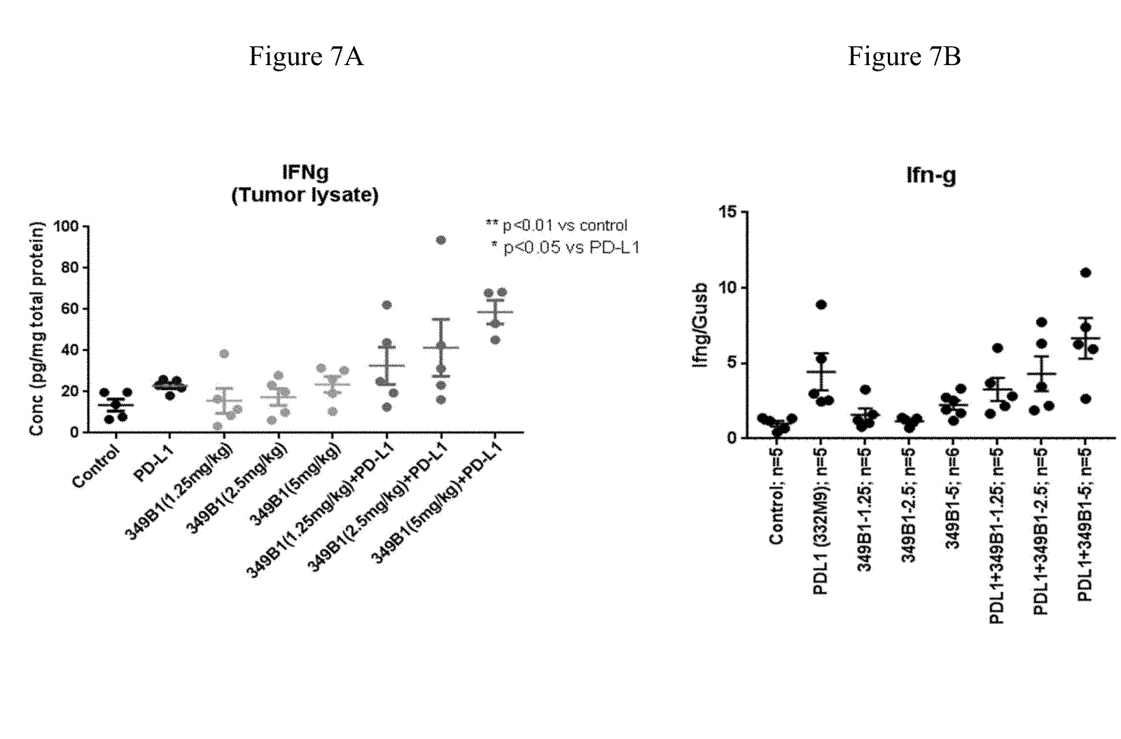

In addition, we measured both expression and protein levels of interferon-γ (IFNγ) in tumors. As shown, IFNγ levels in tumor lysate (FIG. 7A) was measured as follows. Mouse tumor samples were processed using standard NP40 protein lysis buffer containing protease inhibitor cocktails, and solubilized protein lysates were assayed by standard BCA protein assay to determine the protein concentration, cytokine and chemokine 25plex (EMD Millipore) Luminex assay respectively. The luminex assay was performed by following Luminex assay protocol with the adaption of the Drop Array system (Curiox Biosystem). Briefly, plates were blocked with 10 μL 1% BSA/PBS for 30 min at RT. Standards and controls were prepared as instructed following the protocol. 5 μL cytokine and chemokine antibody bead mixture (25 plex) were added to each well of DropArray plate. 5 μL STDs, controls, or tumor protein lysate samples were added in duplicates to the assay plate. Assay buffer was added to each well, to make the final volume of 15 μL per well for all STDs, controls, and samples. The plate was carefully inserted into a shaker and shaken for 10 sec@1000 RPM. The assay plate was placed on top of the magnetic stand in a humidified box and shook O/N at 4° C. The plate was washed three times with a DropArray LT washing station MX96 (Curiox Biosystems), and the detection antibody was added at 5 μL/well and incubated on the shaker for 60 mins. The Streptavidin/PE substrate was added at 5 L/well and incubated for 30 mins with shaking. The plate was washed 3× with DropArray LT washing station MX96. 75 μL Sheath fluid was added, and the plate was read with the adaptor.

Luminex data was analyzed using the EMD Millipore's Milliplex Analyst software. Briefly standard curve and QC controls met the system criteria by passing the % accuracy range (80-120%) and % CV<20% for duplicate. QC controls need to be within the expected concentration range as provided by the assay kit. The concentration of each assay was normalized by the total amount of tumor protein assayed by BCA assay. Statistical analysis was performed using the One-Way ANOVA Bonferroni multiple comparison tests.

The IFNγ expression (FIG. 7B) measured as described above and was observed to be increased in mice receiving 349B1 as compared to controls in a dose-dependent manner, both as a single agent and in combination with anti-PD-L1 Ab. This demonstrates that 349B1 helps promote a robust Th1 immune response in the tumor.

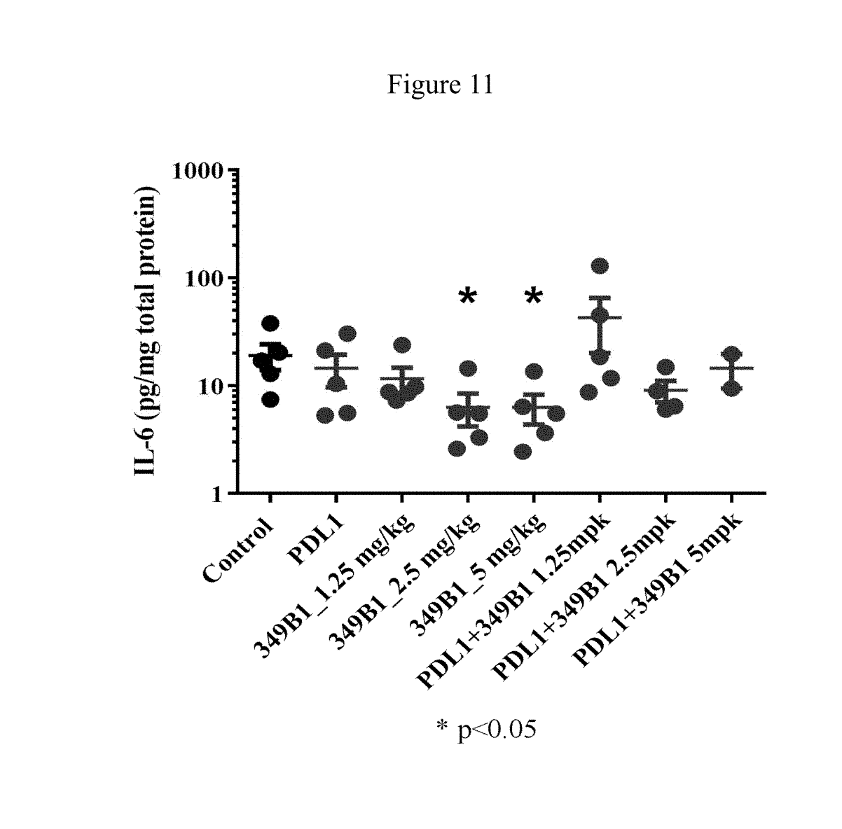

In the same manner as described for IFNγ above, protein levels of L-6 were also measured in tumors of mice receiving anti-mB7-H4/lymphotoxin-αββ 349B1, anti-mPD-L1 antibody, a combination of 349B1 and anti-mPD-L1 antibody, or a control antibody. As shown in FIG. 11, IL-6 protein level was reduced in mice treated with 50 μg and 100 μg of 349B1. IL-6 is an immunosuppressive cytokine in the context of a tumor. Therefore, anti-mB7-H4/lymphotoxin-αββ 349B1 increased Th1 response as evidenced by the increase in IFNγ production, while decreasing Th2 response.

### Example 6

Generation of a B7-H4 Antibody that Binds Human and Mouse B7-H4

We used standard techniques to generate a mouse monoclonal antibody directed against B7-H4. Following screening and binding, the 278M1 murine antibody was generated. Heavy and light chain sequences of this antibody are provided as indicated below.

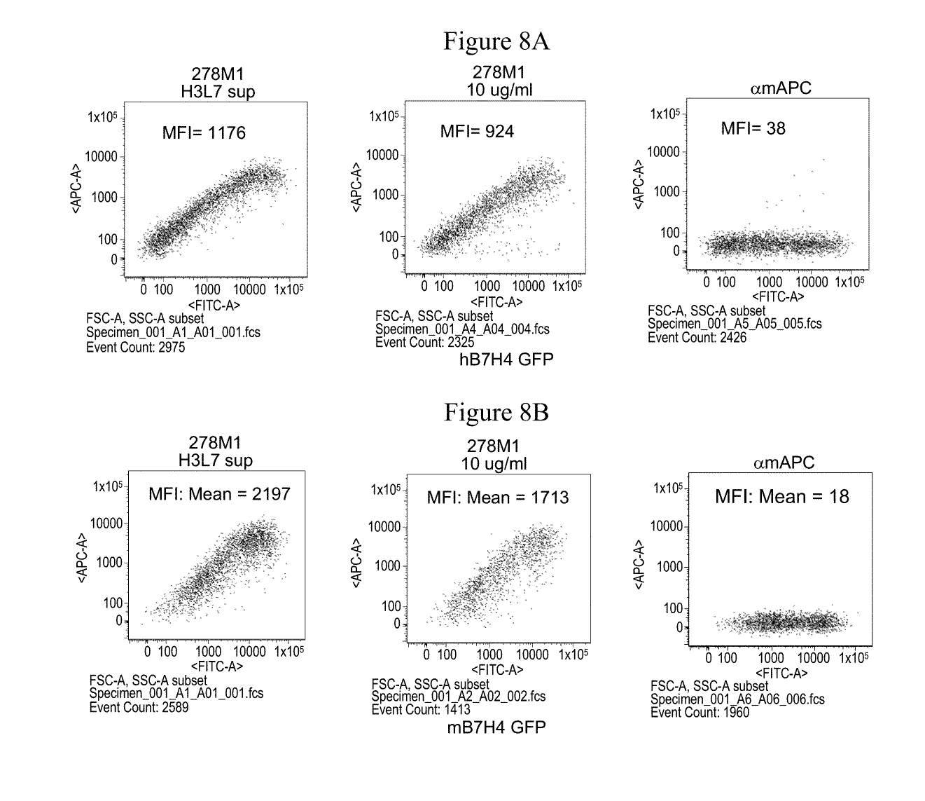

We tested 278M1 for binding to both human and mouse B7-H4. These studies were performed essentially as described in Example 2. As shown in FIGS. 8A and 8B, FACS analysis demonstrated that 278M1 was capable of binding both human and mouse B7-H4.

### Example 7

### B7-H4 is Expressed in Breast and Ovarian Tumors

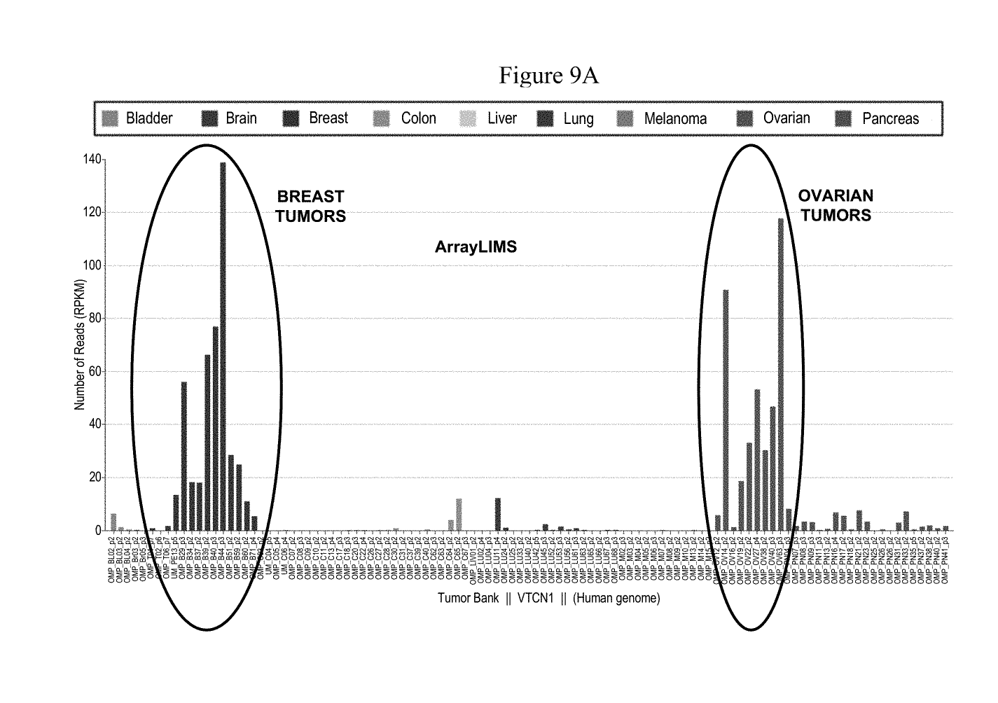

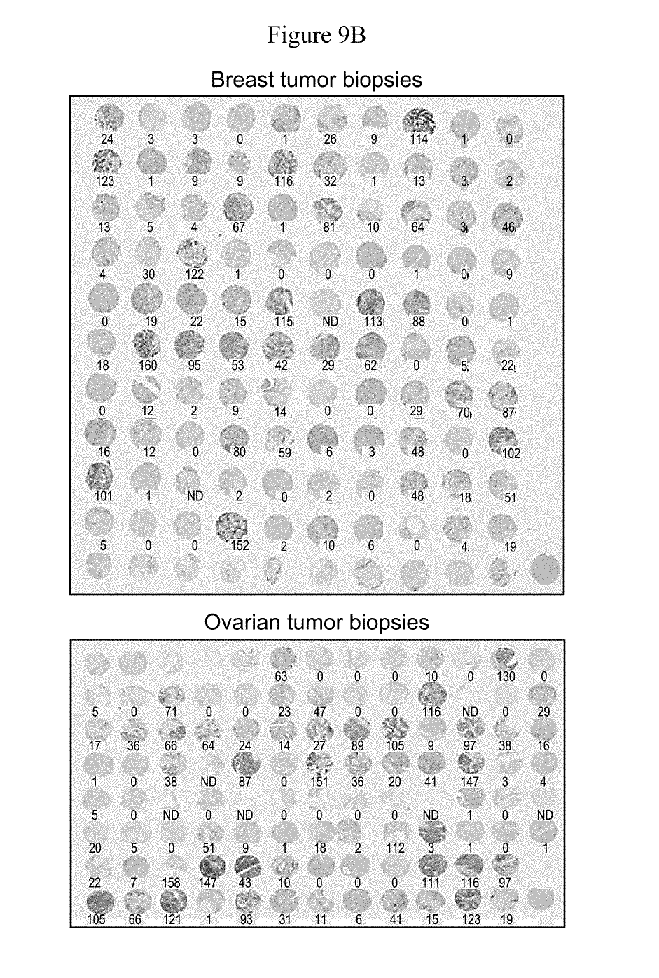

We determined the expression of B7-H4 in various human tumors in OncoMed's tumor bank by microarray. As shown in FIG. 9 (left panel), B7-H4 was highly expressed in many breast and ovarian tumors, suggesting its use as a tumor-associated antigen for these cancer types.

Measurement of B7-H4 expression from tumor biopsies in the breast (FIG. 9, upper right panel) and ovarian (FIG. 9, lower right panel) cancer measured by immunohistochemistry also showed B7-H4 expression in many of these tumors.

### Example 8

### P-CADHERIN/CDH3 is Expressed in Many Tumors

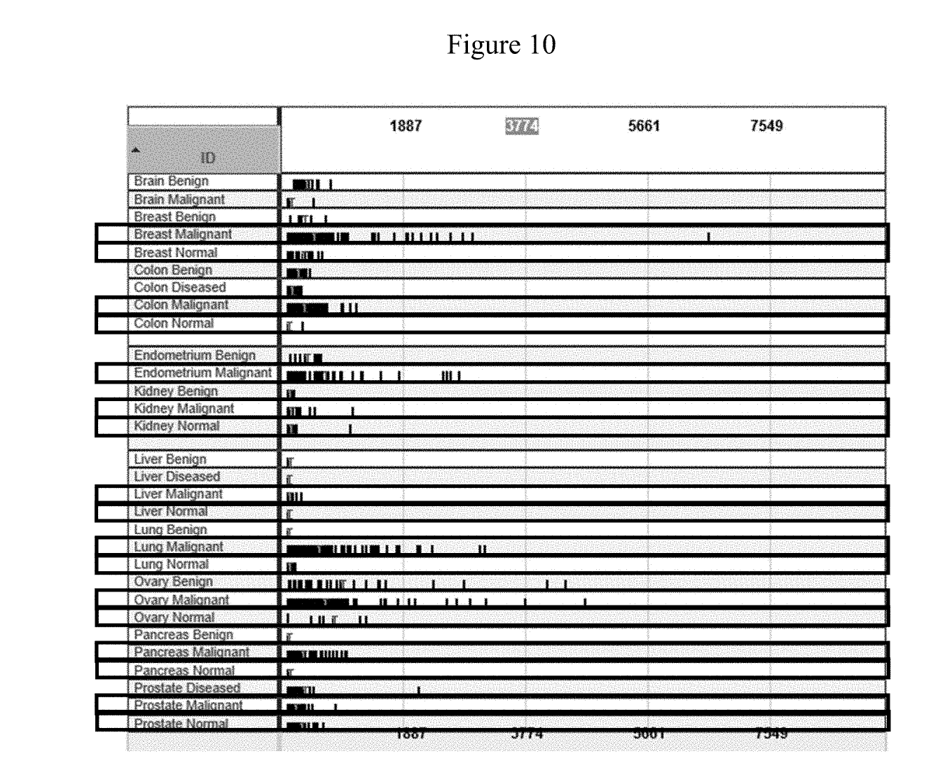

We analyzed expression patterns of P-CADHERIN across many tissues in a Gene Logic microarray dataset that includes Affymetrix microarray data of human tissue samples, both in normal tissues and in malignant tissues (FIG. 10). As shown, P-CADHERIN is expressed in many tumors. Expression in normal tissue is also observed in ovary, breast, and prostate tissues. As such, P-CADHERIN may also be useful as a tumor-associated antigen.

### Example 9

### Significance of In Vivo Tumor Targeting in Anti-Tumor Activity by Anti-mB7-H4/Lymphotoxin-αββ

The murine colon tumor line MC38 was transduced with a lentivirus expressing full-length mouse B7-H4 construct and a cell line over-expressing mB7-H4 was established (MC38-B7H4). MC38 parental line or MC38-B7H4 cells were implanted subcutaneously (4×10⁵ cells per mouse) in C57BL/6N mice. Mice were treated with a flat dose of anti-mB7-H4/lymphotoxin-αββ (349B1), an anti-mouse PD-L1 antibody (332M9), a combination of 349B1 and 332M9, or a control antibody (n=10 per group). 349B1 was administered at 50, 100, and 200 μg per mouse, 3 times a week for 2 weeks (6 total doses). 332M9 was administered at 200 μg per mouse weekly for 3 weeks. Drugs were administered by intraperitoneal injection. Tumor growth was monitored, and tumor volumes were measured with electronic calipers at the indicated time points.

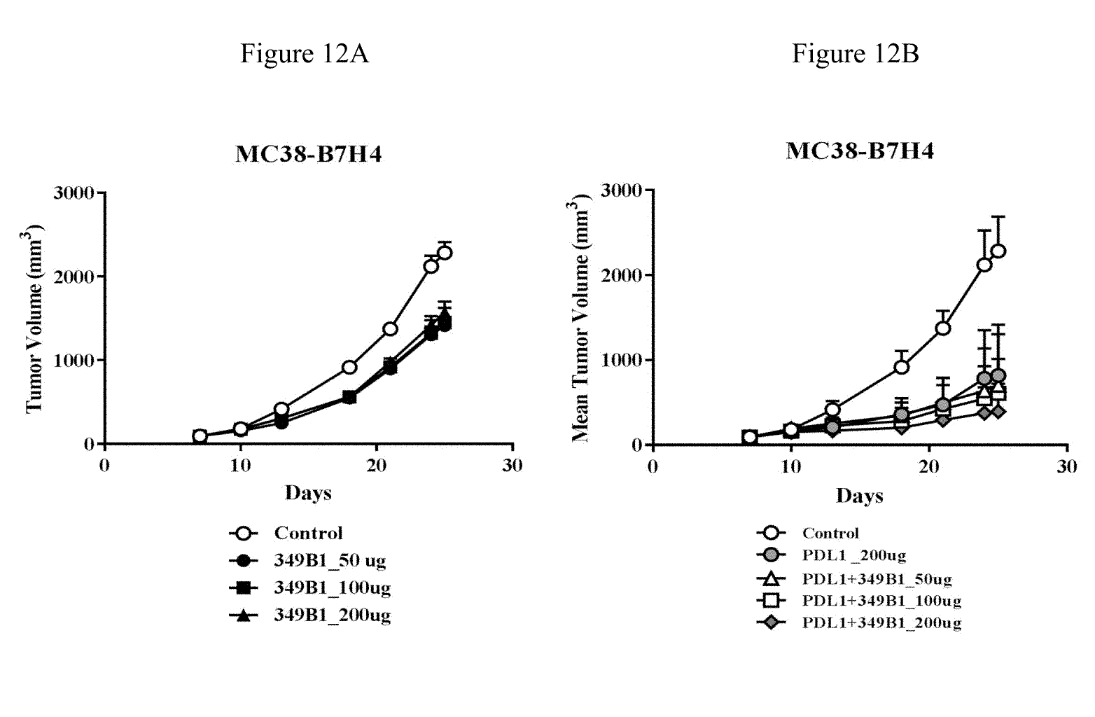

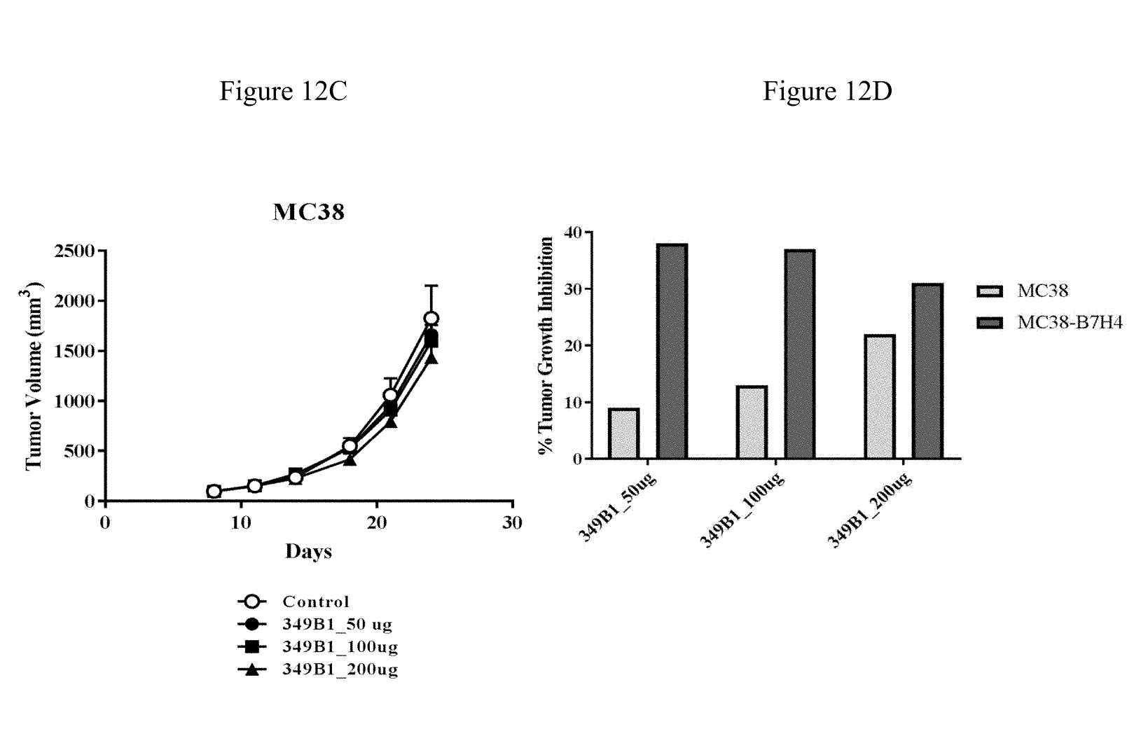

As is shown in FIG. 12A, treatment with anti-mB7-H4/lymphotoxin-up 349B1 inhibited the growth of the MC38-B7H4 tumor. Tumor growth inhibition (TGI) for 349B1 was calculated to be 38% at 2.5 mg/kg, 37% at 5 mg/kg, and 31% at 10 mg/kg. Treatment with 349B1 in combination with an anti-PD-L1 antibody also inhibited tumor growth and at a greater level than either agent alone (FIG. 12B). TGI for anti-PD-L1 Ab as a single agent was calculated to be 64%; combination with 349B1 to be 69% at 50 μg, 73% at 100 μg, and 83% at 200 μg. These results indicate that anti-mB7-H4/lymphotoxin-αββ 349B1 is active as an immunotherapeutic agent and has the potential to be more effective in combination with additional therapeutic agents, such as checkpoint inhibitors.

While anti-mB7-H4/lymphotoxin-αββ 349B1 inhibited the growth of the MC38-B7H4 tumor as a single agent, there was no significant anti-tumor activity when MC38 parental cells were used (FIG. 12C), indicating that tumor targeting is required for anti-tumor activity of anti-mB7-H4/lymphotoxin-αββ 349B1 (FIG. 12D).

Tumor targeting by anti-mB7-H4/lymphotoxin-αββ molecule 349B1 was also assessed by comparing the anti-tumor activity of 349B1 with the anti-tumor activity of lymphotoxin-αββ without the tumor targeting portion of the molecule in the MC38-B7H4 model described above. MC38-B7H4 cells were implanted subcutaneously (4×10⁵ cells per mouse) in C57BL/6N mice. Mice were treated with 363F1 (lymphotoxin-αββ), 278M24 (anti-mB7-H4 antibody), 349B1 (anti-mB7-H4/lymphotoxin-αββ), an anti-mouse PD-L1 antibody (332M9), a combination of 332M9 and 363F1, a combination of 332M9 and 349B1, or control antibody (n=10 per group). 363F1 and 278M24 were administered at 62.5 μg per mouse, and 349B1 at 100 μg per mouse twice weekly for two weeks, and 332M9 was administered at 50 μg per mouse weekly for two weeks. Drugs were administered by intraperitoneal injection. Tumor growth was monitored, and tumor volumes were measured with electronic calipers at the indicated time points.

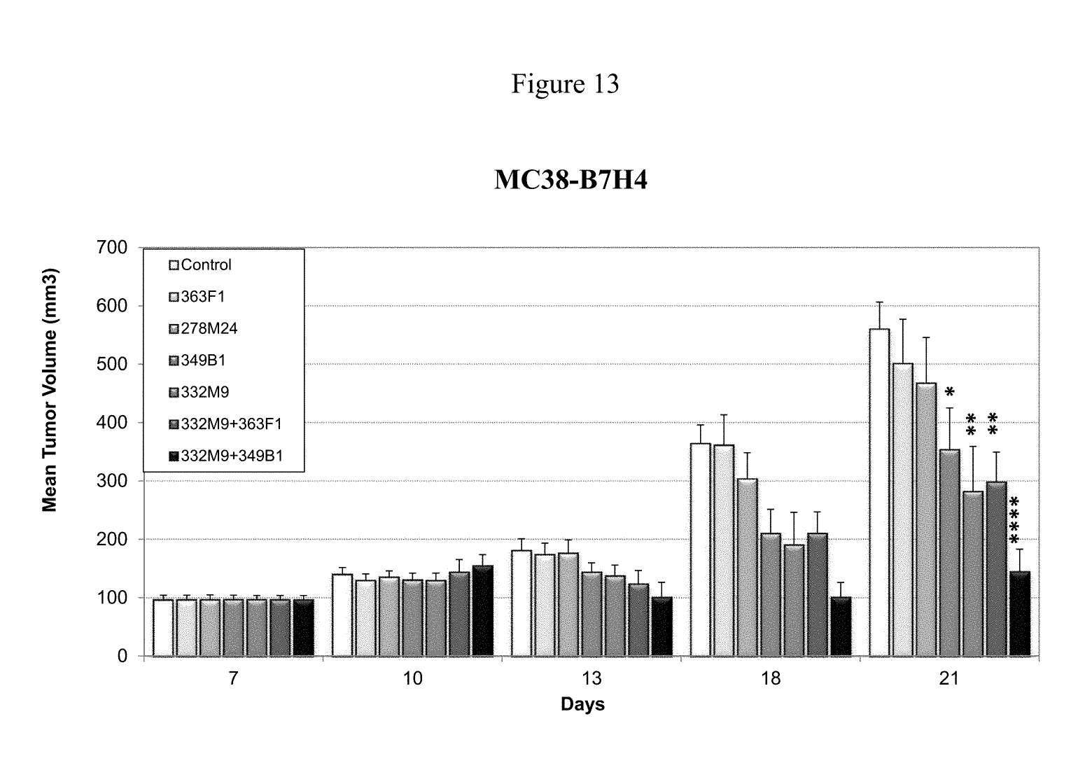

As is shown in FIG. 13, anti-mB7-H4/lymphotoxin-αββ 349B1, but not lymphotoxin-αββ 363F1 alone, inhibited the growth of the MC38-B7H4 tumor. Antibody against B7H4 278M24 showed no anti-tumor activity. These results indicate that lymphotoxin-αββ needs to be delivered to the tumor site via tumor targeting antibody for anti-tumor activity.

### Example 10

### Anti-mB7-H4/Lymphotoxin-αββ Increases T-Cell Infiltration in Tumors

T-cell infiltration into tumors after treatment with anti-mB7-H4/lymphotoxin-αββ 349B1 was assessed by immunohistochemistry. CT26.WT-B7H4 tumor-bearing mice were treated with 25 μg, 50 μg, and 100 μg 349B1 per mouse three times weekly for two weeks. Tumors were fixed in formalin and embedded paraffin. Tumor sections (4 microns) mounted on glass slides were immunostained in automated assays using Ventana Discovery Instrument (Roche) with Ventana reagents. CD3′ total T cells were quantified using the Definiens software and expressed as % CD3′ cell in the non-necrotic tumor area as well as total immune cells (CD45⁺ cells).

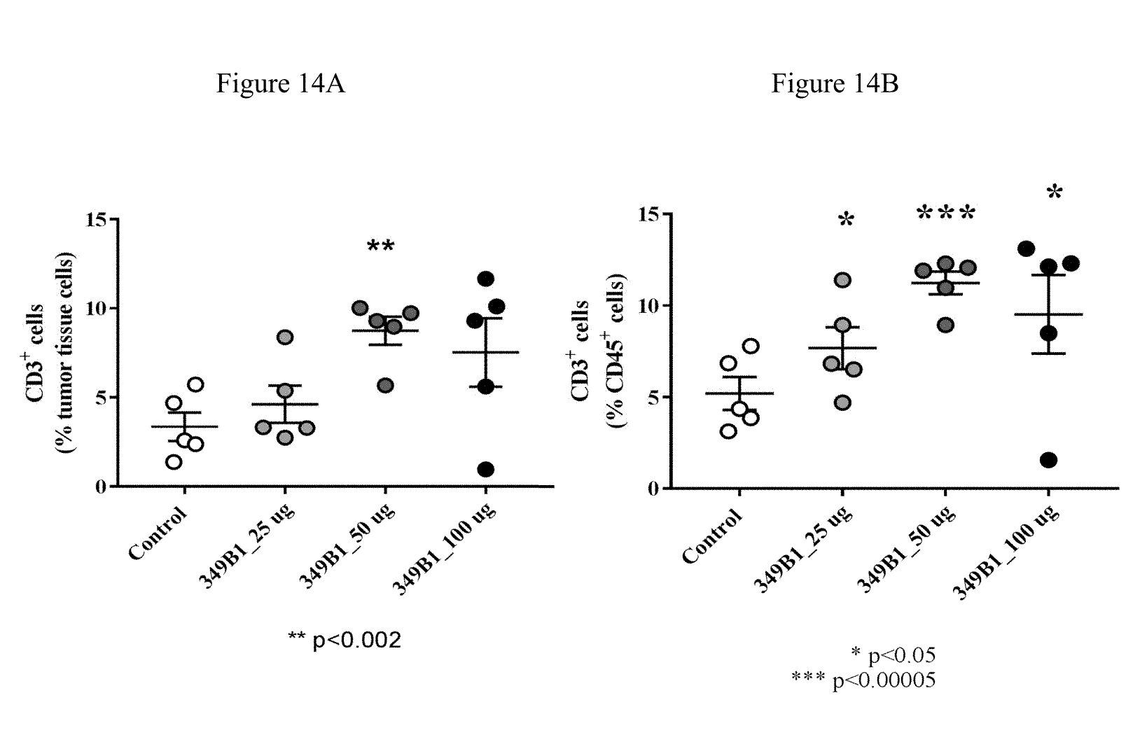

As shown FIG. 14A, more T cells were infiltrated in the tumor area after treatment with anti-mB7-H4/lymphotoxin-αββ 349B1 compared to the control group. The increase in total T-cell infiltration was more pronounced when gated on CD45⁺ total immune cells (FIG. 14B), indicating that T cells are enriched in the tumors treated with anti-mB7-H4/lymphotoxin-αββ 349B1.

### Example 11

Immune Cell Infiltration into Tumor Induced by a Single Dose of Anti-mB7-H4/Lymphotoxin-αββ 349B1

To understand the immune-mediated mechanism employed by 349B1 for its anti-tumor activity, immune cell infiltration was monitored at different time points upon single dose administration of 349B1 by analyzing immune cell population in tumors by flow cytometry. CT26WT-B7H4 cells were implanted into BALB/c mice (3×10⁵ cells per mouse) and dosed once on the 7th day after the cell implantation at 10 μg and 100 μg per mouse. Tumors were harvested from multiple animals at 10, 14, and 21-day post-cell implantation, single cell suspensions were prepared, and immunostained with antibodies against mouse CD45, CD11b, CD4, CD8, NKp46, CD11c, CD19, F4/80, and Gr-1, and analyzed by flow cytometry. Data were processed using FACS Diva Software.

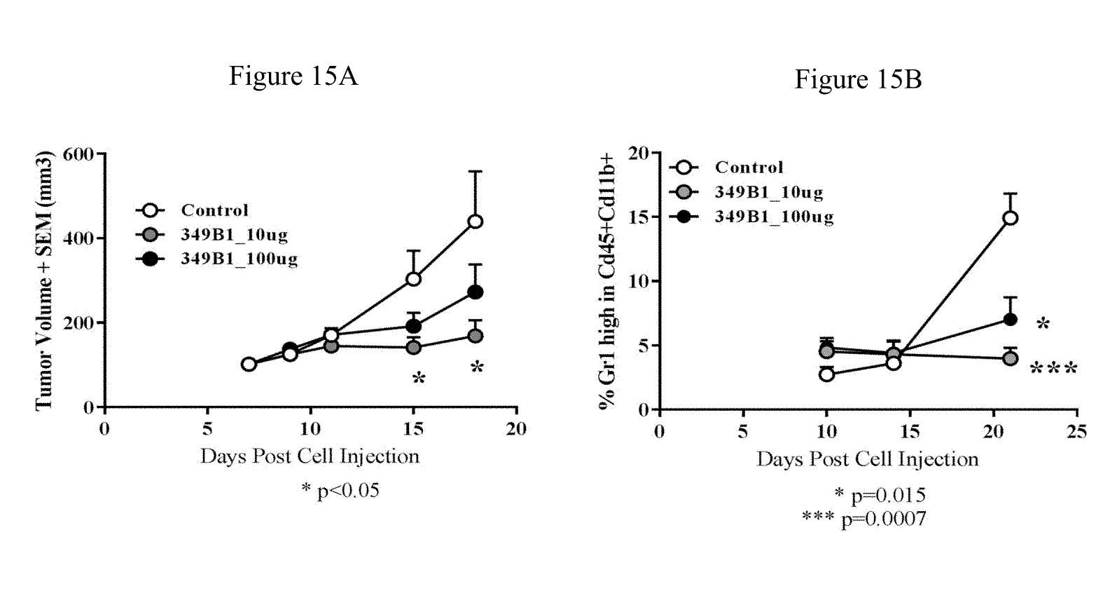

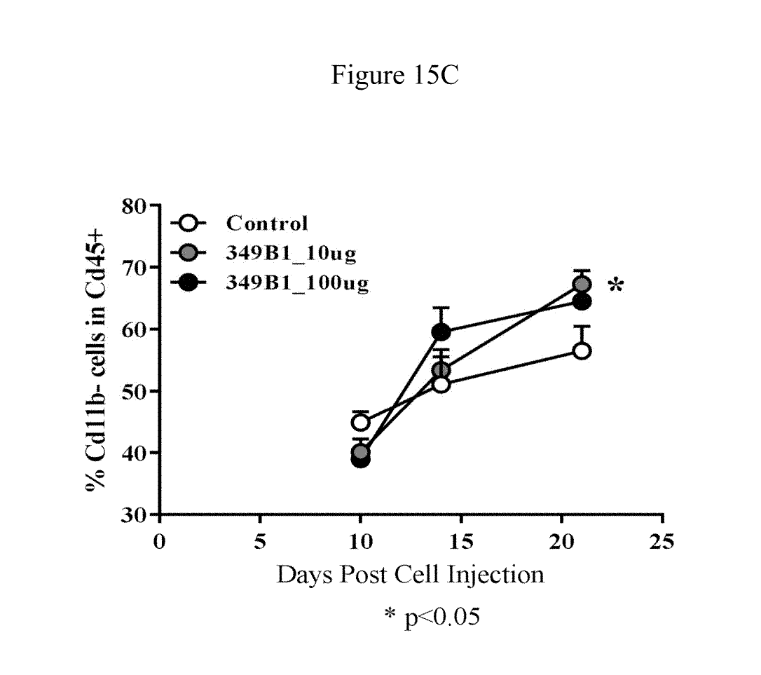

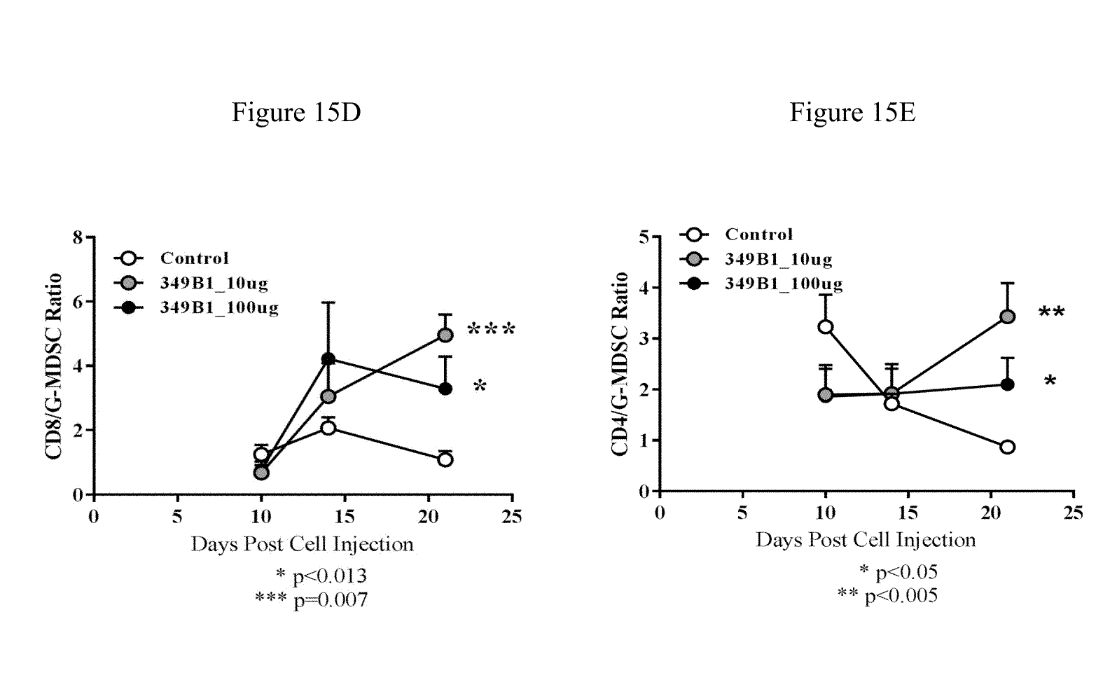

Single doses of anti-mB7-H4/lymphotoxin-αββ 349B1 as low as 10 μg showed anti-tumor activity (FIG. 15A). Flow cytometry data indicated that the Gr1 high (Granulocytic) MDSCs (Granulocytic Myeloid-Derived Suppressor Cells) population was reduced after the anti-mB7-H4/lymphotoxin-αββ 349B1 treatment (FIG. 15B). This reduction was more pronounced with 10 μg than with 100 μg anti-mB7-H4/lymphotoxin-αββ 349B1. FACS analysis indicated that myeloid-derived suppressor cells (G-MDSCs; Cd45⁺/Cd11b⁺/Gr1 high cells) were increased as tumors grow in the control group, whereas the G-MDSCs were reduced by 349B1 treatment. The reduction was greater with a lower dose of 349B1 compared to the 10 times high dose. G-MDSCs are potent suppressors of T-cell activation and therefore are a significant impediment to cancer therapy. Reduction of the G-MDSC population would be beneficial for anti-tumor activity and inversely correlated with tumor volume.

Analysis of lymphocyte populations indicated that although the relative amounts of individual populations of lymphocytes were not increased, the overall lymphoid cell population (CD45⁺Cd11b⁻) was increased after anti-mB7-H4/lymphotoxin-αββ 349B1 treatment (FIG. 15C). A significant reduction of MDSCs, and G-MDSCs in particular, as well as a slight increase in total lymphoid cells, led to an overall increase in the CD8⁺ cytotoxic T cell to G-MDSC ratio (FIG. 15D) and the CD4⁺ effector T cell to G-MDSC ratio (FIG. 15E).

### Example 12

### Induction of Tertiary Lymphoid Structure Induced by Anti-mB7-H4/Lymphotoxin-αββ

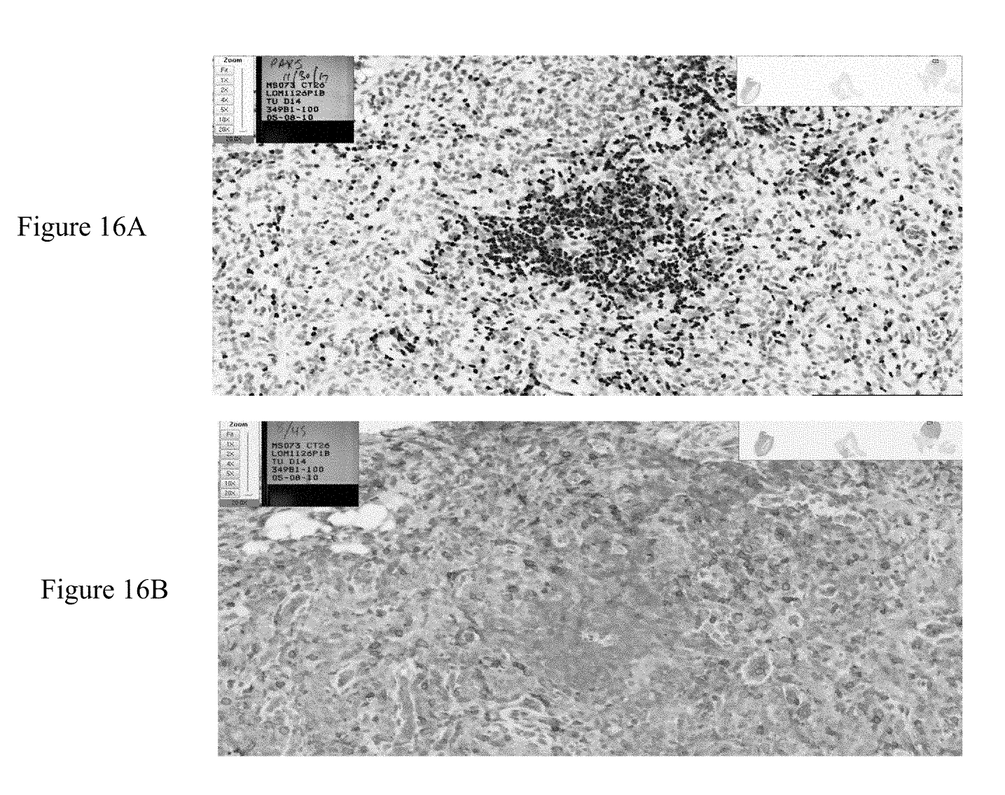

The main function of lymphotoxin is to generate germinal center. Therefore, immunohistochemistry was performed to detect immune cell aggregated containing B- and CD8 T-lymphocytes as well as CD45+ total immune cells. BALB/c mice inoculated subcutaneously with CT26WT-B7H4 cells (3×105 cells per mouse) and dosed once on the 7th day after the cell implantation at 100 μg 349B1 anti-mB7-H4/lymphotoxin-αββ per mouse. Tumors were harvested from multiple animals at 7 days after the dosing and subjected to immunohistochemistry. As shown in FIG. 16A, B-cell clusters were detected by the Pax5 antibody, which is detected in B cell nucleus. CD8 T cells were localized surrounding area of the B-cell clusters (FIG. 16B). This indicates that anti-mB7-H4/lymphotoxin-αββ can home to the tumor site and generated tertiary lymphoid structures.

### Example 13

Activity of anti-mB7-H4/lymphotoxin-αββ on TC1-mB7-H4 tumors

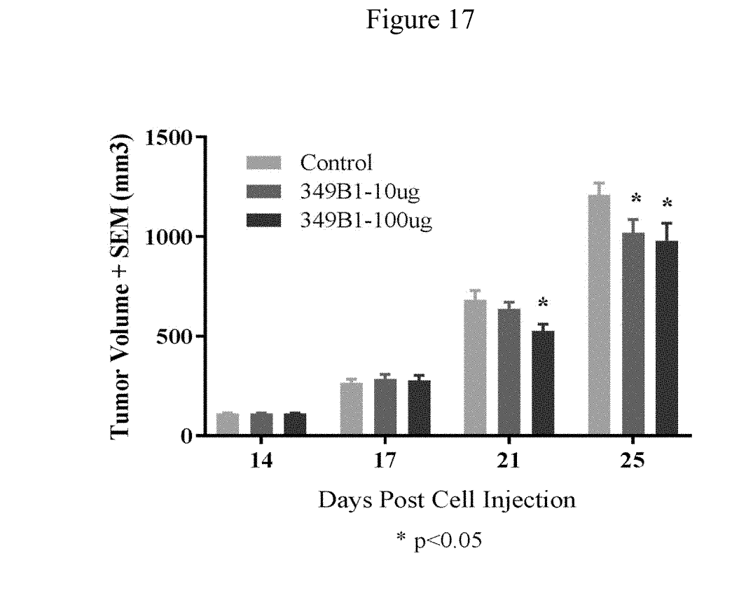

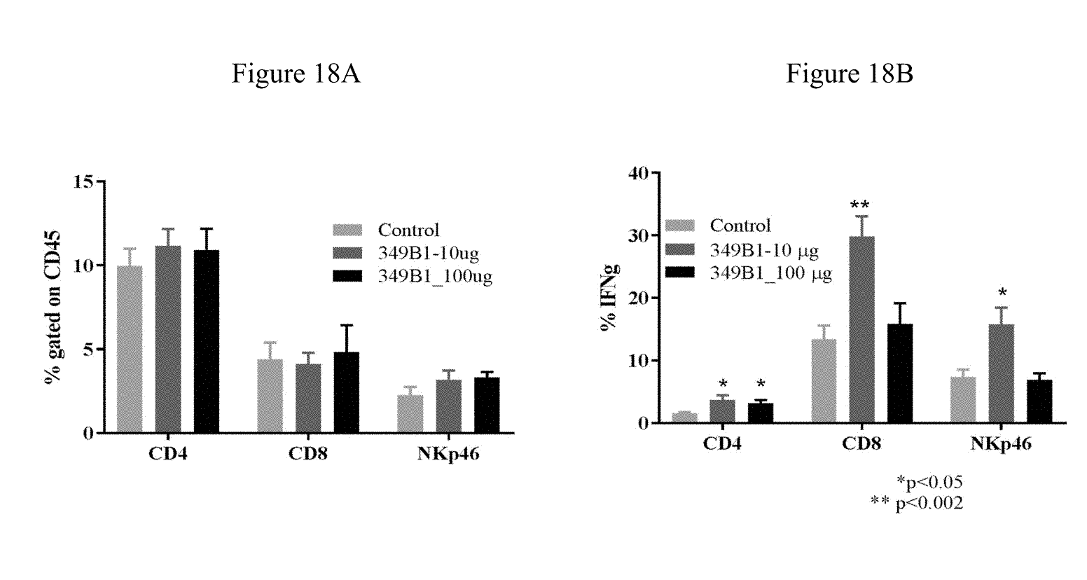

The TC1 tumor line has been derived by expression of HPV16 E16 and E17 in lung epithelial cell of C57BL/6 mice and has been used as a model for vaccine therapy for head and neck cancer. To determine the anti-tumor activity of anti-mB7-H4/lymphotoxin-αββ, TC1-B7H4 tumor-bearing mice were dosed one time, and tumor growth and immune infiltration into tumor were monitored. TC1-B7H4 cells were implanted into C57BL/6J mice (5×105 cells per mouse) and dosed once on the 7th day after the cell implantation at 10 μg and 100 μg 349B1 per mouse. Tumors were harvested from multiple animals at 10, 14, and 21-day post-cell implantation, single cell suspensions were prepared, and immunostained with antibodies against mouse CD45, CD11b, CD4, CD8, NKp46, CD11c, CD19, F4/80, and Gr-1, and analyzed by flow cytometry. Small but significant tumor growth inhibition was observed (FIG. 17). Analysis of flow cytometry data indicated that CD4, CD8 and NK cell population was not significantly increased by 349B1 treatment (FIG. 18A). However, IFNγ production was higher in CD4, CD8 and NK cells in the tumors from 10 μg of 349B1 treated mice (FIG. 18B), indicating that TC1-B7H4 tumors respond differently than CT26-B7H4 tumors to 349B1.

### Example 14

### Activity of Anti-mB7-H4/Lymphotoxin-αββ 349B1 on EMT6-mB7-H4 Tumors

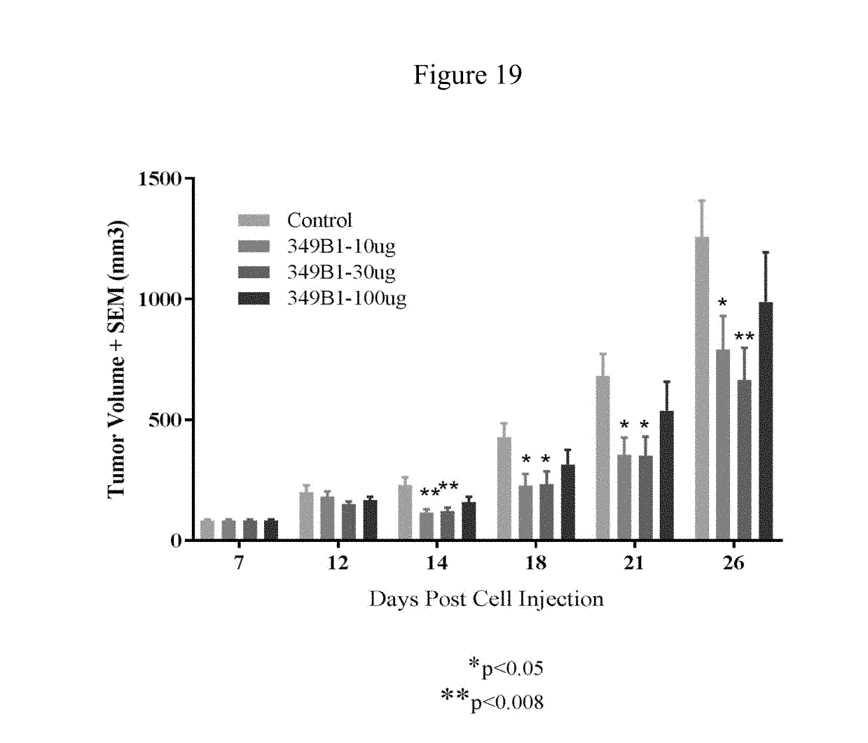

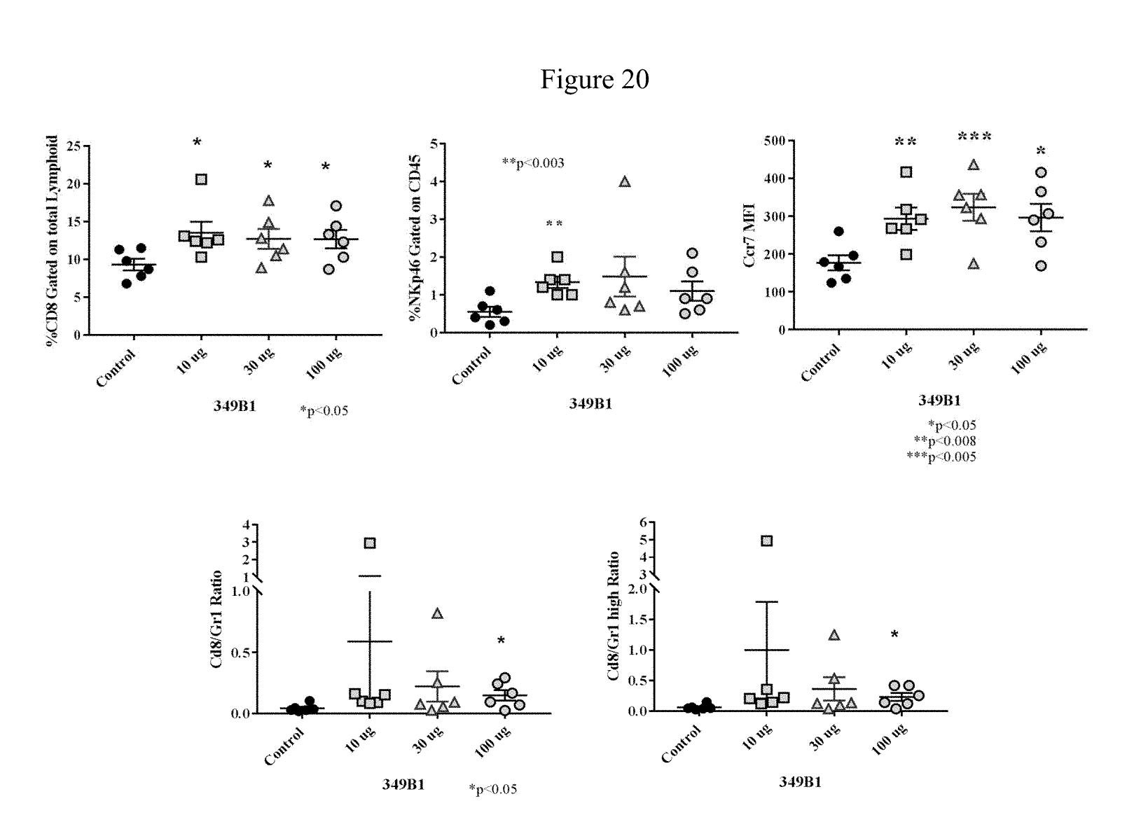

Different types of tumors may have different immune composition. Murine breast carcinoma EMT6 tumors have high infiltration of myeloid cells and fewer T cells. To determine the anti-tumor activity of anti-mB7-H4/lymphotoxin-αββ, EMT6-B7H4 tumor-bearing mice were dosed weekly, and tumor growth and immune infiltration into tumor were monitored. EMT6-B7H4 cells were implanted into BALB/c mice (1.5×10⁴ cells per mouse) and dosed weekly on the 7th day after the cell implantation at 10 μg, 30 μg, and 100 μg per mouse. Tumors were harvested from multiple animals at 6 days after the third dose, single cell suspensions were prepared, and immunostained with antibodies against mouse CD45, CD11b, CD4, CD8, NKp46, CD11c, CD19, F4/80, and Gr-1, and analyzed by flow cytometry. Weekly dosing of 349B1 showed anti-tumor activity at 10 μg and 30 μg 349B1 (FIG. 19). Flow cytometry data showed an overall increase in CD8 T lymphocytes (FIG. 20 top left panel) and NK cells with activating receptor NKp46 (FIG. 20 top center panel). Furthermore, Ccr7 expression was increased in NK cells, consistent with NK cell migration into tumor site through the endothelium (FIG. 20 top right panel). The ratio of CD8 lymphocyte to total Gr1 (total MDSC) (FIG. 20 bottom left panel) and to Gr1 high (Granulocytic MDSC) (FIG. 20 bottom right panel) was also slightly increased with anti-mB7-H4/lymphotoxin-αββ 349B1 treatment. The much larger ratio of CD8 T lymphocyte to Gr1 high MDSC was observed in CT26-B7H4 tumor model (FIG. 15D) and seems to correlate with tumor growth inhibition.

### Example 15

### Tumor-Specific Antigen Expression in Patient-Derived Tumors.

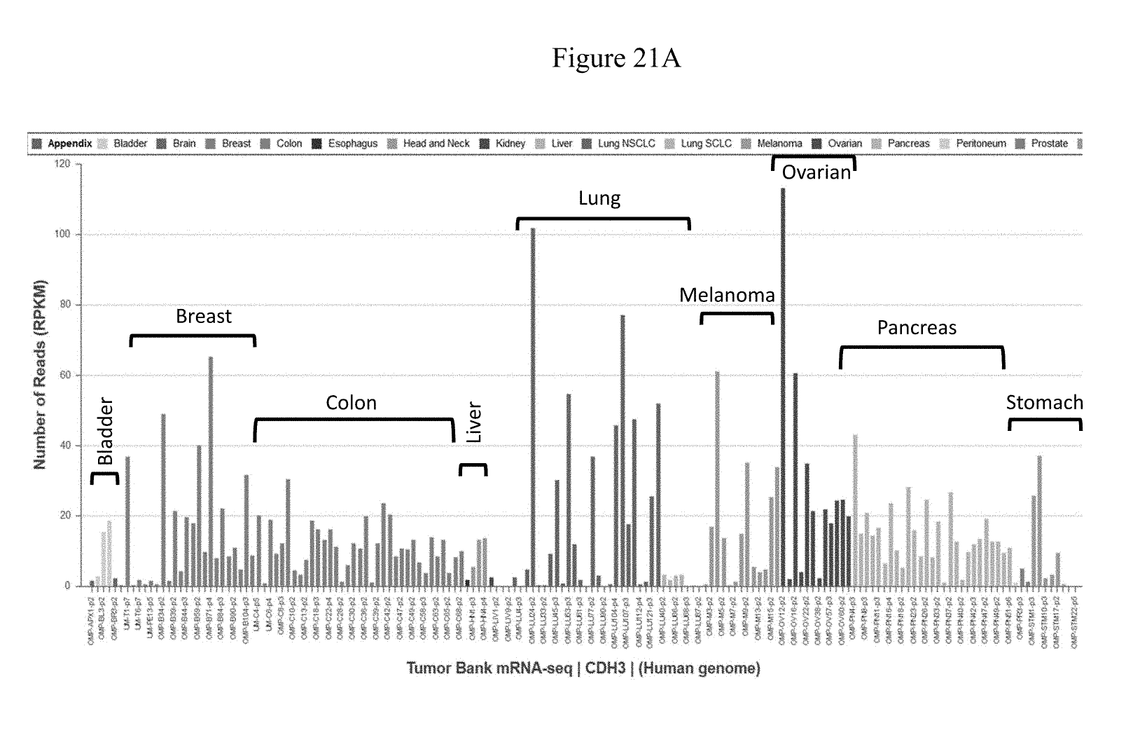

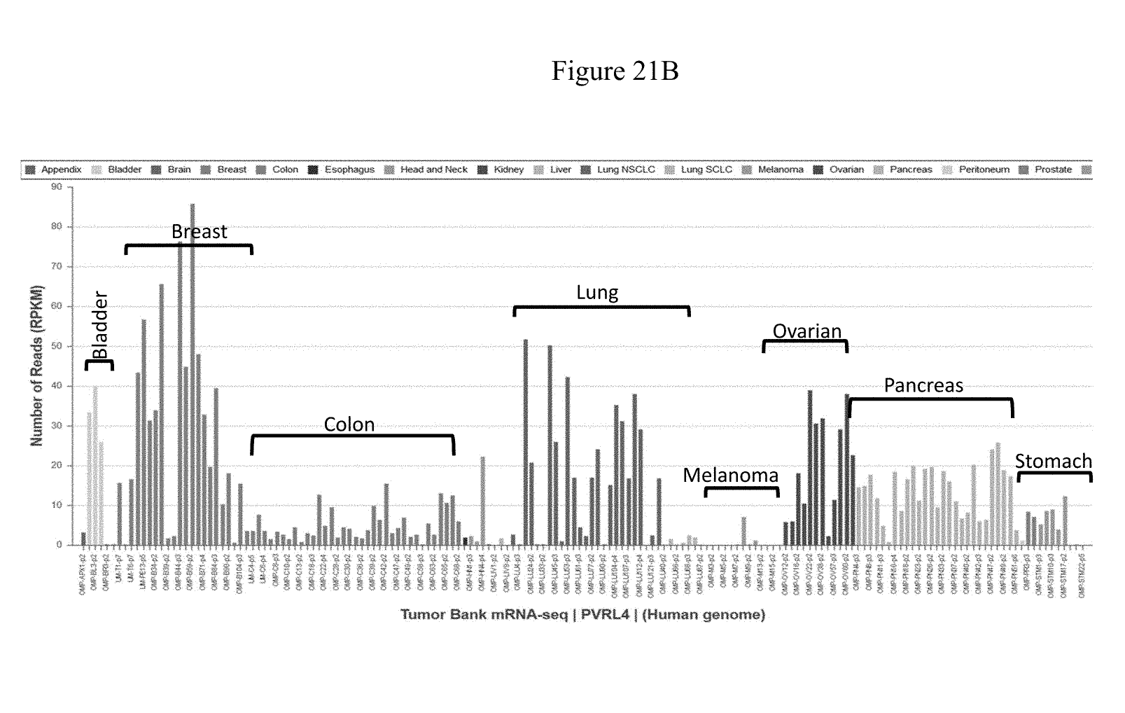

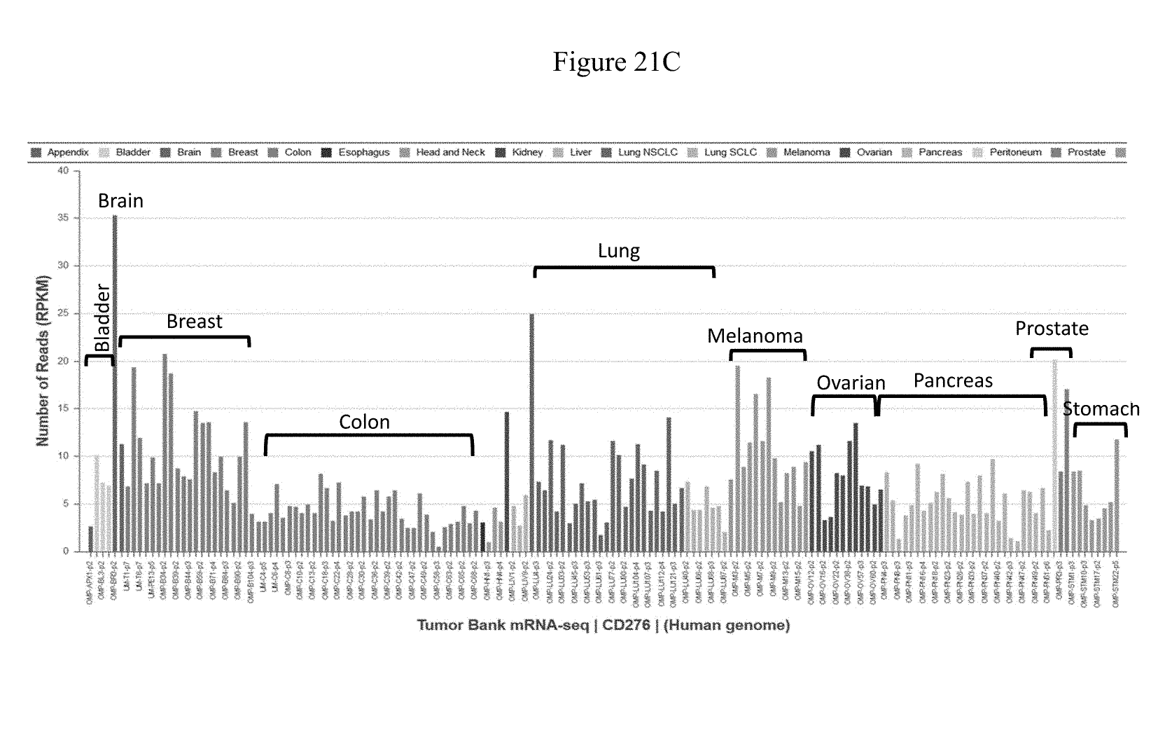

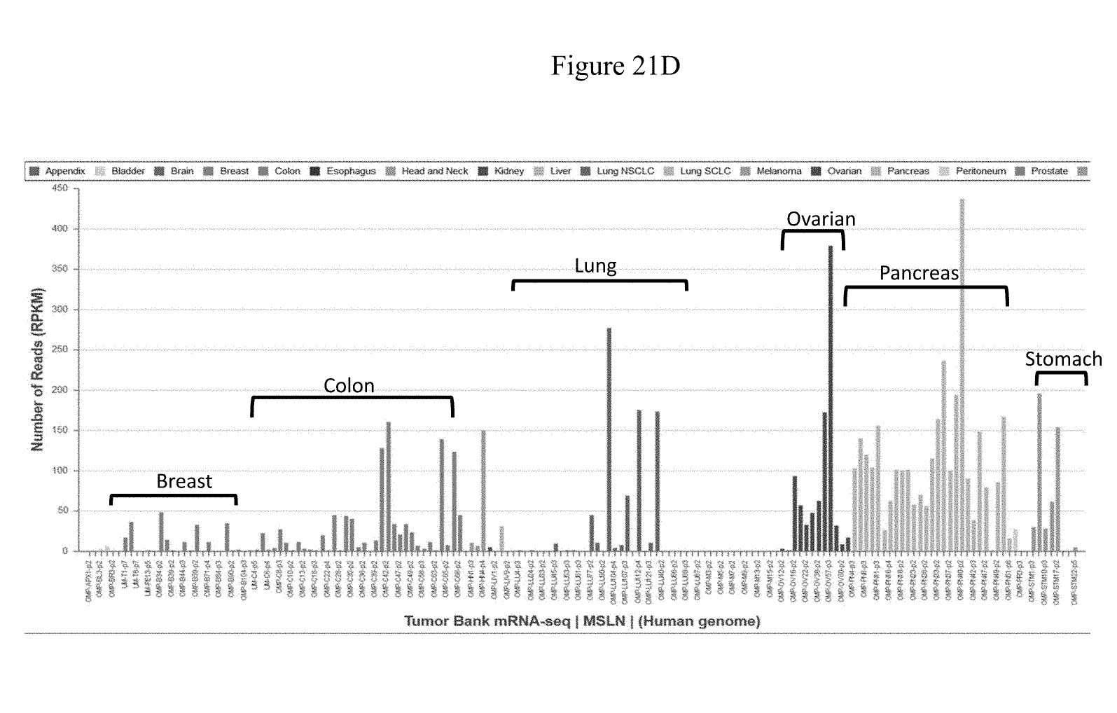

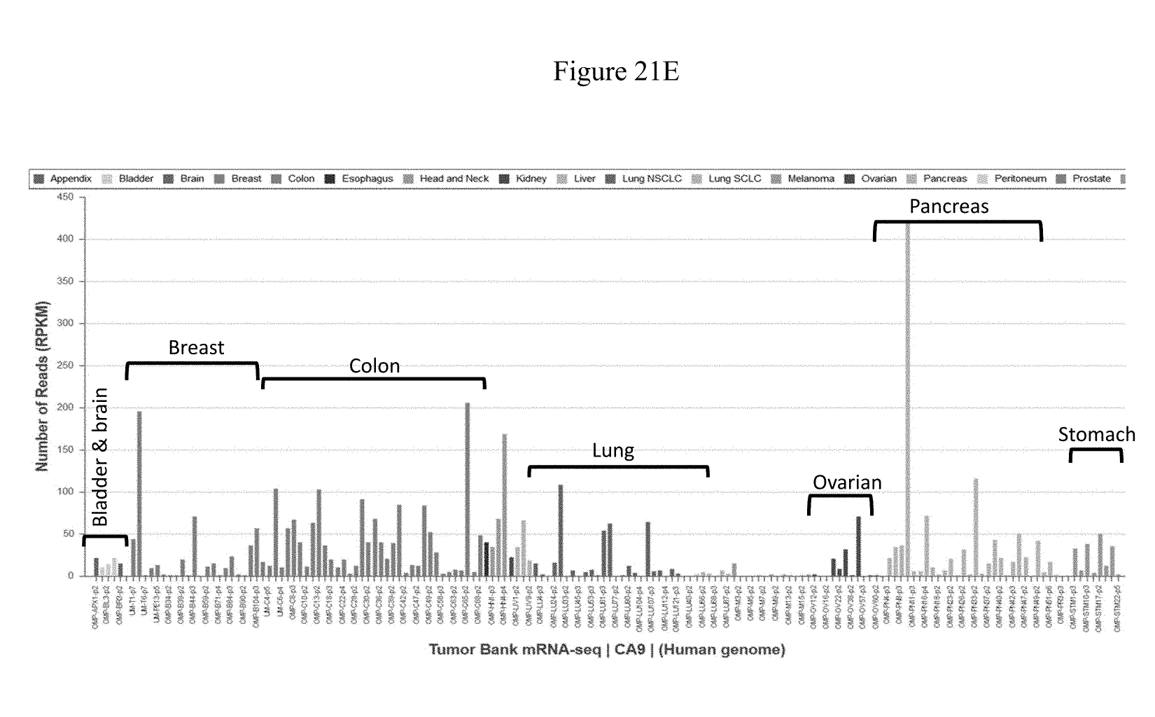

We determined the expression of several tumor antigens in various patient-derived tumors in OncoMed's tumor bank using RNA-transcript sequencing. The results are shown in FIGS. 21A-21E.

As shown in FIG. 21A, P-CADHERIN (CDH3) was highly expressed in many bladder, breast, colon, lung, melanoma, ovarian, pancreatic, and stomach tumors, suggesting its use as a tumor-associated antigen for these cancer types. Similarly, PVRL4 was shown to be highly expressed in many bladder, breast, lung, and ovarian tumors, in addition to being expressed in many pancreatic and stomach tumors (FIG. 21B). PVRL4 expression was also observed in a number of colon tumors. As shown in FIG. 21C, CD276 (B7-H3) was expressed in many brain, bladder, breast, lung, melanoma, ovarian, colon, pancreatic, prostate, and stomach tumors. As shown in FIG. 21D, mesothelin was expressed in many ovarian and pancreatic tumors, as well as certain colon, lung and stomach tumors. FIG. 21E shows the levels of CA9 expression measured in the tumors of the tumor bank. CA9 was shown to be expressed in tumors such as bladder, brain, breast, colon, lung, ovarian, pancreatic, and stomach tumors.

### Example 16

Generation and Characterization of a Humanized B7-H4 Antibody that Binds Human and Mouse B7-H4

Standard techniques were used to generate a humanized antibody 278M1 L2H2 from the 278M1 murine antibody. The heavy and light chain variable region sequences of the humanized antibody are indicated in Table 4 below.

TABLE 4

| | 278M1 | 278M1 L2H2 |
|---|---|---|
| VH | SEQ ID NO: 50 | SEQ ID NO: 66 |
| VL | SEQ ID NO: 51 | SEQ ID NO: 62 |
| VH CDR1 | TSYYMH (SEQ ID NO: 42) | TSYYMH (SEQ ID NO: 42) |
| VH CDR2 | YVDPFNGGTSYNQKFKG (SEQ ID NO: 43) | YVDPFNGGTSYNQKFKG (SEQ ID NO: 43) |
| VH CDR3 | FIAGFAN (SEQ ID NO: 44) or IAGFAN (SEQ | FIAGFAN (SEQ ID NO: 44) or IAGFAN (SEQ |
| | ID NO: 45) | ID NO: 45) |
| VL CDRI | KASQDIKSYLS (SEQ ID NO: 46) | KASQDIKSYLS (SEQ ID NO: 46) |
| VL CDR2 | YATSLAD (SEQ ID NO: 47) | YATSLAD (SEQ ID NO: 47) |
| VL CDR3 | LQHGESPYT (SEQ ID NO: 48) or | LQHGESPYT (SEQ ID NO: 48) or LQHGESPY |
| | LQHGESPY (SEQ ID NO: 49) | (SEQ ID NO: 49) |

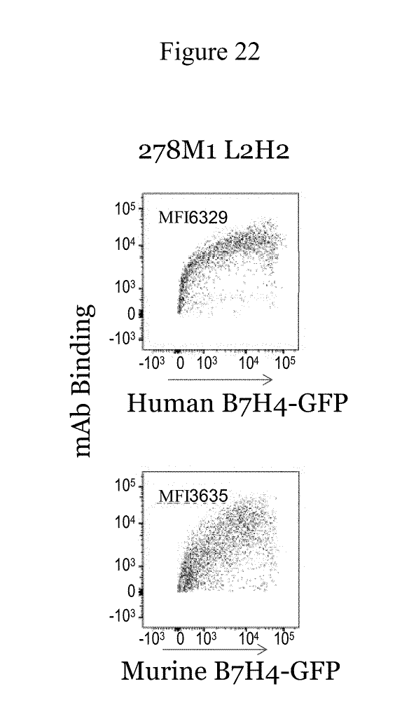

The humanized antibody 278M1 L2H2 was tested for binding to human and mouse B7-H4. HEK 293T cells were transiently transfected with cDNA expression vectors encoding cDNA for human or mouse B7H4 ECD-CD4TM-GFP. Cells were then incubated with supernatant containing the humanized anti-B7-H4 antibody, washed and stained with an APC-conjugated Fc secondary antibody. Binding of the 278M1 L2H2 antibody was then assessed by flow cytometry using a Canto II FACS instrument (BD Biosciences) and analyzed using FlowJo software. As shown in FIG. 22, analysis by flow cytometry demonstrated that the humanized antibody 278M1 L2H2 was capable of binding both human and mouse B7-H4.

### Example 17

Generation and Characterization of Antibodies that Bind Human and Mouse P-CADHERIN (CDH3)

Standard techniques were used to generate a mouse monoclonal antibody directed against CDH3. Following screening and binding, the 173M36 murine antibody was generated. From this murine antibody, two humanized antibodies were generated, 173M36 L1H2 and 173M36 L3H2. The sequences of the humanized CDH3 antibodies are indicated in Table 5 below.

TABLE 5

| | 173M36 L1H2 | 173M36 L3H2 |
|---|---|---|
| VH | SEQ ID NO: 79 | SEQ ID NO: 79 |
| VL | SEQ ID NO: 72 | SEQ ID NO: 93 |
| VH CDR1 | STYGMS (SEQ ID NO: 80) | STYGMS (SEQ ID NO: 80) |
| VH CDR2 | ATISDGGSYTYYPDSVKGR (SEQ ID | ATISDGGSYTYYPSVKGR (SEQ ID NO: 81) |
| | NO: 81) | |
| VH CDR3 | ARHYYGSDWYFDV (SEQ ID NO: 82) | ARHYYGSDWYFDV (SEQ ID NO: 82) |
| VL CDR1 | RSSQSIVQSNGNTYLE (SEQ ID NO: 73) | RSSQSIVQSNGNTYLE (SEQ ID NO: 73) |
| VL CDR2 | KVSNQFS (SEQ ID NO: 74) | KVSNQFS (SEQ ID NO: 74) |
| VL CDR3 | QGSHVPL (SEQ ID NO: 75) | QGSHVPL (SEQ ID NO: 75) |

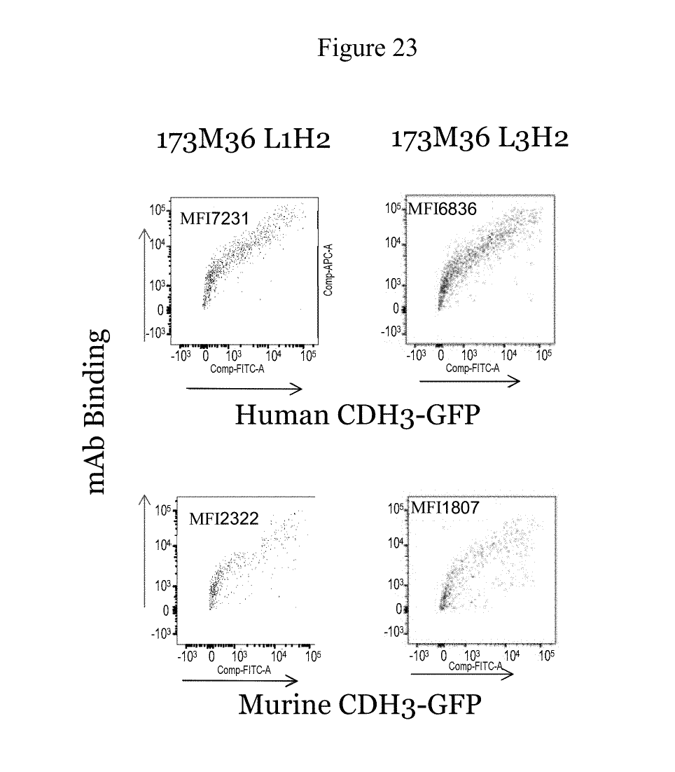

The humanized CDH3 antibodies were tested for binding to human and mouse CDH3. HEK 293T cells were transiently transfected with cDNA expression vectors encoding cDNA for human or mouse CDH3 ecd-CD4TM-GFP. Cells were then incubated with supernatant containing the humanized anti-CDH3 antibody, washed and stained with an APC-conjugated Fc secondary antibody. Binding of the 173M36 L1H2 and 173M36 L3H2 antibodies were then assessed by flow cytometry using a Canto II FACS instrument (BD Biosciences) and analyzed using FlowJo software. As shown in FIG. 23, analysis by flow cytometry demonstrated that both of the humanized CDH3 antibodies 173M36 L1H2 and 173M36 L3H2 were capable of binding both human and mouse CDH3.

### Example 18

### Generation and Characterization of Alternative Single-Chain Lymphotoxin Heterotrimer Sequences

A variety of deglycosylated human IgG1 Fc-human lymphotoxin heterotrimer fusion proteins were generated to test the three different potential orderings of the lymphotoxin-αββ and lymphotoxin-β components activity within the single-chain lymphotoxin heterotrimer. In a first construct, 363F2 (hFc-hLTαββ), the C-terminus of the human deglycosylated IgG1 Fc sequence is linked to the N-terminus of the lymphotoxin-αββ sequence which is then linked to the two lymphotoxin-β sequences. In a second construct, 363F3 (hFc-hLTβαβ), the C-terminus of the human deglycosylated IgG1 Fe sequence is linked to the N-terminus of a lymphotoxin-β-lymphotoxin-β-lymphotoxin-α-lymphotoxin-β sequence. In a third construct, 363F4 (hFc-hLTββα), the C-terminus of the human deglycosylated IgG1 Fc sequence is linked to the N-terminus of a lymphotoxin-β-lymphotoxin-β-lymphotoxin-α sequence.

To test the binding of the different c-terminal human lymphotoxin heterotrimer constructs to human and mouse LTβR, HEK 293T cells were transiently transfected with cDNA expression vectors encoding cDNA for human or mouse LTβR ecd, CD4TM and GFP. Cells were then incubated with the various human lymphotoxin constructs 363F2, 363F3, or 363F4, washed and stained with APC-conjugated Fc secondary antibody. Mouse (deglycosylated) IgG1 Fc-mouse lymphotoxin-αββ heterotrimer fusion protein 363F1 was also tested. Binding of the lymphotoxin constructs was then assessed by flow cytometry using a Canto II FACS instrument (BD Biosciences) and analyzed using FlowJo software.

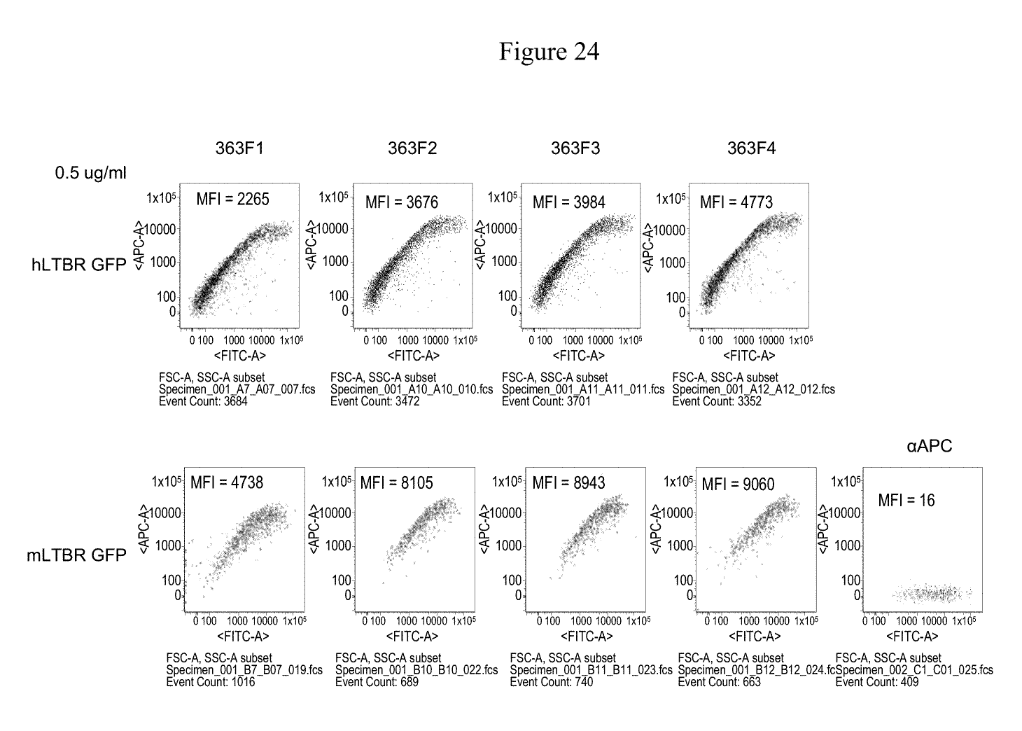

As shown in FIG. 24, all three human lymphotoxin constructs 363F2, 363F3, or 363F4, as well as the mouse lymphotoxin construct 363F1, bound both hLTβR and mLTβR well, indicating that the order of the lymphotoxin-α and lymphotoxin-β components within the single-chain lymphotoxin heterotrimer sequence is not critical for binding of LTβR. The order of lymphotoxin-αββ and lymphotoxin-β in 363F2, namely lymphotoxin-α-lymphotoxin-β-lymphotoxin-β, was selected for further studies.

The ability of various heterotrimer constructs described to activate NFκB signaling was also evaluated. Activation of NFκB signaling by LT heterotrimers was determined by an in vitro luciferase reporter assay. Each of the C-terminal heterotrimer constructs described above in this example, along with 349B1 were tested in the NFκB reporter assay.

HEK-293 cells were stably transfected with an expression vector encoding a full-length mouse LTβR as well as plasmids encoding an NFκB-dependent luciferase reporter construct. LTβR and NFκB luciferase reporter expressing HEK-293 cells were plated to 96 well plates and incubated overnight. The serially diluted recombinant fusion proteins or antibodies were added to the appropriate wells and incubated overnight. Luciferase levels were measured 18 hours later using a Steady Glo luciferase assay kit (Promega).

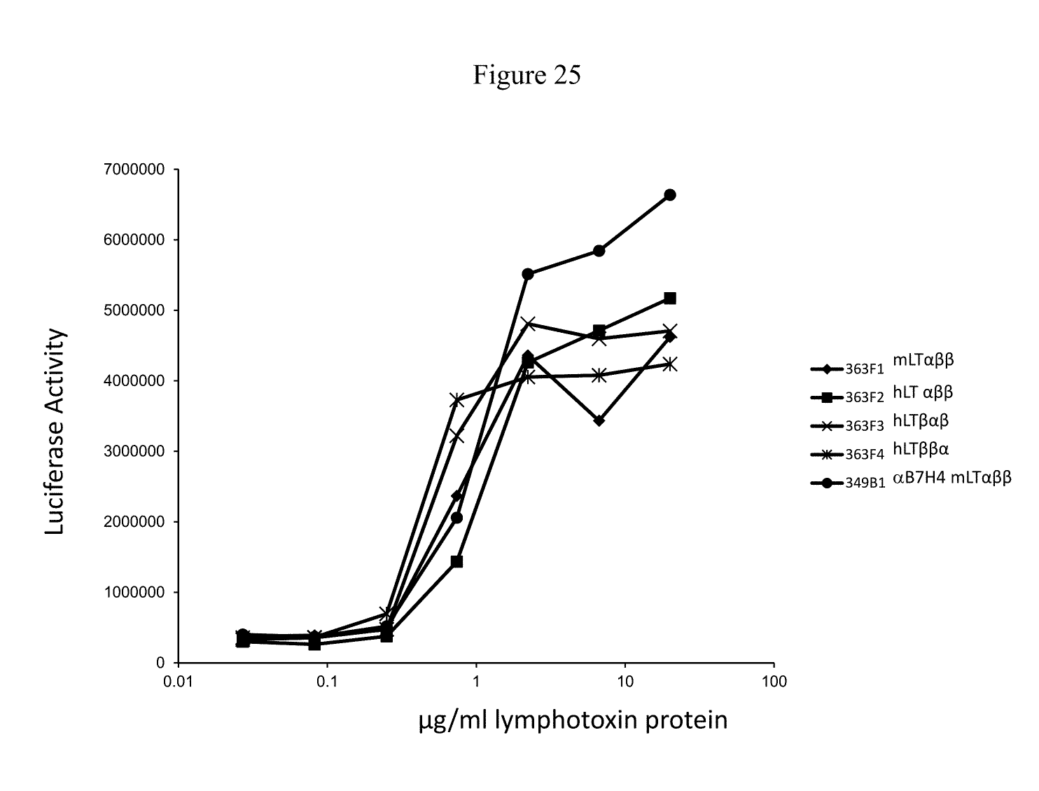

All of the lymphotoxin constructs 363F1, 363F2, 363F3, 363F4 and 349B1 were shown to have agonist activity in the NFκB reporter assay with mouse LTβR (FIG. 25). The human lymphotoxin-αββ heterotrimer sequence utilized in 363F2 was subsequently utilized in the design of the bispecific agents anti-B7-H4/lymphotoxin-αββ (349B4) and anti-CDH3/lymphotoxin-αββ (364B4) which encode humanized antibodies targeting B7-H4 and CDH3 respectively.

To test the binding ability of the 364B4 molecule to bind CHD3 and LTβR, cell-based FACS studies were conducted. Human HEK-293T cells were transiently transfected with expression vectors encoding a membrane-bound extracellular domain of mouse CDH3 (mCDH3-GFP) or human CDH3 (hCDH3-GFP), or a membrane-bound extracellular domain of mouse LTβR (mLThR-GFP) or human LTβR (hLThR-GFP). Forty-eight hours post-transfection, the cells were suspended in ice-cold HBSS containing 2% FBS and incubated in the presence of 364B4 for 30 minutes. The cells were stained with an APC-conjugated anti-human Fc secondary antibody to detect cells bound by the 364B4 molecule. Cells were incubated with an anti-APC antibody as a negative control. The cells were analyzed on a FACS Canto instrument (BD Biosciences), and the data were processed using FlowJo software.

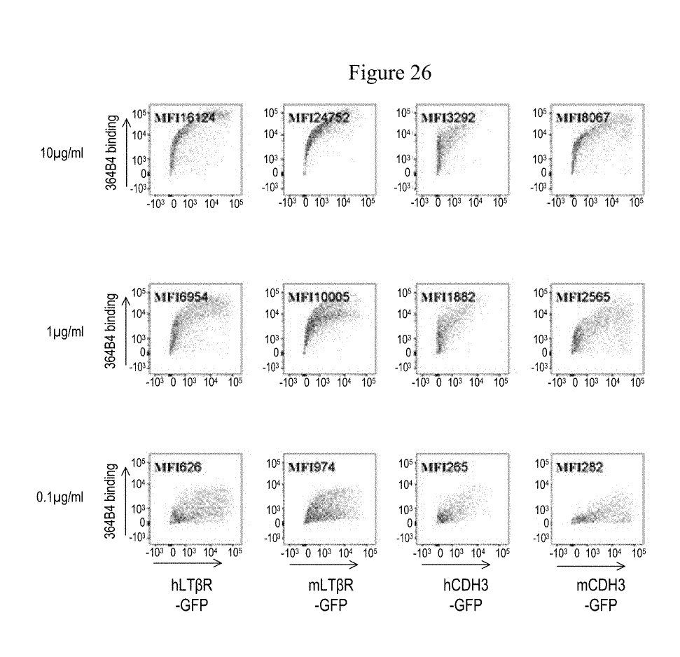

As shown in FIG. 26, 364B4 was able to bind either human or mouse CDH3 at the surface of CDH3-expressing cells. In addition, 364B4 was able to bind either human or mouse LTβR at the surface of LTβR-expressing cells. These results demonstrated that both moieties of 364B4, the anti-CDH3 antibody and the lymphotoxin-αββ trimer, formed into biologically functional structures.

It is understood that the examples and embodiments described herein are for illustrative purposes only and that various modifications or changes in light thereof will be suggested to a person skilled in the art and are to be included within the spirit and purview of this application.

All publications, patents, patent applications, internet sites, and accession numbers/database sequences including both polynucleotide and polypeptide sequences cited herein are hereby incorporated by reference herein in their entirety for all purposes to the same extent as if each individual publication, patent, patent application, internet site, or accession number/database sequence were specifically and individually indicated to be so incorporated by reference.

Sequences disclosed in the application include the following:

Mouse Lymphotoxin-alpha amino acid sequence with predicted signal
sequence underlined (SEQ ID NO: 1):
MTLLGRLHLLRVLGTPPVFLLGLLLALPLGAQGLSGVRFSAARTAHPLPQKHLTHGILKP
AAHLVGYPSKQNSLLWRASTDRAFLRHGFSLSNNSLLIPTSGLYFVYSQVVFSGESCSPR
AIPTPIYLAHEVQLFSSQYPFHVPLLSAQKSVYPGLQGPWVRSMYQGAVFLLSKGDQLST
HTDGISHLHFSPSSVFFGAFAL
Mouse Lymphotoxin-alpha amino acid sequence without predicted signal
sequence (SEQ ID NO: 2):
LSGVRFSAARTAHPLPQKHLTHGILKPAAHLVGYPSKQNSLLWRASTDRAFLRHGFSLSN
NSLLIPTSGLYFVYSQVVFSGESCSPRAIPTPIYLAHEVQLFSSQYPFHVPLLSAQKSVY
PGLQGPWVRSMYQGAVFLLSKGDQLSTHTDGISHLHFSPSSVFFGAFAL
Mouse Lymphotoxin-alpha (aa 59-202) (SEQ ID NO: 3):
KPAAHLVGYPSKQNSLLWRASTDRAFLRHGFSLSNNSLLIPTSGLYFVYSQVVFSGESCS
PRAIPTPIYLAHEVQLFSSQYPFHVPLLSAQKSVYPGLQGPWVRSMYQGAVFLLSKGDQL
STHTDGISHLHFSPSSVFFGAFAL
Mouse Lymphotoxin-beta amino acid sequence (SEQ ID NO: 4):
MGTRGLQGLGGRPQGRGCLLLAVAGATSLVTLLLAVPITVLAVLALVPQDQGRRVEKIIG
SGAQAQKRLDDSKPSCILPSPSSLSETPDPRLHPQRSNASRNLASTSQGPVAQSSREASA
WMTILSPAADSTPDPGVQQLPKGEPETDLNPELPAAHLIGAWMSGQGLSWEASQEEAFLR
SGAQFSPTHGLALPQDGVYYLYCHVGYRGRTPPAGRSRARSLTLRSALYRAGGAYGRGSP
ELLLEGAETVTPVVDPIGYGSLWYTSVGFGGLAQLRSGERVYVNISHPDMVDYRRGKTFF
GAVMVG
Mouse Lymphotoxin-beta extracellular domain (aa 49-306) amino acid
sequence (SEQ ID NO: 5):
QDQGRRVEKIIGSGAQAQKRLDDSKPSCILPSPSSLSETPDPRLHPQRSNASRNLASTSQ
GPVAQSSREASAWMTILSPAADSTPDPGVQQLPKGEPETDLNPELPAAHLIGAWMSGQGL
SWEASQEEAFLRSGAQFSPTHGLALPQDGVYYLYCHVGYRGRTPPAGRSRARSLTLRSAL
YRAGGAYGRGSPELLLEGAETVTPVVDPIGYGSLWYTSVGFGGLAQLRSGERVYVNISHP
DMVDYRRGKTFFGAVMVG
Mouse Lymphotoxin-beta aa 149-306) (SEQ ID NO: 6):
LNPELPAAHLIGAWMSGQGLSWEASQEEAFLRSGAQFSPTHGLALPQDGVYYLYCHVGYR
GRTPPAGRSRARSLTLRSALYRAGGAYGRGSPELLLEGAETVTPVVDPIGYGSLWYTSVG
FGGLAQLRSGERVYVNISHPDMVDYRRGKTFFGAVMVG
Mouse Lymphotoxin αββ single-chain trimer amino acid sequence
without a signal sequence (SEQ ID NO: 7):
KPAAHLVGYPSKQNSLLWRASTDRAFLRHGFSLSNNSLLIPTSGLYFVYSQVVFSGESCS
PRAIPTPIYLAHEVQLFSSQYPFHVPLLSAQKSVYPGLQGPWVRSMYQGAVFLLSKGDQL
STHTDGISHLHFSPSSVFFGAFALLNPELPAAHLIGAWMSGQGLSWEASQEEAFLRSGAQ
FSPTHGLALPQDGVYYLYCHVGYRGRTPPAGRSRARSLTLRSALYRAGGAYGRGSPELLL
EGAETVTPVVDPIGYGSLWYTSVGFGGLAQLRSGERVYVNISHPDMVDYRRGKTFFGAVM
VGLNPELPAAHLIGAWMSGQGLSWEASQEEAFLRSGAQFSPTHGLALPQDGVYYLYCHVG
YRGRTPPAGRSRARSLTLRSALYRAGGAYGRGSPELLLEGAETVTPVVDPIGYGSLWYTS
VGFGGLAQLRSGERVYVNISHPDMVDYRRGKTFFGAVMVG
Mouse Lymphotoxin βαβ single-chain trimer amino acid sequence
without a signal sequence (SEQ ID NO: 8):
LNPELPAAHLIGAWMSGQGLSWEASQEEAFLRSGAQFSPTHGLALPQDGVYYLYCHVGYR
GRTPPAGRSRARSLTLRSALYRAGGAYGRGSPELLLEGAETVTPVVDPIGYGSLWYTSVG
FGGLAQLRSGERVYVNISHPDMVDYRRGKTFFGAVMVGKPAAHLVGYPSKQNSLLWRAST
DRAFLRHGFSLSNNSLLIPTSGLYFVYSQVVFSGESCSPRAIPTPIYLAHEVQLFSSQYP
FHVPLLSAQKSVYPGLQGPWVRSMYQGAVFLLSKGDQLSTHTDGISHLHFSPSSVFFGAF
ALLNPELPAAHLIGAWMSGQGLSWEASQEEAFLRSGAQFSPTHGLALPQDGVYYLYCHVG
YRGRTPPAGRSRARSLTLRSALYRAGGAYGRGSPELLLEGAETVTPVVDPIGYGSLWYTS
VGFGGLAQLRSGERVYVNISHPDMVDYRRGKTFFGAVMGV
Mouse Lymphotoxin ββα single-chain trimer amino acid sequence
without a signal sequence (SEQ ID NO: 9):
LNPELPAAHLIGAWMSGQGLSWEASQEEAFLRSGAQFSPTHGLALPQDGVYYLYCHVGYR
GRTPPAGRSRARSLTLRSALYRAGGAYGRGSPELLLEGAETVTPVVDPIGYGSLWYTSVG
FGGLAQLRSGERVYVNISHPDMVDYRRGKTFFGAVMVGLNPELPAAHLIGAWMSGQGLSW
EASQEEAFLRSGAQFSPTHGLALPQDGVYYLYCHVGYRGRTPPAGRSRARSLTLRSALYR
AGGAYGRGSPELLLEGAETVTPVVDPIGYGSLWYTSVGFGGLAQLRSGERVYVNISHPDM
VDYRRGKTFFGAVMVGKPAAHLVGYPSKQNSLLWRASTDRAFLRHGFSLSNNSLLIPTSG
LYFVYSQVVFSGESCSPRAIPTPIYLAHEVQLFSSQYPFHVPLLSAQKSVYPGLQGPWVR
SMYQGAVFLLSKGDQLSTHTDGISHLHFSPSSVFFGAFAL
Human Lymphotoxin-alpha amino acid sequence with predicted signal
sequence underlined (SEQ ID NO: 10):
MTPPERLFLPRVCGTTLHLLLLGLLLVLLPGAQGLPGVGLTPSAAQTARQHPKMHLAHST
LKPAAHLIGDPSKQNSLLWRANTDRAFLQDGFSLSNNSLLVPTSGIYFVYSQVVFSGKAY
SPKATSSPLYLAHEVQLFSSQYPFHVPLLSSQEMVYPGLQEPWLHSMYHGAAFQLTQGDQ
LSTHTDGIPHLVLSPSTVFFGAFAL
Human Lymphotoxin-alpha amino acid sequence without predicted signal
sequence (SEQ ID NO: 11):
LPGVGLTPSAAQTARQHPKMHLAHSTLKPAAHLIGDPSKQNSLLWRANTDRAFLQDGFSL
SNNSLLVPTSGIYFVYSQVVFSGKAYSPKATSSPLYLAHEVQLFSSQYPFHVPLLSSQKM
VYPGLQEPWLHSMYHGAAFQLTQGDQLSTHTDGIPHLVLSPSTVFFGAFAL
Human Lymphotoxin-alpha (aa 62-205) (SEQ ID NO: 12):
KPAAHLIGDPSKQNSLLWRANTDRAFLQDGFSLSNNSLLVPTSGIYFVYSQVVFSGKAYS
PKATSSPLYLAHEVQLFSSQYPFHVPLLSSQKMVYPGLQEPWLHSMYHGAAFQLTQGDQL
STHTDGIPHLVLSPSTVFFGAFAL
Human Lymphotoxin-beta amino acid sequence (SEQ ID NO: 13):
MGALGLEGRGGRLQGRGSLLLAVAGATSLVTLLLAVPITVLAVLALVPQDQGGLVTETAD
PGAQAQQGLGFQKLPEEEPETDLSPGLPAAHLIGAPLKGQGLGWETTKEQAFLTSGTQFS
DAEGLALPQDGLYYLYCLVGYRGRAPPGGGDPQGRSVTLRSSLYRAGGAYGPGTPELLLE
GAETVTPVLDPARRQGYGPLWYTSVGFGGLVQLRRGERVYVNISHPDMVDFARGKTFFGA
VMVG
Human Lymphotoxin-beta extracellular domain 49-244) amino acid
sequence (SEQ ID NO: 14):
QDQGGLVTETADPGAQAQQGLGFQKLPEEEPETDLSPGLPAAHLIGAPLKGQGLGWETTK
EQAFLTSGTQFSDAEGLALPQDGLYYLYCLVGYRGRAPPGGGDPQGRSVTLRSSLYRAGG
AYGPGTPELLLEGAETVTPVLDPARRQGYGPLWYTSVGFGGLVQLRRGERVYVNISHPDM
VDFARGKTFFGAVMVG
Human Lymphotoxin-beta (aa 83-244) (SEQ ID NO: 15):
LSPGLPAAHLIGAPLKGQGLGWETTKEQAFLTSGTQFSDAEGLALPQDGLYYLYCLVGYR
GRAPPGGGDPQGRSVTLRSSLYRAGGAYGPGTPELLLEGAETVTPVLDPARRQGYGPLWY
TSVGFGGLVQLRRGERVYVNISHPDMVDFARGKTFFGAVMVG
Human Lymphotoxin αββ single-chain trimer amino acid sequence
without a signal sequence (SEQ ID NO: 16):
KPAAHLIGDPSKQNSLLWRANTDRAFLQDGFSLSNNSLLVPTSGIYFVYSQVVFSGKAYS
PKATSSPLYLAHEVQLFSSQYPFHVPLLSSQKMVYPGLQEPWLHSMYHGAAFQLTQGDQL
STHTDGIPHLVLSPSTVFFGAFALLSPGLPAAHLIGAPLKGQGLGWETTKEQAFLTSGTQ

FSDAEGLALPQDGLYYLYCLVGYRGRAPPGGGDPQGRSVTLRSSLYRAGGAYGPGTPELL
LEGAETVTPVLDPARRQGYGPLWYTSVGFGGLVQLRRGERVYVNISHPDMVDFARGKTFF
GAVMVGLSPGLPAAHLIGAPLKGQGLGWETTKEQAFLTSGTQFSDAEGLALPQDGLYYLY
CLVGYRGRAPPGGGDPQGRSVTLRSSLYRAGGAYGPGTPELLLEGAETVTPVLDPARRQG
YGPLWYTSVGFGGLVQLRRGERVYVNISHPDMVDFARGKTFFGAVMVG
Human Lymphotoxin βαβ single-chain trimer amino acid sequence
without a signal sequence SEQ ID NO: 17):
LSPGLPAAHLIGAPLKGQGLGWETTKEQAFLTSGTQFSDAEGLALPQDGLYYLYCLVGYR
GRAPPGGGDPQGRSVTLRSSLYRAGGAYGPGTPELLLEGAETVTPVLDPARRQGYGPLWY
TSVGFGGLVQLRRGERVYVNISHPDMVDFARGKTFFGAVMVGKPAAHLIGDPSKQNSLLW
RANTDRAFLQDGFSLSNNSLLVPTSGIYFVYSQVVFSGKAYSPKATSSPLYLAHEVQLFS
SQYPFHVPLLSSQKMVYPGLQEPWLHSMYHGAAFQLTQGDQLSTHTDGIPHLVLSPSTVF
FGAFALLSPGLPAAHLIGAPLKGQGLGWETTKEQAFLTSGTQFSDAEGLALPQDGLYYLY
CLVGYRGRAPPGGGDPQGRSVTLRSSLYRAGGAYGPGTPELLLEGAETVTPVLDPARRQG
YGPLWYTSVGFGGLVQLRRGERVYVNISHPDMVDFARGKTFFGAVMVG
Human Lymphotoxin ββα single-chain trimer amino acid sequence
without a signal sequence SEQ ID NO: 18):
LSPGLPAAHLIGAPLKGQGLGWETTKEQAFLTSGTQFSDAEGLALPQDGLYYLYCLVGYR
GRAPPGGGDPQGRSVTLRSSLYRAGGAYGPGTPELLLEGAETVTPVLDPARRQGYGPLWY
TSVGFGGLVQLRRGERVYVNISHPDMVDFARGKTFFGAVMVGLSPGLPAAHLIGAPLKGQ
GLGWETTKEQAFLTSGTQFSDAEGLALPQDGLYYLYCLVGYRGRAPPGGGDPQGRSVTLR
SSLYRAGGAYGPGTPELLLEGAETVTPVLDPARRQGYGPLWYTSVGFGGLVQLRRGERVY
VNISHPDMVDFARGKTFFGAVMVGKPAAHLIGDPSKQNSLLWRANTDRAFLQDGFSLSNN
SLLVPTSGIYFVYSQVVFSGKAYSPKATSSPLYLAHEVQLFSSQYPFHVPLLSSQKMVYP
GLQEPWLHSMYHGAAFQLTQGDQLSTHTDGIPHLVLSPSTVFFGAFAL
Human IgG1 Heavy chain constant region (SEQ ID NO: 19):
ASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKKVEPKSCDKTHTCPPCPAPELLGG
PSVFLFPPKPKDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQVYTLPPSRDE
LTKNQVSLTCLVKGFYPSDIAVEWESNGQPENNYKTTPPVLDSDGSFFLYSKLTVDKSRW
QQGNVFSCSVMHEALHNHYTQKSLSLSPGK
Human IgG1 Heavy chain constant region deglycosylated (SEQ ID NO: 20):
ASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKKVEPKSCDKTHTCPPCPAPELLGG
PSVFLFPPKPKDTLMISRTPEVTCVVVDVSHEDPEVKFNWYVDGVEVHNAKTKPREEQYA
STYRVVSVLTVLHQDWLNGKEYKCKVSNKALPAPIEKTISKAKGQPREPQVYTLPPSRDE
LTKNQVSLTCLVKGFYPSDIAVEWESNGQPENNYKTTPPVLDSDGSFFLYSKLTVDKSRW
QQGNVFSCSVMHEALHNHYTQKSLSLSPGK
Human IgG2 Heavy chain constant region (SEQ ID NO: 21):
ASTKGPSVFPLAPCSRSTSESTAALGCLVKDYFPEPVTVSWNSGALTSGVHTFPAVLQSS
GLYSLSSVVTVPSSNFGTQTYTCNVDHKPSNTKVDKTVERKCCVECPPCPAPPVAGPSVF
LFPPKPKDTLMISRTPEVTCVVVDVSHEDPEVQFNWYVDGVEVHNAKTKPREEQFNSTFR
VVSVLTVVHQDWLNGKEYKCKVSNKGLPAPIEKTISKTKGQPREPQVYTLPPSREEMTKN
QVSLTCLVKGFYPSDIAVEWESNGQPENNYKTTPPMLDSDGSFFLYSKLTVDKSRWQQGN
VFSCSVMHEALHNHYTQKSLSLSPGK
Human IgG2 Heavy chain constant region deglycosylated (SEQ ID NO: 22):
ASTKGPSVFPLAPCSRSTSESTAALGCLVKDYFPEPVTVSWNSGALTSGVHTFPAVLQSS
GLYSLSSVVTVPSSNFGTQTYTCNVDHKPSNTKVDKTVERKCCVECPPCPAPPVAGPSVF
LFPPKPKDTLMISRTPEVTCVVVDVSHEDPEVQFNWYVDGVEVHNAKTKPREEQFASTFR
VVSVLTVVHQDWLNGKEYKCKVSNKGLPAPIEKTISKTKGQPREPQVYTLPPSREEMTKN
QVSLTCLVKGFYPSDIAVEWESNGQPENNYKTTPPMLDSDGSFFLYSKLTVDKSRWQQGN
VFSCSVMHEALHNHYTQKSLSLSPGK
Human IgG3 Heavy chain constant region (SEQ ID NO: 23):
ASTKGPSVFPLAPCSRSTSGGTAALGCLVKDYFPEPVTVSWNSGALTSGVHTFPAVLQSS
GLYSLSSVVTVPSSSLGTQTYTCNVNHKPSNTKVDKRVELKTPLGDTTHTCPRCPEPKSC
DTPPPCPRCPEPKSCDTPPPCPRCPEPKSCDTPPPCPRCPAPELLGGPSVFLFPPKPKDT
LMISRTPEVTCVVVDVSHEDPEVQFKWYVDGVEVHNAKTKPREEQYNSTFRVVSVLTVLH
QDWLNGKEYKCKVSNKALPAPIEKTISKTKGQPREPQVYTLPPSREEMTKNQVSLTCLVK
GFYPSDIAVEWESSGQPENNYNTTPPMLDSDGSFFLYSKLTVDKSRWQQGNIFSCSVMHE
ALHNRFTQKSLSLSPGK
Human IgG3 Heavy chain constant region deglycosylated (SEQ ID NO: 24):
ASTKGPSVFPLAPCSRSTSGGTAALGCLVKDYFPEPVTVSWNSGALTSGVHTFPAVLQSS
GLYSLSSVVTVPSSSLGTQTYTCNVNHKPSNTKVDKRVELKTPLGDTTHTCPRCPEPKSC
DTPPPCPRCPEPKSCDTPPPCPRCPEPKSCDTPPPCPRCPAPELLGGPSVFLFPPKPKDT
LMISRTPEVTCVVVDVSHEDPEVQFKWYVDGVEVHNAKTKPREEQYASTFRVVSVLTVLH
QDWLNGKEYKCKVSNKALPAPIEKTISKTKGQPREPQVYTLPPSREEMTKNQVSLTCLVK
GFYPSDIAVEWESSGQPENNYNTTPPMLDSDGSFFLYSKLTVDKSRWQQGNIFSCSVMHE
ALHNRFTQKSLSLSPGK
Human IgG4 Heavy chain constant region (SEQ ID NO: 25):
ASTKGPSVFPLAPCSRSTSESTAALGCLVKDYFPEPVTVSWNSGALTSGVHTFPAVLQSS
GLYSLSSVVTVPSSSLGTKTYTCNVDHKPSNTKVDKRVESKYGPPCPSCPAPEFLGGPSV
FLFPPKPKDTLMISRTPEVTCVVVDVSQEDPEVQFNWYVDGVEVHNAKTKPREEQFNSTY
RVVSVLTVLHQDWLNGKEYKCKVSNKGLPSSIEKTISKAKGQPREPQVYTLPPSQEEMTK
NQVSLTCLVKGFYPSDIAVEWESNGQPENNYKTTPPVLDSDGSFFLYSRLTVDKSRWQEG
NVFSCSVMHEALHNHYTQKSLSLSLGK
Human IgG4 Heavy chain constant region deglycosylated (SEQ ID NO: 26):
ASTKGPSVFPLAPCSRSTSESTAALGCLVKDYFPEPVTVSWNSGALTSGVHTFPAVLQSS
GLYSLSSVVTVPSSSLGTKTYTCNVDHKPSNTKVDKRVESKYGPPCPSCPAPEFLGGPSV
FLFPPKPKDTLMISRTPEVTCVVVDVSQEDPEVQFNWYVDGVEVHNAKTKPREEQFASTY
RVVSVLTVLHQDWLNGKEYKCKVSNKGLPSSIEKTISKAKGQPREPQVYTLPPSQEEMTK
NQVSLTCLVKGFYPSDIAVEWESNGQPENNYKTTPPVLDSDGSFFLYSRLTVDKSRWQEG
NVFSCSVMHEALHNHYTQKSLSLSLGK
Human IgG1 Fc region (SEQ ID NO: 27):
DKTHTCPPCPAPELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPAPIEKTISKAK
GQPREPQVYTLPPSRDELTKNQVSLTCLVKGFYPSDIAVEWESNGQPENNYKTTPPVLDS
DGSFFLYSKLTVDKSRWQQGNVFSCSVMHEALHNHYTQKSLSLSPGK
Human IgG1 Fc region deglycosylated (SEQ ID NO: 28):
DKTHTCPPCPAPELLGGPSVFLFPPKPKDTLMISRTPEVTCVVVDVSHEDPEVKFNWYVD
GVEVHNAKTKPREEQYASTYRVVSVLTVLHQDWLNGKEYKCKVSNKALPAPIEKTISKAK
GQPREPQVYTLPPSRDELTKNQVSLTCLVKGFYPSDIAVEWESNGQPENNYKTTPPVLDS
DGSFFLYSKLTVDKSRWQQGNVFSCSVMHEALHNHYTQKSLSLSPGK
Human IgG1 Fc region (13A Version) (SEQ ID NO: 29):
DKTHTCPPCPAPELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPAPIEKTISKAK
GQPREPQVYTLPPSRDKLTKNQVSLTCLVKGFYPSDIAVEWESNGQPENNYKTTPPVLKS
DGSFFLYSKLTVDKSRWQQGNVFSCSVMHEALHNHYTQKSLSLSPGK
Human IgG1 Fc region (13B Version) (SEQ ID NO: 30):
DKTHTCPPCPAPELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPAPIEKTISKAK
GQPREPQVYTLPPSRDELTKNQVSLTCLVEGFYPSDIAVEWESNGQPENNYKTTPPVLDS
DGSFFLYSELTVDKSRWQQGNVFSCSVMHEALHNHYTQKSLSLSPGK
Human IgG2 Fc region (SEQ ID NO: 31):
CVECPPCPAPPVAGPSVFLFPPKPKDTLMISRIPEVTCVVVDVSHEDPEVQFNWYVDGVE
VHNAKTKPREEQFNSTFRVVSVLTVVHQDWLNGKEYKCKVSNKGLPAPIEKTISKTKGQP
REPQVYTLPPSREEMTKNQVSLTCLVKGFYPSDIAVEWESNGQPENNYKTTPPMLDSDGS
FFLYSKLTVDKSRWQQGNVFSCSVMHEALHNHYTQKSLSLSPGK
Human IgG2 Fc region deglycosylated (SEQ ID NO: 32):
CVECPPCPAPPVAGPSVFLFPPKPKDTLMISRTPEVTCVVVDVSHEDPEVQFNWYVDGVE
VHNAKTKPREEQFASTFRVVSVLTVVHQDWLNGKEYKCKVSNKGLPAPIEKTISKTKGQP
REPQVYTLPPSREEMTKNQVSLTCLVKGFYPSDIAVEWESNGQPENNYKTTPPMLDSDGS
FFLYSKLTVDKSRWQQGNVFSCSVMHEALHNHYTQKSLSLSPGK
Human IgG2 Fc region (13A Version) (SEQ ID NO: 33):
CVECPPCPAPPVAGPSVFLFPPKPKDTLMISRTPEVTCVVVDVSHEDPEVQFNWYVDGVE
VHNAKTKPREEQFNSTFRVVSVLTVVHQDWLNGKEYKCKVSNKGLPAPIEKTISKTKGQP
REPQVYTLPPSREKMTKNQVSLTCLVKGFYPSDIAVEWESNGQPENNYKTTPPMLKSDGS
FFLYSKLTVDKSRWQQGNVFSCSVMHEALHNHYTQKSLSLSPGK
Human IgG2 Fc region (13B Version) (SEQ ID NO: 34):
CVECPPCPAPPVAGPSVFLFPPKPKDTLMISRTPEVTCVVVDVSHEDPEVQFNWYVDGVE
VHNAKTKPREEQFNSTFRVVSVLTVVHQDWLNGKEYKCKVSNKGLPAPIEKTISKTKGQP
REPQVYTLPPSREEMTKNQVSLTCLVEGFYPSDIAVEWESNGQPENNYKTTPPMLDSDGS
FFLYSELTVDKSRWQQGNVFSCSVMHEALHNHYTQKSLSLSPGK
FLAG Tag (SEQ ID NO: 35):
DYKDDDDK.
349B1 Anti-B7-H4 deglycosylated mIgG1-mLTαββ with signal sequence
underlined (SEQ ID NO: 36):
MKHLWFFLLLVAAPRWVLSQVQLQQSGSELVRPGASVKLSSKALGYTFTDYEMHWVKQTP
VHGLEWIGTIHPGTGGTAYNQKFKGKATLTADKSSSTAYMELSSLTSEDSAVYYFTNLDN
WGQGTTLTVSSAKTTPPSVYPLAPGSAAQTNSMVTLGCLVKGYFPEPVTVTWNSGSLSSG
VHTFPAVLQSDLYTLSSSVTVPSSTWPSETVTCNVAHPASSTKVDKKIVPRDCGCKPCIC
TVPEVSSVFIFPPKPKDVLTITLTPKVTCVVVDISKDDPEVQFSWFVDDVEVHTAQTQPR
EEQFASTFRSVSELPIMHQDWLNGKEFKCRVNSAAFPAPIEKTISKTKGRPKAPQVYTIP
PPKEQMAKDKVSLTCMITDFFPEDITVEWQWNGQPAENYKNTQPIMDTDGSYFVYSKLNV
QKSNWEAGNTFTCSVLHEGLHNHHTEKSLSHSPGKKPAAHLVGYPSKQNSLLWRASTDRA
FLRHGFSLSNNSLLIPTSGLYFVYSQVVFSGESCSPRAIPTPIYLAHEVQLFSSQYPFHV
PLLSAQKSVYPGLQGPWVRSMYQGAVFLLSKGDQLSTHTDGISHLHFSPSSVFFGAFALL
NPELPAAHLIGAWMSGQGLSWEASQEEAFLRSGAQFSPTHGLALPQDGVYYLYCHVGYRG
RTPPAGRSRARSLTLRSALYRAGGAYGRGSPELLLEGAETVTPVVDPIGYGSLWYTSVGF
GGLAQLRSGERVYVNISHPDMVDYRRGKTFFGAVMVGLNPELPAAHLIGAWMSGQGLSWE
ASQEEAFLRSGAQFSPTHGLALPQDGVYYLYCHVGYRGRTPPAGRSRARSLTLRSALYRA
GGAYGRGSPELLLEGAETVTPVVDPIGYGSLWYTSVGFGGLAQLRSGERVYVNISHPDMV
DYRRGKTFFGAVMVG
349B1 Anti-B7-H4 deglycosylated mIgG1-mLTαββ without signal sequence
(SEQ ID NO: 37):
QVQLQQSGSELVRPGASVKLSSKALGYTFTDYEMHWVKQTPVHGLEWIGTIHPGTGGTAY
NQKFKGKATLTADKSSSTAYMELSSLTSEDSAVYYFTNLDNWGQGTTLTVSSAKTTPPSV
YPLAPGSAAQTNSMVTLGCLVKGYFPEPVTVTWNSGSLSSGVHTFPAVLQSDLYTLSSSV
TVPSSTWPSETVTCNVAHPASSTKVDKKIVPRDCGCKPCICTVPEVSSVFIFPPKPKDVL
TITLTPKVTCVVVDISKDDPEVQFSWFVDDVEVHTAQTQPREEQFASTFRSVSELPIMHQ
DWLNGKEFKCRVNSAAFPAPIEKTISKTKGRPKAPQVYTIPPPKEQMAKDKVSLTDMITD
FFPEDITVEWQWNGQPAENYKNTQPIMDTDGSYFVYSKLNVQKSNWEAGNTFTCSVLHEG
LHNHHTEKSLSHSPGKKPAAHLVGYPSKQNSLLWRASTDRAFLRHGFSLSNNSLLIPTSG
LYFVYSQVVFSGESCSPRAIPTPIYLAHEVQLFSSQYPFHVPLLSAQKSVYPGLQGPWVR
SMYQGAVFLLSKGDQLSTHTDGISHLHFSPSSVFFGAFALLNPELPAAHLIGAWMSGQGL
SWEASQEEAFLRSGAQFSPTHGLALPQDGVYYLYCHVGYRGRTPPAGRSRARSLTLRSAL
YRAGGAYGRGSPELLLEGAETVTPVVDPIGYGSLWYTSVGFGGLAQLRSGERVYVNISHP
DMVDYRRGKTFFGAVMVGLNPELPAAHLIGAWMSGQGLSWEASQEEAFLRSGAQFSPTHG
LALPQDGVYYLYCHVGYRGRTPPAGRSRARSLTLRSALYRAGGAYGRGSPELLLEGAETV
TPVVDPIGYGSLWYTSVGFGGLAQLRSGERVYVNISHPDMVDYRRGKTFFGAVMVG
Human IgG1 Fc region deglycosylate (13A Version) (SEQ ID NO: 38):
DKTHTCPPCPAPELLGGPSVFLFPPKPKDTLMISRTPEVTCVVVDVSHEDPEVKFNWYVD
GVEVHNAKTKPREEQYASTYRVVSVLTVLHQDWLNGKEYKCKVSNKALPAPIEKTISKAK
GQPREPQVYTLPPSRDKLTKNQVSLTCLVKGFYPSDIAVEWESNGQPENNYKTTPPVLKS
DGSFFLYSKLTVDKSRWQQGNVFSCSVMHEALHNHYTQKSLSLSPGK
Human IgG1 Fc region deglycosylate (13B Version) (SEQ ID NO: 39):
DKTHTCPPCPAPELLGGPSVFLFPPKPKDTLMISRTPEVTCVVVDVSHEDPEVKFNWYVD
GVEVHNAKTKPREEQYASTYRVVSVLTVLHQDWLNGKEYKCKVSNKALPAPIEKTISKAK
GQPREPQVYTLPPSRDELTKNQVSLTCLVEGFYPSDIAVEWESNGQPENNYKTTPPVLDS
DGSFFLYSELTVDKSRWQQGNVFSCSVMHEALHNHYTQKSLSLSPGK
Human IgG2 Fc region deglycosylated (13AVersion) (SEQ ID NO: 40):
CVECPPCPAPPVAGPSVFLFPPKPKDTLMISRTPEVTCVVVDVSHEDPEVQFNWYVDGVE
VHNAKTKPREEQFASTFRVVSVLTVVHQDWLNGKEYKCKVSNKGLPAPIEKTISKTKGQP
REPQVYTLPPSREKMTKNQVSLTCLVKGFYPSDIAVEWESNGQPENNYKTTPPMLKSDGS
FFLYSKLTVDKSRWQQGNVFSCSVMHEALHNHYTQKSLSLSPGK
Human IgG2 Fc region deglycosylated (13B Version) (SEQ ID NO: 41):
CVECPPCPAPPVAGPSVFLFPPKPKDTLMISRTPEVTCVVVDVSHEDPEVQFNWYVDGVE
VHNAKTKPREEQFNSTFRVVSVLTVVHQDWLNGKEYKCKVSNKGLPAPIEKTISKTKGQP
REPQVYTLPPSREKMTKNQVSLTCLVKGFYPSDIAVEWESNGQPENNYKTTPPMLKSDGS
FFLYSELTVDKSRWQQGNVFSCSVMHEALHNHYTQKSLSLSPGK
278M1 heavy chain CDR1 (SEQ ID NO: 42):
ISYYMH
278M1 heavy chain CDR2 (SEQ ID NO: 43):
YVDPFNGGTSYNQKFKG
278M1 heavy chain CDR3 'SEQ ID NO: 44):
FIAGFAN
278M1 heavy chain CDR3 (SEQ ID NO: 45):
IAGFAN
278M1 light chain CDR+ (SEQ ID NO: 46):
KASQDIKSYLS
278M1 light chain CDR2 (SEQ ID NO: 47):
YATSLAD
278M1 light chain CDR3 (SEQ ID NO: 48):
LQHGESPYT
278M1 light chain CDR3 (SEQ ID NO: 49):
LQHGESPY
278M1 heavy chain variable sequence (without signal sequence)
(SEQ ID NO: 50):
QVQLQQSGAELMKPGASVKISCKASDYSFTSYYMHWVKQSHGKSLEWVGYVDPFNGGTSYNQKFKGKATL
TVDKSSSTAYMHLSSLTSEDSGVYYCAFIAGFANWGQGTLVTVSA
278M1 light chain variable sequence (without signal sequence)
(SEQ ID NO: 51):
DIVMTQSPSSMYASLGERVTITCKASQDIKSYLSWYQQKPWKSPKTLIYYATSLADGVPSR
FSGSGSGQDFSLTISSLESDDTATYYCLQHGESPYTFGGGTKLEIK
278M1 heavy chain sequence (without signal sequence)(SEQ ID NO: 52):
QVQLQQSGAELMKPGASVKISCKASDYSFTSYYMHWVKQSHGKSLEWVGYVDPFNGGTSYNQKFKGKATL
TVDKSSSTAYMHLSSLTSEDSGVYYCAFIAGFANWGQGTLVTVSAAKTTPPSVYPLAPGSAAQTNSMVTL
GCLVKGYFPEPVTVTWNSGSLSSGVHTFPAVLQSDLYTLSSSVTVPSSTWPSETVTCNVAHPASSTKVDK
KIVPRDCGCKPCICTVPEVSSVFIFPPKPKDVLTITLTPKVTCVVVDISKDDPEVQFSWFVDDVEVHTAQ
TQPREEQFNSTFRSVSELPIMHQDWLNGKEFKCRVNSAAFPAPIEKTISKTKGRPKAPQVYTIPPPKEQM
AKDKVSLTCMITDFFPEDITVEWQWNGQPAENYENTQPIMDTDGSYFVYSKLNVQKSNWEAGNTFTCSVL
HEGLHNHHTEKSLSHSPGK
278M1 light chain sequence (without signal sequence)(SEQ ID NO: 53):
DIVMTQSPSSMYASLGERVTITCKASQDIKSYLSWYQQKPWKSPKTLIYYATSLADGVPSRFSGSGSGQD
FSLTISSLESDDTATYYCLQHGESPYTFGGGTKLEIKRADAAPTVSIFPPSSEQLTSGGASVVCFLNNFY
PKDINVKWKIDGSERQNGVLNSWTDQDSKDSTYSMSSTLTLTKDEYERHNSYTCEATHKTSTSPIVKSFN
RNEC
278M1 heavy chain sequence (signal sequence underlined)(SEQ ID NO: 54):
MKHLWFFLLLVAAPRWVLSQVQLQQSGAELMEPGASVKISCKASDYSFTSYYMHWVKQSHGKSLEWVGYV
DPFNGGTSYNQKFKGKATLTVDKSSSTAYMHLSSLTSEDSGVYYCAFIAGFANWGQGTLVTVSAAKTTPP
SVYPLAPGSAAQTNSMVTLGCLVKGYFPEPVTVTWNSGSLSSGVHTFPAVLQSDLYTLSSSVTVPSSTWP
SETVTCNVAHPASSTKVDKKIVPRDCGCKPCICTVPEVSSVFIFPPKPKDVLTITLTPKVTCVVVDISKD
DPEVQFSWFVDDVEVHTAQTQPREEQFNSTFRSVSELPIMHQDWLNGKEFKCRVNSAAFPAPIEKTISKT
KGRPKAPQVYTIPPPKEQMAKDKVSLTCMITDFFPEDITVEWQWNGQPAENYKNTQPIMDTDGSYFVYSK
LNVQKSNWEAGNTFTCSVLHEGLHNHHTEKSLSHSPGK
278M1 light chain sequence (signal sequence underlined)(SEQ ID NO: 55):
MKHLWFFLLLVAAPRWVLSDIVMTQSPSSMYASLGERVTITCKASQDIKSYLSWYQQKPWKSPKTLIYYA
TSLADGVPSRFSGSGSGQDFSLTISSLESDDTATYYCLQHGESPYTFGGGTKLEIKRADAAPTVSIFPPS
SEQLTSGGASVVCFLNNFYPKDINVKWKIDGSERQNGVLNSWTDQDSKDSTYSMSSTLTLTKDEYERHNS
YTCEATHKTSTSPIVKSFNRNEC
278M1-LC2 humanized light chain with signal sequence (signal sequence
underlined)(SEQ ID NO: 59):
MVLQTQVFISLLLWISGAYGDIQMTQSPSSLSASVGDRVTITCKASQDIKSYLSWYQQKP
GKAPKTLIYYATSLADGVPSRFSGSGSGTDFTLTISSLQPEDFATYYCLQHGESPYTFGG
GTKVEIKRTVAAPSVFIFPPSDEQLKSGTASVVCLLNNFYPREAKVQWKVDNALQSGNSQ
ESVTEQDSKDSTYSLSSTLTLSKADYEKHKVYACEVTHQGLSSPVTKSFNRGEC
278M1-LC2 humanized light chain without signal sequence (SEQ ID NO: 60):
DIQMTOSPSSLSASVGDRVTITCKASQDIKSYLSWYQQKPGKAPKTLIYYATSLADGVPS
RFSGSGSGTDFTLTISSLQPEDFATYYCLQHGESPYTFGGGTKVEIKRTVAAPSVFIFPP
SDEQLKSGTASVVCLLNNFYPREAKVQWKVDNALQSGNSQESVTEQDSKDSTYSLSSTLT
LSKADYEKHKVYACEVTHQGLSSPVTKSFNRGEC
278M1-VL2 humanized light chain variable domain with the signal sequence
(signal sequence underlined)(SEQ ID NO: 61):
MVLQTQVFISLLLWISGAYGDIQMTQSPSSLSASVGDRVTITCKASQDIKSYLSWYQQKPGKAPKTLIYY
ATSLADGVPSRFSGSGSGTDFTLTISSLQPEDFATYYCLQHGESPYTFGGGTKVEIKR
278M1-VL2 humanized light chain variable domain without signal sequence
(SEQ ID NO: 62):
DIQMTQSPSSLSASVGDRVTITCKASQDIKSYLSWYQQKPGKAPKTLIYYATSLADGVPS
RFSGSGSGTDFTLTISSLQPEDFATYYCLQHGESPYTFGGGTKVEIKR
278M1_VH2-IgG1 humanized heavy chain with signal sequence (signal sequence
underlined)(SEQ ID NO: 63):
MDWTWRILFLVAAATGAHSQVQLVQSGAEVKKPGASVKVSCKASGYTFTSYYMHWVRQAPGQRLEWMGYV
DPFNGGTSYNQKFKGRVTITVDTSSSTAYMELSSLRSEDTAVYYCAFIAGFANWGQGTLVTVSSASTKG
PASTKGPSVFPLAPSSKSTSGGTAALGCLVKDYFPFPVTVSWNSGALTSGVHTFPAVLQSSGLYSLSSVV
TVPSSSLGTQTYICNVNHKPSNTKVDKRVEPKSCDKTHTCPPCPAPELLGGPSVFLFPPKPKDTLMISRT
PEVTCVVVDVSHEDPEVKFNWYVDGVEVHNAKTKPREEQYNSTYRVVSVLTVLHQDWLNGKEYKCKVSNK
ALPAPIEKTISKAKGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK
278M1_VH2-IgG1 humanized heavy chain without signal sequence (SEQ ID NO: 64):
QVQLVQSGAEVKKPGASVKVSCKASGYTFTSYYMHWVRQAPGQRLEWMGYVDPFNGGTSY
NQKFKGRVTITVDTSSSTAYMELSSLRSEDTAVYYCAFIAGFANWGQGTLVTVSSASTKG
PASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKRVEPKSCDKTHTCPPCPAPELLGG
PSVFLFPPKPKDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQVYTLPPSREE
MTKNQVSLTCLVKGFYPSDIAVEWESNGQPENNYKTTPPVLDSDGSFFLYSKLTVDKSRW
QQGNVFSCSVMHEALHNHYTQKSLSLSPGK
278M1-VH42 humanized heavy chain variable domain with the signal sequence
(signal sequence underlined) (SEQ ID NO: 65):
MDWTWRILFLVAAATGAHSQVQLVQSGAEVKKPGASVKVSCKASGYTFTSYYMHWVRQAP
GQRLEWMGYVDPFNGGTSYNQKFKGRVTITVDTSSSTAYMELSSLRSEDTAVYYCAFIAG
FANWGQGTLVTVSS
278M1-VH2 humanized heavy chain variable domain without signal sequence
(SEQ ID NO: 66):
QVQLVQSGAEVKKPGASVKVSCKASGYTFTSYYMHWVRQAPGQRLEWMGYVDPFNGGTSY
NQKFKGRVTITVDTSSSTAYMELSSLRSEDTAVYYCAFIAGFANWGQGTLVTVSSASTKG
P
173M36 LC1 humanized light chain with signal sequence signal sequence
underlined) (SEQ ID NO: 67):
MVLQTQVFISLLLWISGAYGDIVMTQSPLSLPVTPGEPASISCRSSQSIVQSNGNTYLEW
YLQKPGQSPQLLIYKVSNQFSGVPDRFSGSGSGTDFTLKISRVEAEDVGVYYCFQGSHVP
LTFGQGTKVEIKRTVAAPSVFIFPPSDEQLKSGTASVVCLLNNFYPREAKVQWKVDNALQ
SGNSQESVTEQDSKDSTYSLSSTLTLSKADYEKHKVYACEVTHQGLSSPVTKSFNRGEC
173M36 LC1 humanized light chain without signal sequence (SEQ ID NO: 68):
DIVMTQSPLSLPVTPGEPASISCRSSQSIVQSNGNTYLEWYLQKPGQSPQLLIYKVSNQF
SGVPDRFSGSGSGTDFTLKISRVEAEDVGVYYCFQGSHVPLTFGQGTKVEIKRTVAAPSV
FIFPPSDEQLKSGTASVVCLLNNFYPREAKVQWKVDNALQSGNSQESVTEQDSKDSTYSL
SSTLTLSKADYEKHKVYACEVTHQGLSSPVTKSFNRGEC
173M36 LC3 humanized light chain with signal sequence (signal sequence
underlined) (SEQ ID NO: 69):
MVLQTQVFISLLLWISGAYGDIVMTQSPSSLPVTPGEPASISCRSSQSIVQSNGNTYLEW
YLQKPGQSPQLLLYKVSNQFSGVPDRISGSGSGTDFTLKISRVEAEDVGVYYCFQGSHVP
LTFGQGTKVEIKRTVAAPSVFIFPPSDEQLKSGTASVVCLLNNFYPREAKVQWKVDNALQ
SGNSQESVTEQDSKDSTYSLSSTLTLSKADYEKHKVYACEVTHQGLSSPVTKSFNRGEC
173M36 LC3 humanized light chain without signal sequence (SEQ ID NO: 70):
DIVMTQSPSSLPVTPGEPASISCRSSQSIVQSNGNTYLEWYLQKPGQSPQLLLYKVSNQF
SGVPDRISGSGSGTDFTLKISRVEAEDVGVYYCFQGSHVPLTFGQGTKVEIKRTVAAPSV
FIFPPSDEQLKSGTASVVCLLNNFYPREAKVQWKVDNALQSGNSQESVTEQDSKDSTYSL
SSTLTLSKADYEKHKVYACEVTHQGLSSPVTKSFNRGEC
173M36 VL1 light chain variable domain with the signal sequence (signal
sequence underlined) (SEQ ID NO: 71):
MVLQTQVFISLLLWISGAYGDIVMTQSPLSLPVTPGEPASISCRSSQSIVQSNGNTYLEWYLQKPGQSPQ
LLIYKVSNQFSGVPDRFSGSGSGTDFTLKISRVEAEDVGVYYCFQGSHVPLTFGQGTKVEIKR
173M36 VL1 light chain variable domain without signal sequence
(SEQ ID NO: 72):
DIVMTQSPLSLPVTPGEPASISCRSSQSIVQSNGNTYLEWYLQKPGQSPQLLIYKVSNQF
SGVPDRFSGSGSGTDFTLKISRVEAEDVGVYYCFQGSHVPLTFGQGTKVEIKR
173M36 VL3 light chain variable domain with the signal sequence (signal
sequence underlined) (SEQ ID NO: 92):
MVLQTQVFISLLLWISGAYDIVMTQSPSSLPVTPGEPASISCRSSQSIVQSNGNTYLEWYLQKPGQSPQL
LLYKVSNQFSGVPDRISGSGSGTDFTLKISRVEAEDVGVYYCFQGSHVPLTFGQGTKVEIKR
173M36 VL3 light chain variable domain without signal sequence
(SEQ ID NO: 93):
DIVMTQSPSSLPVTPGEPASISCRSSQSIVQSNGNTYLEWYLQKPGQSPQLLLYKVSNQFSGVPDRISGS
GSGTDFTLKISRVEAEDVGVYYCFQGSHVPLTFGQGTKVEIKR
173M36 LC CDR1 (SEQ ID NO: 73):
RSSQSIVQSNGNTYLE
173M36 LC CDR2 (SEQ ID NO: 74):
KVSNQFS
173M36 LC CDR3 (SEQ ID NO: 75):
QGSHVPL
173M36_VH2-IgG1 humanized heavy chain with signal sequence (signal sequence
underlined) (SEQ ID NO: 76):
MDWTWRILFLVAAATGAHSEVQLVQSGGGLVKPGGSLRLSCAASGFTFSTYGMSWVRQAPGKGLEWVATI
SDGGSYTYYPDSVKGRFTISRDNAKNSLYLQMNSLRAEDTAVYYCARHYYGSDWYFDVWGQGTTVTVSSA
STKGPSVFPLAPSSKSTSGGTAALGCLVKDYFPEPVTVSWNSGALTSGVHTFPAVLQSSGLYSLSSVVTV
PSSSLGTQTYICNVNHKPSNTKVDKRVEPKSCDKTHTCPPCPAPELLGGPSVFLFPPKPKDTLMISRTPE
VTCVVVDVSHEDPEVKFNWYVDGVEVHNAKTKPREEQYNSTYRVVSVLTVLHQDWLNGKEYKCKVSNKAL
PAPIEKTISKAKGQPREPQVYTLPPSREEMTKNQVSLTCLVKGFYPSDIAVEWESNGQPENNYKTTPPVL
DSDGSFFLYSKLTVDKSRWQQGNVFSCSVMHEALHNHYTQKSLSLSPGK
173M36_VH2-IgG1 humanized heavy chain without signal sequence
(SEQ ID NO: 77):
EVQLVQSGGGLVKPGGSLRLSCAASGFTFSTYGMSWVRQAPGKGLEWVATISDGGSYTYY
PDSVKGRFTISRDNAKNSLYLQMNSLRAEDTAVYYCARHYYGSDWYFDVWGQGTTVTVSS
ASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKRVEPKSCDKTHTCPPCPAPELLGG
PSVFLFPPKPKDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQVYTLPPSREE
MTKNQVSLTCLVKGFYPSDIAVEWESNGQPENNYKTTPPVLDSDGSFFLYSKLTVDKSRW
QQGNVFSCSVMHEALHNHYTQKSLSLSPGK
173M36 VH2 humanized heavy chain variable domain with the signal sequence
(signal sequence underlined) (SEQ ID NO: 78):
MDWTWRILFLVAAATGAHSEVQLVQSGGGLVKPGGSLRLSCAASGFTFSTYGMSWVRQAP
GKGLEWVATISDGGSYTYYPDSVKGRFTISRDNAKNSLYLQMNSLRAEDTAVYYCARHYY
GSDWYFDVWGQGTTVTVSS
173M36 VH2 humanized heavy chain variable domain without signal sequence
(SEQ ID NO: 79):
EVQLVQSGGGLVKPGGSLRLSCAASGFTFSTYGMSWVRQAPGKDLEWVATISDGGSYTYY
PDSVKGRFTISRDNAKNSLYLQMNSLRAEDTAVYYCARHYYGSDWYFDVWGQGTTVTVSS
173M36 VH CDR1 (SEQ ID NO: 80):
STYGMS
173M36 VH CDR2 (SEQ ID NO: 81):
ATISDGGSYTYYPDSVKGR
173M36 VH CDR3 (SEQ ID NO: 82):
ARHYYGSDWYFDV
Human LIGHT with signal sequence (signal sequence underlined)
(SEQ ID NO: 83):
MEESVVRPSVFVVDGQTDIPFTRLGRSHRRQSCSVARVGLGLLLLLMGAGLAVQGWFLLQLHWRLGEMVT
RLPDGPAGSWEQLIQERRSHEVNPAAHLTGANSSLTGSGGPLLWETQLGLAFLRGLSYHDGALVVTKAGY
YYIYSKVQLGGVGCPLGLASTITHGLYKRTPRYPEELELLVSQQSPCGRATSSSRVWWDSSFLGGVVHLE
AGEKVVVRVLDERLVRLRDGTRSYFGAFMV
Human LIGHT extracellular domain (aa 59-240) (SEQ ID NO: 84):
LQLHWRLGEMVTRLPDGPAGSWEQLIQERRSHEVNPAAHLTGANSSLTGSGGPLLWETQLGLAFLRGLSY
HDGALVVTKAGYYYIYSKVQLGGVGCPLGLASTITHGLYKRTPRYPEELELLVSQQSPCGRATSSSRVWW
DSSFLGGVVHLEAGEKVVVRVLDERLVRLRDGTRSYFGAFMV
Human LIGHT fragment (aa 89-2401) (SEQ ID NO: 85):
SHEVNPAAHLTGANSSLTGSGGPLLWETQLGLAFLRGLSYHDGALVVTKAGYYYIYSKVQLGGVGCPLGL
ASTITHGLYKRTPRYPEELELLVSQQSPCGRATSSSRVWWDSSFLGGVVHLEAGEKVVVRVLDERLVRLR
DGTRSYFGARMV
Single gene human LIGHT homotrimer (SEQ ID NO: 86):
SHEVNPAAHLTGANSSLTGSGGPLLWETQLGLAFLRGLSYHDGALVVTKAGYYYIYSKVQLGGVGCPLGL
ASTITHGLYKRTPRYPEELELLVSQQSPCGRATSSSRVWWDSSFLGGVVHLEAGEKVVVRVLDERLVRLR
DGTRSYFGAFMVSHEVNPAAHLTGANSSLTGSGGPLLWETQLGLAFLRGLSYHDGALVVTKAGYYYIYSK
VQLGGVGCPLGLASTITHGLYKRTPRYPEELELLVSQQSPCGRATSSSRVWWDSSFLGGVVHLEAGEKVV
VRVLDERLVRLRDGTRSYFGAGMVSHEVNPAAHLTGANSSLTGSGGPLLWETQLGLAFLRGLSYHDGALV
VTKAGYYYIYSKVQLGGVGCPLGLASTITHGLYKRTPRYPEELELLVSQQSPCGRATSSSRVWWDSSFLG
GVVHLEAGEKVVVRVLDERLVRLRDGTRSYFGAFMV
Human B7-H4 with signal sequence (signal sequence underlined)
(SEQ ID NO: 87):
MASLGQILFWSIISIIIILAGAIALIIGFGISGRHSITVTTVASAGNIGEDGILSCTFEP
DIKLSDIVIQWLKEGVLGLVHEFKEGKDELSEQDEMFRGRTAVFADQVIVGNASLRLKNV
QLTDAGTYKCYIITSKGKGNANLEYKTGAFSMPEVNVDYNASSETLRCEAPRWFPQPTVV
WASQVDQGANFSEVSNTSFELNSENVTMKVVSVLYNVTINNTYSCMIENDIAKATGDIKV
TESEIKRRSHLQLLNSKASLCVSSFFAISWALLPLSPYLMLK
Human B7-H4 extracellular domain aa 1-257 including signal sequence)
(SEQ ID NO: 56):
MASLGQILFWSIISIIIILAGAIALIIGFGISGRHSITVTTVASAGNIGEDGILSCTFEP
DIKLSDIVIQWLKEGVLGLVHEFKEGKDELSEQDEMFRGRTAVFADQVIVGNASLRLKNV
QLTDAGTYKCYIITSKGKGNANLEYKTGAFSMPEVNVDYNASSETLRCEAPRWFPQPTVV
WASQVDQGANFSEVSNTSFELNSENVTMKVVSVLYNVTINNTYSCMIENDIAKATGDIKV
TESEIKRRSHLQLLNSK
Human B7-H4 extracellular domain without signal sequence (SEQ ID NO: 88):
GISGRHSITVTTVASAGNIGEDGILSCTFEPDIKLSDIVIQWLKEGVLGLVHEFKEGKDELSEQDEMFRG
RTAVFADQVIVGNASLRLKNVQLTDAGTYKCYIITSKGKGNANLEYKTGAFSMPEVNVDYNASSETLRCE
APRWFPQPTVVWASQVDQGANFSEVSNTSFELNSENVTMKVVSVLYNVTINNTYSCMIENDIAKATGDIK
VTESEIKRRSHLQLLNSK
Human CDH3 (signal sequence underlined) (SEQ ID NO: 89):
MGLPRGPLASLLLLQVCWLQCAASEPCRAVFREAEVTLEAGGAEQEPGQALGKVFMGCPG
QEPALFSTDNDDFTVRNGETVQERRSLKERNPLKIFPSKRILRRHKRDWVVAPISVPENG
KGPFPQRLNQLKSNKDRDTKIFYSITGPGADSPPEGVFAVEKETGWLLLNKPLDREEIAK
YELFGHAVSENGASVEDPMNISIIVTDQNDHKPKFTQDTFRGSVLEGVLPGTSVMQVTAT
DEDDAIYTYNGVVAYSIHSQEPKDPHDLMFTIHRSTGTISVISSGLDREKVPEYTLTIQA
TDMDGDGSTTTAVAVVEILDANDNAPMFDPQKYEAHVPENAVGHEVQRLTVTDLDAPNSP
AWRATYLIMGGDDGDHFTITTHPESNQGILTTRKGLDFEAKNQHTLYVEVTNEAPFVLKL
PTSTATIVVHVEDVNEAPVFVPPSKVVEVQEGIPTGEPVCVYTAEDPDKENQKISYRILR
DPAGWLAMDPDSGQVTAVGTLDREDEQFVRNNIYEVMVLAMDNGSPPTTGTGTLLLTLID
VNDHGPVPEPRQITICNQSPVRQVLNITDKDLSPHTSPFQAQLTDDSDIYWTAEVNEEGD
TVVLSLKKFLKQDTYDVHLSLSDNGNKEQLTVIRATVCDCHGHVETCPGPWKGGFILPVL
GAVLALLFLLLVLLLLVRKKRKIKEPLLLPEDDTRDNVFYYGEEGGGEEDQDYDITQLHR
GLEARPEVVLRNDVAPTIIPTPMYRPRPANPDEIGNFIIENLKAANTDPTAPPYDTLLVF
DYEGSGSDAASLSSLTSSASDQDQDYDYLNEWGSRFKKLADMYGGGEDD
Human CDH3 extracellular domain (signal sequence underlined)
(SEQ ID NO: 90):
MGLPRGPLASLLLLQVCWLQCAASEPCRAVFREAEVTLEAGGAEQEPGQALGKVFMGCPG
QEPALFSTDNDDFTVRNGETVQERRSLKERNPLKIFPSKRILRRHKRDWVVAPISVPENG
KGPFPQRLNQLKSNKDRDTKIFYSITGPGADSPPEGVFAVEKETGWLLLNKPLDREEIAK
YELFGHAVSENGASVEDPMNISIIVTDQNDHKPKFTQDTFRGSVLEGVLPGTSVMQVTAT
DEDDAIYTYNGVVAYSIHSQEPKDPHDLMFTIHRSTGTISVISSGLDREKVPEYTLTIQA
TDMDGDGSTTTAVAVVEILDANDNAPMFDPQKYEAHVPENAVGHEVQRLTVTDLDAPNSP
AWRATYLIMGGDDGDHFTITTHPESNQGILTTRKGLDFEAKNQHTLYVEVTNEAPFVLKL
PTSTATIVVHVEDVNEAPVFVPPSKVVEVQEGIPTGEPVCVYTAEDPDKENQKISYRILR
DPAGWLAMDPDSGQVTAVGTLDREDEQFVRNNIYEVMVLAMDNGSPPTTGTGTLLLTLID
VNDHGPVPEPRQITICNQSPVRQVLNITDKDLSPHTSPFQAQLTDDSDIYWTAEVNEEGD
TVVLSLKKFLKQDTYDVHLSLSDNGNKEQLTVIRATVCDCHGHVETCPGPWKG
Mature human CDH3 extracellular domain (SEQ ID NO: 91):
DWVVAPISVPENGKGPFPQRLNQLKSNKDRDTKIFYSITGPGADSPPEGVFAVEKETGWL
LLNKPLDREEIAKYELFGHAVSENGASVEDPMNISIIVTDQNDHKPKFTQDTFRGSVLEG
VLPGTSVMQVTATDEDDAIYTYNGVVAYSIHSQEPKDPHDLMFTIHRSTGTISVISSGLD
REKVPEYTLTIQATDMDGDGSTTTAVAVVEILDANDNAPMFDPQKYEAHVPENAVGHEVQ
RLTVTDLDAPNSPAWRATYLIMGGDDGDHFTITTHPESNQGILTTRKGLDFEAKNQHTLY
VEVTNEAPFVLKLPTSTATIVVHVEDVNEAPVFVPPSKVVEVQEGIPTGEPVCVYTAEDP
DKENQKISYRILRDPAGWLAMDPDSGQVTAVGTLDREDEQFVRNNIYEVMVLAMDNGSPP
TTGTGTLLLTLIDVNDHGPVPEPRQITICNQSPVRQVLNITDKDLSPHTSPFQAQLTDDS
DIYWTAEVNEEGDTVVLSLKKFLKQDTYDVHLSLSDHGNKEQLTVIRATVCDCHGHVETC
PGPWKG
363F1 mFc-mLTαββ (SEQ ID NO: 94):
GCKPCICTVPEVSSVFIFPPKPKDVLTITLTPKVTCVVVDISKDDPEVQFSWFVDDVEVH
TAQTQPREEQFASTFRSVSELPIMHQDWLNGKEFKCRVNSAAFPAPIEKTISKTKGRPKA
PQVYTIPPPKEQMAKDKVSLTCMITDFFPEDITVEWQWNGQPAENYKNTQPIMDTDGSYF
VYSKLNVQKSNWEAGNTFTCSVLHEGLHNHHTEKSLSHSPGKKPAAHLVGYPSKQNSLLW
RASTDRAFLRHGFSLSNNSLLIPTSGLYFVYSQVVFSGESCSPRAIPTPIYLAHEVQLFS
SQYPFHVPLLSAQKSVYPGLQGPWVRSMYQGAVFLLSKGDQLSTHTDGISHLHFSPSSVF
FGAFALLNPELPAAHLIGAWMSGQGLSWEASQEEAFLRSGAQFSPTHGLALPQDGVYYLY
CHVGYRGRTPPAGRSRARSLTLRSALYRAGGAYGRGSPELLLEGAETVTPVVDPIGYGSL
WYTSVGFGGLAQLRSGERVYVNISHPDMVDYRRGKTFFGAVMVGLNPELPAAHLIGAWMS
GQGLSWEASQEEAFLRSGAQFSPTHGLALPQDGVYYLYCHVGYRGRTPPAGRSPARSLTL
RSALYRAGGAYGRGSPELLLEGAETVTPVVDPIGYGSLWYTSVGFGGLAQLRSGERVYVN
ISHPDMVDYRRGKTFFGAVMVG
363F2 hFc-hLTαββ (SEQ ID NO: 95):
DKTHTCPPCPAPELLGGPSVFLFPPKPKDTLMISRTPEVTCVVVDVSHEDPEVKFNWYVD
GVEVHNAKTKPREEQYASTYRVVSVLTVLHQDWLNGKEYKCKVSNKALPAPIEKTISKAK
GQPREPQVYTLPPSREEMTKNQVSLTCLVKGFYPSDIAVEWESNGQPENNYKTTPPVLDS
DGSFFLYSKLTVDKSRWQQGNVFSCSVMHEALHNHYTQKSLSLSPGKKPAAHLIGDPSKQ
NSLLWRANTDRAFLQDGFSLSNNSLLVPTSGIYFVYSQVVFSGKAYSPKATSSPLYLAHE
VQLFSSQYPFHVPLLSSQKMVYPGLQEPWLHSMYHGAAFQLTQGDQLSTHTDGIPHLVLS
PSTVFFGAFALLSPGLPAAHLIGAPLSGQGLGWETTKEQAFLTSGTQFSDAEGLALPQDG
LYYLYCLVGYRGRAPPGGGDPQGRSVTLRSSLYRAGGAYGPGTPELLLEGAETVIPVLDP
ARRQGYGPLWYTSVGFGGLVQLRRGERVYVNISHPDMVDFARGKTFFGAVMVGLSPGLPA
AHLIGAPLKGQGLGWETTKEQAFLTSGTQFSDAEGLALPQDGLYYLYCLVGYRGRAPPGG
GDPQGRSVTLRSSLYRAGGAYGPGTPELLLEGAETVTPVLDPARRQGYGPLWYTSVGFGG
LVQLRRGERVYVNISHPDMVDFARGKTFFGAVMVG
363F3 hFc-hLTβαβ (SEQ ID NO: 96):
DKTHTCPPCPAPELLGGPSVFLFPPKPKDTLMISRTPEVTCVVVDVSHEDPEVKFNWYVD
GVEVHNAKTKPREEQYASTYRVVSVLTVLHQDWLNGKEYKCKVSNKALPAPIEKTISKAK
GQPREPQVYTLPPSREEMTKNQVSLTCLVKGFYPSDIAVEWESNGQPENNYKTTPPVLDS
DGSFFLYSKLTVDKSRWQQGNVFSCSVMHEALHNHYTQKSLSLSPGKLSPGLPAAHLIGA
PLKGQGLGWETTKEQAFLTSGTQFSDAEGLALPQDGLYYLYCLVGYRGRAPPGGGDPQGR
SVTLRSSLYRAGGAYGPGTPELLLEGAETVTPVLDPARRQGYGPLWYTSVGFGGLVQLRR
GERVYVNISHPDMVDFARGKTFFGAVMVGKPAAHLIGDPSKQNSLLWRANTDRAFLQDGF
SLSNNSLLVPTSGIYFVYSQVVFSGKAYSPKATSSPLYLAHEVQLFSSQYPFHVPLLSSQ
KMVYPGLQEPWLHSMYHGAAFQLTQGDQLSTHTDGIPHLVLSPSTVFFGAFALLSPGLPA
AHLIGAPLKGQGLGWETTKEQAFLTSGTQFSDAEGLALPQDGLYYLYCLVGYRGRAPPGG
GDPQGRSVTLRSSLYRAGGAYGPGTPELLLEGAETVTPVLDPARRQGYGPLWYTSVGFGG
LVQLRRGERVYVNISHPDMVDFARGKTFFGAVMVG
363F4 hFc-hLTββα (SEQ ID NO: 97):
DKTHTCPPCPAPELLGGPSVFLFPPKPKDTLMISRTPEVTCVVVDVSHEDPEVKFNWYVD
GVEVHNAKTKPREEQYASTYRVVSVLTVLHQDWLNGKEYKCKVSNKALPAPIEKTISKAK
GQPREPQVYTLPPSREEMTKNQVSLTCLVKGFYPSDIAVEWESNGQPENNYKTTPPVLDS
DGSFFLYSKLTVDKSRWQQGNVFSCSVMHEALHNHYTQKSLSLSPGKLSPGLPAAHLIGA
PLKGQGLGWETTKEQAFLTSGTQFSDAEGLALPQDGLYYLYCLVGYRGRAPPGGGDPQGR
SVTLRSSLYRAGGAYGPGTPELLLEGAETVTPVLDPARRQGYGPLWYTSVGFGGLVQLRR
GERVYVNISHPDMVDFARGKTFFGAVMVGLSPGLPAAHLIGAPLKGQGLGWETTKEQAFL
TSGTQFSDAEGLALPQDGLYYLYCLVGYRGRAPPGGGDPQGRSVTLRSSLYRAGGAYGPG
TPELLLEGAETVTPVLDPARRQGYGPLWYTSVGFGGLVQLRRGERVYVNISHPDMVDFAR
GKTFFGAVMVGKPAAHLIGDPSKQNSLLWRANTDRAFLQDGFSLSNNSLLVPTSGIYFVY
SQVVFSGKAYSPKATSSPLYLAHEVQLFSSQYPFHVPLLSSQKMVYPGLQEPWLHSMYHG
AAFQLTQGDQLSTHTDGIPHLVLSPSTVFFGAFAL
364B4 humanized anti-CDH3/hLTαββ heavy chain precursor protein
(signal sequence underlined) (SEQ ID NO: 98):
MDWTWRILFLVAAATGAHSEVQLVQSGGGLVKPGGSLRLSCAASGFTFSTYGMSWVRQAP
GKGLEWVATISDGGSYTYYPDSVKGRFTISRDNAKNSLYLQMNSLRAEDTAVYYCARHYY
GSDWYFDVWGQGTTVTVSSASTKGPSVFPLAPSSKSTSGGTAALGCLVKDYFPEPVTVSW
NSGALTSGVHTFPAVLQSSGLYSLSSVVTVPSSSLGTQTYICNVNHKPSNTKVDKRVEPK
SCDKTHTCPPCPAPELLGGPSVFLFPPKPKDTLIMSRTPEVTCVVVDVSHEDPEVKFNWY
VDGVEVHNAKTKPREEQYASTYRVVSVLTVLHQDWLNGKEYKCKVSNKALPAPIEKTISK
AKGQPREPQVYTLPPSREELTKNQVSLTCLVKGFYPSDIAVEWESNGQPENNYKTTPPVL
DSDGSFFLYSKLTVDKSRWQQGNVFSCSVMHEALHNHYTQKSLSLSPGKAHSTLKPAAHL
IGDPSKQNSLLWRANTDRAFLQDGFSLSNNSLLVPTSGIYFVYSQVVFSGKAYSPKATSS
PLYLAHEVQLFSSQYPFHVPLLSSQKMVYPGLQEPWLHSMYHGAAFQLTQGDQLSTHTDG
IPHLVLSPSTVFFGAFALLSPGLPAAHLIGAPLKGQGLGWETTKEQAFLTSGTQFSDAEG
LALPQDGLYYLYCLVGYRGRAPPGGGDPQGRSVTLRSSLYRAGGAYGPGTPELLLEGAET
VTPVLDPARRQGYGPLWYTSVGFGGLVQLRRGERVYVNISHPDMVDFARGKTFFGAVMVG
LSPGLPAAHLIGAPLKGQGLGWETTKEQAFLTSGTQFSDAEGLALPQDGLYYLYCLVGYR
GRAPPGGGDPQGRSVTLRSSLYRAGGAYGPGTPELLLEGAETVTPVLDPARRQGYGPLWY
TSVGFGGLVQLRRGERVYVNISHPDMVDFARGKTFFGAVMVG
364B4 humanized anti-CDH3/hLTαββ heavy chain mature protein
(SEQ ID NO: 99):
EVQLVQSGGGLVKPGGSLRLSCAASGFTFSTYGMSWVRQAPGKGLEWVATISDGGSYTYY
PDSVKGRFTISRDNAKNSLYLQMNSLRAEDTAVYYCARHYYGSDWYFDVWGQGTTVTVSS
ASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKRVEPKSCDKTHTCPPCPAPELLGG
PSVFLFPPKPKDTLMISRTPEVTCVVVDVSHEDPEVKFNWYVDGVEVHNAKTKPREEQYA
STYRVVSVLTVLHQDWLNGKEYKCKVSNKALPAPIEKTISKAKGQPREPQVYTLPPSREE
LTKNQVSLTCLVKGFYPSDIAVEWESNGQPENNYKTTPPVLDSDGSFFLYSKLTVDKSRW
QQGNVFSCSVMHEALHNHYTQKSLSLSPGKAHSTLKPAAHLIGDPSKQNSLLWRANTDRA
FLQDGFSLSNNSLLVPTSGIYFVYSQVVFSGKAYSPKATSSPLYLAHEVQLFSSQYPFHV
PLLSSQKMVYPGLQEPWLHSMYHGAAFQLTQGDQLSTHTDGIPHLVLSPSTVFFGAFALL
SPGLPAAHLIGAPLKGQGLGWETTKEQAFLTSGTQFSDAEGLALPQDGLYYLYCLVGYRG
RAPPGGGDPQGRSVTLRSSLYRAGGAYGPGTPELLLEGAETVTPVLDPARRQGYGPLWYT
SVGFGGLVQLRRGERVYVNISHPDMVDFARGKTFFGAVMVGLSPGLPAAHLIGAPLKGQG
LGWETTKEQAFLTSGTQFSDAEGLALPQDGLYYLYCLVGYRGRAPPGGGDPQGRSVTLRS
SLYRAGGAYGPGTPELLLEGAETVTPVLDPARRQGYGPLWYTSVGFGGLVQLRRGERVYV
NISHPDMVDFARGKTFFGAVMVG
364B4 humanized anti-CDH3/hLTαββ heavy chain precursor DNA
(SEQ ID NO: 100):
ATGGACTGGACCTGGAGGATACTCTTTCTCGTGGCTGCAGCCACAGGAGCCCACICCGAG
GTGCAGCTGGTGCAATCTGGGGGAGGACTGGTCAAGCCTGGGGGGTCCCTGAGACTCTCC
TGTGCAGCCTCTGGATTCACCTTCTCTACCTATGGCATGTCTTGGGTCCGCCAAGCTCCA
GGGAAGGGGCTGGAGTGGGTCGCAACCATTTCTGATGGTGGTAGCTACACCTACTATCCA
GACTCCGTGAAGGGGCGGTTCACCATCTCCAGAGACAACGCCAAGAACTCACTGTATCTG
CAAATGAACAGCCTGAGAGCCGAGGACACAGCTGTGTATTACTGTGCTAGACATTACTAC
GGTTCTGACTGGTACTTCGATGTCTGGGGGCAAGGGACCACCGTCACCGTCAGCTCAGCC
AGCACAAAGGGCCCCTCCGTGTTCCCTCTGGCCCCTTCCTCCAAGTCCACCTCCGGCGGC
ACCGCCGCTCTGGGCTGCCTGGTGAAGGACTACTTCCCTGAGCCTGTGACCGTGTCCTGG
AACTCTGGCGCTCTGACCTCTGGCGTCCACACCTTCCCAGCCGTGCTGCAGTCCTCCGGC
CTGTACTCCCTGTCCTCCGTGGTGACTGTGCCTTCCTCCTCCCTGGGCACCCAGACCTAC
ATCTGCAACGTGAACCACAAGCCTTCCAACACCAAGGTGGACAAGCGGGTGGAGCCTAAG
TCCTGCGACAAGACCCACACCTGCCCTCCCTGCCCTGCCCCTGAGCTGCTGGGCGGACCT
TCCGTGTTCCTGTTCCCTCCTAAGCCTAAGGACACCCTGATGATCTCCCGGACCCCTGAG
GTGACATGCGTGGTGGTGGACGTGTCCCACGAGGACCCTGAGGTGAAGTTCAACTGGTAT
GTGGACGGCGTGGAAGTGCATAACGCTAAGACCAAGCCAAGGGAGGAGCAGTACGCCTCC
ACCTACCGGGTGGTGTCTGTGCTGACCGTGCTGCACCAGGACTGGCTGAACGGCAAAGAA
TACAAGTGCAAGGTCTCCAACAAGGCCCTGCCCGCTCCCATCGAGAAAACCATCTCCAAG
GCCAAGGGCCAGCCTCGCGAGCCTCAGGTGTACACCCTGCCACCCAGCCGGGAGGAGCTG
ACCAAGAACCAGGTGTCCCTGACCTGTCTGGTGAAGGGCTTCTACCCTTCCGATATTGCC
GTGGAGTGGGAGTCTAACGGCCAGCCCGAGAACAACTACAAGACCACCCCTCCTGTGCTG
GACTCCGACGGCTCCTTCTTCCTGTACTCCAAGCTGACCGTGGACAAGTCCCGGTGGCAG
CAGGGCAACGTGTTCTCCTGCTCCGTGATGCACGAGGCCCTGCACAACCACTACACCCAG
AAGTCCCTGTCTCTGTCTCCTGGCAAGGCCCACAGCACCCTCAAACCTGCTGCTCACCTC
ATTGGCGACCCCAGCAAGCAAAACTCACTGCTCTGGCGGGCAAACACTGACCGGGCCTTC
CTCCAAGATGGTTTCTCCCTCTCTAACAATTCTCTCCTGGTCCCCACCTCTGGCATCTAC
TTCGTCTACTCCCAAGTGGTCTTCTCTGGGAAAGCCTACTCTCCCAAAGCCACCTCCTCC
CCACTCTACCTGGCCCATGAGGTCCAGCTCTTCTCCTCCCAATACCCCTTCCATGTGCCT
CTCCTCTCTTCCCAAAAAATGGTGTATCCAGGGCTGCAGGAACCCTGGCTGCACTCTATG
TACCACGGGGCTGCTTTCCAACTCACCCAGGGAGACCAGCTCTCCACCCACACTGATGGC
ATCCCCCACCTCGTCCTCTCTCCTTCTACTGTCTTCTTTGGAGCCTTCGCTCTGCTCTCA
CCCGGGCTCCCAGCTGCCCACCTCATCGGCGCTCCACTGAAAGGGCAGGGGCTCGGCTGG
GAGACTACTAAGGAACAGGCTTTTCTGACCAGCGGGACCCAGTTCTCCGACGCCGAGGGG
CTGGCCCTCCCCCAGGACGGCCTCTATTACCTCTACTGTCTCGTCGGCTACCGGGGCCGC
GCCCCCCCTGGCGGCGGGGACCCCCAGGGCCGCTCCGTCACCCTGCGCTCCTCTCTGTAC
CGGGCCGGGGGCGCATACGGCCCCGGCACTCCCGAGCTGCTGCTCGAAGGGGCCGAGACC
GTGACTCCAGTGCTGGACCCCGCCAGGAGACAAGGCTACGGGCCTCTCTGGTACACCAGC
GTGGGGTTCGGCGGCCTGGTGCAGCTCCGGAGGGGCGAGAGGGTGTACGTCAACATCTCC
CACCCCGATATGGTGGACTTCGCCAGAGGGAAGACCTTCTTTGGGGCCGTGATGGTCGGA
CTGTCTCCTGGCCTGCCTGCCGCACATCTGATTGGTGCCCCTCTCAAAGGACAAGGACTC
GGATGGGAAACAACAAAAGAACAAGCATTCCTCACATCCGGAACACAATTTTCTGATGCA
GAAGGGCTCGCACTGCCACAAGATGGGCTGTACTATCTGTATTGCCTGGTTGGGTATCGC
GGTCGCGCACCTCCCGGGGGGGGCGATCCTCAAGGGCGGTCAGTTACCCTCCGGAGCAGC
CTCTATCGGGCAGGCGGGGCTTATGGACCTGGAACCCCTGAACTCCTCCTGGAAGGGGCT
GAAACCGTCACCCCCGTCCTCGATCCCGCTCGGCGGCAAGGCTATGGCCCCCTGTGGTAT
ACCTCCGTCGGCTTTGGGGGGCTCGTCCAACTGCGCCGGGGGGAACGGGTCTATGTGAAT
ATTTCCCATCCTGACATGGTCGATTTTGCCCGGGGCAAAACATTTTTCGGCGCTGTCATG
GTCGGCTGA
364B4 humanized anti-CDH3/hLTαββ light chain precursor protein
(signal sequence underlined)(SEQ ID NO: 101):
MVLQTQVFISLLLWISGAYGDIVMTQSPLSLPVTPGEPASISCRSSQSIVQSNGNTYLEW
YLQKPGQSPQLLIYKVSNQFSGVPDRFSGSGSGTDFTLKISRVEAEDVGVYYCFQGSHVP
LTFGQGTKVEIKRTVAAPSVFIFPPSDEQLKSGTASVVCLLNNFYPREAKVQWKVDNALQ
SGNSQESVTEQDSKDSTYSLSSTLTLSKADYEKHKVYACEVTHQGLSSPVTKSFNRGEC
364B4 humanized anti-CDH3/hLTαββ light chain mature protein
(SEQ ID NO: 102):
DIVMTQSPLSLPVTPGEPASISCRSSQSIVQSNGNTYLEWYLQKPGQSPQLLIYKVSNQF
SGVPDRFSGSGSGTDFTLKISRVEAEDVGVYYCFQGSHVPLTFGQGTKVEIKRTVAAPSV
FIFPPSDEQLKSGTASVVCLLNNFYPREAKVQWKVDNALQSGNSQESVTEQDSKDSTYSL
SSTLTLSKADYEKHKVYACEVTHQGLSSPVTKSFNRGEC
364B4 humanized anti-CDH3/hLTαββ light chain precursor DNA
(SEQ ID NO: 103):
ATGGTGCTCCAGACCCAGGTCTTCATTTCCCTGCTGCTCTGGATCAGCGGAGCCTACGGG
GATATTGTGATGACTCAGTCTCCACTCTCCCTGCCCGTCACCCCTGGAGAGCCTGCCTCC
ATCTCCTGCCGGTCTTCACAATCCATTGTTCAATCTAATGGAAACACCTATCTCGAATGG
TATCTGCAGAAGCCAGGGCAGTCTCCACAGCTCCTGATCTATAAAGTTTCCAACCAATTT
TCTGGGGTCCCTGATCGGTTCTCTGGCTCTGGATCAGGCACAGATTTTACACTGAAAATC
AGCAGAGTGGAGGCTGAGGATGTTGGGGTTTATTACTGCTTTCAAGGTTCACATGTTCCT
CTCACATTCGGCCAAGGGACCAAGGTGGAAATCAAGCGTACGGTGGCTGCACCATCTGTC
TTCATCTTCCCTCCATCTGATGAGCAGCTCAAATCTGGAACTGCCTCTGTTGTGTGCCTG
CTGAATAACTTCTATCCCAGAGAGGCCAAAGTCCAGTGGAAGGTGGATAACGCCCTCCAA
TCCGGCAACTCCCAGGAATCTGTCACAGAGCAGGACAGCAAGGACAGCACCTACAGCCTC
AGCTCCACCCTGACACTGAGCAAAGCAGACTACGAGAAACACAAAGTCTATGCCTGCGAA
GTCACCCATCAGGGCCTGTCTTCCCCCGTCACAAAGAGCTTCAACAGGGGAGAGTGCTAA
349B4 humanized anti-B7-H4/ hLTαββ heavy chain precursor protein
(signal sequence underlined)(SEQ ID NO: 104):
MDWTWRILFLVAAATGAHSQVQLVQSGAEVKKPGASVKVSCKASGYTFTSYYMHWVRQAP
GQRLEWMGYVDPFNGGTSYNQKFKGRVTITVDTSSSTAYMELSSLRSEDTAVYYCAFIAG
FANWGQGTLVTVSSASTKGPSVFPLAPSSKSTSGGTAALGCLVKDYFPEPVTVSWNSGAL
TSGVHTFPAVLQSSGLYSLSSVVTVPSSSLGTQTYICNVNHKPSNTKVDKRVEPKSCDKT
HTCPPCPAPELLGGPSVFLFPPKPKDTLMISRTPEVTCVVVDVSHEDPEVKFNWYVDGVE
VHNAKTKPREEQYASTYRVVSVLTVLHQDWLNGKEYKCKVSNKALPAPIEKTISKAKGQP
REPQVYTLPPSREELTKNQVSLTCLVKGFYPSDIAVEWESNGQPENNYKTTPPVLDSDGS
FFLYSKLTVDKSRWQQGNVFSCSVMHEALHNHYTQKSLSLSPGKAHSTLKPAAHLIGDPS
KQNSLLWRANTDRAFLQDGFSLSNNSLLVPTSGIYFVYSQVVFSGKAYSPKATSSPLYLA
HEVQLFSSQYPFHVPLLSSQKMVYPGLQEPWLHSMYHGAAFQLTQGDQLSTHTDGIPHLV
LSPSTVFFGAFALLSPGLPAAHLIGAPLKGQGLGWETTKEQAFLTSGTQFSDAEGLALPQ
DGLYYLYCLVGYRGRAPPGGGDPQGRSVTLRSSLYRAGGAYGPGTPELLLEGAETVTPVL
DPARRQGYGPLWYTSVGFGGLVQLRRGERVYVNISHPDMVDFARGKTFFGAVMVGLSPGL
PAAHLIGAPLKGQGLGWETTKEQAFLTSGTQFSDAEGLALPQDGLYYLYCLVGYRGRAPP
GGGDPQGRSVTLRSSLYRAGGAYGPGTPELLLEGAETVTPVLDPARRQGYGPLWYTSVGF
GGLVQLRRGERVYVNISHPDMVDFARGKTFFGAVMVG
349B4 humanized anti-B7-H4/hLTαββ mature chain mature protein
(SEQ ID NO: 105):
QVQLVQSGAEVKKPGASVKVSCKASGYTFTSYYMHWVRQAPGQRLEWMGYVDPFNGGTSY
NQKFKGRVTITVDTSSSTAYMELSSLRSEDTAVYYCAFIAGFANWGQGTLVTVSSASTKG
PSVFPLAPSSKSTSGGTAALGCLVKDYFPEPVTVSWNSGALTSGVHTFPAVLQSSGLYSL
SSVVTVPSSSLGTQTYICNVNHKPSNTKVDKRVEPKSCDKTHTCPPCPAPELLGGPSVFL
FPPKPKDTLMISRTPEVTCVVVDVSHEDPEVKFNWYVDGVEVHNAKTKPREEQYASTYRV
VSVLTVLHQDWLNGKEYKCKVSNKALPAPIEKTISKAKGQPREPQVYTLPPSREELTKNQ
VSLTCLVKGFYPSDIAVEWESNGQPENNYKTTPPVLDSDGSFFLYSKLTVDKSRWQQGNV
FSCSVMHEALHNHYTQKSLSLSPGKAHSTLKPAAHLIGDPSKQNSLLWRANTDRAFLQDG
FSLSNNSLLVPTSGIYFVYSQVVFSGKAYSPKATSSPLYLAHEVQLFSSQYPFHVPLLSS
QKMVYPGLQEPWLHSMYHGAAFQLTQGDQLSTHTDGIPHLVLSPSTVFFGAFALLSPGLP
AAHLIGAPLKGQGLGWETTKEQAFLTSGTQFSDAEGLALPQDGLYYLYCLVGYRGRAPPG
GGDPQGRSVTLRSSLYRAGGAYGPGTPELLLEGAETVTPVLDPARRQGYGPLWYTSVGFG
GLVQLRRGERVYVNISHPDMVDFARGKTFFGAVMGLSPGLPAAHLIGAPLKGQGLGWET
TKEQAFLTSGTQFSDAEGLALPQDGLYYLYCLVGYRGRAPPGGGDPQGRSVTLRSSLYRA
GGAYGPGTPELLLEGAETVTPVLDPARRQGYGPLWYTSVGFGGLVQLRRGERVYVNISHP
DMVDFARGKTFFGAVMVG
349B4 humanized anti-B7-H4/ hLTαββ heavy chain precursor DNA
(SEQ ID NO: 57):
ATGGACTGGACCTGGCGGATTCTCTTTCTCGTGGCTGCTGCCACAGGCGCCCACTCTCAG
GTCCAGCTCGTGCAGTCTGGCGCTGAGGTGAAGAAGCCTGGGGCCTCCGTGAAGGTTTCC
TGCAAGGCTTCTGGATACACCTTCACTTCCTACTACATGCACTGGGTGCGCCAGGCCCCC
GGACAAAGGCTCGAATGGATGGGATATGTTGATCCTTTCAATGGCGGAACATCCTACAAC
CAGAAATTCAAGGGCAGAGTCACCATTACCGTTGACACATCCTCTAGCACAGCCTACATG
GAGCTCTCCAGCCTGCGGTCTGAAGACACTGCTGTGTATTACTGTGCCTTTATTGCTGGG
TTTGCTAACTGGGGCCAGGGCACCCTGGTCACCGTCAGCTCAGCCAGCACAAAGGGCCCC
TCCGTGTTCCCTCTGGCCCCTTCCTCCAAGTCCACCTCCGGCGGCACCGCCGCTCTGGGC
TGCCTGGTGAAGGACTACTTCCCTGAGCCTGTGACCGTGTCCTGGAACTCTGGCGCTCTG
ACCTCTGGCGTCCACACCTTCCCAGCCGTGCTGCAGTCCTCCGGCCTGTACTCCCTGTCC
TCCGTGGTGACTGTGCCTTCCTCCTCCCTGGGCACCCAGACCTACATCTGCAACGTGAAC
CACAAGCCTTCCAACACCAAGGTGGACAAGCGGGTGGAGCCTAAGTCCTGCGACAAGACC
CACACCTGCCCTCCCTGCCCTGCCCCTGAGCTGCTGGGCGGACCTTCCGTGTTCCTGTTC
CCTCCTAAGCCTAAGGACACCCTGATGATCTCCCGGACCCCTGAGGTGACATGCGTGGTG
GTGGACGTGTCCCACGAGGACCCTGAGGTGAAGTTCAACTGGTATGTGGACGGCGTGGAA
GTGCATAACGCTAAGACCAAGCCAAGGGAGGAGCAGTACGCCTCCACCTACCGGGTGGTG
TCTGTGCTGACCGTGCTGCACCAGGACTGGCTGAACGGCAAAGAATACAAGTGCAAGGTC
TCCAACAAGGCCCTGCCCGCTCCCATCGAGAAAACCATCTCCAAGGCCAAGGGCCAGCCT
CGCGAGCCTCAGGTGTACACCCTGCCACCCAGCCGGGAGGAGCTGACCAAGAACCAGGTG
TCCCTGACCTGTCTGGTGAAGGGCTTCTACCCTTCCGATATTGCCGTGGAGTGGGAGTCT
AACGGCCAGCCCGAGAACAACTACAAGACCACCCCTCCTGTGCTGGACTCCGACGGCTCC
TTCTTCCTGTACTCCAAGCTGACCGTGGACAAGTCCCGGTGGCAGCAGGGCAACGTGTTC
TCCTGCTCCGTGATGCACGAGGCCCTGCACAACCACTACACCCAGAAGTCCCTGTCTCTG
TCTCCTGGCAAGGCCCACAGCACCCTCAAACCTGCTGCTCACCTCATTGGCGACCCCAGC
AAGCAAAACTCACTGCTCTGGCGGGCAAACACTGACCGGGCCTTCCTCCAAGATGGTTTC
TCCCTCTCTAACAATTCTCTCCTGGTCCCCACCTCTGGCATCTACTTCGTCTACTCCCAA
GTGGTCTTCTCTGGGAAAGCCTACTCTCCCAAAGCCACCTCCTCCCCACTCTACCTGGCC
CATGAGGTCCAGCTCTTCTCCTCCCAATACCCCTTCCATGTGCCTCTCCTCTCTTCCCAA
AAAATGGTGTATCCAGGGCTGCAGGAACCCTGGCTGCACTCTATGTACCACGGGGCTGCT
TTCCAACTCACCCAGGGAGACCAGCTCTCCACCCACACTGATGGCATCCCCCACCTCGTC
CTCTCTCCTTCTACTGTCTTCTTTGGAGCCTTCGCTCTGCTCTCACCCGGGCTCCCAGCT
GCCCACCTCATCGGCGCTCCACTGAAAGGGCAGGGGCTCGGCTGGGAGACTACTAAGGAA
CAGGCTTTTCTGACCAGCGGGACCCAGTTCTCCGACGCCGAGGGGCTGGCCCTCCCCCAG
GACGGCCTCTATTACCTCTACTGTCTCGTCGGCTACCGGGGCCGCGCCCCCCCTGGCGGC
GGGGACCCCCAGGGCCGCTCCGTCACCCTGCGCTCCTCTCTGTACCGGGCCGGGGGCGCA
TACGGCCCCGGCACTCCCGAGCTGCTGCTCGAAGGGGCCGAGACCGTGACTCCAGTGCTG
GACCCCGCCAGGAGACAAGGCTACGGGCCTCTCTGGTACACCAGCGTGGGGTTCGGCGGC
CTGGTGCAGCTCCGGAGGGGCGAGAGGGTGTACGTCAACATCTCCCACCCCGATATGGTG
GACTTCGCCAGAGGGAAGACCTTCTTTGGGGCCGTGATGGTCGGACTGTCTCCTGGCCTG
CCTGCCGCACATCTGATTGGTGCCCCTCTCAAAGGACAAGGACTCGGATGGGAAACAACA
AAAGAACAAGCATTCCTCACATCCGGAACACAATTTTCTGATGCAGAAGGGCTCGCACTG
CCACAAGATGGGCTGTACTATCTGTATTGCCTGGTTGGGTATCGCGGTCGCGCACCTCCC
GGGGGGGGCGATCCTCAAGGGCGGTCAGTTACCCTCCGGAGCAGCCTCTATCGGGCAGGC
GGGGCTTATGGACCTGGAACCCCTGAACTCCTCCTGGAAGGGGCTGAAACCGTCACCCCC
GTCCTCGATCCCGCTCGGCGGCAAGGCTATGGCCCCCTGTGGTATACCTCCGTCGGCTTT
GGGGGGCTCGTCCAACTGCGCCGGGGGGAACGGGTCTATGTGAATATTTCCCATCCTGAC
ATGGTCGATTTTGCCCGGGGCAAAACATTTTTCGGCGCTGTCATGGTCGGCTGA
349B4 humanized anti-B7-H4/hLTαββ light chain precursor protein
(signal sequence underlined)(SEQ ID NO: 106):
MVLQTQVFISLLLWISGAYGDIQMTQSPSSLSASVGDRVTITCKASQDIKSYLSWYQQKP
GKAPKTLIYYATSLADGVPSRFSGSGSGTDFTLTISSLQPEDFATYYCLQHGESPYTFGG
GTKVEIKRTVAAPSVFIFPPSDEQLKSGTASVVCLLNNFYPREAKVQWKVDNALQSGNSQ
ESVTEQDSKDSTYSLSSTLTLSKADYEKHKVYACEVTHQGLSSPVTKSFNRGEC
349B4 humanized anti-B7-H4/hTLαββ light chain mature protein
(SEQ ID NO: 107):
DIQMTQSPSSLSASVGDRVTITCKASQDIKSYLSWYQQKPGKAPKTLIYYATSLADGVPS
RFSGSGSGTDFTLTISSLQPEDFATYYCLQHGESPYTFGGGTKVEIKRTVAAPSVFIFPP
SDEQLKSGTASVVCLLNNFYPREAKVQWKVDNALQSGNSQESVTEQDSKDSTYSLSSTLT
LSKADYEKHKVYACEVTHQGLSSPVTKSFNRGEC
349B4 humanized anti-B7-H4/hLTαββ light chain precursor DNA
(SEQ ID NO: 58):
ATGGTGCTCCAGACCCAGGTCTTCATTTCCCTGCTGCTCTGGATCAGCGGAGCCTACGGG
GACATCCAGATGACCCAGTCTCCATCCTCCCTGTCTGCATCTGTTGGAGACAGAGTCACC
ATCACTTGCAAGGCATCTCAGGACATTAAAAGCTATCTCAGCTGGTATCAGCAGAAACCA
GGGAAAGCCCCTAAGACCCTGATCTATTATGCAACAAGCCTCGCAGATGGGGTCCCATCA
AGGTTCTCTGGCTCTGGATCTGGGACAGATTTCACTCTCACCATCAGCTCTCTGCAACCT
GAAGATTTTGCAACTTACTACTGTCTGCAGCATGGCGAGAGCCCTTACACATTCGGCGGA
GGGACCAAGGTGGAGATCAAACGCACGGTGGCTGCACCATCTGTCTTCATCTTCCCTCCA
TCTGATGAGCAGCTCAAATCTGGAACTGCCTCTGTTGTGTGCCTGCTGAATAACTTCTAT
CCCAGAGAGGCCAAAGTCCAGTGGAAGGTGGATAACGCCCTCCAATCCGGCAACTCCCAG
GAATCTGTCACAGAGCAGGACAGCAAGGACAGCACCTACAGCCTCAGCTCCACCCTGACA
CTGAGCAAAGCAGACTACGAGAAACACAAAGTCTATGCCTGCGAAGTCACCCATCAGGGC
CTGTCTTCCCCCGTCACAAAGAGCTTCAACAGGGGAGAGTGCTAA
Minimal LTβ sequence (aa 87-243) (SEQ ID NO: 108):
LPAAHLIGAPLKGQGLGWETTKEQAFLTSGTQFSDAEGLALPQDGLYYLYCLVGYRGRAP
PGGGDPQGRSVTLRSSLYRAGGAYGPGTPELLLEGAETVTPVLDPARRQGYGPLWYTSVG
FGGLVQLRRGERVYVNISHPDMVDFARGKTFFGAVMV

1. A fusion polypeptide comprising:
(a) a first copy and a second copy of the extracellular domain of human lymphotoxin-beta (LTβ) or a fragment thereof, and
(b) a copy of human lymphotoxin-alpha (LTα) or a fragment thereof,
wherein the copies of lymphotoxin-beta and lymphotoxin-alpha are directly linked to each other and the fusion polypeptide is capable of binding the human lymphotoxin-beta receptor (LTβR).

2-3. (canceled)

4. A single-chain polypeptide comprising:
(a) a first amino acid sequence consisting of the extracellular domain of human lymphotoxin-beta, a variant thereof having at least 80% sequence identity to the extracellular domain of human lymphotoxin-beta, or a fragment thereof;
a second amino acid sequence consisting of the extracellular domain of human lymphotoxin-beta, a variant thereof having at least 80% sequence identity to the extracellular domain of human lymphotoxin-beta, or a fragment thereof; and
a third amino acid sequence consisting of human lymphotoxin-alpha, a variant thereof having at least 80% sequence identity to human lymphotoxin-alpha, or a fragment thereof; or
(b) a first amino acid sequence consisting of a sequence having at least about 90% sequence identity to SEQ ID NO: 15;
a second amino acid sequence consisting of a sequence having at least about 90% sequence identity to SEQ ID NO: 15; and
a third amino acid sequence consisting of a sequence having at least about 90% sequence identity to SEQ ID NO:12;
wherein the polypeptide is capable of binding the human lymphotoxin-beta receptor, and the amino acid sequences are directly linked through a peptide bond.

5-6. (canceled)

7. The polypeptide of claim 1, which is structured sequentially as:
(a) lymphotoxin-alpha-lymphotoxin-beta-lymphotoxin-beta;
(b) lymphotoxin-beta-lymphotoxin-alpha-lymphotoxin-beta; or
(c) lymphotoxin-beta-lymphotoxin-beta-lymphotoxin-alpha.

8-9. (canceled)

10. The polypeptide of claim 1, wherein the first copy and the second copy of human lymphotoxin-beta each comprise SEQ ID NO: 15, and the copy of human lymphotoxin-alpha comprises SEQ ID NO: 12.

11. The polypeptide of claim 1, which comprises SEQ ID NO: 16, SEQ ID NO:17, or SEQ ID NO:18.

12. A polypeptide which comprises an amino acid sequence having at least about 95% sequence identity to SEQ ID NO:16, SEQ ID NO:17, or SEQ ID NO:18, wherein the polypeptide is capable of binding the human lymphotoxin-beta receptor.

13-14. (canceled)

15. An agent comprising: (a) the polypeptide of claim 1; and (b) a targeting moiety linked to the polypeptide.

16. (canceled)

17. An agent comprising:
(a) a heterotrimer comprising:
(i) a first amino acid sequence comprising the extracellular domain of human lymphotoxin-beta, a variant thereof having at least 80% sequence identity to the extracellular domain of human lymphotoxin-beta, or a fragment thereof;
a second amino acid sequence comprising the extracellular domain of lymphotoxin-beta, a variant thereof having at least 80% sequence identity to the extracellular domain of human lymphotoxin-beta, or a fragment thereof; and
a third amino acid sequence comprising lymphotoxin-alpha, a variant thereof having at least 80% sequence identity to human lymphotoxin-alpha, or a fragment thereof; or
(ii) a first amino acid sequence comprising a sequence having at least about 90° % sequence identity to SEQ ID NO: 15 or SEQ ID NO: 108;
a second amino acid sequence comprising a sequence having at least about 90% sequence identity to SEQ ID NO:15 or SEQ ID NO: 108; and
a third amino acid sequence comprising a sequence having at least about 90% sequence identity to SEQ ID NO: 12,
wherein the heterotrimer is capable of binding the human lymphotoxin-beta receptor; and
(b) a targeting moiety linked to the heterotrimer.

18. The agent of claim 17, wherein
(i) the first amino acid sequence comprises the extracellular domain of human lymphotoxin-beta, or a fragment thereof;
(ii) the second amino acid sequence comprises the extracellular domain of lymphotoxin-beta, or a fragment thereof; and
(iii) the third amino acid sequence comprises lymphotoxin-alpha, or a fragment thereof.

19-20. (canceled)

21. The agent of claim 17, wherein the heterotrimer is a single-chain polypeptide.

22. The agent of claim 17, wherein
(a) the first amino acid sequence comprises SEQ ID NO: 15,
(b) the second amino acid sequence comprises SEQ ID NO: 15; and/or
(c) the third amino acid sequence comprises SEQ ID NO: 12.

23. The agent of claim 17, wherein the heterotrimer comprises a polypeptide having the amino acid sequence of SEQ ID NO:16, SEQ ID NO:17, or SEQ ID NO:18.

24. The agent of claim 17, wherein the heterotrimer comprises a polypeptide having at least about 95% sequence identity to SEQ ID NO: 16, SEQ ID NO: 17, or SEQ ID NO:18.

25. The agent of claim 15, wherein the targeting moiety is capable of binding a target cell.

26. (canceled)

27. The agent of claim 15, wherein the targeting moiety comprises a non-lymphotoxin polypeptide.

28-29. (canceled)

30. The agent of claim 27, wherein the N-terminal end of the polypeptide is linked to the C-terminal end of the non-lymphotoxin polypeptide.

31. The agent of claim 27, wherein the C-terminal end of the polypeptide is linked to the N-terminal end of the non-lymphotoxin polypeptide.

32. The agent of claim 27, wherein the non-lymphotoxin polypeptide comprises an immunoglobulin heavy chain.

33-34. (canceled)

35. The agent of claim 32, wherein the immunoglobulin heavy chain is associated with an immunoglobulin light chain, and the immunoglobulin heavy chain and the immunoglobulin light chain form an antigen-binding site.

36. (canceled)

37. The agent of claim 27, wherein the non-lymphotoxin polypeptide comprises a single-chain antibody or a Fab.

38. The agent of claim 35, wherein the antigen-binding site binds a tumor-associated antigen.

39. (canceled)

40. The agent of claim 38, wherein the tumor-associated antigen is selected from the group consisting of B7-H4, P-CADHERIN (CDH3), GABRP, ACPP, SLC45A3, STEAP1, STEAP2, GPA33, GUCY2C, GARP, B7-H3, PVRL4, mesothelin and CA9.

41. (canceled)

42. The agent of claim 40, wherein the tumor-associated antigen is B7-H4.

43. The agent of claim 42, wherein the antigen-binding site binds B7-H4 and comprises:
(a) a heavy chain CDR1 comprising TSYYMH (SEQ ID NO:42), a heavy chain CDR2 comprising YVDPFNGGTSYNQKFKG (SEQ ID NO:43), and a heavy chain CDR3 comprising FIAGFAN (SEQ ID NO:44) or IAGFAN (SEQ ID NO:45); and
a light chain CDR1 comprising KASQDIKSYLS (SEQ ID NO:46), a light chain CDR2 comprising YATSLAD (SEQ ID NO:47), and a light chain CDR3 comprising LQHGESPYT (SEQ ID NO:48) or LQHGESPY (SEQ ID NO:49);
(b) a heavy chain variable region comprising SEQ ID NO:50 and a light chain variable region comprising SEQ ID NO:51; or
(c) a heavy chain variable region comprising SEQ ID NO:66 and a light chain variable region comprising SEQ ID NO:62.

44. (canceled)

45. The agent of claim 40, wherein the tumor-associated antigen is P-CADHERIN (CDH3).

46. The agent of claim 45, wherein the antigen-binding site binds human P-CADHERIN and comprises:
(a) a heavy chain CDR1 comprising STYGMS (SEQ ID NO:80), a heavy chain CDR2 comprising ATISDGGSYTYYPDSVKGR (SEQ ID NO:81), and a heavy chain CDR3 comprising ARHYYGSDWYFDV (SEQ ID NO:82); and
a light chain CDR1 comprising RSSQSIVQSNGNTYLE (SEQ ID NO:73), a light chain CDR2 comprising KVSNQFS (SEQ ID NO:74), and a light chain CDR3 comprising QGSHVPL (SEQ ID NO:75); or
(b) a heavy chain variable region comprising SEQ ID NO:79 and a light chain variable region comprising SEQ ID NO:72 or SEQ ID NO:93.

47. (canceled)

48. The polypeptide of claim 1, which further comprises a non-lymphotoxin polypeptide, wherein the non-lymphotoxin polypeptide comprises a human Fc region.

49-50. (canceled)

51. The polypeptide of claim 48, wherein the N-terminal end of the copies of lymphotoxin-beta and lymphotoxin-alpha is linked to the C-terminal end of the non-lymphotoxin polypeptide.

52. The polypeptide of claim 48, wherein the C-terminal end of the copies of lymphotoxin-beta and lymphotoxin-alpha is linked to the N-terminal end of the non-lymphotoxin polypeptide.

53-58. (canceled)

59. A homodimeric molecule, wherein each monomer comprises the agent of claim 15.

60-112. (canceled)

113. The polypeptide of claim 1, which activates the human lymphotoxin-beta receptor and/or induces human lymphotoxin-beta receptor signaling.

114. (canceled)

115. An isolated antibody that specifically binds B7-H4, which comprises:
(a) a heavy chain CDR1 comprising TSYYMH (SEQ ID NO:42), a heavy chain CDR2 comprising YVDPFNGGTSYNQKFKG (SEQ ID NO:43), and a heavy chain CDR3 comprising FIAGFAN (SEQ ID NO:44) or IAGFAN (SEQ ID NO:45); and
(b) a light chain CDR1 comprising KASQDIKSYLS (SEQ ID NO:46), a light chain CDR2 comprising YATSLAD (SEQ ID NO:47), and a light chain CDR3 comprising LQHGESPYT (SEQ ID NO:48) or LQHGESPY (SEQ ID NO:49).

116-126. (canceled)

127. An isolated antibody that specifically binds the extracellular domain of human P-CADHERIN, which comprises:
(a) a heavy chain CDR1 comprising STYGMS (SEQ ID NO:80), a heavy chain CDR2 comprising ATISDGGSYTYYPDSVKGR (SEQ ID NO:81), and a heavy chain CDR3 comprising ARHYYGSDWYFDV (SEQ ID NO:82); and
(b) a light chain CDR1 comprising RSSQSIVQSNGNTYLE (SEQ ID NO:73), a light chain CDR2 comprising KVSNQFS (SEQ ID NO:74), and a light chain CDR3 comprising QGSHVPL (SEQ ID NO:75).

128-138. (canceled)

139. The antibody of claim 115, which is linked to an LTβR-binding moiety.

140-152. (canceled)

153. An agent comprising:
(a) an antibody that specifically binds human B7-H4 or human P-CADHERIN; and
(b) an LTβR-binding moiety, wherein the LTβR-binding moiety is linked to the antibody.

154-160. (canceled)

161. The agent of claim 153, wherein the LTβR-binding moiety comprises:
(a) a LIGHT homotrimer;
(b) a lymphotoxin αββ heterotrimer; or
(c) an antibody that specifically binds LTβR.

162. (canceled)

163. The agent of claim 161, wherein the LTβR-binding moiety comprises SEQ ID NO:86.

164-168. (canceled)

169. An agent comprising
(a) an antibody that specifically binds a cell-surface antigen; and
(b) an LTβR-binding moiety comprising a single-chain lymphotoxin αββ heterotrimer, wherein the LTβR-binding moiety is linked to the antibody.

170. The agent of claim 169, wherein the cell-surface antigen is a tumor-associated antigen.

171-173. (canceled)

174. A polypeptide comprising (a) a polypeptide having the amino acid sequence selected from the group consisting of SEQ ID NOs: 16-18, SEQ ID NO:86, SEQ ID NOs:95-97, SEQ ID NO:99, SEQ ID NO:102, SEQ ID NO:105, and SEQ ID NO:107; (b) SEQ ID NO:105 and SEQ ID NO:107; or (c) SEQ ID NO:99 and SEQ ID NO:102.

175-176. (canceled)

177. A polynucleotide encoding the polypeptide of claim 174.

178-180. (canceled)

181. A pharmaceutical composition comprising the polypeptide of claim 1, which further comprises a pharmaceutically acceptable carrier.

182. (canceled)

183. A method of activating or enhancing LTβR signaling in a cell, comprising contacting the cell with an effective amount of the polypeptide of claim 1.

184. A method of inducing, activating, promoting, increasing, enhancing, or prolonging an immune response in a subject, comprising administering a therapeutically effective amount of the polypeptide of claim 1.

185. The method of claim 184, wherein the immune response is against a tumor or cancer.

186-197. (canceled)

198. A method of treating cancer or inhibiting the growth of a tumor in a subject, comprising administering to the subject a therapeutically effective amount of the polypeptide of claim 1.

199-201. (canceled)

202. The method of claim 198, which further comprises administering a second immunotherapeutic agent to the subject, wherein the subject has previously failed therapy with a checkpoint inhibitor and the second therapeutic agent is a checkpoint inhibitor.

203-205. (canceled)

206. The method of claim 202, wherein the checkpoint inhibitor is an anti-PD1 antibody, anti-PDL1 antibody or anti-TIGIT antibody.

207-210. (canceled)
